SEQUENCE LISTING (250 sequences)

SEQ ID NO: 1 - protein, 122 aa, Zika virus
  REGION 1..122: note = protein
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMA

SEQ ID NO: 2 - protein, 104 aa, Zika virus
  REGION 1..104: note = PROTEIN
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRR

SEQ ID NO: 3 - protein, 75 aa, Zika virus
AVTLPSHSTRKLQTRSQTWLESREYTKHLIRVENWIFRNPGFALAAAAIAWLLGSSTSQKVIYLVMILLIAPAYS

SEQ ID NO: 4 - protein, 75 aa, Zika virus; identical to SEQ ID NO: 3

SEQ ID NO: 5 - protein, 504 aa, Zika virus
IRCIGVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 6 - DNA, 106 bp, Zika virus
  source 1..106: mol_type = other DNA
agttgttgatctgtgtgaatcagactgcgacagttcgagtttgaagcgaaagctagcaaagtatcaacaggttttattttggatttggaaacgagagtttctggtc

SEQ ID NO: 7 - DNA, 429 bp, Zika virus
  source 1..429: mol_type = other DNA
gcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccatgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtctt

SEQ ID NO: 8 - RNA, 21 nt, Homo sapiens
  source 1..21: mol_type = other RNA
ctttttgcggtctgggcttgc

SEQ ID NO: 9 - RNA, 21 nt, Homo sapiens
  source 1..21: mol_type = other RNA
tgattgtccaaacgcaattct

SEQ ID NO: 10 - RNA, 22 nt, Homo sapiens
  misc_feature 1..22
  source 1..22: mol_type = other RNA
ctgggacaggaggaggaggcag

SEQ ID NO: 11 - RNA, 22 nt, Homo sapiens
  source 1..22: mol_type = other RNA
gtgggtacggcccagtgggggg

SEQ ID NO: 12 - RNA, 22 nt, Homo sapiens
  source 1..22: mol_type = other RNA
atcacacaaaggcaacttttgt

SEQ ID NO: 13 - RNA, 23 nt, Homo sapiens
  misc_feature 1..23
  source 1..23: mol_type = other RNA
tagaattgtggctggacatctgt

SEQ ID NO: 14 - DNA, 37 bp, Zika virus
  source 1..37: mol_type = other DNA
ctgatgagtccgtgaggacgaaacgagtaagctcgtc

SEQ ID NO: 15 - DNA, 68 bp, Zika virus
  source 1..68: mol_type = other DNA
ggccggcatggtcccagcctcctcgctggcgccggctgggcaacatgcttcggcatggcgaatgggac

SEQ ID NO: 16 - DNA, 19 bp, Zika virus
  source 1..19: mol_type = other DNA
taatacgactcactatagg

SEQ ID NO: 17 - DNA, 3889 bp, synthetic construct
  source 1..3889: mol_type = other DNA
taatacgactcactatagggcgcatgctatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggagaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatattatctccgatgaagcctccgttatcccaggttccttgggtttgttgccatctgcgtccttggcctctttgccagacaagaacaccgcatttggtttgtacgaaccatgtcacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaaagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcccctcagcctatgcggatccgatcggaccgctcgagaccggtatcgatgttaacacaactctgatgagtccgtgaggacgaaacgagtaagctcgtcagttgttactgttgctgactcagactgcgacagttcgagtttgaagcgaaagctagcaacagtatcaacaggttttatttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttga

SEQ ID NO: 18 - DNA, 3889 bp, synthetic construct
  source 1..3889: mol_type = other DNA
taatacgactcactatagggtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcccctcagcctatgcggatccgatcggaccgctcgagaccggtatcgatgttaacacaactctgatgagtccgtgaggacgaaacgagtaagctcgtcagttgttactgttgctgactcagactgcgacagttcgagtttgaagcgaaagctagcaacagtatcaacaggttttatttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacac

SEQ ID NO: 19 - DNA, 3889 bp, synthetic construct; identical to SEQ ID NO: 18
  misc_feature 1..3889: note = Synthetic Oligonucleotide
  source 1..3889: mol_type = other DNA

SEQ ID NO: 20 - DNA, 3889 bp, synthetic construct
  misc_feature 1..3889: note = Synthetic Oligonucleotide
  source 1..3889: mol_type = other DNA
actgattaatacgactcactataggctcgagaccggtatcgatgttaacacaactctgatgagtccgtgaggacgaaacgagtaagctcgtcagttgttactgttgctgactcagactgcgacagttcgagtttgaagcgaaagctagcaacagtatcaacaggttttatttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaag

SEQ ID NO: 21 - DNA, 3889 bp, synthetic construct; identical to SEQ ID NO: 20
  misc_feature 1..3889: note = Synthetic Oligonucleotide
  source 1..3889: mol_type = other DNA

SEQ ID NO: 22 - DNA, 3889 bp, synthetic construct; identical to SEQ ID NO: 20
  misc_feature 1..3889: note = Synthetic Oligonucleotide
  source 1..3889: mol_type = other DNA

SEQ ID NO: 23 - DNA, 3889 bp, synthetic construct; identical to SEQ ID NO: 18
  misc_feature 1..3889: note = Synthetic Oligonucleotide
  source 1..3889: mol_type = other DNA

SEQ ID NO: 24 - DNA, 3889 bp, synthetic construct; identical to SEQ ID NO: 18
  misc_feature 1..3889: note = Synthetic Oligonucleotide
  source 1..3889: mol_type = other DNA

SEQ ID NO: 25 - DNA, 3889 bp, synthetic construct
  misc_feature 1..3889: note = Synthetic Oligonucleotide
  source 1..3889: mol_type = other DNA
taatacgactcactatagggtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcccctcagcctatgcggatccgatcggaccgctcgagaccggtatcgatgttaacacaactctgatgagtccgtgaggacgaaacgagtaagctcgtcagttgttactgttgctgactcagactgcgacagttcgagtttgaagcgaaagctagcaacagtatcaacaggttttatttggatttggaaacgagagtttctggtcgtacgacgccggtcgtacgatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaa

SEQ ID NO: 26 - DNA, 51 bp, synthetic construct
  misc_feature 1..51: note = Synthetic Oligonucleotide
  source 1..51: mol_type = other DNA
tctccccaggacatgcatccgacctgtatgcataaccaaatggcaatcatc

SEQ ID NO: 27 - DNA, 51 bp, synthetic construct
  misc_feature 1..51: note = Synthetic Oligonucleotide
  source 1..51: mol_type = other DNA
gatgattgccatttggttatgcatacaggtcggatgcatgtcctggggaga

SEQ ID NO: 28 - DNA, 15 bp, Zika virus
  misc_feature 1..15
  source 1..15: mol_type = other DNA
acacctggagtgctg

SEQ ID NO: 29 - DNA, 15 bp, Zika virus
  misc_feature 1..15
  source 1..15: mol_type = other DNA
cagcactccaggtgt

SEQ ID NO: 30 - DNA, 3889 bp, synthetic construct; identical to SEQ ID NO: 18
  misc_feature 1..3889: note = Synthetic Oligonucleotide
  source 1..3889: mol_type = other DNA

SEQ ID NO: 31 - DNA, 31 bp, synthetic construct
  misc_feature 1..31: note = Synthetic Oligonucleotide
  source 1..31: mol_type = other DNA
ggacatgcatcttaacaccatgcataaccaa

SEQ ID NO: 32 - DNA, 31 bp, synthetic construct
  misc_feature 1..31: note = Synthetic Oligonucleotide
  source 1..31: mol_type = other DNA
ggacatgcatccgacctgtatgcataaccaa

SEQ ID NO: 33 - DNA, 31 bp, synthetic construct
  misc_feature 1..31: note = Synthetic Oligonucleotide
  source 1..31: mol_type = other DNA
tggtcgtacgcttaacacccgtacgatgaaa

SEQ ID NO: 34 - DNA, 30 bp, synthetic construct
  misc_feature 1..30: note = Synthetic Oligonucleotide
  source 1..30: mol_type = other DNA
tgtaacgcgtttaacaccgacgcgtgcacc

SEQ ID NO: 35 - DNA, 3889 bp, synthetic construct; identical to SEQ ID NO: 18
  misc_feature 1..3889: note = Synthetic Oligonucleotide
  source 1..3889: mol_type = other DNA

SEQ ID NO: 36 - DNA, 31 bp, synthetic construct
  misc_feature 1..31: note = Synthetic Oligonucleotide
  source 1..31: mol_type = other DNA
ggacatgcatctaacaggtatgcataaccaa

SEQ ID NO: 37 - DNA, 31 bp, synthetic construct
  misc_feature 1..31: note = Synthetic Oligonucleotide
  source 1..31: mol_type = other DNA
ggacatgcattaacaggttatgcataaccaa

SEQ ID NO: 38 - DNA, 31 bp, synthetic construct
  misc_feature 1..31: note = Synthetic Oligonucleotide
  source 1..31: mol_type = other DNA
tggtcgtacgactaacaggcgtacgatgaaa

SEQ ID NO: 39 - DNA, 30 bp, synthetic construct
  misc_feature 1..30: note = Synthetic Oligonucleotide
  source 1..30: mol_type = other DNA
tgtaacgcgtctaacaggtacgcgtgcacc

SEQ ID NO: 40 - DNA, 31 bp, synthetic construct
  misc_feature 1..31: note = Synthetic Oligonucleotide
  source 1..31: mol_type = other DNA
ggacatgcataaaaacgccatgcataaccaa

SEQ ID NO: 41 - DNA, 31 bp, synthetic construct
  misc_feature 1..31: note = Synthetic Oligonucleotide
  source 1..31: mol_type = other DNA
ggacatgcataaacgccagatgcataaccaa

SEQ ID NO: 42 - DNA, 31 bp, synthetic construct
  misc_feature 1..31: note = Synthetic Oligonucleotide
  source 1..31: mol_type = other DNA
tggtcgtacgaaaaacgcccgtacgatgaaa

SEQ ID NO: 43 - DNA, 30 bp, synthetic construct
  misc_feature 1..30: note = Synthetic Oligonucleotide
  source 1..30: mol_type = other DNA
tgtaacgcgtaaaaacgccacgcgtgcacc

SEQ ID NO: 44 - DNA, 3889 bp, synthetic construct; identical to SEQ ID NO: 18
  misc_feature 1..3889: note = Synthetic Oligonucleotide
  source 1..3889: mol_type = other DNA

SEQ ID NO: 45 - DNA, 31 bp, synthetic construct
  misc_feature 1..31: note = Synthetic Oligonucleotide
  source 1..31: mol_type = other DNA
ggacatgcatcacccatgcatgcataaccaa

SEQ ID NO: 46 - DNA, 31 bp, synthetic construct
  misc_feature 1..31: note = Synthetic Oligonucleotide
  source 1..31: mol_type = other DNA
ggacatgcatacccatgccatgcataaccaa

SEQ ID NO: 47 - DNA, 31 bp, synthetic construct
  misc_feature 1..31: note = Synthetic Oligonucleotide
  source 1..31: mol_type = other DNA
tggtcgtacgcccatgccgcgtacgatgaaa

SEQ ID NO: 48 - DNA, 30 bp, synthetic construct
  misc_feature 1..30: note = Synthetic Oligonucleotide
  source 1..30: mol_type = other DNA
tgtaacgcgtacccatgccacgcgtgcacc

SEQ ID NO: 49 - DNA, 31 bp, synthetic construct
  misc_feature 1..31: note = Synthetic Oligonucleotide
  source 1..31: mol_type = other DNA
ggacatgcatagtgtgtttatgcataaccaa

SEQ ID NO: 50 - DNA, 31 bp, synthetic construct
  misc_feature 1..31: note = Synthetic Oligonucleotide
  source 1..31: mol_type = other DNA
tggtcgtacgagtgtgtttcgtacgatgaaa

SEQ ID NO: 51 - DNA, 30 bp, synthetic construct
  misc_feature 1..30: note = Synthetic Oligonucleotide
  source 1..30: mol_type = other DNA
tgtaacgcgtagtgtgtttacgcgtgcacc

SEQ ID NO: 52 - DNA, 240 bp, synthetic construct
  misc_feature 1..240: note = Synthetic Oligonucleotide
  source 1..240: mol_type = other DNA
attgcatgtgtcctcattgttgtgttcctattgctggtggtgctcatacctgagccagaaaagcaaagatctccccaggacatgcatgcaggtggtcacctgcatgcataaccaaatggcaatcatcatcatggtagcagtaggtcttctgggcttgattaccgccaatgaactcggatggttggagagaacaaagagtgacctaagccatctaatgggaaggagagaggagggggcaac

SEQ ID NO: 53 - DNA, 10 bp, synthetic construct
  misc_feature 1..10: note = Synthetic Oligonucleotide
  source 1..10: mol_type = other DNA
aggcgaacca

SEQ ID NO: 54 - DNA, 10 bp, synthetic construct
  misc_feature 1..10: note = Synthetic Oligonucleotide
  source 1..10: mol_type = other DNA
tggcggtcct

SEQ ID NO: 55 - RNA, 22 nt, Homo sapiens
  misc_feature 1..22
  source 1..22: mol_type = other RNA
tgaggtagtaggttgtatggtt

SEQ ID NO: 56 - RNA, 22 nt, Homo sapiens
  misc_feature 1..22
  source 1..22: mol_type = other RNA
agaggtagtaggttgcatagtt

SEQ ID NO: 57 - RNA, 22 nt, Homo sapiens
  source 1..22: mol_type = other RNA
tgaggtaggaggttgtatagtt

SEQ ID NO: 58 - RNA, 22 nt, Homo sapiens
  misc_feature 1..22
  source 1..22: mol_type = other RNA
tgaggtagtagtttgtacagtt

SEQ ID NO: 59 - RNA, 22 nt, Homo sapiens
  source 1..22: mol_type = other RNA
aacccgtagatccgaacttgtg

SEQ ID NO: 60 - RNA, 21 nt, Homo sapiens
  source 1..21: mol_type = other RNA
tacagtactgtgataactgaa

SEQ ID NO: 61 - RNA, 23 nt, Homo sapiens
  source 1..23: mol_type = other RNA
tcaaatgctcagactcctgtggt

SEQ ID NO: 62 - RNA, 23 nt, Homo sapiens
  source 1..23: mol_type = other RNA
agcagcattgtacagggctatca

SEQ ID NO: 63 - RNA, 23 nt, Homo sapiens
  source 1..23: mol_type = other RNA
taccctgtagaaccgaatttgtg

SEQ ID NO: 64 - RNA, 21 nt, Homo sapiens
  source 1..21: mol_type = other RNA
gtgccagctgcagtgggggag

SEQ ID NO: 65 - RNA, 22 nt, Homo sapiens
  source 1..22: mol_type = other RNA
tggagtgtgacaatggtgtttg

SEQ ID NO: 66 - RNA, 22 nt, Homo sapiens
  source 1..22: mol_type = other RNA
taaggcacgcggtgaatgccaa

SEQ ID NO: 67 - RNA, 21 nt, Homo sapiens
  source 1..21: mol_type = other RNA
acggtgctggatgtggccttt

SEQ ID NO: 68 - RNA, 22 nt, Homo sapiens
  source 1..22: mol_type = other RNA
tcacaagtcaggctcttgggac

SEQ ID NO: 69 - RNA, 22 nt, Homo sapiens
  source 1..22: mol_type = other RNA
tccctgagaccctaacttgtga

SEQ ID NO: 70 - RNA, 22 nt, Homo sapiens
  source 1..22: mol_type = other RNA
caggatgtggtcaagtgttgtt

SEQ ID NO: 71 - RNA, 21 nt, Homo sapiens
  source 1..21: mol_type = other RNA
cattattacttttggtacgcg

SEQ ID NO: 72 - RNA, 21 nt, Homo sapiens
  source 1..21: mol_type = other RNA
tcacagtgaaccggtctcttt

SEQ ID NO: 73 - RNA, 22 nt, Homo sapiens
  source 1..22: mol_type = other RNA
aagcccttaccccaaaaagtat

SEQ ID NO: 74 - RNA, 22 nt, Homo sapiens
  source 1..22: mol_type = other RNA
aagcccttaccccaaaaagcat

SEQ ID NO: 75 - RNA, 21 nt, Homo sapiens
  source 1..21: mol_type = other RNA
ctttttgcggtctgggcttgc

SEQ ID NO: 76 - RNA, 22 nt, Homo sapiens
  source 1..22: mol_type = other RNA
taacagtctacagccatggtcg

SEQ ID NO: 77 - RNA, 23 nt, Homo sapiens
  source 1..23: mol_type = other RNA
tatggctttttattcctatgtga

SEQ ID NO: 78 - RNA, 23 nt, Homo sapiens
  source 1..23: mol_type = other RNA
ttattgcttaagaatacgcgtag

SEQ ID NO: 79 - RNA, 23 nt, Homo sapiens
  source 1..23: mol_type = other RNA
acgggtattcttgggtggataat

SEQ ID NO: 80 - RNA, 23 nt, Homo sapiens
  source 1..23: mol_type = other RNA
agctggtgttgtgaatcaggccg

SEQ ID NO: 81 - RNA, 23 nt, Homo sapiens
  source 1..23: mol_type = other RNA
tggagacgcggccctgttggagt

SEQ ID NO: 82 - RNA, 23 nt, Homo sapiens
  source 1..23: mol_type = other RNA
tctacagtgcacgtgtctccagt

SEQ ID NO: 83 - RNA, 22 nt, Homo sapiens
  source 1..22: mol_type = other RNA
tggaatgtaaagaagtatgtat

SEQ ID NO: 84 - RNA, 22 nt, Homo sapiens
  source 1..22: mol_type = other RNA
cagtggttttaccctatggtag

SEQ ID NO: 85 - RNA, 21 nt, Homo sapiens
  source 1..21: mol_type = other RNA
tgagatgaagcactgtagctc

SEQ ID NO: 86 - RNA, 23 nt, Homo sapiens
  source 1..23: mol_type = other RNA
gtccagttttcccaggaatccct

SEQ ID NO: 87 - RNA, 22 nt, Homo sapiens
  source 1..22: mol_type = other RNA
tcagtgcactacagaactttgt

SEQ ID NO: 88 - RNA, 22 nt, Homo sapiens
  source 1..22: mol_type = other RNA
tcagtgcatcacagaactttgt

SEQ ID NO: 89 - RNA, 23 nt, Homo sapiens
  source 1..23: mol_type = other RNA
tctggctccgtgtcttcactccc

SEQ ID NO: 90 - RNA, 22 nt, Homo sapiens
  source 1..22: mol_type = other RNA
ctggtacaggcctgggggacag

SEQ ID NO: 91 - RNA, 22 nt, Homo sapiens
  source 1..22: mol_type = other RNA
tctcccaacccttgtaccagtg

SEQ ID NO: 92 - RNA, 21 nt, Homo sapiens
  source 1..21: mol_type = other RNA
tcgaggagctcacagtctagt

SEQ ID NO: 93 - RNA, 22 nt, Homo sapiens
  source 1..22: mol_type = other RNA
ttgcatagtcacaaaagtgatc

SEQ ID NO: 94 - RNA, 22 nt, Homo sapiens
  source 1..22: mol_type = other RNA
taggttatccgtgttgccttcg

SEQ ID NO: 95 - RNA, 23 nt, Homo sapiens
  source 1..23: mol_type = other RNA
aacattcaacgctgtcggtgagt

SEQ ID NO: 96 - RNA, 23 nt, Homo sapiens
  source 1..23: mol_type = other RNA
aacattcattgctgtcggtgggt

SEQ ID NO: 97 - RNA, 22 nt, Homo sapiens
  source 1..22: mol_type = other RNA
aacattcaacctgtcggtgagt

SEQ ID NO: 98 - RNA, 22 nt, Homo sapiens
  source 1..22: mol_type = other RNA
tggacggagaactgataagggt

SEQ ID NO: 99 - RNA, 22 nt, Homo sapiens
  source 1..22: mol_type = other RNA
tggagagaaaggcagttcctga

SEQ ID NO: 100 - RNA, 22 nt, Homo sapiens
  source 1..22: mol_type = other RNA
caaagaattctccttttgggct

SEQ ID NO: 101 - RNA, 22 nt, Homo sapiens
  source 1..22: mol_type = other RNA
tcgtgtcttgtgttgcagccgg

SEQ ID NO: 102 - RNA, 21 nt, Homo sapiens
  source 1..21: mol_type = other RNA
catcccttgcatggtggaggg

SEQ ID NO: 103 - RNA, 23 nt, Homo sapiens
  source 1..23: mol_type = other RNA
taaggtgcatctagtgcagatag

SEQ ID NO: 104 - RNA, 23 nt, Homo sapiens
  source 1..23: mol_type = other RNA
taaggtgcatctagtgcagttag

SEQ ID NO: 105 - RNA, 22 nt, Homo sapiens
  source 1..22: mol_type = other RNA
tgatatgtttgatatattaggt

SEQ ID NO: 106 - RNA, 22 nt, Homo sapiens
  source 1..22: mol_type = other RNA
ggaggggtcccgcactgggagg

SEQ ID NO: 107 - RNA, 23 nt, Homo sapiens
  source 1..23: mol_type = other RNA
caacggaatcccaaaagcagctg

SEQ ID NO: 108 - RNA, 22 nt, Homo sapiens
  misc_feature 1..22
  source 1..22: mol_type = other RNA
tgtaacagcaactccatgtgga

SEQ ID NO: 109 - RNA, 22 nt, Homo sapiens
  source 1..22: mol_type = other RNA
ttcaccaccttctccacccagc

SEQ ID NO: 110 - RNA, 23 nt, Homo sapiens
  source 1..23: mol_type = other RNA
cgggtagagagggcagtgggagg

SEQ ID NO: 111 - RNA, 22 nt, Homo sapiens
  source 1..22: mol_type = other RNA
taacactgtctggtaacgatgt

SEQ ID NO: 112 - RNA, 22 nt, Homo sapiens
  misc_feature 1..22
  source 1..22: mol_type = other RNA
taatactgcctggtaatgatga

SEQ ID NO: 113 - RNA, 20 nt, Homo sapiens
  source 1..20: mol_type = other RNA
agaggtatagggcatgggaa

SEQ ID NO: 114 - RNA, 22 nt, Homo sapiens
  source 1..22: mol_type = other RNA
gtgaaatgtttaggaccactag

SEQ ID NO: 115 - RNA, 22 nt, Homo sapiens
  source 1..22: mol_type = other RNA
tccttcattccaccggagtctg

SEQ ID NO: 116 - RNA, 22 nt, Homo sapiens
  source 1..22: mol_type = other RNA
tggaatgtaaggaagtgtgtgg

SEQ ID NO: 117 - RNA, 23 nt, Homo sapiens
  source 1..23: mol_type = other RNA
taaagtgcttatagtgcaggtag

SEQ ID NO: 118 - RNA, 22 nt, Homo sapiens
  source 1..22: mol_type = other RNA
ttccctttgtcatccttcgcct

SEQ ID NO: 119 - RNA, 21 nt, Homo sapiens
  source 1..21: mol_type = other RNA
taacagtctccagtcacggcc

SEQ ID NO: 120 - RNA, 21 nt, Homo sapiens
  source 1..21: mol_type = other RNA
atgacctatgaattgacagac

SEQ ID NO: 121 - RNA, 21 nt, Homo sapiens
  source 1..21: mol_type = other RNA
ttgtgcttgatctaaccatgt

SEQ ID NO: 122 - RNA, 21 nt, Homo sapiens
  source 1..21: mol_type = other RNA
tgattgtccaaacgcaattct

SEQ ID NO: 123 - RNA, 25 nt, Homo sapiens
  source 1..25: mol_type = other RNA
tcaagtcactagtggttccgtttag

SEQ ID NO: 124 - RNA, 21 nt, Homo sapiens
  source 1..21: mol_type = other RNA
ttcaagtaattcaggataggt

SEQ ID NO: 125 - RNA, 22 nt, Homo sapiens
  source 1..22: mol_type = other RNA
aaggagctcacagtctattgag

SEQ ID NO: 126 - RNA, 21 nt, Homo sapiens
  source 1..21: mol_type = other RNA
atgtatgtgtgcatgtgcatg

SEQ ID NO: 127 - RNA, 24 nt, Homo sapiens
  source 1..24: mol_type = other RNA
agcagaagcagggaggttctccca

SEQ ID NO: 128 - RNA, 22 nt, Homo sapiens
  source 1..22: mol_type = other RNA
tggtttaccgtcccacatacat

SEQ ID NO: 129 - RNA, 23 nt, Homo sapiens
  source 1..23: mol_type = other RNA
tagcaccatttgaaatcagtgtt

SEQ ID NO: 130 - RNA, 22 nt, Homo sapiens
  source 1..22: mol_type = other RNA
tagcaccatttgaaatcggtta

SEQ ID NO: 131 - RNA, 22 nt, Homo sapiens
  source 1..22: mol_type = other RNA
tatacaagggcagactctctct

SEQ ID NO: 132 - RNA, 23 nt, Homo sapiens
  source 1..23: mol_type = other RNA
taagtgcttccatgttttagtag

SEQ ID NO: 133 - RNA, 22 nt, Homo sapiens
  source 1..22: mol_type = other RNA
tgtaaacatcctcgactggaag

SEQ ID NO: 134 - RNA, 23 nt, Homo sapiens
  source 1..23: mol_type = other RNA
tgtaaacatcctacactctcagc

SEQ ID NO: 135 - RNA, 22 nt, Homo sapiens
  source 1..22: mol_type = other RNA
tgtaaacatccttgactggaag

SEQ ID NO: 136 - RNA, 22 nt, Homo sapiens
  source 1..22: mol_type = other RNA
ctttcagtcggatgtttacagc

SEQ ID NO: 137 - RNA, 21 nt, Homo sapiens
  source 1..21: mol_type = other RNA
aggcaagatgctggcatagct

SEQ ID NO: 138 - RNA, 22 nt, Homo sapiens
  source 1..22: mol_type = other RNA
gccttctcttcccggttcttcc

SEQ ID NO: 139 - RNA, 22 nt, Homo sapiens
  source 1..22: mol_type = other RNA
aaaagctgggttgagagggcga

SEQ ID NO: 140 - RNA, 22 nt, Homo sapiens
  source 1..22: mol_type = other RNA
aaaagctgggttgagagggcaa

SEQ ID NO: 141 - RNA, 22 nt, Homo sapiens
  source 1..22: mol_type = other RNA
aggtggtccgtggcgcgttcgc

SEQ ID NO: 142 - RNA, 22 nt, Homo sapiens
  source 1..22: mol_type = other RNA
aggtggtccgtggcgcgttcgc

SEQ ID NO: 143 - RNA, 22 nt, Homo sapiens
  source 1..22: mol_type = other RNA
cgcatcccctagggcattggtg

SEQ ID NO: 144 - RNA, 22 nt, Homo sapiens
  source 1..22: mol_type = other RNA
ctggccctctctgcccttccgt

SEQ ID NO: 145 - RNA, 23 nt, Homo sapiens
  source 1..23: mol_type = other RNA
gaggttttctgggtttctgtttc

SEQ ID NO: 146 - RNA, 23 nt, Homo sapiens
  source 1..23: mol_type = other RNA
gcaaagcacacggcctgcagaga

SEQ ID NO: 147 - RNA, 21 nt, Homo sapiens
  source 1..21: mol_type = other RNA
gcccctgggcctatcctagaa

SEQ ID NO: 148 - RNA, 22 nt, Homo sapiens
  source 1..22: mol_type = other RNA
tccagcatcagtgattttgttg

SEQ ID NO: 149 - RNA, 22 nt, Homo sapiens
  source 1..22: mol_type = other RNA
aacaatatcctggtgctgagtg

SEQ ID NO: 150 - RNA, 23 nt, Homo sapiens
  source 1..23: mol_type = other RNA
tccctgtcctccaggagctcacg

SEQ ID NO: 151 - RNA, 21 nt, Homo sapiens
  source 1..21: mol_type = other RNA
gtgcattgtagttgcattgca

SEQ ID NO: 152 - RNA, 22 nt, Homo sapiens
  source 1..22: mol_type = other RNA
ttataaagcaatgagactgatt

SEQ ID NO: 153 - RNA, 23 nt, Homo sapiens
  source 1..23: mol_type = other RNA
tgtctgcccgcatgcctgcctct

SEQ ID NO: 154 - RNA, 23 nt, Homo sapiens
  source 1..23: mol_type = other RNA
aggcagtgtagttagctgattgc

SEQ ID NO: 155 - RNA, 22 nt, Homo sapiens
  source 1..22: mol_type = other RNA
agatcgaccgtgttatattcgc

SEQ ID NO: 156 - RNA, 22 nt, Homo sapiens
  source 1..22: mol_type = other RNA
gcctgctggggtggaacctggt

SEQ ID NO: 157 - RNA, 23 nt, Homo sapiens
  source 1..23: mol_type = other RNA
aaagtgctgcgacatttgagcgt

SEQ ID NO: 158 - RNA, 23 nt, Homo sapiens
  source 1..23: mol_type = other RNA
gaagtgcttcgattttggggtgt

SEQ ID NO: 159 - RNA, 22 nt, Homo sapiens
  source 1..22: mol_type = other RNA
ttataatacaacctgataagtg

SEQ ID NO: 160 - RNA, 22 nt, Homo sapiens
  source 1..22: mol_type = other RNA
tttgttcgttcggctcgcgtga

SEQ ID NO: 161 - RNA, 23 nt, Homo sapiens
  source 1..23: mol_type = other RNA
gcgacgagcccctcgcacaaacc

SEQ ID NO: 162 - RNA, 22 nt, Homo sapiens
  source 1..22: mol_type = other RNA
atcatagaggaaaatccatgtt

SEQ ID NO: 163 - RNA, 22 nt, Homo sapiens
  source 1..22: mol_type = other RNA
cgtggatattccttctatgttt

SEQ ID NO: 164 - RNA, 22 nt, Homo sapiens
  source 1..22: mol_type = other RNA
atcacacaaaggcaacttttgt

SEQ ID NO: 165 - RNA, 22 nt, Homo sapiens
  source 1..22: mol_type = other RNA
actggacttggagtcagaaggc

SEQ ID NO: 166 - RNA, 21 nt, Homo sapiens
  source 1..21: mol_type = other RNA
tggtagactatggaacgtagg

SEQ ID NO: 167 - RNA, 22 nt, Homo sapiens
  source 1..22: mol_type = other RNA
tatgtaatatggtccacatctt

SEQ ID NO: 168 - RNA, 22 nt, Homo sapiens
  source 1..22: mol_type = other RNA
tatacaagggcaagctctctgt

SEQ ID NO: 169 - RNA, 22 nt, Homo sapiens
  source 1..22: mol_type = other RNA
gaagttgttcgtggtggattcg

SEQ ID NO: 170 - RNA, 22 nt, Homo sapiens
  source 1..22: mol_type = other RNA
agatcagaaggtgattgtggct

SEQ ID NO: 171 - RNA, 22 nt, Homo sapiens
  source 1..22: mol_type = other RNA
gaatgttgctcggtgaacccct

SEQ ID NO: 172 - RNA, 21 nt, Homo sapiens
  source 1..21: mol_type = other RNA
aatataacacagatggcctgt

SEQ ID NO: 173 - RNA, 21 nt, Homo sapiens
  source 1..21: mol_type = other RNA
tagtagaccgtatagcgtacg

SEQ ID NO: 174 - RNA, 22 nt, Homo sapiens
  source 1..22: mol_type = other RNA
tatgtaacacggtccactaacc

SEQ ID NO: 175 - RNA, 21 nt, Homo sapiens
  source 1..21: mol_type = other RNA
tagtagaccgtatagcgtacg

SEQ ID NO: 176 - RNA, 21 nt, Homo sapiens
  source 1..21: mol_type = other RNA
tgtcttgcaggccgtcatgca

SEQ ID NO: 177 - RNA, 23 nt, Homo sapiens
  source 1..23: mol_type = other RNA
tcttggagtaggtcattgggtgg

SEQ ID NO: 178 - RNA, 22 nt, Homo sapiens
  source 1..22: mol_type = other RNA
atcatgatgggctcctcggtgt

SEQ ID NO: 179 - RNA, 22 nt, Homo sapiens
  source 1..22: mol_type = other RNA
ttgcatatgtaggatgtcccat

SEQ ID NO: 180 - RNA, 22 nt, Homo sapiens
  source 1..22: mol_type = other RNA
aactgtttgcagaggaaactga

SEQ ID NO: 181 - RNA, 22 nt, Homo sapiens
  source 1..22: mol_type = other RNA
agaggctggccgtgatgaattc

SEQ ID NO: 182 - RNA, 22 nt, Homo sapiens
  source 1..22: mol_type = other RNA
tcctgtactgagctgccccgag

SEQ ID NO: 183 - RNA, 22 nt, Homo sapiens
  source 1..22: mol_type = other RNA
aatcgtacagggtcatccactt

SEQ ID NO: 184 - RNA, 22 nt, Homo sapiens
  source 1..22: mol_type = other RNA
aaacaaacatggtgcacttctt

SEQ ID NO: 185 - RNA, 21 nt, Homo sapiens
  source 1..21: mol_type = other RNA
cagcagcacactgtggtttgt

SEQ ID NO: 186 - RNA, 22 nt, Homo sapiens
  source 1..22: mol_type = other RNA
cgtcaacacttgctggtttcct

SEQ ID NO: 187 - RNA, 18 nt, Homo sapiens
  source 1..18: mol_type = other RNA
ttcacagggaggtgtcat

SEQ ID NO: 188 - RNA, 22 nt, Homo sapiens
  source 1..22: mol_type = other RNA
gagtgccttcttttggagcgtt

SEQ ID NO: 189 - RNA, 24 nt, Homo sapiens
  source 1..24: mol_type = other RNA
ttctccaaaagaaagcactttctg

SEQ ID NO: 190 - RNA, 22 nt, Homo sapiens
  source 1..22: mol_type = other RNA
atcgtgcatccctttagagtgt

SEQ ID NO: 191 - RNA, 22 nt, Homo sapiens
  source 1..22: mol_type = other RNA
caaagcgctcccctttagaggt

SEQ ID NO: 192 - RNA, 23 nt, Homo sapiens
  source 1..23: mol_type = other RNA
tctctggagggaagcactttctg

SEQ ID NO: 193 - RNA, 22 nt, Homo sapiens
  source 1..22: mol_type = other RNA
caaagtgcctccctttagagtg

SEQ ID NO: 194 - RNA, 23 nt, Homo sapiens
  source 1..23: mol_type = other RNA
gaacgcgcttccctatagagggt

SEQ ID NO: 195 - RNA, 22 nt, Homo sapiens
  source 1..22: mol_type = other RNA
gaaggcgcttccctttagagcg

SEQ ID NO: 196 - RNA, 22 nt, Homo sapiens
  source 1..22: mol_type = other RNA
ggagaaattatccttggtgtgt

SEQ ID NO: 197 - RNA, 23 nt, Homo sapiens
  source 1..23: mol_type = other RNA
gtttgcacgggtgggccttgtct

SEQ ID NO: 198 - RNA, 19 nt, Homo sapiens
  source 1..19: mol_type = other RNA
aggcacggtgtcagcaggc

SEQ ID NO: 199 - RNA, 20 nt, Homo sapiens
  source 1..20: mol_type = other RNA
gtccgctcggcggtggccca

SEQ ID NO: 200 - RNA, 19 nt, Homo sapiens
  source 1..19: mol_type = other RNA
gagccagttggacaggagc

SEQ ID NO: 201 - RNA, 22 nt, Homo sapiens
  source 1..22: mol_type = other RNA
ttatggtttgcctgggactgag

SEQ ID NO: 202 - RNA, 19 nt, Homo sapiens
  source 1..19: mol_type = other RNA
tgggcgtatctgtatgcta

SEQ ID NO: 203 - RNA, 23 nt, Homo sapiens
  source 1..23: mol_type = other RNA
atcccttgcaggggctgttgggt

SEQ ID NO: 204 - RNA, 21 nt, Homo sapiens
  source 1..21: mol_type = other RNA
tctagtaagagtggcagtcga

SEQ ID NO: 205 - RNA, 25 nt, Homo sapiens
  source 1..25: mol_type = other RNA
agggatcgcgggcgggtggcggcct

SEQ ID NO: 206 - RNA, 20 nt, Homo sapiens
  source 1..20: mol_type = other RNA
accaggaggctgaggcccct

SEQ ID NO: 207 - RNA, 23 nt, Homo sapiens
  source 1..23: mol_type = other RNA
tgtcactcggctcggcccactac

SEQ ID NO: 208 - RNA, 22 nt, Homo sapiens
  source 1..22: mol_type = other RNA
tgcggggctagggctaacagca

SEQ ID NO: 209 - RNA, 24 nt, Homo sapiens
  source 1..24: mol_type = other RNA
tggaagactagtgattttgttgtt

SEQ ID NO: 210 - RNA, 20 nt, Homo sapiens
  source 1..20: mol_type = other RNA
cggctctgggtctgtgggga

SEQ ID NO: 211 - RNA, 21 nt, Homo sapiens
  source 1..21: mol_type = other RNA
tggaggagaaggaaggtgatg

SEQ ID NO: 212 - RNA, 22 nt, Homo sapiens
  source 1..22: mol_type = other RNA
actccagccccacagcctcagc

SEQ ID NO: 213 - RNA, 22 nt, Homo sapiens
  source 1..22: mol_type = other RNA
tgagacctctgggttctgagct

SEQ ID NO: 214 - RNA, 21 nt, Homo sapiens
  source 1..21: mol_type = other RNA
gcaggaacttgtgagtctcct

SEQ ID NO: 215 - RNA, 22 nt, Homo sapiens
  source 1..22: mol_type = other RNA
ctgccctggcccgagggaccga

SEQ ID NO: 216 - RNA, 23 nt, Homo sapiens
  source 1..23: mol_type = other RNA
caaagtgctgttcgtgcaggtag

SEQ ID NO: 217 - RNA, 24 nt, Homo sapiens
  source 1..24: mol_type = other RNA
tggggagctgaggctctgggggtg

SEQ ID NO: 218 - RNA, 21 nt, Homo sapiens
  source 1..21: mol_type = other RNA
aaggcagggcccccgctcccc

SEQ ID NO: 219 - RNA, 22 nt, Homo sapiens
  source 1..22: mol_type = other RNA
ttcaacgggtatttattgagca

SEQ ID NO: 220 - RNA, 22 nt, Homo sapiens
  source 1..22: mol_type = other RNA
tgaggtagtaagttgtattgtt

SEQ ID NO: 221 - RNA, 22 nt, Homo sapiens
  source 1..22: mol_type = other RNA
aacccgtagatccgatcttgtg

SEQ ID NO: 222 - skipped

SEQ ID NO: 223 - DNA, 30 bp, Zika virus
  source 1..30: mol_type = other DNA
tctccccaggacaaccaaatggcaatcatc

SEQ ID NO: 224 - DNA, 30 bp, Zika virus
  source 1..30: mol_type = other DNA
gatgattgccatttggttgtcctggggaga

SEQ ID NO: 225 - DNA, 3887 bp, synthetic construct
  misc_feature 1..3887: note = Synthetic Oligonucleotide
  source 1..3887: mol_type = other DNA
taatacgactcactatagggtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcccctcagcctatgcggatccgatcggaccgctcgagaccggtatcgatgttaacacaactctgatgagtccgtgaggacgaaacgagtaagctcgtcagttgttactgttgctgactcagactgcgacagttcgagtttgaagcgaaagctagcaacagtatcaacaggttttatttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtac

SEQ ID NO: 226 - DNA, 21 bp, synthetic construct
  misc_feature 1..21: note = Synthetic Oligonucleotide
  source 1..21: mol_type = other DNA
atgcatccgacctgtatgcat

SEQ ID NO: 227 - DNA, 21 bp, synthetic construct
  misc_feature 1..21: note = Synthetic Oligonucleotide
  source 1..21: mol_type = other DNA
atgcatacaggtcggatgcat

SEQ ID NO: 228 - DNA, 12 bp, Zika virus
  source 1..12: mol_type = other DNA
tctccccaggac

SEQ ID NO: 229 - DNA, 12 bp, Zika virus
  source 1..12: mol_type = other DNA
gtcctggggaga

SEQ ID NO: 230 - DNA, 18 bp, Zika virus
  source 1..18: mol_type = other DNA
aaccaaatggcaatcatc

SEQ ID NO: 231 - DNA, 17 bp, Zika virus
  source 1..17: mol_type = other DNA
gatgattgccatttggt

SEQ ID NO: 232 - DNA, 15 bp, Zika virus
  source 1..15: mol_type = other DNA
acacctggagtgctg

SEQ ID NO: 233 - DNA, 15 bp, Zika virus
  source 1..15: mol_type = other DNA
cagcactccaggtgt

SEQ ID NO: 234 - DNA, 33 bp, Zika virus
  source 1..33: mol_type = other DNA
gcaccaatcttaatgttgtcaggcctgctagtc

SEQ ID NO: 235 - DNA, 33 bp, Zika virus
  source 1..33: mol_type = other DNA
gactagcaggcctgacaacattaagattggtgc

SEQ ID NO: 236 - DNA, 56 bp, synthetic construct
  misc_feature 1..56: note = Synthetic Oligonucleotide
  source 1..56: mol_type = other DNA
taacgcgttgccggtcaacgcgtgcaccaatcttaatgttgtcaggcctgctagtc

SEQ ID NO: 237 - DNA, 56 bp, synthetic construct
  misc_feature 1..56: note = Synthetic Oligonucleotide
  source 1..56: mol_type = other DNA
gactagcaggcctgacaacattaagattggtgcacgcgttgaccggcaacgcgtta

SEQ ID NO: 238 - DNA, 2792 bp, synthetic construct
  misc_feature 1..2792: note = Synthetic Oligonucleotide
  source 1..2792: mol_type = other DNA
gcgcatgctatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggagaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatattatctccgatgaagcctccgttatcccaggttccttgggtttgttgccatctgcgtccttggcctctttgccagacaagaacaccgcatttggtttgtacgaaccatgtcacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaaagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcccctcagcctatgcggatccgatcggaccgctcgagaccggtatcgatgttaacacaact

SEQ ID NO: 239 - DNA, 106 bp, synthetic construct
  misc_feature 1..106: note = Synthetic Oligonucleotide
  source 1..106: mol_type = other DNA
agttgttactgttgctgactcagactgcgacagttcgagtttgaagcgaaagctagcaacagtatcaacaggttttatttggatttggaaacgagagtttctggtc

SEQ ID NO: 240 - DNA, 10269 bp, synthetic construct
  source 1..10269: mol_type = other DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtggggctgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggattattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccatgggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacgcccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatggcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggtactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagtcctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctg

SEQ ID NO: 241 - DNA, 431 bp, synthetic construct
  source 1..431: mol_type = other DNA
taagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 242 - DNA, 760 bp, Synthetic construct
  source 1..760: mol_type = other DNA
gtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcccctcagcctatgcggatccgatcggaccgctcgagaccggtatcgatgttaacacaact

SEQ ID NO: 243 - DNA, 10272 bp, synthetic construct
  source 1..10272: mol_type = other DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtggggctgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggattattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccatgggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacgcccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatggcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggtactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagtcctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaa

SEQ ID NO: 244 - DNA, 428 bp, synthetic construct
  source 1..428: mol_type = other DNA
gcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 245 - DNA, 10271 bp, synthetic construct
  source 1..10271: mol_type = other DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtggggctgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggattattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccatgggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacgcccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatggcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggtactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagtcctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgta

SEQ ID NO: 246 - DNA, 88 bp, synthetic construct
  source 1..88: mol_type = other DNA
gagacccaagctggctagcgtttaaacttaagcttggtaccgagctcggatccctcgagctcgaaccggtatcgatgttaacacaact

SEQ ID NO: 247 - DNA, 1527 bp, synthetic construct
  source 1..1527: mol_type = other DNA
atgaaaaacccaaaaaagaaatccggaggattccggattgtcaatatgctaaaacgcggagtagcccgtgtgagcccctttgggggcttgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgcatcatcaccatcaccactaa

SEQ ID NO: 248 - DNA, 10272 bp, synthetic construct
  source 1..10272: mol_type = other DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtggggctgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagctggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggattattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccatgggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacgcccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatggcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggtactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagtcctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaa

SEQ ID NO: 249 - DNA, 10277 bp, synthetic construct
  source 1..10277: mol_type = other DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtggggctgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggattattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccatgggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacgcccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatggcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggtactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagtcctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaacgcgg

SEQ ID NO: 250 - DNA, 10272 bp, synthetic construct; identical to SEQ ID NO: 248
  source 1..10272: mol_type = other DNA

(19) United States
(12) Patent Application Publication
(10) Pub. No.: US 2025/0154509 A1
(43) Pub. Date: May 15, 2025

(54) ONCOLYTIC VIRUSES AND METHODS OF USE

(71) Applicant: Vyro Bio Inc., Newark, DE (US)

(72) Inventor: Carolini Kaid DAVILA, Osasco, São Paulo (BR)

(21) Appl. No.: 18/840,018

(22) PCT Filed: Feb. 21, 2023
(86) PCT No.: PCT/US2023/062967

§ 371 Date: Aug. 20, 2024

Related U.S. Application Data
(60) Provisional application No. 63/312,782, filed on Feb. 22, 2022.

(51) Int. Cl. C12N 15/113; A61K 35/768; A61P 35/00; C12N 7/00

(52) U.S. Cl. CPC C12N 15/1135; A61K 35/768; A61P 35/00; C12N 7/00; C12N 2310/141; C12N 2770/24122; C12N 2770/24132; C12N 2770/24143; C12N 2770/24171

(57) ABSTRACT

Disclosed herein are nucleic acid viruses for use in the treatment of cancer.

### CROSS-REFERENCE TO RELATED APPLICATION

This application claims priority to U.S. provisional application 63/312,782 filed Feb. 22, 2022, the entirety of which is incorporated by reference herein.

### BACKGROUND OF THE INVENTION

Cancer is the second leading cause of mortality worldwide and overall, the prevalence of cancer has increased. Thus, there is an unmet need for a new therapeutic approach that can impact the course of the disease.

### INCORPORATION BY REFERENCE OF SEQUENCE LISTING

The present application is being filed along with a Sequence Listing in electronic format. The Sequence Listing is provided as a file entitled 61771-701601.xml, created Feb. 21, 2023, which is 357,386 bytes in size. The information in the electronic format of the Sequence Listing is incorporated by reference in its entirety.

### SUMMARY OF THE INVENTION

In one aspect, provided herein are viruses that address the need for a new therapeutic approach to cancer. Methods of use involve administering a virus to a patient having cancer such that the virus impairs cell division, impairs cell growth, and/or induces cell death of cancerous cells. In some cases, viruses having one or more anti-cancerous effects are oncolytic viruses.

In one aspect, provided herein is a method of treating cancer, comprising administering to a subject in need thereof a recombinant zika virus. Further aspects include recombinant zika viruses and nucleic acid compositions.

In some embodiments, the recombinant zika virus produces a higher level of subgenomic flaviviral RNA (sfRNA) than a wild-type zika virus in a cancer cell of the subject. In some embodiments, the higher level of sfRNA is by at least 10%, at least 20%, at least 30%, at least 40% at least 50%, at least 60%, at least 70%, at least 80%, at least 90%, at least 100%.

In some embodiments, the cancer is a brain cancer, a retinal cancer, a testicle cancer, or a prostate cancer. In some embodiments, the cancer is a CNS cancer. In some embodiments, the cancer is brain cancer. In some specific embodiments, the brain cancer is an astrocytoma, an oligodendroglioma, an ependymoma, a meningioma, a schwannoma, a craniopharyngioma, a germinoma, a pineocytoma, or a combination thereof. In some embodiments, the cancer is a breast cancer. In some embodiments, the cancer is colon cancer. In some embodiments, the cancer is prostate cancer.

In some embodiments, the recombinant zika virus is produced from a nucleic acid composition described herein.

In some embodiments, the recombinant zika virus is produced from a nucleic acid composition comprising a polynucleotide encoding a zika virus capsid protein (ancC/C) or a derivative of the zika virus ancC/C, a polynucleotide encoding a zika virus viral membrane protein (prM/M) or a derivative of the zika virus prM/M, a polynucleotide encoding a zika virus viral envelope protein (E) or a derivative of the zika virus E, or any combination thereof. In some embodiments, the nucleic acid composition comprises the polynucleotide encoding the zika virus ancC/C or the derivative of the zika virus ancC/C, the polynucleotide encoding the zika virus prM/M or the derivative of the zika virus prM/M, and the polynucleotide encoding the zika virus E or the derivative of the zika virus E. In some embodiments, the polynucleotide encoding the zika virus ancC/C or the derivative of the zika virus ancC/C, the polynucleotide encoding the zika virus prM/M or the derivative of the zika virus prM/M, and the polynucleotide encoding the zika virus E or the derivative of the zika virus E, are expressed on one or two or more separate nucleic acids. In some embodiments, the polynucleotide encoding the derivative of the zika virus ancC/C comprises at least one substitution, at least one deletion, and/or at least one insertion as compared to a wild-type zika virus ancC/C. In some embodiments, the zika virus ancC/C or the derivative of the zika virus ancC/C comprises an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 1. In some embodiments, the polynucleotide encoding the derivative of the zika virus prM/M comprises at least one substitution, at least one deletion, and/or at least one insertion as compared to a wild-type zika virus prM/M. In some embodiments, the zika virus prM/M or the derivative of the zika virus prM/M comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 2. In some embodiments, the polynucleotide encoding the derivative of the zika virus E comprises at least one substitution, at least one deletion, and/or at least one insertion as compared to a wild-type zika virus E. In some embodiments, the polynucleotide encoding E is translated into a wild zika virus type E. In some embodiments, the zika virus E or the derivative of the zika virus E comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to sequence of Table 3.

In some embodiments, the nucleic acid composition comprises a 5′ untranslated region (5′ UTR) of the zika virus. In some embodiments, the 5′ untranslated region (5′ UTR) of the zika virus comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 4 or Table 12. In some embodiments, the nucleic acid composition comprises a 3′ untranslated region (3′ UTR) of the zika virus. In some embodiments, the 3′ untranslated region (3′ UTR) of the zika virus comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 4 or Table 13.

In some embodiments, the nucleic acid composition does not comprise a polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). In other embodiments, the nucleic acid composition comprises polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). In some embodiments, the NS1 is a zika virus NS1, the NS2A is a zika virus NS2A, the NS2B is a zika virus NS2B, the NS3 is a zika virus NS3, the NS4A is a zika virus NS4A, the NS4B is a zika virus NS4B, or the NS5 is a zika virus NS5, or any combination of two or more thereof.

In some embodiments, the components of the nucleic acid composition comprise African zika virus components, Asian zika virus components, or Brazilian zika virus components, or a combination thereof. In one specific embodiment, the zika virus is African MR766 strain.

In some embodiments, the nucleic acid composition is expressed on one or two or more separate nucleic acids. In some embodiments, the nucleic acid composition comprises one or more expression control elements in operable linkage that confers expression of the nucleic acid composition in vitro or in vivo. In some embodiments, the expression control element is a promoter that drives expression of the nucleic acid composition in vitro. In specific embodiments, the promoter is T7, T3, SP6 or any phage promoter.

In some embodiments, the expression control element is a promoter that drives expression of the nucleic acid composition in a target cell. In some specific embodiments, the promoter is CMV, SV40 or any eukaryotic promoter.

In some embodiments, the target cell is a neuron, or a non-neuron cell, Vero, COS, CHO, CHLA, C6/36, HeLa, HEK, HepG2. In some embodiments, the target cell is an oligodendrocyte, microglia, or astrocyte.

In some embodiments, a recombinant zika virus is generated from expressing the nucleic acid composition described herein in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus.

In some embodiments, the second zika virus is a wild-type zika virus. In some embodiments, the wild-type zika virus is an African, an Asian and a Brazilian strain. In other embodiments, the second zika virus is a modified zika virus. In some specific embodiments, the modified zika virus comprises one or more microRNA-based gene-silencing machineries. In some specific embodiments, the one or more microRNA-based gene-silencing machineries control viral replication.

In some embodiments, a recombinant zika virus that is generated from expressing the nucleic acid composition as described herein in a producer cell, without an infection of a second zika virus, wherein the zika virus ancC/C or the derivative of the zika virus ancC/C, the zika virus prM/M or the derivative of the zika virus prM/M, and the zika virus E or the derivative of the zika virus E are present in the recombinant zika virus. In some embodiment, the producer cell is a Vero E6 or C6/36 cell.

In some embodiments, the recombinant zika virus is replication competent. In other embodiments, the recombinant zika virus is replication incompetent without lowering the vector titer. In some embodiments, the recombinant zika virus has decreased insertional mutagenesis. In other embodiments, the recombinant zika virus has decreased immune response.

Non-limiting example nucleic acid compositions provided herein comprise a zika virus 5′ untranslated region (5′ UTR), a sequence complementary to a regulatory polynucleotide, and a zika virus 3′ untranslated region (3′UTR), wherein the regulatory polynucleotide comprises (i) miR-219a-2-3p, hsa-miR-377-3p, hsa-miR-1225-5p, hsa-miR-4298, hsa-miR-219a-5p, or hsa-miR-129-5p, (ii) a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 6A or Table 6B, or (iii) (i) and (ii). In some embodiments, the sequence complementary to the regulatory polynucleotide comprises CCGACCTGT, AAAAACGCC, AAACGCCAG, GGCGTTTT, CTAACAGGT, TAACAGGTT, ACTAACAGG, ACCCTGTCC, CACCCATGC, CCCATGCCG, ACCCATGCC, AGTGTGTTT, CTTAACACC, TTAACACCG, CTTAACAGC, or TGCCGGTCA, or a combination of two or more thereof. In some embodiments, the 5′ UTR is at least 80%, 81%, 82%, 83%, 84%, 85%, 86%, 87%, 88%, 89%, 90%, 91%, 92%, 93%, 94%, 95%, 96%, 97%, 98%, or 99% homologous or identical to SEQ ID NO: 239 (AGTTGTTACTGTTGCTGACTCAGACTGCGACAGTTCGAGTTTGAAGCGAAAGCTAGC AACAGTATCAACAGGTTTTATTTGGATTTGGAAACGAGAGTTTCTGGTC). In some embodiments, the 3′ UTR is at least 80%, 81%, 82%, 83%, 84%, 85%, 86%, 87%, 88%, 89%, 90%, 91%, 92%, 93%, 94%, 95%, 96%, 97%, 98%, or 99% homologous or identical to SEQ ID NO: 241 (TAAGCACCAATCTTAATGTTGTCAGGCCTGCTAGTCAGCCACAGCTTGGGGAAAGC TGTGCAGCCTGTGACCCCCCCAGGAGAAGCTGGGAAACCAAGCCTATAGTCAGGCC GAGAACGCCATGGCACGGAAGAAGCCATGCTGCCTGTGAGCCCCTCAGAGGACACT GAGTCAAAAAACCCCACGCGCTTGGAGGCGCAGGATGGGAAAAGAAGGTGGCGAC CTTCCCCACCCTTCAATCTGGGGCCTGAACTGGAGATCAGCTGTGGATCTCCAGAAG AGGGACTAGTGGTTAGAGGAGACCCCCCGGAAAACGCAAAACAGCATATTGACGCT GGGAAAGACCAGAGACTCCATGAGTTTCCACCACGCTGGCCGCCAGGCACAGATCG CCGAATAGCGGCGGCCGGTGTGGGGAAATCCATGGGTCT). In some embodiments, the 5′ UTR is selected from a zika virus of Table 12, and the 3′ UTR is selected from a zika virus of Table 13. In some embodiments, the regulatory polynucleotide is expressed in non-cancer cells, optionally wherein the regulatory polynucleotide has higher expression in non-cancer cells as compared to cancer cells of a subject. In some embodiments, the regulatory polynucleotide is a microRNA. In some embodiments, the sequence complementary to the regulatory polynucleotide is between 9 nucleotides and 22 nucleotides in length, optionally about 9, 10, 11, 12, 13, 14, 15, 16, 17, 18, 19, 20, 21, or 22 nucleotides in length.

Non-limiting example nucleic acid compositions provided herein comprise (a) a zika virus 5′ untranslated region (5′ UTR); (b) a polynucleotide encoding at least one zika virus structural protein selected from capsid protein (ancC/C), membrane protein (prM/M), and envelope protein (E), and/or a polynucleotide encoding at least one zika virus non-structural protein selected from NS1, NS2A, NS2B, NS3, NS4A, 2k peptide, NS4B, and NS5; (c) a zika virus 3′ untranslated region (3′UTR); and (d) a sequence complementary to a regulatory polynucleotide; wherein: (i) the nucleic acid comprises the polynucleotide encoding at least one zika virus non-structural protein, and the polynucleotide encoding the at least one zika virus non-structural protein comprises a polynucleotide encoding the 2k peptide, wherein the sequence complementary to the regulatory polynucleotide is positioned within the polynucleotide encoding the 2k peptide; (ii) the nucleic acid comprises the polynucleotide encoding at least one zika virus non-structural protein, and the polynucleotide encoding the at least one zika virus non-structural protein comprises a polynucleotide encoding the NS4A and a polynucleotide encoding the NS4B, and the sequence complementary to the regulatory polynucleotide is positioned between the polynucleotide encoding the NS4A and the polynucleotide encoding the NS4B; (iii) the sequence complementary to the regulatory polynucleotide is positioned between the 5′ UTR and the polynucleotide encoding at least one zika virus structural protein and/or the polynucleotide encoding at least one zika virus non-structural protein; and/or (iv) the sequence complementary to the regulatory polynucleotide is positioned between the 3′ UTR and the polynucleotide encoding at least one zika virus structural protein and/or the polynucleotide encoding at least one zika virus non-structural protein.

In some embodiments, the nucleic acid comprises the polynucleotide encoding at least one zika virus non-structural protein, and the polynucleotide encoding the at least one zika virus non-structural protein comprises a polynucleotide encoding the 2k peptide, wherein the sequence complementary to the regulatory polynucleotide is positioned within the polynucleotide encoding the 2k peptide. In some embodiments, the nucleic acid comprises the polynucleotide encoding at least one zika virus non-structural protein, and the polynucleotide encoding the at least one zika virus non-structural protein comprises a polynucleotide encoding the NS4A and a polynucleotide encoding the NS4B, and the sequence complementary to the regulatory polynucleotide is positioned between the polynucleotide encoding the NS4A and the polynucleotide encoding the NS4B. In some embodiments, the sequence complementary to the regulatory polynucleotide is positioned between the 5′ UTR and the polynucleotide encoding at least one zika virus structural protein and/or the polynucleotide encoding at least one zika virus non-structural protein. In some embodiments, the sequence complementary to the regulatory polynucleotide is positioned between the 3′ UTR and the polynucleotide encoding at least one zika virus structural protein and/or the polynucleotide encoding at least one zika virus non-structural protein.

In some embodiments, the 5′ UTR is at least 80%, 81%, 82%, 83%, 84%, 85%, 86%, 87%, 88%, 89%, 90%, 91%, 92%, 93%, 94%, 95%, 96%, 97%, 98%, or 99% homologous or identical to SEQ ID NO: 239. In some embodiments, the 3′ UTR is at least 80%, 81%, 82%, 83%, 84%, 85%, 86%, 87%, 88%, 89%, 90%, 91%, 92%, 93%, 94%, 95%, 96%, 97%, 98%, or 99% homologous or identical to SEQ ID NO: 241. In some embodiments, the 5′ UTR is selected from a zika virus of Table 12, and the 3′ UTR is selected from a zika virus of Table 13. In some embodiments, the nucleic acid comprises the polynucleotide encoding at least one zika virus structural protein, and the nucleic acid comprising the polynucleotide encoding at least one zika virus structural protein comprises: a polynucleotide encoding the capsid protein (ancC/C), a polynucleotide encoding the membrane protein (prM/M), and a polynucleotide encoding the envelope protein (E). In some embodiments, the nucleic acid comprises the polynucleotide encoding at least one zika virus non-structural protein, and the polynucleotide encoding at least one zika virus non-structural protein comprises: a polynucleotide encoding the NS1, a polynucleotide encoding the NS2A, a polynucleotide encoding the NS2B, a polynucleotide encoding the NS3, a polynucleotide encoding the NS4A, a polynucleotide encoding the 2k peptide, a polynucleotide encoding the NS4B, and a polynucleotide encoding the NS5. In some embodiments, the regulatory polynucleotide is expressed in non-cancer cells, optionally wherein the regulatory polynucleotide has higher expression in non-cancer cells as compared to cancer cells of a subject. In some embodiments, the regulatory polynucleotide is a microRNA. In some embodiments, the sequence complementary to the regulatory polynucleotide is between 9 nucleotides and 22 nucleotides in length, optionally about 9, 10, 11, 12, 13, 14, 15, 16, 17, 18, 19, 20, 21, or 22 nucleotides in length.

In some embodiments, a recombinant zika virus is produced from a nucleic acid composition described herein. In some embodiments, the recombinant zika virus is an oncolytic zika virus having oncolytic activity.

Non-limiting example recombinant zika viruses provided herein comprise a polynucleotide complementary to a regulatory polynucleotide, the regulatory polynucleotide comprising (i) miR-219a-2-3p, hsa-miR-377-3p, hsa-miR-1225-5p, hsa-miR-4298, hsa-miR-219a-5p, or hsa-miR-129-5p, (ii) a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 6A or Table 6B, or (iii) (i) and (ii). In some embodiments, the sequence complementary to the regulatory polynucleotide comprises CCGACCTGT, AAAAACGCC, AAACGCCAG, GGCGTTTT, CTAACAGGT, TAACAGGTT, ACTAACAGG, ACCCTGTCC, CACCCATGC, CCCATGCCG, ACCCATGCC, AGTGTGTTT, CTTAACACC, TTAACACCG, CTTAACAGC, or TGCCGGTCA, or a combination of two or more thereof. In some embodiments, the recombinant zika virus is an oncolytic zika virus having oncolytic activity.

Non-limiting example methods herein include a method of treating cancer in a subject in need thereof, the method comprising administering to the subject a recombinant zika virus. In some embodiments, the recombinant zika virus is a recombinant zika virus provided herein. In some embodiments, the cancer is a central nervous system (CNS) cancer. In some embodiments, the CNS cancer is a brain cancer. In some embodiments, the brain cancer is an astrocytoma, an oligodendroglioma, an ependymoma, a meningioma, a schwannoma, a craniopharyngioma, a germinoma, or a pineocytoma. In some embodiments, the cancer is breast cancer, prostate cancer, colon cancer, retinal cancer, or testicular cancer. In some embodiments, the recombinant zika virus does not infect and/or does not replicate in a non-cancer cell of the subject; or wherein the recombinant zika virus infects and/or replicates less in a non-cancer cell of the subject as compared to a cancer cell of the subject.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1D illustrate example recombinant zika virus structural features and DNA/RNA elements for transcription and viral genome processing. FIG. 1A shows an unmodified zika virus flanked by ribozymes and a promoter. FIG. 1B shows recombinant zika virus with a miRNA target inserted into 2k region. FIG. 1C shows the recombinant zika virus with a miRNA target inserted between the 5′ UTR and the coding sequence. FIG. 1D shows the recombinant zika virus with a miRNA target inserted between the coding sequence and the 3′ UTR.

FIGS. 1E-1G are representative images highlighting an example 2k and 3′UTR modification of FIG. 1B and FIG. 1D, respectively. FIG. 1E shows the sequence of nucleotides and amino acids at the NS4A (black) (SEQ ID NO: 24 and SEQ ID NO: 25) and 2k region (SEQ ID NO: 223, SEQ ID NO: 224). FIG. 1F shows insertion of miR-1225-5p target sequence (miRNA target 13). In FIG. 1F, a miR-1225-5p target sequence (miRNA target 13) flanked by EcoT221 (or AvaIII) sites was inserted into the 2k region, resulting in a modified 2k region (SEQ ID NO: 26, SEQ ID NO: 27) comprising a first 2k region (SEQ ID NO: 228, SEQ ID NO: 229) and a second 2k region (SEQ ID NO: 230 and SEQ ID NO:231). In FIG. 1G, a miR-1225-5p target sequence (miRNA target 16) was inserted at the junction of NS5 (SEQ ID NO: 232, 233) and 3′ UTR (SEQ ID NO: 234, 235), where the target sequence was flanked by MluI sites. The insertion of miR-1225-5p resulted in a modified sequence (SEQ ID NO: 239, 240).

FIG. 1H shows the location of a polyprotein according to embodiments of the disclosure in relation to the endoplasmic reticulum membrane and its cleavage sites. FIGS. 1IA-1IB provide an example schematic of a proposed mechanism of action of an example oncolytic zika virus comprising an exogenous polynucleotide provided herein.

FIGS. 2A-2F show the expression profiles of miRNAs in normal and tumoral tissue. FIG. 2A shows miR219a-2-3p high expression in neural progenitor cell and low/zero expression in CNS tumor cell lines (glioblastoma, medulloblastoma and ATRT) and other non-tumoral cell lines. FIG. 2B shows miR219a-5p high expression in neural progenitor cell and low/zero expression in CNS tumor cell lines (glioblastoma, medulloblastoma and ATRT) and other non-tumoral cell lines. FIG. 2C shows miR 129a-5p high expression in neural progenitor cells and certain medulloblastoma cells (Daoy, CHLA06) and low/zero expression in CNS tumor cell lines (glioblastoma, medulloblastoma and ATRT) and other non-tumoral cell lines. FIG. 2D shows miR4298 (SEQ ID NO: 10) low/zero expression in all cell lines. FIG. 2E shows miR377-3p high expression in normal cells and a glioblastoma cell line (U251), modest expression in medulloblastoma cells (Daoy, CHLA06), and low/zero expression in CNS tumor cell lines (glioblastoma, medulloblastoma and ATRT). FIG. 2F shows miR 1225-5p high expression in a normal cell line (Vero) and a medulloblastoma cell line (Daoy), modest expression in glioblastoma cell lines (HCB151, U138), and low/zero expression in all other cell lines.

FIGS. 3A-3E show example exogenous miRNA target sequences (or seed-complementary target sequences) inserted in the recombinant zika virus genome.

FIG. 3A shows example exogenous miR219a-2-3p target sequences (or seed-complementary target sequences) (CTTAACACC (miRNA target 12), CCGACCTGT (miRNA target 13), TTAACACCG (miRNA target 14)) that are complementary to miR-219a-2-ep (SEQ ID NO: 13) and were inserted in or adjacent to example 2k, 5′ UTR, or 3′ UTR modification locations of the recombinant zika virus genome. In one modification, miRNA target 12 was inserted into the 2k region and flanked by AvaIII sites to give a modified 2k region (SEQ ID NO: 31). In one modification, miRNA target 13 was inserted into the 2k region and flanked by AvaIII sites to give a modified 2k region (SEQ ID NO: 32). In one modification, miRNA target 12 was inserted adjacent to the 5′ UTR region and flanked by BsiWI sites (SEQ ID NO: 33). In one modification, miRNA target 14 was inserted adjacent to the 3′ UTR region and flanked by MluI sites (SEQ ID NO: 34).

FIG. 3B shows example exogenous miR219a-5p target sequences (or seed-complementary target sequences) (CTAACAGGT (miRNA target 4), TAACAGGTT (miRNA target 5), ACTAACAGG (miRNA target 6)) that are complementary to miR219a-5p (SEQ ID NO: 9) and were inserted in or adjacent to example 2k, 5′ UTR, or 3′ UTR modification locations of the recombinant zika virus genome. In one modification, miRNA target 4 was inserted into the 2k region and flanked by AvaIII sites to give a modified 2k region (SEQ ID NO: 36). In one modification, miRNA target 5 was inserted into the 2k region and flanked by AvaIII sites to give a modified 2k region (SEQ ID NO: 37). In one modification, miRNA target 6 was inserted adjacent to the 5′ UTR region and flanked by BsiWI sites (SEQ ID NO: 38). In one modification, miRNA target 4 was inserted adjacent to the 3′ UTR region and flanked by MluI sites (SEQ ID NO: 39).

FIG. 3C shows example exogenous miR-129-5p target sequences (or seed-complementary target sequences) (AAAAACGCC (miRNA target 1), AAACGCCAG (miRNA target 2)) that are complementary to miR-129-5p (SEQ ID NO: 8) and were inserted in or adjacent to example 2k, 5′ UTR or 3′ UTR modification locations of the recombinant zika virus genome (SEQ ID NO: 40, SEQ ID NO: 41, SEQ ID NO: 42, SEQ ID NO: 43). In one modification, miRNA target 1 was inserted into the 2k region and flanked by AvaIII sites to give a modified 2k region (SEQ ID NO: 40). In one modification, miRNA target 2 was inserted into the 2k region and flanked by AvaIII sites to give a modified 2k region (SEQ ID NO: 41). In one modification, miRNA target 1 was inserted adjacent to the 5′ UTR region and flanked by BsiWI sites (SEQ ID NO: 42). In one modification, miRNA target 1 was inserted adjacent to the 3′ UTR region and flanked by MluI sites (SEQ ID NO: 43).

FIG. 3D shows example exogenous miR 1225-5p target sequences (or seed-complementary target sequences) (CACCCATGC (miRNA target 8), ACCCATGCC (miRNA target 10), CCCATGCCG (miRNA target 9) that are complementary to miR 1225-5p (SEQ ID NO: 11) and were inserted in or adjacent to example 2k, 5′ UTR, or 3′ UTR modification locations of the recombinant zika virus genome. In one modification, miRNA target 8 was inserted into the 2k region and flanked by AvaIII sites to give a modified 2k region (SEQ ID NO: 45). In one modification, miRNA target 10 was inserted into the 2k region and flanked by AvaIII sites to give a modified 2k region (SEQ ID NO: 46). In one modification, miRNA target 9 was inserted adjacent to the 5′ UTR region and flanked by BsiWI sites (SEQ ID NO: 47). In one modification, miRNA target 10 was inserted adjacent to the 3′ UTR region and flanked by MluI sites to give (SEQ ID NO: 48).

FIG. 3E shows an example miR377-3p target sequence (or seed-complementary target sequence) (AGTGTGTTT (miRNA target 11)) that is complementary to miR377-3p ((SEQ ID NO: 12) and was inserted in or adjacent to example 2k, 5′ UTR or 3′ UTR modification locations of the recombinant zika virus genome. In one modification, miRNA target 11 was inserted into the 2k region and flanked by AvaIII sites to give a modified 2k region (SEQ ID NO: 49). In one modification, miRNA target 11 was inserted adjacent to the 5′ UTR region and flanked by BsiWI sites (SEQ ID NO: 50). In one modification, miRNA target 11 was inserted adjacent to the 3′ UTR region and flanked by MluI sites (SEQ ID NO: 51).

FIGS. 4A-4B illustrate an example recombinant zika virus (oZIKV_0P) (pCC1-ZIKV-0P, Table 8, Table 9B) generation and effect in cancer cell lines. FIG. 4A shows the recombinant zika virus derived from an in vitro transcription and Vero cell transfection. FIG. 4B shows the recombinant zika virus oncolytic effect in medulloblastoma cell lines 48 hours after infection.

FIGS. 5A-5B illustrate the cytotoxicity of recombinant zika virus (oZIKV_0P) (pCC1-ZIKV-0P, Table 8, Table 9B) in glioblastoma and medulloblastoma cell lines. FIG. 5A shows the glioblastoma cells (U138MG, U251MG, and U343MG cell lines) and the medulloblastoma cells (ONS-76 and Daoy cell lines) viability decrease after recombinant zika virus infection in MOI 2 when compared with control (CT). FIG. 5B shows a comparison of the infection of recombinant zika virus oZIKV_0P as compared with the African wild-type zika virus in medulloblastoma cells (Daoy).

FIG. 6 illustrates a representative zika Replicon plasmid (pRep) comprising a T7 promoter, hammerhead ribozyme, 5′ UTR, enhanced green fluorescent protein-coding region (“EGFP”), portions of the ZIKV genome (last 60 bases of NS4A, first 60 bases of NS4B) flanking HA (hemagglutinin), a luciferase-coding region (“Nluc”), 3′ UTR, HDV ribozyme, multiple cloning site (MCS), a bGH poly(A) signal; and a recombinant zika virus comprising ZIKV genome (NS4A, NS4B). The miRNA target sequence may be positioned within the pRep or recombinant zika virus.

FIGS. 7A-7F show proof-of-concept of miRNA regulation in the target sequence inserted in a zika Replicon plasmid pRep. FIG. 7A shows the results of NanoLuc reporter assays of Vero cells having the pRep with different miRNA target inserts. FIGS. 7B-7F show the construct maps of pRep constructs used in the NanoLuc reporter assays with their respective miRNA target sequence (FIG. 7B: pRep-129-5p-BsiWi-1c comprising miR 129-5p (1c) target sequence; FIG. 7C: pRep-129-5p-BsiWI-1e comprising miR 129-5p (1e) target sequence; FIG. 7D: pRep-219a-2-3p-BsiWI-2b comprising miR219a-2-3p (2b) target sequence; FIG. 7E: pRep-377-3p-BsiWI-4c comprising miR377-5p (4c) target sequence; FIG. 7F: pRep-377-3p-BsiWI-4e comprising miR377-5p (4e) target sequence; see Tables 7 and 9A-9B for construct details). The overexpression of miR-129-5p decreased NanoLuc expression, confirming the inhibitory mechanism in pRep with the corresponding miRNA target sequence designed (miRNA target 1, miRNA target 2).

FIG. 8 shows the results of NanoLuc reporter assays of CHLA cells having the pRep with different miRNA target inserts (see description of FIGS. 7A-7F for pRep construct details). The overexpression of miRNAs decreased NanoLuc expression of all pREP, confirming the inhibitory mechanism in all pRep designed.

FIG. 9 shows a representative plasmid pCC1-ZIKV-0P comprising a 5′ UTR, a ZIKV genome, ribozymes (e.g., hammerhead and HDV), T7 promoter, and 3′ UTR.

FIG. 10 shows an example plasmid with a miRNA target inserted adjacent to the 5′ UTR region using BsiWI sites.

FIG. 11 shows an example plasmid with a miRNA target inserted adjacent to the 5′ UTR region using BsiWI and XhoI sites.

FIG. 12 shows an example plasmid with an exogenous miRNA target sequence inserted into the 2k region using AarI sites (SEQ ID NO: 52, SEQ ID NO: 53, SEQ ID NO: 54). The exogenous polynucleotide sequence (SEQ ID NOs: 226, 227) comprises two restriction sites at the 5′ and 3′ ends.

FIG. 13 shows a representative image of a plasmid pCC1-ZIKV-2P having ZIKV genome components, ribozymes (e.g., hammerhead and HDV), the T7 promoter near the 5′ UTR, 3′ UTR, and miRNA target 219a-2-3p inserted into the 2k region.

FIG. 14 shows a representative image of a plasmid pCC1-ZIKV-3P having ZIKV genome components, ribozymes (e.g., hammerhead, HDV), the T7 promoter near the 5′ UTR, 3′ UTR, and miRNA target miR 129-5p inserted between the NS5 and the 3′ UTR.

FIG. 15 shows a representative image of a plasmid pCC1-ZIKV-5P-T7 having ZIKV genome components, ribozymes (e.g., hammerhead, HDV), the T7 promoter next to the 5′ UTR, 3′ UTR, and miRNA target miR-1225 inserted between the 5′ UTR and the ancC.

FIG. 16 shows an image of RNA gel electrophoresis of samples analyzed using the T7-HIGH kit (first three wells from the left), where samples were pretreated with DNase, and the HiScribe™ T7 ARCA mRNA Kit (three wells from the right), and the RNA ladder in the left-most column. The bands confirm the efficiency of in vitro transcription with aa production of an expressive amount of RNA.

FIG. 17 shows a comparison of the concentration of ZIKV copies created (copies/μL) at various time points in the cell pellet and supernatant. The graph confirms the same amount of the beginning and the end of the ZIKV genome, confirming the presence of the entire ZIKV genome after in vitro transcription.

FIG. 18 shows micrographs of Vero cell cultures transfected with modified ZIKV RNA (TI_14, TI_15, TI_16, TI_17, TI_18) over the course of 1-, 4-, 5-, and 6-days post infection. The cytopathic effect (*) was observed in all RNA samples (TI 14-18) 5-6 days after re-infection, confirming active virus production.

FIG. 19 shows antigen test results of the supernatant of the Vero cells transfected with modified ZIKV RNA (pCC1-ZIKV-0P “0p”, pCC1-ZIKV-2P-T7 “2p”, pCC1-ZIKV-3P-T7 “3p”), each showing the presence of zika virus in the supernatant.

FIGS. 20A and 20B show cell viability test results of CHLA-06-ATRT cells 3 days post infection (FIG. 20A) and 5 days post infection (FIG. 20B) with wild-type or recombinant zika viruses (oZIKV_4 (pCC1-ZIKV-0P), oZIKV_16 (pCC1-ZIKV-0P), oZIKV_5 (pCC1-ZIKV-2P-T7), oZIKV_14 (pCC1-ZIKV-2P-T7), oZIKV_17 (pCC1-ZIKV-2P-T7), oZIKV_6 (pCC1-ZIKV-3P-T7), oZIKV_15 (pCC1-ZIKV-2P-T7), oZIKV_18 (pCC1-ZIKV-2P-T7), see Table 10). Four recombinant viruses (oZIKV_4, oZIKV_14, oZIKV_17, oZIKV_18) kept their oncolytic effect in CHLA cell line 5 days post infection.

FIG. 21 shows micrographs of neural progenitor cells (NPC) as models for testing the safety of recombinant zika viruses (oZIKV_4, oZIKV_14, oZIKV_17, oZIKV_18) in non-tumoral cells. NPC cells were transfected with wild-type zika virus and recombinant zika viruses, and cell cultures were imaged 5 days post-infection. The results indicate that the oZIKV_14 and oZIKV_18 recombinant viruses demonstrated an oncolytic effect on CHLA tumor cells (FIGS. 20A-20B) while exhibiting little to no cytotoxic effect on normal NPCs (FIGS. 21-22B). NPCs transfected with wild-type Zika and oZIKV_17 decreased viability in tumor cells. By contrast, the two recombinant zika viruses oZIKV_14 and oZIKV_18 illustrate that these recombinant viruses allowed for NPC cells to survive (i.e., are safe in normal cells).

FIGS. 22A-22B show neurosphere analysis of NPCs transfected with wild-type zika virus and recombinant zika viruses, where neurosphere area (FIG. 22A) and perimeter (FIG. 22B) were measured 5 days post infection.

FIG. 23 shows the results of cell viability assays of medulloblastoma cells (Daoy) and ATRT cells (CHLA) infected with recombinant zika viruses having a miRNA target insert (oZIKV_14) after miRNA modulation by cell transfection 24h before virus infection. These results show that the oZIKV_14 replication inhibition after miR-219a-2-3p overexpression by target sequence ligation, confirms the recombinant viruses' inhibition mechanism functionality.

FIG. 24 shows the results of cell viability assays of cerebral microvessels (hCMEC) infected with recombinant zika viruses having a miRNA target insert (oZIKV14) and wild-type zika virus at MOI 5, three days after infection. The relative viability did not decrease after oZIKV_14 infection, confirming the safety of recombinant viruses oZIKV_14 in normal cerebral microvessels cell lines, in comparison with the wild-type ZIKV. The sample is statistically different from the mock, by ANOVA multiple comparisons, when: a p<0.0001.

FIG. 25 shows the results of cell viability assays of microglia cells (hMC3) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and wild-type virus decreased the relative viability in microglia cell line after infection, confirming immunoresponse after cell infection. The sample was statistically different from the MOCK, by ANOVA multiple comparisons, when: a p<0.0001; b p<0.0009.

FIG. 26 shows the results of cell viability assays of testis cells (Hs1.Tes) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and wild-type virus decreased the relative viability in the testis cell line after infection.

FIG. 27 shows the results of cell viability assays of medulloblastoma cells (Daoy) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and Wild-type virus decreased the relative viability in the Daoy cell line after infection, confirming the oncolytic effect in the medulloblastoma cell line. The sample is statistically different from the MOCK, by ANOVA multiple comparisons, when: a p<0.0001.

FIG. 28 shows the results of cell viability assays of medulloblastoma cells (ONS) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and wild-type virus decreased the relative viability in the ONS cell line after infection, confirming the oncolytic effect in the medulloblastoma cell line. The sample was statistically different from the MOCK, by ANOVA multiple comparisons, when: a p<0.0001.

FIG. 29 shows the results of cell viability assays of ATRT cells (CHLA-06-ATRT) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and Wild-type virus decreased the relative viability in the CHLA-06-ATRT cell line after infection, confirming the oncolytic effect in the medulloblastoma cell line. The sample was statistically different from the MOCK, by ANOVA multiple comparisons, when: a p<0.0001.

FIG. 30 shows the results of cell viability assays of glioblastoma cells (U138 and LN18) infected with recombinant zika viruses having a miRNA target insert (oZIKV14) and wild-type ZIKA virus at MOI 5 and MOI 1.667, three days after infection. The oZIKV_14 decreased the relative viability in the lowest MOIs (virus concentration) in both glioblastoma cell lines after infection, when compared with the wild-type ZIKV, confirming the highest oncolytic effect of oZIKV_14. The sample was statistically different from the MOCK, by ANOVA multiple comparisons, when: a p<0.0001; d p<0.05.

FIG. 31 shows the results of cell viability assays of prostate tumor cells (DU-145) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and Wild-type virus decreased the relative viability in the DU-145 cell line after infection, confirming the oncolytic effect in the prostate tumor cell line. The sample was statistically different from the MOCK, by ANOVA multiple comparisons, when: a p<0.0001.

FIG. 32 shows the results of cell viability assays of triple negative breast tumor cells (MDA-MB-231) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and Wild-type virus decreased the relative viability in the MDA-MB-231 cell line after infection, confirming the oncolytic effect in triple-negative breast tumor cell line. The sample was statistically different from the MOCK, by ANOVA multiple comparisons, when: a p<0.0001; b p<0.0009; c p<0.009.

FIG. 33 shows the results of cell viability assays of colon tumor cells (hCT-8) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and wild-type virus did not decrease the relative viability in the hCT-8 cell line after infection, confirming the oncolytic effect in colon tumor cell line. The sample is statistically different from the mock, by ANOVA multiple comparisons, when: a p<0.0001; c p<0.009; d p<0.05.

FIG. 34 shows the results of cell viability assays of luminal breast tumor cells (MCF7) infected with recombinant zika viruses having a miRNA target insert (oZIKV14) and wild-type ZIKA virus at MOI 5, 1.667, 0.556, 0.185, 0.062, 0.021, 0.007, 0.002, three days after infection. Both oZIKV_14 and wild-type virus did not decrease the relative viability in the MCF7 cell line after infection, confirming the oncolytic effect in luminal breast tumor cell line. The sample was statistically different from MOCK, by ANOVA multiple comparisons, when: a p<0.0001; b p<0.0009; c p<0.009; d p<0.05.

FIG. 35 shows the oncolytic effect of oZIKV14 on the development of central nervous system tumors and their metastases in the spinal cord. Control=animals that received intracerebroventricular (i.c.v.) vehicle injection; zika=animals that received i.c.v. of zika virus (6×10³ PFU). T0=images demonstrating the existence and location of tumors before the injection; T1=images of the same animals, one-week post-injection; T2=images of the same animals, two-week post-injection. Injection of oZIKV_14 caused partial and total remission of cerebral and metastatic tumors, confirming in vivo effectiveness of oZIKV_14 against CNS tumors.

### DETAILED DESCRIPTION OF THE INVENTION

In one aspect, viruses are provided that impair cell division, cell growth, and/or induce cell death in cancerous cells, referred to in some embodiments as oncolytic viruses. A non-limiting example of an oncolytic virus is a recombinant zika virus as described herein. Another aspect of this disclosure relates to methods of cancer treatment comprising administering a recombinant zika virus as described herein. In some embodiments, the oncolytic virus does not replicate in non-cancerous cells. In some embodiments, the oncolytic virus does not impair cell division, cell growth, and/or induce cell death in non-cancerous cells. In some embodiments, the non-cancerous cells express a microRNA (miRNA), and the oncolytic virus comprises a target of the miRNA such that the miRNA is capable of binding to the target. The cancerous cells may not express the miRNA, or express less of the miRNA than the non-cancerous cells. A non-limiting example of a non-cancerous cell is a neuron. Further provided are nucleic acids and constructs for preparation of viruses, such as oncolytic viruses herein.

Before the present methods and compositions are described, it is to be understood that this disclosure is not limited to a particular method or composition described, and as such may vary. Unless defined otherwise, all technical and scientific terms used herein have the same meaning as commonly understood by one of ordinary skill in the art. All publications mentioned herein are incorporated by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Where a range of values is provided, unless otherwise indicated, each intervening value to the tenth of the unit of the lower limit between the upper and lower limits of that range is also specifically disclosed. Each smaller range between any stated value or intervening value in a stated range is encompassed herein. The upper and lower limits of these smaller ranges may independently be included or excluded in the range, and each range where either, neither or both limits are included in the smaller ranges is also encompassed herein, subject to any specifically excluded limit in the stated range. Where the stated range includes one or both of the limits, ranges excluding either or both of those included limits are also included.

The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value, such as +10% of the value modified by the term “about”.

The terms “individual,” “patient,” or “subject” can be used interchangeably. None of the terms require or are limited to a situation characterized by the supervision (e.g. constant or intermittent) of a health care worker (e.g. a doctor, a registered nurse, a nurse practitioner, a physician's assistant, an orderly, or a hospice worker). In some embodiments, patients, subjects, or individuals can be under the supervision of a health care worker. The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “heterologous nucleic acid sequence,” or “exogenous nucleic acid sequence,” or “transgenes,” as used herein, in relation to a specific virus can refer to a nucleic acid sequence that originates from a source other than the specified virus.

The terms “inhibiting,” “reducing” or “prevention,” or any variation of these terms, referred to herein, can include any measurable decrease or complete inhibition to achieve a desired result.

A “promoter,” as used herein, can be a control sequence that is a region of a nucleic acid sequence at which initiation and rate of transcription are controlled. In certain embodiments, a promoter may contain genetic elements at which regulatory proteins and molecules may bind such as RNA polymerase and other transcription factors. The terms “operatively positioned,” “operatively linked,” “under control” and “under transcriptional control” can mean that a promoter is in a correct functional location and/or orientation in relation to a nucleic acid sequence to control transcriptional initiation and/or expression of that sequence. In certain embodiments, a promoter may or may not be used in conjunction with an “enhancer,” which refers to a cis-acting regulatory sequence involved in the transcriptional activation of a nucleic acid sequence.

Percent (%) sequence identity with respect to a reference polypeptide or polynucleotide sequence is the percentage of amino acid or nucleotide residues in a candidate sequence that are identical with the amino acid or nucleotide residues in the reference polypeptide or polynucleotide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid or polynucleotide sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid or polynucleotide sequence comparisons, the % amino acid or polynucleotide sequence identity of a given sequence A to, with, or against a given sequence B (which can alternatively be phrased as a given sequence A that has or comprises a certain % sequence identity to, with, or against a given sequence B) is calculated as follows: 100 times the fraction X/Y, where X is the number of residues scored as identical matches by the sequence alignment program ALIGN-2 in that program's alignment of A and B, and where Y is the total number of residues in B. It will be appreciated that where the length of sequence A is not equal to the length of sequence B, the % sequence identity of A to B will not equal the % sequence identity of B to A. Unless specifically stated otherwise, all % sequence identity values used herein are obtained as described in the immediately preceding paragraph using the ALIGN-2 computer program.

The terms “treat,” “treating,” and “treatment” can be meant to include alleviating or abrogating a disorder, disease, or condition; or one or more of the symptoms associated with the disorder, disease, or condition; or alleviating or eradicating the cause(s) of the disorder, disease, or condition itself. Desirable effects of treatment can include but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” can refer to the amount of a compound that, when administered, can be sufficient to prevent the development of, or alleviate to some extent, one or more of the symptoms of the disorder, disease, or condition being treated. The term “therapeutically effective amount” can also refer to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” can refer to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21^{st} Edition; Lippincott Williams & Wilkins: Philadelphia, PA, 2005; Handbook of Pharmaceutical Excipients, 5^{th} Edition; Rowe et al., Eds., The Pharmaceutical Press and the American Pharmaceutical Association: 2005; and Handbook of Pharmaceutical Additives, 3^{rd} Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” can refer to a mixture of a compound disclosed herein with other chemical components, such as diluents or carriers. The pharmaceutical composition can facilitate the administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, and the like.

An “anti-cancer agent,” as used herein, can refer to an agent or therapy that is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Non-limiting examples of anti-cancer agents can include biological agents (biotherapy), chemotherapy agents, and radiotherapy agents.

The term “oncolytic,” as used herein, can refer to the killing of cancer or tumor cells by an agent, through the direct lysis of said cells, by stimulating immune response towards said cells, apoptosis, expression of toxic proteins, autophagy, and shut-down of protein synthesis, induction of anti-tumoral immunity, or any combinations thereof. The direct lysis of cancer or tumor cells infected by the agent can be a result of replication of the virus within said cells. In certain examples, the term “oncolytic,” can refer to the killing of cancer or tumor cells without lysis of said cells.

The term “oncolytic virus” as used herein can refer to a virus that preferentially infects and kills tumor cells. Under certain non-limiting circumstances, it is understood that oncolytic viruses can promote anti-tumor responses through dual mechanisms dependent on not only the selective killing of tumor cells but also the stimulation of host anti-tumor immune responses.

In some embodiments, as used here a “derivative” of a polypeptide or polynucleotide refers to a sequence at least 80%, 81%, 82%, 83%, 84%, 85%, 86%, 87%, 88%, 89%, 90%, 91%, 92%, 93%, 94%, 95%, 96%, 97%, 98% or 99% identical to the polypeptide or polynucleotide, respectively. In some embodiments, as used here a “derivative” of a polypeptide or polynucleotide refers to a sequence at least 80%, 81%, 82%, 83%, 84%, 85%, 86%, 87%, 88%, 89%, 90%, 91%, 92%, 93%, 94%, 95%, 96%, 97%, 98% or 99% homologous to the polypeptide or polynucleotide, respectively. In some embodiments, as used herein, a “derivative” of a polypeptide or polynucleotide refers to a sequence with no or no more than 1, 2, 3, 4, 5, 6, 7, 8, 9, or 10 amino acid or nucleotide substitutions, insertions, or deletions as compared to the polypeptide or polynucleotide, respectively.

### Oncolytic Viruses and Nucleic Acid Compositions

In one aspect, provided herein are zika viruses and methods of treating cancer comprising administering to a subject in need thereof a zika virus. In some embodiments, the zika virus is a recombinant zika virus. In some embodiments, the zika viruses are produced from a nucleic acid composition described herein.

In some embodiments, zika viruses herein have selectivity for cells undergoing cell division, such as cancerous cells, and as such are useful for killing said cancerous cells and/or inhibiting the growth of a tumor. Accordingly, an oncolytic virus provided herein is not limited to a virus having a lytic effect on cancerous cells, and instead encompasses any virus that acts on a cancer cell to inhibit cell growth, prevent cell growth, inhibit cell replication, prevent cell replication, cause cell death, or any combination thereof. The oncolytic virus may be a clinical isolate of a virus, or a clone or recombinant virus derived or engineered therefrom.

In some embodiments, zika viruses herein have tropism for cells of the central nervous system. As a non-limiting example, the zika virus has tropism for cells in the central nervous system, e.g., brain cancer cells.

In another aspect, a composition is provided comprising one or more portions of a zika virus. For example, such portions of the virus include nucleic acids (e.g. nucleic acids coding for the zika virus or portions thereof) and proteins of the virus. Non-limiting examples of viral proteins include capsid proteins, membrane proteins, non-structural proteins, and envelope proteins. Non-limiting examples of viral nucleic acids include protein coding sequences and non-protein coding sequences.

In some embodiments, a nucleic acid composition is provided encoding a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 1. In some embodiments, a nucleic acid composition is provided encoding a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 2. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 3. In some embodiments, a nucleic acid composition is provided encoding a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 4. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 5. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 6A. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence complementary to one or more sequences of Table 6B. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more recombinant zika virus constructs of Table 8. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more recombinant zika virus constructs of Table 9A. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 9B. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more recombinant zika virus constructs of Table 10. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of a zika virus from Table 12. In some embodiments, a nucleic acid composition is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of a zika virus from Table 13.

In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 1. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 2. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 3. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 4. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 5. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 6A. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence complementary to one or more sequences of Table 6B. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more recombinant zika virus constructs of Table 8. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more recombinant zika virus constructs of Table 9A. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of Table 9B. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more recombinant zika virus constructs of Table 10. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of a zika virus from Table 12. In some embodiments, a recombinant zika virus is provided comprising a sequence at least 80%, 81%, 82%, 83%, 84%, 85%, 86%, 87%, 88%, 89%, 90%, 91%, 92%, 93%, 94%, 95%, 96%, 97%, 98% or 99% identical to one or more sequences of a zika virus from Table 13.

In some embodiments, a recombinant zika virus described herein is one comprising a nucleic acid sequence that is derived from a zika viral genome, for example, by mutation, insertion, and/or deletion of one or more nucleobases. An insertion includes a nucleic acid sequence encoding for one or more proteins. An insertion also includes a promoter, such as a promoter inducible in mammalian cells. A deletion includes the removal of a coding sequence that allows for replication of the virus. Accordingly, in some cases, the recombinant zika virus described herein indicates the virus has been modified by the introduction of a heterologous nucleic acid or protein and/or the alteration of a native nucleic acid sequence.

In some embodiments, a recombinant zika virus described herein comprises one or more molecules, such as an exogenous nucleic acid, that impart to the virus an enhanced level of tumor cell specificity. In this way, the virus is targeted to specific tumor types using tumor cell-specific molecules or biomarkers. In some embodiments, the recombinant zika virus comprises an exogenous nucleic acid complementary to a miRNA. The miRNA may be expressed in non-tumor cells, and not expressed in tumor cells. The miRNA may be expressed more in non-tumor cells than in tumor cells, for instance, the miRNA is expressed at least 2-fold more in non-tumor cells than in tumor cells. A non-limiting example of a non-tumor cell is a neuron. In some embodiments, the recombinant zika virus is an oncolytic zika virus having oncolytic activity, and the recombinant zika virus retains oncolytic activity with the exogenous nucleic acid. For example, if the exogenous nucleic acid is inserted within the polynucleotide encoding the 2k protein, the 2k protein is still expressed and the zika virus retains oncolytic activity. As another example, if the exogenous nucleic acid is inserted between the 5′ UTR and the polynucleotide encoding for the ancC/C protein, the ancC/C protein is still expressed and the zika virus retains oncolytic activity. As another example, if the exogenous nucleic acid is inserted between the 3′ UTR and the polynucleotide encoding for the nonstructural protein NS5, the NS5 protein is still expressed and the zika virus retains oncolytic activity.

In some embodiments, a recombinant zika virus described herein comprises a nucleic acid sequence encoding for a polypeptide that is heterologous to the virus, where the polypeptide is expressible under an inducible promoter. As such, the virus may also be an expression vector from which the polypeptide may be expressed. For example, the recombinant zika virus described herein comprises a suicide gene expressible under an inducible promoter, such as CMV. Methods of cancer treatment involving the recombinant zika virus described herein optionally comprise inducing expression of the suicide gene to kill or otherwise prevent the virus from propagating. Accordingly, the viral infection may be controlled by a physician according to the needs of the patient.

In some embodiments, a recombinant zika virus described herein comprises a genetic modification that affects the expression of a viral gene. For example, a mutation in a virulence gene that contributes to the pathogenicity of the virus to a host organism such that the expression of that gene is significantly decreased, or wherein the gene product is rendered nonfunctional, or its ability to function is significantly decreased. In some cases, the genetic modification compromises the ability of the virus to replicate or to replicate in non-cancerous or non-dividing cells.

In some embodiments, a recombinant zika virus described herein comprises at least one viral protein necessary for viral replication under the control of a tumor-specific promoter. In some embodiments, a gene that encodes a cytotoxic agent is placed under the control of a tumor-specific promoter. For example, cytotoxic agents include toxins, prodrugs, cytokines, and chemokines.

In some cases, a recombinant zika virus described herein includes at least about 10, 20, 30, 40, 50, 60, 70, 80, 90, 100, 120, 140, 160, 180, 200, 500, 1000, 5000, 10000, or 11000 nucleobases of the wild-type zika genome. These nucleobases do not necessarily have 100% sequence identity to the wild-type zika virus and may vary, e.g., the identity is at least about 80%, 85%, 90%, 95%, or 98%. In some cases, a portion refers to a region of the virus that encodes for one or more proteins. Additionally, or alternatively, a portion refers to a region of the virus that does not encode for a protein.

Further provided herein is a nucleic acid composition comprising the genome of a recombinant zika virus described herein. Another aspect relates to a host cell comprising a recombinant zika virus described herein.

In some embodiments, a recombinant zika virus can exhibit enhanced intratumoral and intertumoral spreading, enhanced immune evasion, enhanced tumor-specific replication, and/or enhanced tumor-targeted delivery. In some embodiments, a recombinant zika virus as described herein exhibits enhanced intratumoral and intertumoral spreading, enhanced immune evasion, enhanced tumor-specific replication, and enhanced tumor-targeted delivery.

Zika virus produces highly abundant noncoding RNA known as subgenomic flaviviral RNA (sfRNA) in infected cells. In some embodiments, a recombinant zika virus produces higher level of sfRNA than a wild-type zika virus in a cancer cell of the subject. In some embodiments, the higher level of sfRNA is by at least 10%, at least 20%, at least 30%, at least 40% at least 50%, at least 60%, at least 70%, at least 80%, at least 90%, at least 100%.

In some embodiments, a recombinant zika virus described herein comprises a 3′ UTR to increase sfRNA production, for example, as a result of a longer stall of exoribonuclease XRN1 in the cancer cell. In some embodiments, a recombinant zika virus described herein comprises a 5′ UTR to increase sfRNA production, for example, as a result of a longer stall of exoribonuclease XRN1 in the cancer cell. In some embodiments, a recombinant zika virus described herein comprises a 5′ UTR and a 3′ UTR to increase sfRNA production, for example, as a result of a longer stall of exoribonuclease XRN1 in the cancer cell.

In some embodiments, methods of administering a recombinant zika virus further comprise administering to a subject in need thereof an extra exoribonuclease-resistant sfRNA.

In some embodiments, a recombinant zika virus is produced from a nucleic acid composition comprising a polynucleotide encoding a zika virus capsid protein C or the derivative of the zika virus C, a polynucleotide encoding a zika virus viral membrane protein (prM/M) or the derivative of the zika virus prM/M, a polynucleotide encoding a zika virus viral envelope protein (E) or the derivative of the zika virus E, or any combination thereof. Non-limiting example sequences include those of Tables 5 and 9B.

In certain embodiments, a recombinant zika virus is produced from a nucleic acid composition comprising a polynucleotide encoding a zika virus C or the derivative of the zika virus C only. Accordingly, in some embodiments the recombinant zika virus described herein is generated from expressing such nucleic acid composition in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus.

In certain embodiments, a recombinant zika virus is produced from a nucleic acid composition comprising a polynucleotide encoding a zika virus prM/M or the derivative of the zika virus prM/M only. Accordingly, in some embodiments the recombinant zika virus described herein is generated from expressing such nucleic acid composition in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus.

In certain embodiments, a recombinant zika virus is produced from a nucleic acid composition comprising a polynucleotide encoding a zika virus viral E or the derivative of the zika virus E only. Accordingly, in some embodiments the recombinant zika virus described herein is generated from expressing such nucleic acid composition in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus.

In certain embodiments, a recombinant zika virus is produced from a nucleic acid composition comprising a polynucleotide encoding a zika virus C or the derivative of the zika virus C and a polynucleotide encoding a zika virus prM/M or the derivative of the zika virus prM/M only. Accordingly, in some embodiments the recombinant zika virus described herein is generated from expressing such nucleic acid composition in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus.

In certain embodiments, a recombinant zika virus is produced from a nucleic acid composition comprising a polynucleotide encoding a zika virus C or the derivative of the zika virus C and a polynucleotide encoding a zika virus viral E or the derivative of the zika virus E only. Accordingly, in some embodiments the recombinant zika virus described herein is generated from expressing such nucleic acid composition in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus.

In certain embodiments, a recombinant zika virus is produced from a nucleic acid composition comprising a polynucleotide encoding a zika virus prM/M or the derivative of the zika virus prM/M and a polynucleotide encoding a zika virus viral E or the derivative of the zika virus E only. Accordingly, in some embodiments the recombinant zika virus described herein is generated from expressing such nucleic acid composition in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus.

In some embodiments, a nucleic acid composition herein comprises the polynucleotide encoding the zika virus C, the polynucleotide encoding the zika virus prM/M, and the polynucleotide encoding the zika virus E. When three structural proteins are expressed as the nucleic acid composition described herein, in some embodiments, they could be expressed in one nucleic acid. When three structural proteins are expressed as the nucleic acid composition described herein, in other embodiments, they could be expressed in more than one nucleic acid.

In some embodiments, the polynucleotide encoding the derivative of the zika virus C comprises at least one substitution, at least one deletion, and/or at least one insertion as compared to a wild-type zika virus C. In some embodiments, the zika virus C or the derivative of the zika virus C comprises an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 1.

TABLE 1
Example sequences

| Name | |
|---|---|
| (SEQ ID NO) | Sequence |
| ZIKA virus ancC | MKNPKKKSGGFRIVNMLKRGVARVSPFGGLKRLPAGLLLGHGPIRM |
| (SEQ ID NO: 1) | VLAILAFLRFTAIKPSLGLINRWGSVGKKEAMEIIKKFKKDLAAMLRI |
| | INARKEKKRRGADTSVGIVGLLLTTAMA |
| ZIKA virus C | MKNPKKKSGGFRIVNMLKRGVARVSPFGGLKRLPAGLLLGHGPIRM |
| (SEQ ID NO: 2) | VLAILAFLRFTAIKPSLGLINRWGSVGKKEAMEIIKKFKKDLAAMLRI |
| | INARKEKKRR |

In some embodiments, the polynucleotide encoding the derivative of the zika virus prM/M comprises at least one substitution, at least one deletion, and/or at least one insertion as compared to a wild-type zika virus prM/M. In some embodiments, the zika virus prM/M or the derivative of the zika virus prM/M comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 2.

TABLE 2
Example sequences

| Name | |
|---|---|
| (SEQ ID NO) | Sequence |
| ZIKA virus prM | AEVTRRGSAYYMYLDRNDAGEAISFPTTLGMNKCYIQIMDLGHMC |
| (SEQ ID NO: 3) | DATMSYECPMLDEGVEPDDVDCWCNTTSTWVVYGTCHHKKGEAR |
| | RSRRAVTLPSHSTRKLQTRSQTWLESREYTKHLIRVENWIFRNPGFA |
| | LAAAAIAWLLGSSTSQKVIYLVMILLIAPAYS |
| ZIKA virus M | AVTLPSHSTRKLQTRSQTWLESREYTKHLIRVENWIFRNPGFALAAA |
| (SEQ ID NO: 4) | AIAWLLGSSTSQKVIYLVMILLIAPAYS |

In some embodiments, the polynucleotide encoding the derivative of the zika virus E comprises at least one substitution, at least one deletion, and/or at least one insertion as compared to a wild-type zika virus E. In some embodiments, the polynucleotide encoding E is translated into a wild zika virus type E. In some embodiments, the zika virus E or the derivative of the zika virus E comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to sequence of Table 3.

TABLE 3
Example sequence

| Name | |
|---|---|
| (SEQ ID NO) | Sequence |
| ZIKA virus E | IRCIGVSNRDFVEGMSGGTWVDIVLEHGGCVTVMAQDKPTVDIELV |
| (SEQ ID NO: 5) | TTTVSNMAEVRSYCYEASISDMASDSRCPTQGEAYLDKQSDTQYVC |
| | KRTLVDRGWGNGCGLFGKGSLVTCAKFACSKKMTGKSIQPENLEY |
| | RIMLSVHGSQHSGMIVNDTGHETDENRAKVEITPNSPRAEATLGGFG |
| | SLGLDCEPRTGLDFSDLYYLTMNNKHWLVHKEWFHDIPLPWHAGA |
| | DTGTPHWNNKEALVEFKDAHAKRQTVVVLGSQEGAVHTALAGALE |
| | AEMDGAKGRLSSGHLKCRLKMDKLRLKGVSYSLCTAAFTFTKIPAE |
| | TLHGTVTVEVQYAGTDGPCKVPAQMAVDMQTLTPVGRLITANPVIT |
| | ESTENSKMMLELDPPFGDSYIVIGVGEKKITHHWHRSGSTIGKAFEA |
| | TVRGAKRMAVLGDTAWDFGSVGGALNSLGKGIHQIFGAAFKSLFG |
| | GMSWFSQILIGTLLMWLGLNTKNGSISLMCLALGGVLIFLSTAVSA |

In some embodiments, the nucleic acid composition comprises a 5′ untranslated region (5′ UTR) of the zika virus. In some embodiments, the nucleic acid composition comprises a 3′ untranslated region (3′ UTR) of the zika virus. Non-limiting example untranslated regions includes those from the viruses disclosed in Tables 4 and 12-13. In some embodiments, the 5′ untranslated region (5′ UTR) of the zika virus comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 4 or Table 12.

TABLE 4
Example zika UTR sequences

| Name | |
|---|---|
| (SEQ ID NO) | Sequence |
| ZIKA 5′ UTR | AGTTGTTGATCTGTGTGAATCAGACTGCGACAGTTCGAGTTTGAA |
| (SEQ ID NO: 6) | GCGAAAGCTAGCAAAGTATCAACAGGTTTTATTTTGGATTTGGAA |
| | ACGAGAGTTTCTGGTC |
| ZIKA 3′ UTR | GCACCAATCTTAATGTTGTCAGGCCTGCTAGTCAGCCACAGCTTG |
| (SEQ ID NO: 7) | GGGAAAGCTGTGCAGCCTGTGACCCCCCCAGGAGAAGCTGGGAA |
| | ACCAAGCCTATAGTCAGGCCGAGAACGCCATGGCACGGAAGAAG |
| | CCATGCTGCCTGTGAGCCCCTCAGAGGACACTGAGTCAAAAAAC |
| | CCCATGCGCTTGGAGGCGCAGGATGGGAAAAGAAGGTGGCGACC |
| | TTCCCCACCCTTCAATCTGGGGCCTGAACTGGAGATCAGCTGTGG |
| | ATCTCCAGAAGAGGGACTAGTGGTTAGAGGAGACCCCCCGGAAA |
| | ACGCAAAACAGCATATTGACGCTGGGAAAGACCAGAGACTCCAT |
| | GAGTTTCCACCACGCTGGCCGCCAGGCACAGATCGCCGAATAGC |
| | GGCGGCCGGTGTGGGGAAATCCATGGGTCTT |

TABLE 12
Non-limiting example Zika 5′ UTR sequences

NC_035889.1 Zika virus isolate ZIKV/H. sapiens/Brazil/Natal/2015, complete genome
NC_012532.1 Zika virus, complete genome
OL450364.1 Zika virus isolate ZIKV/Haiti/1238/2015, complete genome
OL450365.1 Zika virus isolate ZIKV/Haiti/0478/2015, complete genome
OL450366.1 Zika virus isolate ZIKV/Haiti/1332/2015, complete genome
OL414716.1 Zika virus isolate 15555, complete genome
OK571913.1 Zika virus isolate Homo sapiens/Haiti/0728/2014, complete genome
OK054351.1 Zika virus isolate 211784, complete genome
MT377491.1 Zika virus isolate MU-DMSC-1/2017, complete genome
MT377492.1 Zika virus isolate MU-DMSC-1/2016, complete genome
MT377493.1 Zika virus isolate MU-DMSC-2/2017, complete genome
MT377494.1 Zika virus isolate MU-DMSC-3/2017, complete genome
MT377495.1 Zika virus isolate MU-DMSC-4/2017, complete genome
MT377496.1 Zika virus isolate MU-DMSC-2/2016, complete genome
MT377497.1 Zika virus isolate MU-DMSC-3/2016, complete genome
MT377498.1 Zika virus isolate MU-DMSC-4/2016, complete genome
MT377499.1 Zika virus isolate MU-DMSC-5/2016, complete genome
MT377500.1 Zika virus isolate MU-DMSC-6/2016, complete genome
MT377501.1 Zika virus isolate MU-DMSC-5/2017, complete genome
MT377502.1 Zika virus isolate NS-274/2006, complete genome
MT377503.1 Zika virus isolate 15-1144, complete genome
MT377504.1 Zika virus isolate NS-110/2006, complete genome
MZ008356.1 Zika virus isolate ZIKV/Homo_sapiens/Cambodia/2019/SZ1901, complete
genome
MW143022.1 Zika virus strain MR766, complete genome
MW680969.1 Zika virus isolate rGZ02a/2018, complete genome
MW680970.1 Zika virus isolate rGZ02p/2018, complete genome
MW015936.1 Zika virus isolate Zika virus/H.sapiens-tc/THA/2006/CVD_06-020, complete
genome
MN101548.1 Zika virus isolate Salvador, complete genome
MT505349.1 Mutant Zika virus isolate DK, complete genome
MT505350.1 Mutant Zika virus isolate DK23, complete genome
MT636065.1 Zika virus isolate ARCB116141, complete genome
MT483911.1 Zika virus isolate 800/16, complete genome
MT507047.1 Zika virus isolate S-542/Yucatan/2017, complete genome
MT507048.1 Zika virus isolate S-542/Yucatan/2017 clone 6C, complete genome
MT507049.1 Zika virus isolate S-542/Yucatan/2017 clone 6V, complete genome
MT507050.1 Zika virus isolate S-542/Yucatan/2017 clone 6A, complete genome
MK566202.1 Zika virus isolate 15098, complete genome
MK241415.1 Zika virus isolate ZIKV/Homo_sapiens/CPV/2015/CV448c, complete genome
MK241416.1 Zika virus isolate ZIKV/Homo_sapiens/CPV/2015/CV487, complete genome
MK241417.1 Zika virus isolate ZIKV/Homo_sapiens/CPV/2016/CV907u, complete genome
MN611472.1 Zika virus isolate 2019YNZIKV02, complete genome
MN566104.1 Zika virus isolate Haiti/0011/2016, complete genome
MN566105.1 Zika virus isolate Haiti/0375/2016, complete genome
MN566106.1 Zika virus isolate Haiti/0395/2016, complete genome
MN566107.1 Zika virus isolate Haiti/0165/2016, complete genome
MN566108.1 Zika virus isolate Haiti/0866/2016, complete genome
MN577543.1 Zika virus isolate Haiti/1327/2014, complete genome
MN577544.1 Zika virus isolate Haiti/0148/2016, complete genome
MN577550.1 Zika virus isolate ZIKV-Nicaragua/2016-UCB7420, complete genome
MN100039.1 Zika virus isolate ZIKV/Homo sapiens/PAN/CDC-259249_V1-V3/2015,
complete genome
MN124090.1 Zika virus isolate ZIKV/Homo sapiens/PAN/CDC-259249_V1-V3/2015,
complete genome
MN124091.1 Zika virus isolate ZIKV/Homo sapiens/PAN/CDC-259249_V1-V3/2015,
complete genome
MH675619.1 Zika virus isolate CTS-30-16p, complete genome
MH675620.1 Zika virus isolate CTS-36-16p, complete genome
MH675621.1 Zika virus isolate CTS-47-16p, complete genome
MH675622.1 Zika virus isolate CTS-50-16p, complete genome
MH675623.1 Zika virus isolate CTS-56-16p, complete genome
MH675624.1 Zika virus isolate CTS-61-16p, complete genome
MH675625.1 Zika virus isolate CTS-178-16p, complete genome
MH675626.1 Zika virus isolate CTS-183-16p, complete genome
MH675627.1 Zika virus isolate CTS-193-16p, complete genome
MH675628.1 Zika virus isolate CTS-223-16p, complete genome
MH675629.1 Zika virus isolate QTX-02, complete genome
MH675630.1 Zika virus isolate QTX-04, complete genome
MN025403.1 Zika virus isolate 15555, complete genome
MK713748.1 Zika virus isolate PRVABC59, complete genome
MH900227.1 Zika virus isolate 31N, complete genome
MH882527.1 Zika virus isolate Zika virus/H.sapiens-wt/BRA/2016/ZKV17Asem_2016 Apr. 26,
complete genome
MH882528.1 Zika virus isolate Zika virus/H.sapiens-wt/BRA/2016/ZKV17Auri_2016 Apr. 26,
complete genome
MH882529.1 Zika virus isolate Zika virus/H.sapiens-wt/BRA/2016/ZKV17Bsem_2016 May 3,
complete genome
MH882530.1 Zika virus isolate Zika virus/H.sapiens-wt/BRA/2016/ZKV17Csem_2016 May 10,
complete genome
MH882531.1 Zika virus isolate Zika virus/H.sapiens-wt/BRA/2016/ZKV17Dsem_2016 May 17,
complete genome
MH882532.1 Zika virus isolate Zika virus/H.sapiens-wt/BRA/2016/ZKV17Esem_2016 May 24,
complete genome
MH882533.1 Zika virus isolate Zika virus/H.sapiens-wt/BRA/2016/ZKV17Fsem_2016 May 31,
complete genome
MH882534.1 Zika virus isolate Zika virus/H.sapiens-wt/BRA/2016/ZKV17Gsem_2016 Jun. 8,
complete genome
MH882535.1 Zika virus isolate Zika virus/H.sapiens-wt/BRA/2016/ZKV17Hsem_2016 Jun. 16,
complete genome
MH882536.1 Zika virus isolate Zika virus/H.sapiens-wt/BRA/2016/ZKV17Jsem_2016 Jun. 28,
complete genome
MH882538.1 Zika virus isolate Zika virus/H.sapiens-wt/BRA/2016/ZKV17Lsem_2016 Jul. 12,
complete genome
MH882539.1 Zika virus isolate Zika virus/H.sapiens-wt/BRA/2016/ZKV17Msem_2016 Jul. 19,
complete genome
MH882540.1 Zika virus isolate Zika virus/H.sapiens-wt/BRA/2016/ZKV17Nsem_2016 Jul. 27,
complete genome
MH882541.1 Zika virus isolate Zika virus/H.sapiens-wt/BRA/2016/ZKV17sem_2016 Apr. 19,
complete genome
MH882542.1 Zika virus isolate Zika virus/H.sapiens-wt/BRA/2016/ZKV17uri_2016 Apr. 19,
complete genome
MH882543.1 Zika virus isolate Zika virus/H.sapiens-wt/BRA/2016/ZKV19Auri_2016 May 5,
complete genome
MH882544.1 Zika virus isolate Zika virus/H.sapiens-wt/BRA/2016/ZKV19Bsem_2016 May 12,
complete genome
MH882545.1 Zika virus isolate Zika virus/H.sapiens-wt/BRA/2016/ZKV19Csem_2016 May 19,
complete genome
MH882546.1 Zika virus isolate Zika virus/H.sapiens-wt/BRA/2016/ZKV19Dsem_2016 May 24,
complete genome
MH882547.1 Zika virus isolate Zika virus/H.sapiens-wt/BRA/2016/ZKV19Esem_2016 Jun. 2,
complete genome
MH882548.1 Zika virus isolate Zika virus/H.sapiens-wt/BRA/2016/ZKV19Fsem_2016 Jun. 9,
complete genome
MK105975.1 Zika virus strain MR766, complete genome
MF783072.1 Zika virus strain Zika virus/mosquito/Haiti/1855/2016, complete genome
MF783073.1 Zika virus strain Zika virus/mosquito/Haiti/1919/2016, complete genome
MG770187.1 Zika virus isolate SV0127-14, complete genome
MG770188.1 Zika virus isolate SV0127-14, complete genome
MH916802.1 Zika virus strain ZIKV/Homo sapiens/PRI/PRVABC59_17/2015, complete
genome
MH916803.1 Zika virus strain ZIKV/Homo sapiens/PRI/PRVABC59_1/2015, complete
genome
MH916806.1 Zika virus strain ZIKV/Homo sapiens/PRI/PRVABC59_8/2015, complete
genome
MF510857.1 Zika virus isolate Zika virus/Aedes taylori/Senegal/1984/DakAr41667, complete
genome
MH513598.1 Zika virus isolate BR/Sinop/H355/2015, complete genome
MH513599.1 Zika virus isolate BR/Sinop/H366/2015, complete genome
MH513600.1 Zika virus isolate BR/Sinop/H366 2P/2015, complete genome
MH544701.2 Zika virus isolate 459148 Meta Colombia 2016, complete genome
MF996804.1 Zika virus isolate SI-BKK02, complete genome
MG548660.1 Zika virus isolate BKK03, complete genome
MG548661.1 Zika virus isolate BKK04, complete genome
MG807646.1 Zika virus isolate SI-BKK05, complete genome
MG807647.1 Zika virus isolate SI-BKK06, complete genome
MH013290.1 Zika virus isolate BKK07, complete genome
MF073357.1 Zika virus isolate 16288, complete genome
MF073358.1 Zika virus isolate 15261, complete genome
MF073359.1 Zika virus isolate 15098, complete genome
MG758785.1 Zika virus isolate 41525, complete genome
MG758786.1 Zika virus isolate 41525, complete genome
MH158236.1 Zika virus isolate FSS13025, complete genome
MH158237.1 Zika virus isolate PRVABC59, complete genome
MH130094.1 Zika virus strain ZIKV/Macaca mulatta/UGA/MR-766-VEROE6-AC4-
P12_05/1947, complete genome
MH130095.1 Zika virus strain ZIKV/Macaca mulatta/UGA/MR-766-VEROE6-AC4-
P12_07/1947, complete genome
MH130096.1 Zika virus strain ZIKV/Macaca mulatta/UGA/MR-766-VEROE6-AC10-
P12_05/1947, complete genome
MH130097.1 Zika virus strain ZIKV/Macaca mulatta/UGA/MR-766-VEROE6-AC10-
P12_03/1947, complete genome
MH130098.1 Zika virus strain ZIKV/Macaca mulatta/UGA/MR-766-VEROE6-AC10-
P12_01/1947, complete genome
MH130099.1 Zika virus strain ZIKV/Macaca mulatta/UGA/MR-766-VEROE6-AC4-
P12_02/1947, complete genome
MH130100.1 Zika virus strain ZIKV/Macaca mulatta/UGA/MR-766-VEROE6-AC4-
P12_01/1947, complete genome
MH130101.1 Zika virus strain ZIKV/Macaca mulatta/UGA/MR-766-VEROE6-AC4-
P12_08/1947, complete genome
MH130102.1 Zika virus strain ZIKV/Macaca mulatta/UGA/MR-766-VEROE6-AC10-
P12_02/1947, complete genome
MH130103.1 Zika virus strain ZIKV/Macaca mulatta/UGA/MR-766-VEROE6-AC10-
P12_06/1947, complete genome
MH130104.1 Zika virus strain ZIKV/Macaca mulatta/UGA/MR-766-VEROE6-AC10-
P12_07/1947, complete genome
MH130105.1 Zika virus strain ZIKV/Macaca mulatta/UGA/MR-766-VEROE6-AC10-
P12_04/1947, complete genome
MH130106.1 Zika virus strain ZIKV/Macaca mulatta/UGA/MR-766-VEROE6-AC10-
P12_08/1947, complete genome
MH130107.1 Zika virus strain ZIKV/Macaca mulatta/UGA/MR-766-VEROE6-AC4-
P12_03/1947, complete genome
MH130108.1 Zika virus strain ZIKV/Macaca mulatta/UGA/MR-766-VEROE6-AC4-
P12_04/1947, complete genome
MH130109.1 Zika virus strain ZIKV/Macaca mulatta/UGA/MR-766-VEROE6-AC4-
P12_06/1947, complete genome
MH157202.1 Zika virus strain ZIKV/Homo sapiens/MEX/41-017-F_V3_O/2016, complete
genome
MH157208.1 Zika virus strain ZIKV/Homo sapiens/MEX/41-022-F_VO_O/2016, complete
genome
MH157213.1 Zika virus strain ZIKV/Homo sapiens/MEX/41-001-F_V3_O/2016, complete
genome
MH119185.1 Zika virus isolate DMSc05684-16, complete genome
MH061852.1 Zika virus strain ZIKV/Macaca mulatta/UGA/MR-766-VEROE6-AC11-
P5_08/1947, complete genome
MH061853.1 Zika virus strain ZIKV/Macaca mulatta/UGA/MR-766-VEROE6-GD12-
P10_04/1947, complete genome
MH061854.1 Zika virus strain ZIKV/Macaca mulatta/UGA/MR-766-VEROE6-AC11-
P5_06/1947, complete genome
MH061855.1 Zika virus strain ZIKV/Macaca mulatta/UGA/MR-766-VEROE6-FA12-
P7_03/1947, complete genome
MH061856.1 Zika virus strain ZIKV/Macaca mulatta/UGA/MR-766-VEROE6-FC3-
P8_03/1947, complete genome
MH061857.1 Zika virus strain ZIKV/Macaca mulatta/UGA/MR-766-VEROE6-AC11-
P5_07/1947, complete genome
MH061858.1 Zika virus strain ZIKV/Macaca mulatta/UGA/MR-766-VEROE6-FC11-
P7_05/1947, complete genome
MH061859.1 Zika virus strain ZIKV/Macaca mulatta/UGA/MR-766-VEROE6-AC11-
P5_01/1947, complete genome
MH061860.1 Zika virus strain ZIKV/Macaca mulatta/UGA/MR-766-VEROE6-FC11-
P7_06/1947, complete genome
MH061861.1 Zika virus strain ZIKV/Macaca mulatta/UGA/MR-766-VEROE6-GA3-
P10_08/1947, complete genome
MH061862.1 Zika virus strain ZIKV/Macaca mulatta/UGA/MR-766-VEROE6-GD12-
P10_01/1947, complete genome
MH061863.1 Zika virus strain ZIKV/Macaca mulatta/UGA/MR-766-VEROE6-FA12-
P7_02/1947, complete genome
MH061864.1 Zika virus strain ZIKV/Macaca mulatta/UGA/MR-766-VEROE6-FA12-
P7_07/1947, complete genome
MH061865.1 Zika virus strain ZIKV/Macaca mulatta/UGA/MR-766-VEROE6-AA1-
P8_02/1947, complete genome
MH061866.1 Zika virus strain ZIKV/Macaca mulatta/UGA/MR-766-VEROE6-GD12-
P10_07/1947, complete genome
MH061867.1 Zika virus strain ZIKV/Macaca mulatta/UGA/MR-766-VEROE6-GA3-
P10_04/1947, complete genome
MH061868.1 Zika virus strain ZIKV/Macaca mulatta/UGA/MR-766-VEROE6-AA1-
P8_07/1947, complete genome
MH061869.1 Zika virus strain ZIKV/Macaca mulatta/UGA/MR-766-VEROE6-AA1-
P8_06/1947, complete genome
MH061870.1 Zika virus strain ZIKV/Macaca mulatta/UGA/MR-766-VEROE6-AC11-
P5_05/1947, complete genome
MH061871.1 Zika virus strain ZIKV/Macaca mulatta/UGA/MR-766-VEROE6-FC11-
P7_04/1947, complete genome
MH061872.1 Zika virus strain ZIKV/Macaca mulatta/UGA/MR-766-VEROE6-AA1-
P8_05/1947, complete genome
MH061873.1 Zika virus strain ZIKV/Macaca mulatta/UGA/MR-766-VEROE6-FA12-
P7_01/1947, complete genome
MH061874.1 Zika virus strain ZIKV/Macaca mulatta/UGA/MR-766-VEROE6-AA1-
P8_03/1947, complete genome
MH061875.1 Zika virus strain ZIKV/Macaca mulatta/UGA/MR-766-VEROE6-AC11-
P5_03/1947, complete genome
MH061876.1 Zika virus strain ZIKV/Macaca mulatta/UGA/MR-766-VEROE6-P12_07/1947,
complete genome
MH061877.1 Zika virus strain ZIKV/Macaca mulatta/UGA/MR-766-VEROE6-FC11-
P7_07/1947, complete genome
MH061878.1 Zika virus strain ZIKV/Macaca mulatta/UGA/MR-766-VEROE6-FC3-
P8_05/1947, complete genome
MH061879.1 Zika virus strain ZIKV/Macaca mulatta/UGA/MR-766-VEROE6-GD12-
P10_05/1947, complete genome
MH061880.1 Zika virus strain ZIKV/Macaca mulatta/UGA/MR-766-VEROE6-P12 08/1947,
complete genome
MH061881.1 Zika virus strain ZIKV/Macaca mulatta/UGA/MR-766-VEROE6-GD12-
P10_02/1947, complete genome
MH061882.1 Zika virus strain ZIKV/Macaca mulatta/UGA/MR-766-VEROE6-GD12-
P10_08/1947, complete genome
MH061883.1 Zika virus strain ZIKV/Macaca mulatta/UGA/MR-766-VEROE6-FC11-
P7_08/1947, complete genome
MH061884.1 Zika virus strain ZIKV/Macaca mulatta/UGA/MR-766-VEROE6-GA3-
P10_01/1947, complete genome
MH061885.1 Zika virus strain ZIKV/Macaca mulatta/UGA/MR-766-VEROE6-FC3-
P8_06/1947, complete genome
MH061886.1 Zika virus strain ZIKV/Macaca mulatta/UGA/MR-766-VEROE6-FA12-
P7_05/1947, complete genome
MH061887.1 Zika virus strain ZIKV/Macaca mulatta/UGA/MR-766-VEROE6-P12_05/1947,
complete genome
MH061888.1 Zika virus strain ZIKV/Macaca mulatta/UGA/MR-766-VEROE6-AC11-
P5_02/1947, complete genome
MH061889.1 Zika virus strain ZIKV/Macaca mulatta/UGA/MR-766-VEROE6-FC3-
P8_07/1947, complete genome
MH061890.1 Zika virus strain ZIKV/Macaca mulatta/UGA/MR-766-VEROE6-FC3-
P8_08/1947, complete genome
MH061891.1 Zika virus strain ZIKV/Macaca mulatta/UGA/MR-766-VEROE6-FA12-
P7_06/1947, complete genome
MH061892.1 Zika virus strain ZIKV/Macaca mulatta/UGA/MR-766-VEROE6-P12_03/1947,
complete genome
MH061893.1 Zika virus strain ZIKV/Macaca mulatta/UGA/MR-766-VEROE6-GD12-
P10_03/1947, complete genome
MH061894.1 Zika virus strain ZIKV/Macaca mulatta/UGA/MR-766-VEROE6-FC11-
P7_03/1947, complete genome
MH061895.1 Zika virus strain ZIKV/Macaca mulatta/UGA/MR-766-VEROE6-P12_01/1947,
complete genome
MH061896.1 Zika virus strain ZIKV/Macaca mulatta/UGA/MR-766-VEROE6-FC3-
P8_04/1947, complete genome
MH061897.1 Zika virus strain ZIKV/Macaca mulatta/UGA/MR-766-VEROE6-FC3-
P8_01/1947, complete genome
MH061898.1 Zika virus strain ZIKV/Macaca mulatta/UGA/MR-766-VEROE6-FC11-
P7_02/1947, complete genome
MH061899.1 Zika virus strain ZIKV/Macaca mulatta/UGA/MR-766-VEROE6-AA1-
P8_08/1947, complete genome
MH061900.1 Zika virus strain ZIKV/Macaca mulatta/UGA/MR-766-VEROE6-P12_04/1947,
complete genome
MH061901.1 Zika virus strain ZIKV/Macaca mulatta/UGA/MR-766-VEROE6-GA3-
P10_07/1947, complete genome
MH061902.1 Zika virus strain ZIKV/Macaca mulatta/UGA/MR-766-VEROE6-FA12-
P7_08/1947, complete genome
MH061903.1 Zika virus strain ZIKV/Macaca mulatta/UGA/MR-766-VEROE6-AA1-
P8_04/1947, complete genome
MH061904.1 Zika virus strain ZIKV/Macaca mulatta/UGA/MR-766-VEROE6-FC3-
P8_02/1947, complete genome
MH061905.1 Zika virus strain ZIKV/Macaca mulatta/UGA/MR-766-VEROE6-AA1-
P8_01/1947, complete genome
MH061906.1 Zika virus strain ZIKV/Macaca mulatta/UGA/MR-766-VEROE6-FC11-
P7_01/1947, complete genome
MH061907.1 Zika virus strain ZIKV/Macaca mulatta/UGA/MR-766-VEROE6-GA3-
P10_06/1947, complete genome
MH061908.1 Zika virus strain ZIKV/Macaca mulatta/UGA/MR-766-VEROE6-P12_06/1947,
complete genome
MH061909.1 Zika virus strain ZIKV/Macaca mulatta/UGA/MR-766-VEROE6-AC11-
P5_04/1947, complete genome
MH061910.1 Zika virus strain ZIKV/Macaca mulatta/UGA/MR-766-VEROE6-FA12-
P7_04/1947, complete genome
MH061911.1 Zika virus strain ZIKV/Macaca mulatta/UGA/MR-766-VEROE6-GD12-
P10_06/1947, complete genome
MH061912.1 Zika virus strain ZIKV/Macaca mulatta/UGA/MR-766-VEROE6-GA3-
P10_03/1947, complete genome
MH061913.1 Zika virus strain ZIKV/Macaca mulatta/UGA/MR-766-VEROE6-GA3-
P10_05/1947, complete genome
MH061914.1 Zika virus strain ZIKV/Macaca mulatta/UGA/MR-766-VEROE6-GA3-
P10_02/1947, complete genome
MH061915.1 Zika virus strain ZIKV/Macaca mulatta/UGA/MR-766-VEROE6-P12_02/1947,
complete genome
LC369584.1 Zika virus ZIKV/Hu/Thai/KngSG/17-D501 genomic RNA, nearly complete
genome
MG827392.1 Zika virus strain PF13/251013-18, complete genome
MG674718.1 Zika virus isolate ZKC2P4, complete genome
MG674719.1 Zika virus isolate ZKC2P6, complete genome
KY766069.1 Zika virus isolate Pf13/251013-18, complete genome
MF794971.1 Zika virus strain ZIKV/EC/Esmeraldas/121/2016, complete genome
KY553111.1 Zika virus isolate AFMC-U, complete genome
MF384325.1 Zika virus isolate mosquito/Haiti/1682/2016, complete genome
MF352141.1 Zika virus isolate PE243, complete genome
KY415986.1 Zika virus isolate Haiti/0029/2014, complete genome
KY415987.1 Zika virus isolate Haiti/0033/2014, complete genome
KY415988.1 Zika virus isolate Haiti/0054/2014, complete genome
KY415989.1 Zika virus isolate Haiti/0074/2014, complete genome
KY415990.1 Zika virus isolate Haiti/0036/2014, complete genome
KY415991.1 Zika virus isolate Haiti/0097/2014, complete genome
KY989511.1 Zika virus strain MR 766, complete genome
KY989971.1 Zika virus isolate FLA, complete genome
KX051560.1 Zika virus isolate SK364/13AS, complete genome
KX051561.1 Zika virus isolate SK403/13AS, complete genome
KX051562.1 Zika virus isolate SV0010/15, complete genome
KY765317.1 Zika virus strain ZIKV/Homo sapiens/NIC/7252_12A1/2016, complete genome
KY765318.1 Zika virus strain ZIKV/Homo sapiens/NIC/4886_12A1/2016, complete genome
KY765320.1 Zika virus strain ZIKV/Homo sapiens/NIC/6406_13A1_SP/2016, complete
genome
KY765321.1 Zika virus strain ZIKV/Homo sapiens/NIC/4886_12A1_SP/2016, complete
genome
KY765322.1 Zika virus strain ZIKV/Homo sapiens/NIC/7252_12A1_SP/2016, complete
genome
KY765323.1 Zika virus strain ZIKV/Homo sapiens/NIC/6188_13A1/2016, complete genome
KY765324.1 Zika virus strain ZIKV/Homo sapiens/NIC/8610_13A1/2016, complete genome
KY765325.1 Zika virus strain ZIKV/Homo sapiens/NIC/5005_13A1/2016, complete genome
KY765326.1 Zika virus strain ZIKV/Homo sapiens/NIC/6188_13A1_SP/2016, complete
genome
KY765327.1 Zika virus strain ZIKV/Homo sapiens/NIC/5005_13A1_SP/2016, complete
genome
KY631493.1 Zika virus isolate MEX ENCB165, complete genome
KY631494.1 Zika virus isolate MEX_ENCB165P4, complete genome
LC219720.1 Zika virus genomic RNA, complete genome, strain: ZIKV/Hu/NIID123/2016
KY648934.1 Zika virus strain ZIKV/Aedes aegypti/MEX/MEX I-44/2016, complete genome
KX827268.1 Zika virus strain ZIKV/Homo sapiens/USA/UT-1/2016, complete genome
KY348640.1 Zika virus strain SL1602, complete genome
KU761560.1 Zika virus isolate ZJ03, complete genome
KU761561.1 Zika virus isolate ZJ02, complete genome
KY272991.1 Zika virus isolate RIO-BM1, complete genome
KY288905.1 Zika virus strain MP1751, complete genome
KY328289.1 Zika virus isolate HN16, complete genome
KY272987.1 Zika virus isolate SI-BKK01, complete genome
KY120348.1 Zika virus strain MEX_CIENI551P4, complete genome
KY120349.2 Zika virus strain MEX_CIENI551, complete genome
KX269878.1 Zika virus isolate Haiti/2016/PD, complete genome
LC191864.1 Zika virus genomic RNA, complete genome, strain: ZIKV/Hu/Chiba/S36/2016
LC190723.1 Zika virus genomic RNA, complete genome, strain: ZIKV/Hu/Yokohama/1/2016
KX893855.1 Zika virus strain Zika virus/Homo sapiens/VEN/UF-2/2016, complete genome
KX879603.1 Zika virus strain ZIKV/EC/Esmeraldas/062/2016, complete genome
KX879604.1 Zika virus strain ZIKV/EC/Esmeraldas/089/2016, complete genome
KX830960.1 Zika virus culture ATCC:VR-84 isolate MR776, complete genome
KX811222.1 Zika virus isolate Brazil_2015_MG, complete genome
KX856011.1 Zika virus strain ZIKV/Aedes sp./MEX I-44/2016, complete genome
KX197205.1 Zika virus isolate 9, complete genome
KX806557.3 Zika virus isolate TS17-2016, complete genome
KX766028.1 Zika virus isolate R114916, complete genome
KX766029.1 Zika virus isolate R116265, complete genome
KX702400.1 Zika virus strain Zika virus/Homo sapiens/VEN/UF-1/2016, complete genome
KX694532.2 Zika virus strain ZIKV/Homo sapiens/THA/PLCal ZV/2013, complete genome
KX694533.2 Zika virus strain ZIKV/Aedes aegypti/MYS/P6-740/1966, complete genome
KX694534.2 Zika virus strain ZIKV/Homo sapiens/HND/R103451/2015, complete genome
KX673530.1 Zika virus isolate PHE_semen_Guadeloupe, complete genome
KX266255.1 Zika virus isolate ZIKV_SMGC-1, complete genome
KX601166.2 Zika virus strain ZIKV/Aedes africanus/SEN/DakAr41524/1984, complete
genome
KX601167.1 Zika virus strain ZIKV/Aedes sp./MYS/P6-740/1966, complete genome
KX601168.1 Zika virus strain ZIKV/Homo Sapiens/PRI/PRVABC59/2015, complete genome
KX601169.1 Zika virus strain ZIKV/Macaca mulatta/UGA/MR-766/1947, complete genome
KX369547.1 Zika virus strain PF13/251013-18, complete genome
KX446950.2 Zika virus strain ZIKV/Aedes.sp/MEX/MEX_2-81/2016, complete genome
KX446951.2 Zika virus strain ZIKV/Aedes.sp/MEX/MEX_I-7/2016, complete genome
KX377335.1 Zika virus strain MR-766, complete genome
KX377336.1 Zika virus strain P6-740, complete genome
KX377337.1 Zika virus strain PRVABC-59, complete genome
KX280026.1 Zika virus isolate Paraiba 01, complete genome
KX262887.1 Zika virus isolate 103451, complete genome
KX253996.1 Zika virus isolate ZKC2/2016, complete genome
KX247646.1 Zika virus isolate Zika virus/Homo sapiens/COL/UF-1/2016, complete genome
KX197192.1 Zika virus isolate ZIKV/H.sapiens/Brazil/PE243/2015, complete genome
KX198134.2 Zika virus strain ZIKV/Aedes africanus/SEN/DAK-AR-41524_A1C1-V2/1984,
complete genome
KX198135.2 Zika virus strain ZIKV/Homo sapiens/PAN/BEI-259634_V4/2016, complete
genome
KX156774.2 Zika virus strain ZIKV/Homo sapiens/PAN/CDC-259359_V1-V3/2015, complete
genome
KX156775.2 Zika virus strain ZIKV/Homo sapiens/PAN/CDC-259249_V1-V3/2015, complete
genome
KX156776.2 Zika virus strain ZIKV/Homo sapiens/PAN/CDC-259364_V1-V2/2015, complete
genome
KX117076.1 Zika virus isolate Zhejiang04, complete genome
KX087101.3 Zika virus strain ZIKV/Homo sapiens/PRI/PRVABC59/2015, complete genome
KX087102.2 Zika virus strain ZIKV/Homo sapiens/COL/FLR/2015, complete genome
KX051563.1 Zika virus isolate Haiti/1/2016, complete genome
KU955591.1 Zika virus isolate Zika virus/A.africanus-tc/SEN/1984/41525-DAK, complete
genome
KU955592.1 Zika virus isolate Zika virus/A.taylori-tc/SEN/1984/41662-DAK, complete
genome
KU955593.1 Zika virus isolate Zika virus/H.sapiens-tc/KHM/2010/FSS13025, complete
genome
KU955594.1 Zika virus isolate Zika virus/M.mulatta-tc/UGA/1947/MR-766, complete genome
KU955595.1 Zika virus isolate Zika virus/A.taylori-tc/SEN/1984/41671-DAK, complete
genome
KU870645.1 Zika virus isolate FB-GWUH-2016, complete genome
KU926309.2 Zika virus isolate Rio-U1, complete genome
KU926310.2 Zika virus isolate Rio-S1, complete genome
KU922923.1 Zika virus isolate MEX/InDRE/Lm/2016, complete genome
KU922960.1 Zika virus isolate MEX/InDRE/Sm/2016, complete genome
KU853012.1 Zika virus isolate Dominican Republic/2016/PD1, complete genome
KU853013.1 Zika virus isolate Dominican Republic/2016/PD2, complete genome
KU820899.2 Zika virus isolate ZJ03, complete genome
KU744693.1 Zika virus isolate VE Ganxian, complete genome
KU497555.1 Zika virus isolate Brazil-ZKV2015, complete genome
KU707826.1 Zika virus isolate SSABRI, complete genome
KU527068.1 Zika virus strain Natal RGN, complete genome
KU681081.3 Zika virus isolate Zika virus/H.sapiens-tc/THA/2014/SV0127- 14, complete
genome
KU681082.3 Zika virus isolate Zika virus/H.sapiens-tc/PHL/2012/CPC-0740, complete
genome
KU509998.3 Zika virus strain Haiti/1225/2014, complete genome
KU501215.1 Zika virus strain PRVABC59, complete genome
KU321639.1 Zika virus strain ZikaSPH2015, complete genome
LC002520.1 Zika virus genomic RNA, complete genome, strain: MR766-NIID
KJ776791.2 Zika virus strain H/PF/2013, complete genome
AY632535.2 Zika virus strain MR 766, complete genome

TABLE 13
Non-limiting example Zika 3′ UTR sequences

>NC_035889.1 Zika virus isolate ZIKV/H. sapiens/Brazil/Natal/2015, complete genome
>NC_012532.1 Zika virus, complete genome
>OL450364.1 Zika virus isolate ZIKV/Haiti/1238/2015, complete genome
>OL450365.1 Zika virus isolate ZIKV/Haiti/0478/2015, complete genome
>OL450366.1 Zika virus isolate ZIKV/Haiti/1332/2015, complete genome
>OL414716.1 Zika virus isolate 15555, complete genome
>OK571913.1 Zika virus isolate Homo sapiens/Haiti/0728/2014, complete genome
>OK054351.1 Zika virus isolate 211784, complete genome
>MT377491.1 Zika virus isolate MU-DMSC-1/2017, complete genome
>MT377492.1 Zika virus isolate MU-DMSC-1/2016, complete genome
>MT377493.1 Zika virus isolate MU-DMSC-2/2017, complete genome
>MT377494.1 Zika virus isolate MU-DMSC-3/2017, complete genome
>MT377495.1 Zika virus isolate MU-DMSC-4/2017, complete genome
>MT377496.1 Zika virus isolate MU-DMSC-2/2016, complete genome
>MT377497.1 Zika virus isolate MU-DMSC-3/2016, complete genome
>MT377498.1 Zika virus isolate MU-DMSC-4/2016, complete genome
>MT377499.1 Zika virus isolate MU-DMSC-5/2016, complete genome
>MT377500.1 Zika virus isolate MU-DMSC-6/2016, complete genome
>MT377501.1 Zika virus isolate MU-DMSC-5/2017, complete genome
>MT377502.1 Zika virus isolate NS-274/2006, complete genome
>MT377503.1 Zika virus isolate 15-1144, complete genome
>MT377504.1 Zika virus isolate NS-110/2006, complete genome
>MZ008356.1 Zika virus isolate ZIKV/Homo_sapiens/Cambodia/2019/SZ1901, complete
genome
>MW143022.1 Zika virus strain MR766, complete genome
>MW680969.1 Zika virus isolate rGZ02a/2018, complete genome
>MW680970.1 Zika virus isolate rGZ02p/2018, complete genome
>MW015936.1 Zika virus isolate Zika virus/H.sapiens-tc/THA/2006/CVD_06-020, complete
genome
>MN101548.1 Zika virus isolate Salvador, complete genome
>MT505349.1 Mutant Zika virus isolate DK, complete genome
>MT505350.1 Mutant Zika virus isolate DK23, complete genome
>MT636065.1 Zika virus isolate ARCB116141, complete genome
>MT483911.1 Zika virus isolate 800/16, complete genome
>MT507047.1 Zika virus isolate S-542/Yucatan/2017, complete genome
>MT507048.1 Zika virus isolate S-542/Yucatan/2017 clone 6C, complete genome
>MT507049.1 Zika virus isolate S-542/Yucatan/2017 clone 6V, complete genome
>MT507050.1 Zika virus isolate S-542/Yucatan/2017 clone 6A, complete genome
>MK566202.1 Zika virus isolate 15098, complete genome
>MK241415.1 Zika virus isolate ZIKV/Homo_sapiens/CPV/2015/CV448c, complete genome
>MK241416.1 Zika virus isolate ZIKV/Homo_sapiens/CPV/2015/CV487, complete genome
>MK241417.1 Zika virus isolate ZIKV/Homo_sapiens/CPV/2016/CV907u, complete genome
>MN611472.1 Zika virus isolate 2019YNZIKV02, complete genome
>MN566104.1 Zika virus isolate Haiti/0011/2016, complete genome
>MN566105.1 Zika virus isolate Haiti/0375/2016, complete genome
>MN566106.1 Zika virus isolate Haiti/0395/2016, complete genome
>MN566107.1 Zika virus isolate Haiti/0165/2016, complete genome
>MN566108.1 Zika virus isolate Haiti/0866/2016, complete genome
>MN577543.1 Zika virus isolate Haiti/1327/2014, complete genome
>MN577544.1 Zika virus isolate Haiti/0148/2016, complete genome
>MN577550.1 Zika virus isolate ZIKV-Nicaragua/2016-UCB7420, complete genome
>MN100039.1 Zika virus isolate ZIKV/Homo sapiens/PAN/CDC-259249_V1-V3/2015,
complete genome
>MN124090.1 Zika virus isolate ZIKV/Homo sapiens/PAN/CDC-259249_V1-V3/2015,
complete genome
>MN124091.1 Zika virus isolate ZIKV/Homo sapiens/PAN/CDC-259249_V1-V3/2015,
complete genome
>MH675619.1 Zika virus isolate CTS-30-16p, complete genome
>MH675620.1 Zika virus isolate CTS-36-16p, complete genome
>MH675621.1 Zika virus isolate CTS-47-16p, complete genome
>MH675622.1 Zika virus isolate CTS-50-16p, complete genome
>MH675623.1 Zika virus isolate CTS-56-16p, complete genome
>MH675624.1 Zika virus isolate CTS-61-16p, complete genome
>MH675625.1 Zika virus isolate CTS-178-16p, complete genome
>MH675626.1 Zika virus isolate CTS-183-16p, complete genome
>MH675627.1 Zika virus isolate CTS-193-16p, complete genome
>MH675628.1 Zika virus isolate CTS-223-16p, complete genome
>MH675629.1 Zika virus isolate QTX-02, complete genome
>MH675630.1 Zika virus isolate QTX-04, complete genome
>MN025403.1 Zika virus isolate 15555, complete genome
>MK713748.1 Zika virus isolate PRVABC59, complete genome
>MH900227.1 Zika virus isolate 31N, complete genome
>MH882527.1 Zika virus isolate Zika virus/H.sapiens-wt/BRA/2016/ZKV17Asem_2016 Apr. 26,
complete genome
>MH882528.1 Zika virus isolate Zika virus/H.sapiens-wt/BRA/2016/ZKV17Auri_2016 Apr. 26,
complete genome
>MH882529.1 Zika virus isolate Zika virus/H.sapiens-wt/BRA/2016/ZKV17Bsem_2016 May 3,
complete genome
>MH882530.1 Zika virus isolate Zika virus/H.sapiens-wt/BRA/2016/ZKV17Csem_2016 May 10,
complete genome
>MH882531.1 Zika virus isolate Zika virus/H.sapiens-wt/BRA/2016/ZKV17Dsem_2016 May 17,
complete genome
>MH882532.1 Zika virus isolate Zika virus/H.sapiens-wt/BRA/2016/ZKV17Esem_2016 May 24,
complete genome
>MH882533.1 Zika virus isolate Zika virus/H.sapiens-wt/BRA/2016/ZKV17Fsem_2016 May 31,
complete genome
>MH882534.1 Zika virus isolate Zika virus/H.sapiens-wt/BRA/2016/ZKV17Gsem_2016 Jun. 8,
complete genome
>MH882535.1 Zika virus isolate Zika virus/H.sapiens-wt/BRA/2016/ZKV17Hsem_2016 Jun. 16,
complete genome
>MH882536.1 Zika virus isolate Zika virus/H.sapiens-wt/BRA/2016/ZKV17Jsem_2016 Jun. 28,
complete genome
>MH882538.1 Zika virus isolate Zika virus/H.sapiens-wt/BRA/2016/ZKV17Lsem_2016 Jul. 12,
complete genome
>MH882539.1 Zika virus isolate Zika virus/H.sapiens-wt/BRA/2016/ZKV17Msem_2016 Jul. 19,
complete genome
>MH882540.1 Zika virus isolate Zika virus/H.sapiens-wt/BRA/2016/ZKV17Nsem_2016 Jul. 27,
complete genome
>MH882541.1 Zika virus isolate Zika virus/H.sapiens-wt/BRA/2016/ZKV17sem_2016 Apr. 19,
complete genome
>MH882542.1 Zika virus isolate Zika virus/H.sapiens-wt/BRA/2016/ZKV17uri_2016 Apr. 19,
complete genome
>MH882543.1 Zika virus isolate Zika virus/H.sapiens-wt/BRA/2016/ZKV19Auri_2016 May 5,
complete genome
>MH882544.1 Zika virus isolate Zika virus/H.sapiens-wt/BRA/2016/ZKV19Bsem_2016 May 12,
complete genome
>MH882545.1 Zika virus isolate Zika virus/H.sapiens-wt/BRA/2016/ZKV19Csem_2016 May 19,
complete genome
>MH882546.1 Zika virus isolate Zika virus/H.sapiens-wt/BRA/2016/ZKV19Dsem_2016 May 24,
complete genome
>MH882547.1 Zika virus isolate Zika virus/H.sapiens-wt/BRA/2016/ZKV19Esem_2016 Jun. 2,
complete genome
>MH882548.1 Zika virus isolate Zika virus/H.sapiens-wt/BRA/2016/ZKV19Fsem_2016 Jun. 9,
complete genome
>MK105975.1 Zika virus strain MR766, complete genome
>MF783072.1 Zika virus strain Zika virus/mosquito/Haiti/1855/2016, complete genome
>MF783073.1 Zika virus strain Zika virus/mosquito/Haiti/1919/2016, complete genome
>MG770187.1 Zika virus isolate SV0127-14, complete genome
>MG770188.1 Zika virus isolate SV0127-14, complete genome
>MH916802.1 Zika virus strain ZIKV/Homo sapiens/PRI/PRVABC59_17/2015, complete
genome
>MH916803.1 Zika virus strain ZIKV/Homo sapiens/PRI/PRVABC59_1/2015, complete
genome
>MH916806.1 Zika virus strain ZIKV/Homo sapiens/PRI/PRVABC59_8/2015, complete
genome
>MF510857.1 Zika virus isolate Zika virus/Aedes taylori/Senegal/1984/DakAr41667, complete
genome
>MH513598.1 Zika virus isolate BR/Sinop/H355/2015, complete genome
>MH513599.1 Zika virus isolate BR/Sinop/H366/2015, complete genome
>MH513600.1 Zika virus isolate BR/Sinop/H366 2P/2015, complete genome
>MH544701.2 Zika virus isolate 459148_Meta_Colombia_2016, complete genome
>MF996804.1 Zika virus isolate SI-BKK02, complete genome
>MG548660.1 Zika virus isolate BKK03, complete genome
>MG548661.1 Zika virus isolate BKK04, complete genome
>MG807646.1 Zika virus isolate SI-BKK05, complete genome
>MG807647.1 Zika virus isolate SI-BKK06, complete genome
>MH013290.1 Zika virus isolate BKK07, complete genome
>MF073357.1 Zika virus isolate 16288, complete genome
>MF073358.1 Zika virus isolate 15261, complete genome
>MF073359.1 Zika virus isolate 15098, complete genome
>MG758785.1 Zika virus isolate 41525, complete genome
>MG758786.1 Zika virus isolate 41525, complete genome
>MH158236.1 Zika virus isolate FSS13025, complete genome
>MH158237.1 Zika virus isolate PRVABC59, complete genome
>MH130094.1 Zika virus strain ZIKV/Macaca mulatta/UGA/MR-766-VEROE6-AC4-
P12_05/1947, complete genome
>MH130095.1 Zika virus strain ZIKV/Macaca mulatta/UGA/MR-766-VEROE6-AC4-
P12_07/1947, complete genome
>MH130096.1 Zika virus strain ZIKV/Macaca mulatta/UGA/MR-766-VEROE6-AC10-
P12_05/1947, complete genome
>MH130097.1 Zika virus strain ZIKV/Macaca mulatta/UGA/MR-766-VEROE6-AC10-
P12_03/1947, complete genome
>MH130098.1 Zika virus strain ZIKV/Macaca mulatta/UGA/MR-766-VEROE6-AC10-
P12_01/1947, complete genome
>MH130099.1 Zika virus strain ZIKV/Macaca mulatta/UGA/MR-766-VEROE6-AC4-
P12_02/1947, complete genome
>MH130100.1 Zika virus strain ZIKV/Macaca mulatta/UGA/MR-766-VEROE6-AC4-
P12_01/1947, complete genome
>MH130101.1 Zika virus strain ZIKV/Macaca mulatta/UGA/MR-766-VEROE6-AC4-
P12_08/1947, complete genome
>MH130102.1 Zika virus strain ZIKV/Macaca mulatta/UGA/MR-766-VEROE6-AC10-
P12_02/1947, complete genome
>MH130103.1 Zika virus strain ZIKV/Macaca mulatta/UGA/MR-766-VEROE6-AC10-
P12_06/1947, complete genome
>MH130104.1 Zika virus strain ZIKV/Macaca mulatta/UGA/MR-766-VEROE6-AC10-
P12_07/1947, complete genome
>MH130105.1 Zika virus strain ZIKV/Macaca mulatta/UGA/MR-766-VEROE6-AC10-
P12_04/1947, complete genome
>MH130106.1 Zika virus strain ZIKV/Macaca mulatta/UGA/MR-766-VEROE6-AC10-
P12_08/1947, complete genome
>MH130107.1 Zika virus strain ZIKV/Macaca mulatta/UGA/MR-766-VEROE6-AC4-
P12_03/1947, complete genome
>MH130108.1 Zika virus strain ZIKV/Macaca mulatta/UGA/MR-766-VEROE6-AC4-
P12_04/1947, complete genome
>MH130109.1 Zika virus strain ZIKV/Macaca mulatta/UGA/MR-766-VEROE6-AC4-
P12_06/1947, complete genome
>MH157202.1 Zika virus strain ZIKV/Homo sapiens/MEX/41-017-F_V3_O/2016, complete
genome
>MH157208.1 Zika virus strain ZIKV/Homo sapiens/MEX/41-022-F_VO_O/2016, complete
genome
>MH157213.1 Zika virus strain ZIKV/Homo sapiens/MEX/41-001-F_V3_O/2016, complete
genome
>MH119185.1 Zika virus isolate DMSc05684-16, complete genome
>MH061852.1 Zika virus strain ZIKV/Macaca mulatta/UGA/MR-766-VEROE6-AC11-
P5_08/1947, complete genome
>MH061853.1 Zika virus strain ZIKV/Macaca mulatta/UGA/MR-766-VEROE6-GD12-
P10_04/1947, complete genome
>MH061854.1 Zika virus strain ZIKV/Macaca mulatta/UGA/MR-766-VEROE6-AC11-
P5_06/1947, complete genome
>MH061855.1 Zika virus strain ZIKV/Macaca mulatta/UGA/MR-766-VEROE6-FA12-
P7_03/1947, complete genome
>MH061856.1 Zika virus strain ZIKV/Macaca mulatta/UGA/MR-766-VEROE6-FC3-
P8_03/1947, complete genome
>MH061857.1 Zika virus strain ZIKV/Macaca mulatta/UGA/MR-766-VEROE6-AC11-
P5_07/1947, complete genome
>MH061858.1 Zika virus strain ZIKV/Macaca mulatta/UGA/MR-766-VEROE6-FC11-
P7_05/1947, complete genome
>MH061859.1 Zika virus strain ZIKV/Macaca mulatta/UGA/MR-766-VEROE6-AC11-
P5_01/1947, complete genome
>MH061860.1 Zika virus strain ZIKV/Macaca mulatta/UGA/MR-766-VEROE6-FC11-
P7_06/1947, complete genome
>MH061861.1 Zika virus strain ZIKV/Macaca mulatta/UGA/MR-766-VEROE6-GA3-
P10_08/1947, complete genome
>MH061862.1 Zika virus strain ZIKV/Macaca mulatta/UGA/MR-766-VEROE6-GD12-
P10_01/1947, complete genome
>MH061863.1 Zika virus strain ZIKV/Macaca mulatta/UGA/MR-766-VEROE6-FA12-
P7_02/1947, complete genome
>MH061864.1 Zika virus strain ZIKV/Macaca mulatta/UGA/MR-766-VEROE6-FA12-
P7_07/1947, complete genome
>MH061865.1 Zika virus strain ZIKV/Macaca mulatta/UGA/MR-766-VEROE6-AA1-
P8_02/1947, complete genome
>MH061866.1 Zika virus strain ZIKV/Macaca mulatta/UGA/MR-766-VEROE6-GD12-
P10_07/1947, complete genome
>MH061867.1 Zika virus strain ZIKV/Macaca mulatta/UGA/MR-766-VEROE6-GA3-
P10_04/1947, complete genome
>MH061868.1 Zika virus strain ZIKV/Macaca mulatta/UGA/MR-766-VEROE6-AA1-
P8_07/1947, complete genome
>MH061869.1 Zika virus strain ZIKV/Macaca mulatta/UGA/MR-766-VEROE6-AA1-
P8_06/1947, complete genome
>MH061870.1 Zika virus strain ZIKV/Macaca mulatta/UGA/MR-766-VEROE6-AC11-
P5_05/1947, complete genome
>MH061871.1 Zika virus strain ZIKV/Macaca mulatta/UGA/MR-766-VEROE6-FC11-
P7_04/1947, complete genome
>MH061872.1 Zika virus strain ZIKV/Macaca mulatta/UGA/MR-766-VEROE6-AA1-
P8_05/1947, complete genome
>MH061873.1 Zika virus strain ZIKV/Macaca mulatta/UGA/MR-766-VEROE6-FA12-
P7_01/1947, complete genome
>MH061874.1 Zika virus strain ZIKV/Macaca mulatta/UGA/MR-766-VEROE6-AA1-
P8_03/1947, complete genome
>MH061875.1 Zika virus strain ZIKV/Macaca mulatta/UGA/MR-766-VEROE6-AC11-
P5_03/1947, complete genome
>MH061876.1 Zika virus strain ZIKV/Macaca mulatta/UGA/MR-766-VEROE6-P12_07/1947,
complete genome
>MH061877.1 Zika virus strain ZIKV/Macaca mulatta/UGA/MR-766-VEROE6-FC11-
P7_07/1947, complete genome
>MH061878.1 Zika virus strain ZIKV/Macaca mulatta/UGA/MR-766-VEROE6-FC3-
P8_05/1947, complete genome
>MH061879.1 Zika virus strain ZIKV/Macaca mulatta/UGA/MR-766-VEROE6-GD12-
P10_05/1947, complete genome
>MH061880.1 Zika virus strain ZIKV/Macaca mulatta/UGA/MR-766-VEROE6-P12_08/1947,
complete genome
>MH061881.1 Zika virus strain ZIKV/Macaca mulatta/UGA/MR-766-VEROE6-GD12-
P10_02/1947, complete genome
>MH061882.1 Zika virus strain ZIKV/Macaca mulatta/UGA/MR-766-VEROE6-GD12-
P10_08/1947, complete genome
>MH061883.1 Zika virus strain ZIKV/Macaca mulatta/UGA/MR-766-VEROE6-FC11-
P7_08/1947, complete genome
>MH061884.1 Zika virus strain ZIKV/Macaca mulatta/UGA/MR-766-VEROE6-GA3-
P10_01/1947, complete genome
>MH061885.1 Zika virus strain ZIKV/Macaca mulatta/UGA/MR-766-VEROE6-FC3-
P8_06/1947, complete genome
>MH061886.1 Zika virus strain ZIKV/Macaca mulatta/UGA/MR-766-VEROE6-FA12-
P7_05/1947, complete genome
>MH061887.1 Zika virus strain ZIKV/Macaca mulatta/UGA/MR-766-VEROE6-P12_05/1947,
complete genome
>MH061888.1 Zika virus strain ZIKV/Macaca mulatta/UGA/MR-766-VEROE6-AC11-
P5_02/1947, complete genome
>MH061889.1 Zika virus strain ZIKV/Macaca mulatta/UGA/MR-766-VEROE6-FC3-
P8_07/1947, complete genome
>MH061890.1 Zika virus strain ZIKV/Macaca mulatta/UGA/MR-766-VEROE6-FC3-
P8_08/1947, complete genome
>MH061891.1 Zika virus strain ZIKV/Macaca mulatta/UGA/MR-766-VEROE6-FA12-
P7_06/1947, complete genome
>MH061892.1 Zika virus strain ZIKV/Macaca mulatta/UGA/MR-766-VEROE6-P12_03/1947,
complete genome
>MH061893.1 Zika virus strain ZIKV/Macaca mulatta/UGA/MR-766-VEROE6-GD12-
P10_03/1947, complete genome
>MH061894.1 Zika virus strain ZIKV/Macaca mulatta/UGA/MR-766-VEROE6-FC11-
P7_03/1947, complete genome
>MH061895.1 Zika virus strain ZIKV/Macaca mulatta/UGA/MR-766-VEROE6-P12_01/1947,
complete genome
>MH061896.1 Zika virus strain ZIKV/Macaca mulatta/UGA/MR-766-VEROE6-FC3-
P8_04/1947, complete genome
>MH061897.1 Zika virus strain ZIKV/Macaca mulatta/UGA/MR-766-VEROE6-FC3-
P8_01/1947, complete genome
>MH061898.1 Zika virus strain ZIKV/Macaca mulatta/UGA/MR-766-VEROE6-FC11-
P7_02/1947, complete genome
>MH061899.1 Zika virus strain ZIKV/Macaca mulatta/UGA/MR-766-VEROE6-AA1-
P8_08/1947, complete genome
>MH061900.1 Zika virus strain ZIKV/Macaca mulatta/UGA/MR-766-VEROE6-P12_04/1947,
complete genome
>MH061901.1 Zika virus strain ZIKV/Macaca mulatta/UGA/MR-766-VEROE6-GA3-
P10_07/1947, complete genome
>MH061902.1 Zika virus strain ZIKV/Macaca mulatta/UGA/MR-766-VEROE6-FA12-
P7_08/1947, complete genome
>MH061903.1 Zika virus strain ZIKV/Macaca mulatta/UGA/MR-766-VEROE6-AA1-
P8_04/1947, complete genome
>MH061904.1 Zika virus strain ZIKV/Macaca mulatta/UGA/MR-766-VEROE6-FC3-
P8_02/1947, complete genome
>MH061905.1 Zika virus strain ZIKV/Macaca mulatta/UGA/MR-766-VEROE6-AA1-
P8_01/1947, complete genome
>MH061906.1 Zika virus strain ZIKV/Macaca mulatta/UGA/MR-766-VEROE6-FC11-
P7_01/1947, complete genome
>MH061907.1 Zika virus strain ZIKV/Macaca mulatta/UGA/MR-766-VEROE6-GA3-
P10_06/1947, complete genome
>MH061908.1 Zika virus strain ZIKV/Macaca mulatta/UGA/MR-766-VEROE6-P12_06/1947,
complete genome
>MH061909.1 Zika virus strain ZIKV/Macaca mulatta/UGA/MR-766-VEROE6-AC11-
P5_04/1947, complete genome
>MH061910.1 Zika virus strain ZIKV/Macaca mulatta/UGA/MR-766-VEROE6-FA12-
P7_04/1947, complete genome
>MH061911.1 Zika virus strain ZIKV/Macaca mulatta/UGA/MR-766-VEROE6-GD12-
P10_06/1947, complete genome
>MH061912.1 Zika virus strain ZIKV/Macaca mulatta/UGA/MR-766-VEROE6-GA3-
P10_03/1947, complete genome
>MH061913.1 Zika virus strain ZIKV/Macaca mulatta/UGA/MR-766-VEROE6-GA3-
P10_05/1947, complete genome
>MH061914.1 Zika virus strain ZIKV/Macaca mulatta/UGA/MR-766-VEROE6-GA3-
P10_02/1947, complete genome
>MH061915.1 Zika virus strain ZIKV/Macaca mulatta/UGA/MR-766-VEROE6-P12_02/1947,
complete genome
>LC369584.1 Zika virus ZIKV/Hu/Thai/KngSG/17-D501 genomic RNA, nearly complete
genome
>MG827392.1 Zika virus strain PF13/251013-18, complete genome
>MG674718.1 Zika virus isolate ZKC2P4, complete genome
>MG674719.1 Zika virus isolate ZKC2P6, complete genome
>KY766069.1 Zika virus isolate Pf13/251013-18, complete genome
>MF794971.1 Zika virus strain ZIKV/EC/Esmeraldas/121/2016, complete genome
>KY553111.1 Zika virus isolate AFMC-U, complete genome
>MF384325.1 Zika virus isolate mosquito/Haiti/1682/2016, complete genome
>MF352141.1 Zika virus isolate PE243, complete genome
>KY415986.1 Zika virus isolate Haiti/0029/2014, complete genome
>KY415987.1 Zika virus isolate Haiti/0033/2014, complete genome
>KY415988.1 Zika virus isolate Haiti/0054/2014, complete genome
>KY415989.1 Zika virus isolate Haiti/0074/2014, complete genome
>KY415990.1 Zika virus isolate Haiti/0036/2014, complete genome
>KY415991.1 Zika virus isolate Haiti/0097/2014, complete genome
>KY989511.1 Zika virus strain MR 766, complete genome
>KY989971.1 Zika virus isolate FLA, complete genome
>KX051560.1 Zika virus isolate SK364/13AS, complete genome
>KX051561.1 Zika virus isolate SK403/13AS, complete genome
>KX051562.1 Zika virus isolate SV0010/15, complete genome
>KY765317.1 Zika virus strain ZIKV/Homo sapiens/NIC/7252_12A1/2016, complete genome
>KY765318.1 Zika virus strain ZIKV/Homo sapiens/NIC/4886_12A1/2016, complete genome
>KY765320.1 Zika virus strain ZIKV/Homo sapiens/NIC/6406_13A1_SP/2016, complete
genome
>KY765321.1 Zika virus strain ZIKV/Homo sapiens/NIC/4886_12A1_SP/2016, complete
genome
>KY765322.1 Zika virus strain ZIKV/Homo sapiens/NIC/7252_12A1_SP/2016, complete
genome
>KY765323.1 Zika virus strain ZIKV/Homo sapiens/NIC/6188_13A1/2016, complete genome
>KY765324.1 Zika virus strain ZIKV/Homo sapiens/NIC/8610_13A1/2016, complete genome
>KY765325.1 Zika virus strain ZIKV/Homo sapiens/NIC/5005_13A1/2016, complete genome
>KY765326.1 Zika virus strain ZIKV/Homo sapiens/NIC/6188_13A1_SP/2016, complete
genome
>KY765327.1 Zika virus strain ZIKV/Homo sapiens/NIC/5005_13A1_SP/2016, complete
genome
>KY631493.1 Zika virus isolate MEX_ENCB165, complete genome
>KY631494.1 Zika virus isolate MEX_ENCB165P4, complete genome
>LC219720.1 Zika virus genomic RNA, complete genome, strain: ZIKV/Hu/NIID123/2016
>KY648934.1 Zika virus strain ZIKV/Aedes aegypti/MEX/MEX_I-44/2016, complete genome
>KX827268.1 Zika virus strain ZIKV/Homo sapiens/USA/UT-1/2016, complete genome
>KY348640.1 Zika virus strain SL1602, complete genome
>KU761560.1 Zika virus isolate ZJ03, complete genome
>KU761561.1 Zika virus isolate ZJ02, complete genome
>KY272991.1 Zika virus isolate RIO-BM1, complete genome
>KY288905.1 Zika virus strain MP1751, complete genome
>KY328289.1 Zika virus isolate HN16, complete genome
>KY272987.1 Zika virus isolate SI-BKK01, complete genome
>KY120348.1 Zika virus strain MEX_CIENI551P4, complete genome
>KY120349.2 Zika virus strain MEX_CIENI551, complete genome
>KX269878.1 Zika virus isolate Haiti/2016/PD, complete genome
>LC191864.1 Zika virus genomic RNA, complete genome, strain: ZIKV/Hu/Chiba/S36/2016
>LC190723.1 Zika virus genomic RNA, complete genome, strain: ZIKV/Hu/Yokohama/1/2016
>KX893855.1 Zika virus strain Zika virus/Homo sapiens/VEN/UF-2/2016, complete genome
>KX879603.1 Zika virus strain ZIKV/EC/Esmeraldas/062/2016, complete genome
>KX879604.1 Zika virus strain ZIKV/EC/Esmeraldas/089/2016, complete genome
>KX830960.1 Zika virus culture ATCC:VR-84 isolate MR776, complete genome
>KX811222.1 Zika virus isolate Brazil_2015_MG, complete genome
>KX856011.1 Zika virus strain ZIKV/Aedes sp./MEX_I-44/2016, complete genome
>KX197205.1 Zika virus isolate 9, complete genome
>KX806557.3 Zika virus isolate TS17-2016, complete genome
>KX766028.1 Zika virus isolate R114916, complete genome
>KX766029.1 Zika virus isolate R116265, complete genome
>KX702400.1 Zika virus strain Zika virus/Homo sapiens/VEN/UF-1/2016, complete genome
>KX694532.2 Zika virus strain ZIKV/Homo sapiens/THA/PLCal_ZV/2013, complete genome
>KX694533.2 Zika virus strain ZIKV/Aedes aegypti/MYS/P6-740/1966, complete genome
>KX694534.2 Zika virus strain ZIKV/Homo sapiens/HND/R103451/2015, complete genome
>KX673530.1 Zika virus isolate PHE_semen_Guadeloupe, complete genome
>KX266255.1 Zika virus isolate ZIKV_SMGC-1, complete genome
>KX601166.2 Zika virus strain ZIKV/Aedes africanus/SEN/DakAr41524/1984, complete
genome
>KX601167.1 Zika virus strain ZIKV/Aedes sp./MYS/P6-740/1966, complete genome
>KX601168.1 Zika virus strain ZIKV/Homo Sapiens/PRI/PRVABC59/2015, complete genome
>KX601169.1 Zika virus strain ZIKV/Macaca mulatta/UGA/MR-766/1947, complete genome
>KX369547.1 Zika virus strain PF13/251013-18, complete genome
>KX446950.2 Zika virus strain ZIKV/Aedes.sp/MEX/MEX_2-81/2016, complete genome
>KX446951.2 Zika virus strain ZIKV/Aedes.sp/MEX/MEX_I-7/2016, complete genome
>KX377335.1 Zika virus strain MR-766, complete genome
>KX377336.1 Zika virus strain P6-740, complete genome
>KX377337.1 Zika virus strain PRVABC-59, complete genome
>KX280026.1 Zika virus isolate Paraiba_01, complete genome
>KX262887.1 Zika virus isolate 103451, complete genome
>KX253996.1 Zika virus isolate ZKC2/2016, complete genome
>KX247646.1 Zika virus isolate Zika virus/Homo sapiens/COL/UF-1/2016, complete genome
>KX197192.1 Zika virus isolate ZIKV/H.sapiens/Brazil/PE243/2015, complete genome
>KX198134.2 Zika virus strain ZIKV/Aedes africanus/SEN/DAK-AR-41524_A1C1-V2/1984,
complete genome
>KX198135.2 Zika virus strain ZIKV/Homo sapiens/PAN/BEI-259634_V4/2016, complete
genome
>KX156774.2 Zika virus strain ZIKV/Homo sapiens/PAN/CDC-259359_V1-V3/2015, complete
genome
>KX156775.2 Zika virus strain ZIKV/Homo sapiens/PAN/CDC-259249_V1-V3/2015, complete
genome
>KX156776.2 Zika virus strain ZIKV/Homo sapiens/PAN/CDC-259364_V1-V2/2015, complete
genome
>KX117076.1 Zika virus isolate Zhejiang04, complete genome
>KX087101.3 Zika virus strain ZIKV/Homo sapiens/PRI/PRVABC59/2015, complete genome
>KX087102.2 Zika virus strain ZIKV/Homo sapiens/COL/FLR/2015, complete genome
>KX051563.1 Zika virus isolate Haiti/1/2016, complete genome
>KU955591.1 Zika virus isolate Zika virus/A.africanus-tc/SEN/1984/41525-DAK, complete
genome
>KU955592.1 Zika virus isolate Zika virus/A.taylori-tc/SEN/1984/41662-DAK, complete
genome
>KU955593.1 Zika virus isolate Zika virus/H.sapiens-tc/KHM/2010/FSS13025, complete
genome
>KU955594.1 Zika virus isolate Zika virus/M.mulatta-tc/UGA/1947/MR-766, complete genome
>KU955595.1 Zika virus isolate Zika virus/A.taylori-tc/SEN/1984/41671-DAK, complete
genome
>KU870645.1 Zika virus isolate FB-GWUH-2016, complete genome
>KU926309.2 Zika virus isolate Rio-U1, complete genome
>KU926310.2 Zika virus isolate Rio-S1, complete genome
>KU922923.1 Zika virus isolate MEX/InDRE/Lm/2016, complete genome
>KU922960.1 Zika virus isolate MEX/InDRE/Sm/2016, complete genome
>KU853012.1 Zika virus isolate Dominican Republic/2016/PD1, complete genome
>KU853013.1 Zika virus isolate Dominican Republic/2016/PD2, complete genome
>KU820899.2 Zika virus isolate ZJ03, complete genome
>KU744693.1 Zika virus isolate VE_Ganxian, complete genome
>KU497555.1 Zika virus isolate Brazil-ZKV2015, complete genome
>KU707826.1 Zika virus isolate SSABRI, complete genome
>KU527068.1 Zika virus strain Natal RGN, complete genome
>KU681081.3 Zika virus isolate Zika virus/H.sapiens-tc/THA/2014/SV0127- 14, complete
genome
>KU681082.3 Zika virus isolate Zika virus/H.sapiens-tc/PHL/2012/CPC-0740, complete genome
>KU509998.3 Zika virus strain Haiti/1225/2014, complete genome
>KU501215.1 Zika virus strain PRVABC59, complete genome
>KU321639.1 Zika virus strain ZikaSPH2015, complete genome
>LC002520.1 Zika virus genomic RNA, complete genome, strain: MR766-NIID
>KJ776791.2 Zika virus strain H/PF/2013, complete genome
>AY632535.2 Zika virus strain MR 766, complete genome

In some embodiments, the nucleic acid composition does not comprise a polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). In other embodiments, the nucleic acid composition comprises polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). Non-limiting example non-structural proteins include those of Tables 5 and 9B. In some embodiments, the nucleic acid does not comprise a polynucleotide encoding 2k peptide. In some embodiments, the nucleic acid comprises a polynucleotide encoding 2k peptide. In some embodiments, the nucleic acid comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 5.

In certain embodiments, the nucleic acid composition comprises a polynucleotide encoding a NS1 protein. In certain embodiments, the nucleic acid composition comprises a polynucleotide encoding a NS2A protein. In certain embodiments, the nucleic acid composition comprises a polynucleotide encoding a NS2B protein. In certain embodiments, the nucleic acid composition comprises a polynucleotide encoding a NS3 protein. In certain embodiments, the nucleic acid composition comprises a polynucleotide encoding a NS4A protein. In certain embodiments, the nucleic acid composition comprises a polynucleotide encoding a NS4B protein. In certain embodiments, the nucleic acid composition comprises a polynucleotide encoding a NS5 protein. In certain embodiments, the nucleic acid composition comprises a polynucleotide encoding a 2k peptide.

In certain embodiments, the nucleic acid composition comprises a polynucleotide encoding a NS1 and a NS2A. In certain embodiments, the nucleic acid composition comprises a polynucleotide encoding a NS1 and a NS2B. In certain embodiments, the nucleic acid composition comprises a polynucleotide encoding a NS1 and a NS3. In certain embodiments, the nucleic acid composition comprises a polynucleotide encoding a NS1 and a NS4A. In certain embodiments, the nucleic acid composition comprises a polynucleotide encoding a NS1 and a NS4B. In certain embodiments, the nucleic acid composition comprises a polynucleotide encoding a NS1 and a NS5.

In certain embodiments, the nucleic acid composition comprises a polynucleotide encoding a NS2A and a NS2B. In certain embodiments, the nucleic acid composition comprises a polynucleotide encoding a NS2A and a NS3. In certain embodiments, the nucleic acid composition comprises a polynucleotide encoding a NS2A and a NS4A. In certain embodiments, the nucleic acid composition comprises a polynucleotide encoding a NS2A and a NS4B. In certain embodiments, the nucleic acid composition comprises a polynucleotide encoding a NS2A and a NS5.

In certain embodiments, the nucleic acid composition comprises a polynucleotide encoding a NS2B and a NS3. In certain embodiments, the nucleic acid composition comprises a polynucleotide encoding a NS2B and a NS4A. In certain embodiments, the nucleic acid composition comprises a polynucleotide encoding a NS2B and a NS4B. In certain embodiments, the nucleic acid composition comprises a polynucleotide encoding a NS2B and a NS5.

In certain embodiments, the nucleic acid composition comprises a polynucleotide encoding a NS3 and a NS4A. In certain embodiments, the nucleic acid composition comprises a polynucleotide encoding a NS3 and a NS4B. In certain embodiments, the nucleic acid composition comprises a polynucleotide encoding a NS3 and a NS5.

In certain embodiments, the nucleic acid composition comprises a polynucleotide encoding a NS4A and a NS4B. In certain embodiments, the nucleic acid composition comprises a polynucleotide encoding a NS4A and a NS5.

In certain embodiments, the nucleic acid composition comprises a polynucleotide encoding a NS4B and a NS5.

In certain embodiments, the nucleic acid composition comprises a polynucleotide encoding a NS1, a NS2A, and NS2B. In certain embodiments, the nucleic acid composition comprises a polynucleotide encoding a NS1, a NS2A, and NS3. In certain embodiments, the nucleic acid composition comprises a polynucleotide encoding a NS1, a NS2A, and NS4A. In certain embodiments, the nucleic acid composition comprises a polynucleotide encoding a NS1, a NS2A, and NS4B. In certain embodiments, the nucleic acid composition comprises a polynucleotide encoding a NS1, a NS2A, and NS5.

In certain embodiments, the nucleic acid composition comprises a polynucleotide encoding a NS1, a NS2B, and NS3. In certain embodiments, the nucleic acid composition comprises a polynucleotide encoding a NS1, a NS2B, and NS4A. In certain embodiments, the nucleic acid composition comprises a polynucleotide encoding a NS1, a NS2B, and NS4B. In certain embodiments, the nucleic acid composition comprises a polynucleotide encoding a NS1, a NS2B, and NS5.

In certain embodiments, the nucleic acid composition comprises a polynucleotide encoding a NS1, a NS3, and NS4A. In certain embodiments, the nucleic acid composition comprises a polynucleotide encoding a NS1, a NS3, and NS4B. In certain embodiments, the nucleic acid composition comprises a polynucleotide encoding a NS1, a NS3, and NS5.

In certain embodiments, the nucleic acid composition comprises a polynucleotide encoding a NS1, a NS4A, and a NS4B. In certain embodiments, the nucleic acid composition comprises a polynucleotide encoding a NS1, a NS4A, and a NS5.

In certain embodiments, the nucleic acid composition comprises a polynucleotide encoding a NS1, a NS4B, and a NS5.

In certain embodiments, the nucleic acid composition comprises a polynucleotide encoding a NS2A, a NS2B, and a NS3. In certain embodiments, the nucleic acid composition comprises a polynucleotide encoding a NS2A, a NS2B, and a NS4A. In certain embodiments, the nucleic acid composition comprises a polynucleotide encoding a NS2A, a NS2B, and a NS4B. In certain embodiments, the nucleic acid composition comprises a polynucleotide encoding a NS2A, a NS2B, and a NS5.

In certain embodiments, the nucleic acid composition comprises a polynucleotide encoding a NS2A, a NS3, and a NS4A. In certain embodiments, the nucleic acid composition comprises a polynucleotide encoding a NS2A, a NS3, and a NS4B. In certain embodiments, the nucleic acid composition comprises a polynucleotide encoding a NS2A, a NS3, and a NS5.

In certain embodiments, the nucleic acid composition comprises a polynucleotide encoding a NS2A, a NS4A, and a NS4B. In certain embodiments, the nucleic acid composition comprises a polynucleotide encoding a NS2A, a NS4A, and a NS5.

In certain embodiments, the nucleic acid composition comprises a polynucleotide encoding a NS2A, a NS4B, and a NS5.

In certain embodiments, the nucleic acid composition comprises a polynucleotide encoding a NS2B, a NS3, and a NS4A. In certain embodiments, the nucleic acid composition comprises a polynucleotide encoding a NS2B, a NS3, and a NS4B. In certain embodiments, the nucleic acid composition comprises a polynucleotide encoding a NS2B, a NS3, and a NS5.

In certain embodiments, the nucleic acid composition comprises a polynucleotide encoding a NS2B, a NS4A, and NS5.

In certain embodiments, the nucleic acid composition comprises a polynucleotide encoding a NS2B, a NS4B, and a NS5.

In certain embodiments, the nucleic acid composition comprises a polynucleotide encoding a NS3 and a NS4A, and a NS5.

In certain embodiments, the nucleic acid composition comprises a polynucleotide encoding a NS3, a NS4B, and a NS5.

In certain embodiments, the nucleic acid composition comprises a polynucleotide encoding a NS4A, a NS4B, and a NS5.

In certain embodiments, the nucleic acid composition comprises a polynucleotide encoding all NS proteins except a NS1, a NS2A, and NS2B. In certain embodiments, the nucleic acid composition comprises a polynucleotide encoding all NS proteins except a NS1, a NS2A, and NS3. In certain embodiments, the nucleic acid composition comprises a polynucleotide encoding all NS proteins except a NS1, a NS2A, and NS4A. In certain embodiments, the nucleic acid composition comprises a polynucleotide encoding all NS proteins except a NS1, a NS2A, and NS4B. In certain embodiments, the nucleic acid composition comprises a polynucleotide encoding all NS proteins except a NS1, a NS2A, and NS5.

In certain embodiments, the nucleic acid composition comprises a polynucleotide encoding all NS proteins except a NS1, a NS2B, and NS3. In certain embodiments, the nucleic acid composition comprises a polynucleotide encoding all NS proteins except a NS1, a NS2B, and NS4A. In certain embodiments, the nucleic acid composition comprises a polynucleotide encoding all NS proteins except a NS1, a NS2B, and NS4B. In certain embodiments, the nucleic acid composition comprises a polynucleotide encoding all NS proteins except a NS1, a NS2B, and NS5.

In certain embodiments, the nucleic acid composition comprises a polynucleotide encoding all NS proteins except a NS1, a NS3, and NS4A. In certain embodiments, the nucleic acid composition comprises a polynucleotide encoding all NS proteins except a NS1, a NS3, and NS4B. In certain embodiments, the nucleic acid composition comprises a polynucleotide encoding all NS proteins except a NS1, a NS3, and NS5.

In certain embodiments, the nucleic acid composition comprises a polynucleotide encoding all NS proteins except a NS1, a NS4A, and a NS4B. In certain embodiments, the nucleic acid composition comprises a polynucleotide encoding all NS proteins except a NS1, a NS4A, and a NS5.

In certain embodiments, the nucleic acid composition comprises a polynucleotide encoding all NS proteins except a NS1, a NS4B, and a NS5.

In certain embodiments, the nucleic acid composition comprises a polynucleotide encoding all NS proteins except a NS2A, a NS2B, and a NS3. In certain embodiments, the nucleic acid composition comprises a polynucleotide encoding all NS proteins except a NS2A, a NS2B, and a NS4A. In certain embodiments, the nucleic acid composition comprises a polynucleotide encoding all NS proteins except a NS2A, a NS2B, and a NS4B. In certain embodiments, the nucleic acid composition comprises a polynucleotide encoding all NS proteins except a NS2A, a NS2B, and a NS5.

In certain embodiments, the nucleic acid composition comprises a polynucleotide encoding all NS proteins except a NS2A, a NS3, and a NS4A. In certain embodiments, the nucleic acid composition comprises a polynucleotide encoding all NS proteins except a NS2A, a NS3, and a NS4B. In certain embodiments, the nucleic acid composition comprises a polynucleotide encoding all NS proteins except a NS2A, a NS3, and a NS5.

In certain embodiments, the nucleic acid composition comprises a polynucleotide encoding all NS proteins except a NS2A, a NS4A, and a NS4B. In certain embodiments, the nucleic acid composition comprises a polynucleotide encoding all NS proteins except a NS2A, a NS4A, and a NS5.

In certain embodiments, the nucleic acid composition comprises a polynucleotide encoding all NS proteins except a NS2A, a NS4B, and a NS5.

In certain embodiments, the nucleic acid composition comprises a polynucleotide encoding all NS proteins except a NS2B, a NS3, and a NS4A. In certain embodiments, the nucleic acid composition comprises a polynucleotide encoding all NS proteins except a NS2B, a NS3, and a NS4B. In certain embodiments, the nucleic acid composition comprises a polynucleotide encoding all NS proteins except a NS2B, a NS3, and a NS5.

In certain embodiments, the nucleic acid composition comprises a polynucleotide encoding all NS proteins except a NS2B, a NS4A, and NS5.

In certain embodiments, the nucleic acid composition comprises a polynucleotide encoding all NS proteins except all NS proteins except a NS2B, a NS4B, and a NS5.

In certain embodiments, the nucleic acid composition comprises a polynucleotide encoding all NS proteins except a NS3 and a NS4A, and a NS5.

In certain embodiments, the nucleic acid composition comprises a polynucleotide encoding all NS proteins except a NS3, a NS4B, and a NS5.

In certain embodiments, the nucleic acid composition comprises a polynucleotide encoding all NS proteins except a NS4A, a NS4B, and a NS5.

In certain embodiments, the nucleic acid composition comprises a polynucleotide encoding all NS proteins except a NS1 and a NS2A. In certain embodiments, the nucleic acid composition comprises a polynucleotide encoding all NS proteins except a NS1 and a NS2B. In certain embodiments, the nucleic acid composition comprises a polynucleotide encoding all NS proteins except a NS1 and a NS3. In certain embodiments, the nucleic acid composition comprises a polynucleotide encoding all NS proteins except all NS proteins except a NS1 and a NS4A. In certain embodiments, the nucleic acid composition comprises a polynucleotide encoding all NS proteins except a NS1 and a NS4B. In certain embodiments, the nucleic acid composition comprises a polynucleotide encoding all NS proteins except a NS1 and a NS5.

In certain embodiments, the nucleic acid composition comprises a polynucleotide encoding all NS proteins except a NS2A and a NS2B. In certain embodiments, the nucleic acid composition comprises a polynucleotide encoding all NS proteins except a NS2A and a NS3. In certain embodiments, the nucleic acid composition comprises a polynucleotide encoding all NS proteins except a NS2A and a NS4A. In certain embodiments, the nucleic acid composition comprises a polynucleotide encoding all NS proteins except a NS2A and a NS4B. In certain embodiments, the nucleic acid composition comprises a polynucleotide encoding all NS proteins except a NS2A and a NS5.

In certain embodiments, the nucleic acid composition comprises a polynucleotide encoding all NS proteins except a NS2B and a NS3. In certain embodiments, the nucleic acid composition comprises a polynucleotide encoding all NS proteins except a NS2B and a NS4A. In certain embodiments, the nucleic acid composition comprises a polynucleotide encoding all NS proteins except a NS2B and a NS4B. In certain embodiments, the nucleic acid composition comprises a polynucleotide encoding all NS proteins except a NS2B and a NS5.

In certain embodiments, the nucleic acid composition comprises a polynucleotide encoding all NS proteins except a NS3 and a NS4A. In certain embodiments, the nucleic acid composition comprises a polynucleotide encoding all NS proteins except a NS3 and a NS4B. In certain embodiments, the nucleic acid composition comprises a polynucleotide encoding all NS proteins except a NS3 and a NS5.

In certain embodiments, the nucleic acid composition comprises a polynucleotide encoding all NS proteins except a NS4A and a NS4B. In certain embodiments, the nucleic acid composition comprises a polynucleotide encoding all NS proteins except a NS4A and a NS5.

In certain embodiments, the nucleic acid composition comprises a polynucleotide encoding all NS proteins except a NS4B and a NS5.

In certain embodiments, the nucleic acid composition comprises a polynucleotide encoding all NS proteins except a NS1. In certain embodiments, the nucleic acid composition comprises a polynucleotide encoding all NS proteins except a NS2A. In certain embodiments, the nucleic acid composition comprises a polynucleotide encoding all NS proteins except a NS2B. In certain embodiments, the nucleic acid composition comprises a polynucleotide encoding all NS proteins except a NS3. In certain embodiments, the nucleic acid composition comprises a polynucleotide encoding all NS proteins except a NS4A. In certain embodiments, the nucleic acid composition comprises a polynucleotide encoding all NS proteins except a NS4B. In certain embodiments, the nucleic acid composition comprises a polynucleotide encoding all NS proteins except a NS5.

In some embodiments, the NS1 is a zika virus NS1. In some embodiments, the NS2A is a zika virus NS2A. In some embodiments, the NS2B is a zika virus NS2B. In some embodiments, the NS3 is a zika virus NS3. In some embodiments, the NS4A is a zika virus NS4A. In some embodiments, the NS4B is a zika virus NS4B. In some embodiments, the NS5 is a zika virus NS5.

In some embodiments, the components of the nucleic acid composition comprise African zika virus components, Asian zika virus components, or Brazilian zika virus components, or a combination of two or more thereof. In one embodiment, the zika virus is African MR766 strain.

In some embodiments, components of the nucleic acid composition are expressed on one or more separate nucleic acids. In some embodiments, the nucleic acid composition comprises one or more expression control elements in operable linkage that confers expression of the nucleic acid composition in vitro or in vivo. In some embodiments, the expression control element is a promoter that drives expression of the nucleic acid composition in vitro. In certain embodiments, the promoter is T7, T3, SP6, or any phage promoter.

In some embodiments, the expression control element is a promoter that drives expression of the nucleic acid composition in a target cell. In some embodiments, the promoter is CMV, SV40, or any eukaryotic promoter.

In some embodiments, the target cell is a neuron, or a non-neuron cell, Vero, COS, CHO, C6/36, HeLa, HEK, HepG2. In some embodiments, the target cell is an oligodendrocyte, microglia, or astrocyte.

In some embodiments, the recombinant zika virus is generated from expressing the nucleic acid composition described herein in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus.

In some embodiments, the second zika virus used in the further infection is a wild-type zika virus. In some embodiments, the wild-type zika virus is an African, an Asian or a Brazilian strain. In other embodiments, the second zika virus used in the further infection is a modified zika virus. In some specific embodiments, the modified zika virus comprises one or more microRNA-based gene-silencing machineries. In some specific embodiments, the one or more microRNA-based gene-silencing machineries control viral replication.

In some embodiments, the recombinant zika virus is generated from expressing the nucleic acid composition in a producer cell. In some embodiments it is generated without infection of a second zika virus. In some embodiments, the zika virus C or the derivative of the zika virus C, the zika virus prM/M or the derivative of the zika virus prM/M, and the zika virus E or the derivative of the zika virus E are present in the recombinant zika virus or a zika-virus-like particle. In some embodiment, the producer cell is a Vero E6 or C6/36 cell.

In some embodiments, the recombinant zika virus is replication competent. In other embodiments, the recombinant zika virus is replication incompetent without lowering the vector titer. In some embodiments, the recombinant zika virus has decreased insertional mutagenesis. In other embodiments, the recombinant zika virus has decreased immune response.

Some embodiments of this disclosure can include the recombinant zika virus that can comprise a modification in the genome of the virus. In some embodiments, the recombinant zika virus can comprise at least one modification in the genome of the virus. In some embodiments, the recombinant zika virus can comprise at least 2, 3, 4, 5, 6, 7, 8, 9, 10, 11, 12, 13, 14, 15, 20, or even more modifications in the genome of the virus. The modification of the viral genome can comprise a mutation, a deletion, or both of a viral gene. A deletion of a viral gene may include a partial or a complete deletion of the viral gene. It should be noted that as used herein, “partial deletion” or “mutation” may refer to an in situ partial deletion or mutation of an endogenous viral gene, respectively. Alternatively, they may refer to replacing the endogenous viral gene with an otherwise identical exogenous nucleic acid that lacks a portion of the gene (“partial deletion”) or has one or more nucleotide change in the gene (“mutation”).

One method of generating the recombinant zika virus described herein involves introducing a viral genome or portion thereof into a cell in whole or in part, for example, in two or more fragments that assemble into the desired viral genome or portion thereof within the cell. A non-limiting method comprises cloning and amplifying nucleic acid fragments covering the genome of a virus, or portion thereof. Nucleic acid fragments or entire viral genomes may also be produced de novo. A promoter sequence, such as a cytomegalovirus (CMV) promoter may be inserted at the end of the first fragment. For the recombinant zika virus having a heterologous nucleic acid sequence, this sequence is fused into or adjoining one of the fragments. In one method to produce an oncolytic virus, amplified fragments are introduced into cells, for example, by electroporation, and the cells are then transferred into growth medium. Cell supernatants are then recovered and stored or used to infect cells from which clarified virus is obtainable from culture supernatants.

Any suitable method is useful for generating genetic modifications in the recombinant zika virus described herein, including mutagenesis, polymerase chain reaction, homologous recombination, or any other genetic engineering technique available in the art. Mutagenesis includes modification of a nucleotide sequence, a single gene, or blocks of genes, and involves removal, addition and/or substitution of a single or plurality of nucleotide bases. In some cases, genetic modification comprises use of genetic recombination techniques to delete or replace at least part of native viral sequence. For example, a polynucleotide replaces part or all of a region of native viral sequence of interest. Alternatively or additionally, a polynucleotide is inserted into the native viral sequence. This inserted polynucleotide may be functional and encode, e.g., a suicide protein, therapeutic agent, and/or reporter protein. Exemplary non-limiting polynucleotides encoding for reporter proteins include green fluorescent protein, enhanced green fluorescent protein, beta-galactosidase, luciferase, and HSV-tk.

### Exogenous Polynucleotides

In some embodiments, disclosed herein are recombinant zika viruses comprising an exogenous polynucleotide. The exogenous polynucleotide may be the target of a regulatory polynucleotide, e.g., the exogenous polynucleotide target is at least 80% complementary to the regulatory polynucleotide. The target may be at least about 80%, 85%, 90%, 91%, 92%, 93%, 94%, 95%, 96%, 97%, 98%, 99%, or 100% complementary to the regulatory polynucleotide. An example regulatory polynucleotide is a ribonucleic acid (RNA) molecule. For instance, the RNA is a microRNA or miRNA and an oncolytic virus comprises a target of the miRNA (“target miRNA” or “target sequence”). Non-limiting example exogenous polynucleotides that are targets of miRNA are provided in column 3 of Table 6A. Non-limiting example miRNA are provided in Table 6B and column 2 of Table 6A. In some embodiments, the exogenous polynucleotide is at least 80%, 85%, 90%, 91%, 92%, 93%, 94%, 95%, 96%, 97%, 98%, 99%, or 100% identical to a sequence of column 3 of Table 6A or to a complementary sequence of Table 6B. In some embodiments, the exogenous polynucleotide anneals to a polynucleotide at least 80%, 85%, 90%, 91%, 92%, 93%, 94%, 95%, 96%, 97%, 98%, 99%, or 100% identical to a sequence in column 2 of Table 6A. In some embodiments, the exogenous polynucleotide anneals to a polynucleotide at least 80%, 85%, 90%, 91%, 92%, 93%, 94%, 95%, 96%, 97%, 98%, 99%, or 100% identical to a complementary sequence of Table 6B. In some embodiments, the exogenous polynucleotide comprises about 6 bases, about 9 bases, about 12 bases, about 15 bases, or about 18 bases.

The exogenous polynucleotide may be positioned within the oncolytic virus such that the oncolytic virus comprising the exogenous polynucleotide retains oncolytic activity. The retained oncolytic activity may be measured by an in vitro or in vivo assay. The retained oncolytic activity may be at least 50%, 60%, 70%, 80%, 90%, or 100% of the oncolytic activity of the oncolytic virus without the exogenous polynucleotide.

In some embodiments, the exogenous polynucleotide comprises a target of a miRNA (e.g., the target anneals to the miRNA), where the miRNA expresses in non-tumor cells, but has lower or no expression in tumor cells. In example embodiments, an oncolytic virus comprising the target of the miRNA has lower or no viral activity in non-tumor cells as compared to viral activity in tumor cells. In example embodiments, an oncolytic virus comprising the target of a miRNA has lower or no viral activity in non-tumor cells as compared to the oncolytic virus without the target of the miRNA. In example embodiments, an oncolytic virus comprising the target of a miRNA has lower or no replication in non-tumor cells as compared to replication in tumor cells. In example embodiments, an oncolytic virus comprising a target of a miRNA has lower or no replication in non-tumor cells as compared to the oncolytic virus without the target of the miRNA. Non-limiting example miRNA are provided in column 2 of Table 6A. Non-limiting example miRNA targets are provided in column 3 of Table 6A. Non-limiting examples of miRNAs are provided in Table 6B, from which miRNA targets may be derived.

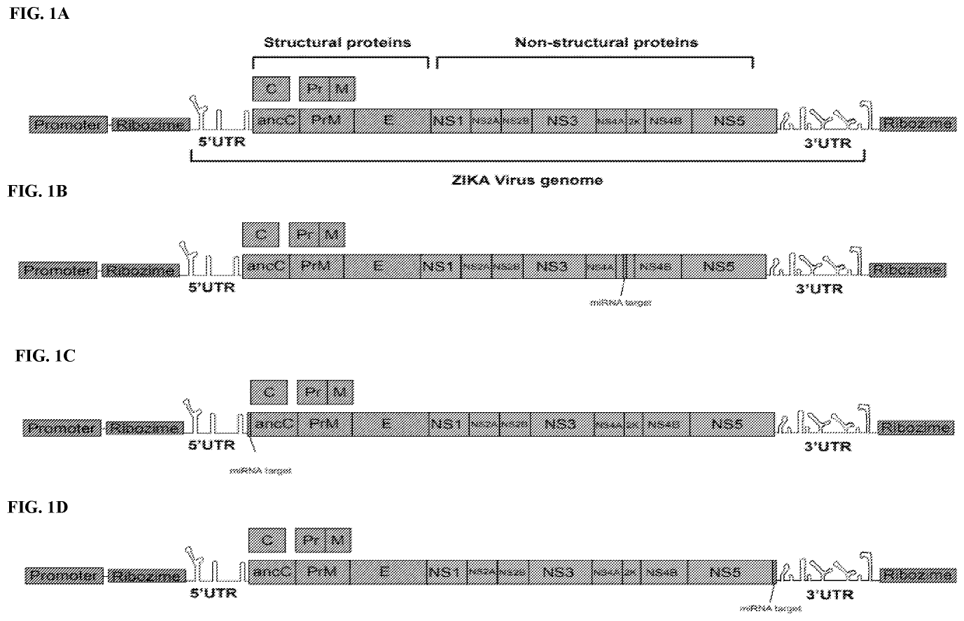

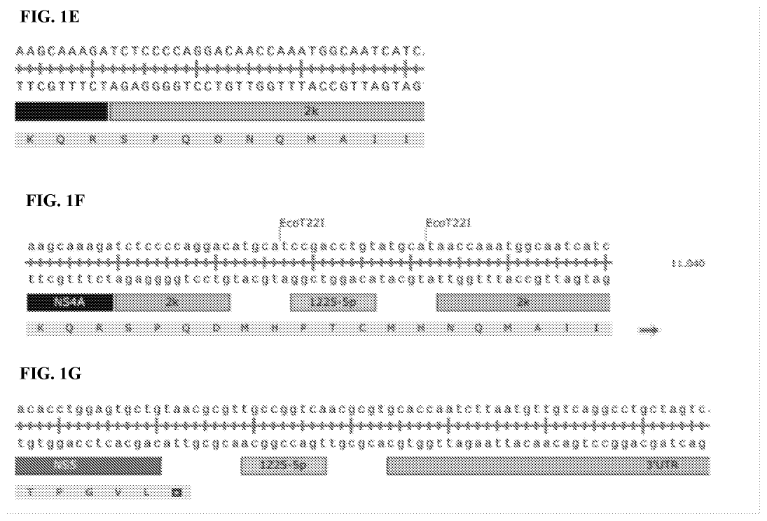

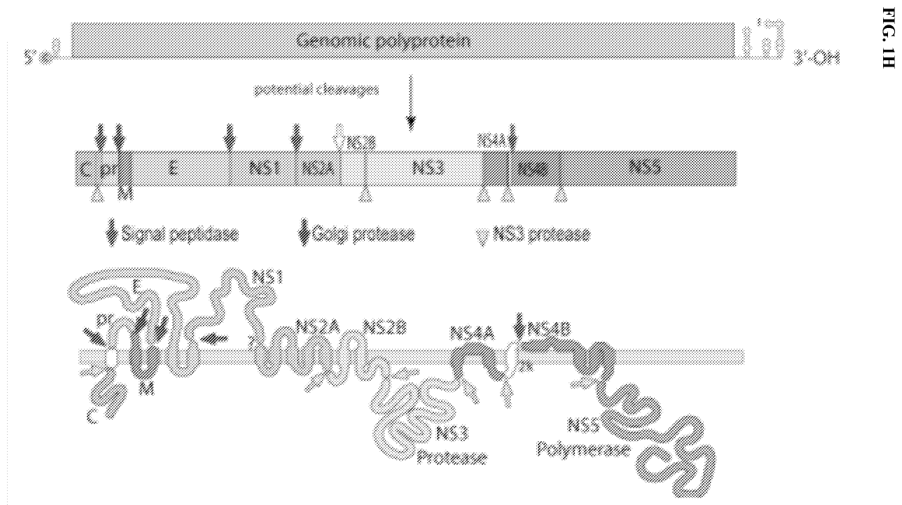

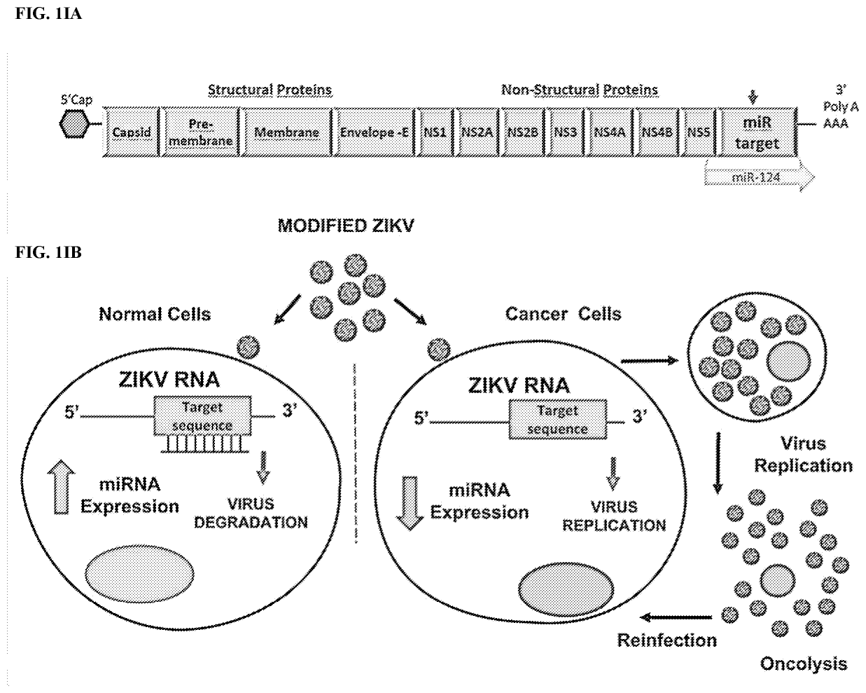

In some embodiments, the exogenous polynucleotide is inserted into an untranslated region of the oncolytic virus. As an example, the exogenous polynucleotide is inserted into the region between the coding sequences and the 3′ UTR (e.g., as shown in FIG. 1D). As another example, the exogenous polynucleotide is inserted into the region between the 5′ UTR and the coding sequences (e.g., as shown in FIG. 1C). In some embodiments, the exogenous polynucleotide is inserted into the coding sequence for non-structural proteins (e.g., as shown in FIG. 1B). In some embodiments, the exogenous polynucleotide is inserted between coding sequences for non-structural proteins and the 3′ UTR (e.g., as shown in FIG. 1G). A non-limiting example provides for insertion of the polynucleotide sequence in the 2k coding sequence (e.g., as shown in FIG. 1F).

A non-limiting example oncolytic virus is shown in FIG. 1IA. In this figure, the exogenous polynucleotide is a miRNA target positioned adjacent to the 3′ UTR region of the viral genetic material. However, the exogeneous polynucleotide may be a target of a non-miRNA polynucleotide, such as an alternative regulatory polynucleotide. The exogeneous polynucleotide may also be positioned elsewhere, e.g., as shown in FIGS. 1B or 1C. In this example figure, the miRNA target attributes a selective tropism and specific replication in certain tissues. In some embodiments, the exogenous polynucleotide is a target of (e.g., at least 80% complementary to) a miRNA that is highly expressed in normal tissues, such as neurons, and has low expression in tumor cells. As such, the presence of the exogenous polynucleotide allows for replication of the modified oncolytic virus in neoplastic cells. The miRNA silencing of modified oncolytic viruses in non-tumoral cells was accomplished when endogenously expressed miRNA targets the exogenous polynucleotide sequence in the virus. The tumoral cell lines that do not endogenously express the miRNA allow virus replication by not targeting exogenous polynucleotide sequence. This mechanism of action is shown in FIG. 1IA-1IB.

### Regulatory Regions

In some embodiments, a recombinant zika virus, such as an oncolytic virus described herein, comprises one or more ribozymes. Ribozymes are self-cleaving RNAs. Small ribozyme motifs mainly fall within four types: hammerhead, hairpin, Varkud satellite (VS), and hepatitis delta virus (HDV). In some embodiments, the recombinant zika virus comprises a hammerhead ribozyme. In some embodiments, the recombinant zika virus comprises a HDV ribozyme.

In some embodiments, the recombinant zika virus is produced from a nucleic acid composition comprising a polynucleotide encoding for one or more ribozymes. In some embodiments, the polynucleotide encodes for a ribozyme upstream of the 5′ UTR. In some embodiments, the ribozyme upstream of the 5′ UTR is a hammerhead ribozyme. In some embodiments, the polynucleotide encodes for a ribozyme downstream of the 3′ UTR. In some embodiments, the ribozyme downstream from the 3′ UTR is a HDV ribozyme.

### Methods of Use

In one aspect, disclosed herein are oncolytic viruses for treating or preventing one or more diseases, conditions, and/or symptoms of cancer. Methods of use involve contacting a cancerous cell with an effective amount of the recombinant zika virus described herein. The contacting may be carried out in vitro (e.g., in biochemical and/or cellular assays), in vivo in a non-human animal, and in vivo in mammals, including humans. In some cases, contacting involves bringing a cancer cell and a composition comprising the recombinant zika virus described herein into sufficient proximity such that the composition exerts an effect on the cancer cell. Contacting includes physical interaction between the composition and a cancer cell, as well as interactions that do not require physical interaction. Contacting includes administering an effective amount of a composition to a subject comprising the cancer cell such that the composition impairs cancer cell growth, division and/or induces cancer cell death. In some embodiments, contacting by administration includes intravenous administration, intraperitoneal administration, intramuscular administration, intracoronary administration, intraarterial administration, subcutaneous administration, transdermal delivery, intratracheal administration, subcutaneous administration, intraarticular administration, intraventricular administration, inhalation, intracerebral, nasal, oral, pulmonary administration, impregnation of a catheter, and direct injection into a tissue or tumor of the subject.

Subjects include animals, such as humans, other higher primates, lower primates, and animals of veterinary importance, such as dogs, cats, horses, sheep, goats, and cattle and the like. Subjects also include animals for use in studies, for example, mice, rats and other rodents.

Provided is a method for treating cancer in a subject comprising administration of an effective amount of a pharmaceutical composition that includes the recombinant zika virus described herein to said subject. An effective amount or therapeutically effective amount of a composition is a quantity sufficient to achieve a desired therapeutic and/or prophylactic effect, e.g., an amount which results in the prevention of, or a decrease in, the symptoms associated with a cancer. The amount of a composition administered to the subject may depend on the type and severity of the cancer and on the characteristics of the individual, such as general health, age, sex, body weight and tolerance to drugs. In some embodiments, an effective amount of an oncolytic virus is administered to a subject having cancer in an amount sufficient to induce oncolysis, the disruption or lysis of a cancer cell, slowing, inhibition and/or reduction in the growth or size of a tumor, and includes the eradication of the tumor in certain instances. In some embodiments, an effective amount of an oncolytic virus is administered to a subject having cancer in an amount sufficient to attenuate or halt division of a cancer cell.

Treatment of cancer includes amelioration, cure, and/or maintenance of a cure (i.e. the prevention or delay of relapse) and/or its associated symptoms. For example, a subject is successfully treated for a cancer if after receiving a therapeutic amount of the composition described herein, the subject shows observable and/or measurable reduction in or absence of one or more signs and symptoms of the cancer, e.g., reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, of one or more of the symptoms associated with the specific cancer; reduced morbidity and mortality, and improvement in quality of life issues. Treatment also includes preventing the cancer from becoming worse, slowing the rate of progression, and/or preventing the cancer from re-occurrence after initial elimination. A suitable dose and therapeutic regimen may vary depending upon the specific oncolytic virus used, the mode of delivery of the oncolytic virus, and whether it is used alone or in combination with one or more other oncolytic viruses.

In some embodiments, a method comprises administration of the recombinant zika virus described herein to a subject having a tumor, such that tumor cell growth or proliferation is inhibited. In some cases, tumor growth or proliferation is reduced by at least about 10%, 20%, 30%, 40%, 50%, 60%, 70%, 80%, 90% 95% or 100%, and includes inhibition of tumor cell division and/or induction of tumor cell death.

In some embodiments, a method comprises administration of the recombinant zika virus described herein to a subject having a tumor, such that tumor cell progression (e.g., tumorigenesis, tumor growth and proliferation, invasion and metastasis) is inhibited. In some cases, inhibiting tumor progression refers to inhibiting the development, growth, proliferation, or spreading of a tumor, including without limitation the following effects: inhibition of growth of cells in a tumor, (2) inhibition, to some extent, of tumor growth, including slowing down or complete growth arrest; (3) reduction in the number of tumor cells; (4) reduction in tumor size; (5) inhibition (i.e., reduction, slowing down or complete stopping) of tumor cell infiltration into adjacent peripheral organs and/or tissues; (6) inhibition (i.e. reduction, slowing down or complete stopping) of metastasis; (7) increase in the length of survival of a patient or patient population following treatment for a tumor; and/or (8) decreased mortality of a patient or patient population at a given time point following treatment for a tumor. In some cases, tumor progression is reduced by at least about 10%, 20%, 30%, 40%, 50%, 60%, 70%, 80%, 90%, 95% or 100%.

In some embodiments, a therapeutic benefit is achieved after administration of the recombinant zika virus described herein. A therapeutic benefit includes anything that promotes or enhances the well-being of the subject with respect to the medical treatment of his/her condition, which includes treatment of pre-cancer, cancer, and hyperproliferative diseases. For example, extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition.

Administration of a pharmaceutical composition to a subject is by means which the recombinant zika virus described herein contained therein will contact a target cell. The specific route will depend upon certain variables such as the cancer cell, and can be determined by the skilled practitioner. Suitable methods of administering a composition comprising a pharmaceutical composition of the present invention to a patient include any route of in vivo administration that is suitable for delivering a virus to a patient. Exemplary methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In an embodiment where the target cells are in or near a tumor, a preferred route of administration is by direct injection into the tumor or tissue surrounding the tumor.

Various routes of administration are contemplated for various tumor types. Where discrete tumor mass, or solid tumor, may be identified, a variety of direct, local and regional approaches may be taken. For example, the tumor is directly injected with the composition. A tumor bed may be treated prior to, during or after resection and/or other treatment(s). Following resection or other treatment(s), one generally will deliver the adenovirus by a catheter having access to the tumor or the residual tumor site following surgery. Methods of treating cancer include treatment of a tumor as well as treatment of the region near or around the tumor. This includes body cavities in which the tumor lies, as well as cells and tissue that are next to the tumor.

In some embodiments, the recombinant zika virus as described herein can be suitable for systemic delivery.

In some embodiments, the recombinant zika virus as described herein may be capable of immune evasion.

In some embodiments, the systemic delivery can comprise oral administration, parenteral administration, intranasal administration, sublingual administration, rectal administration, transdermal administration, or any combinations thereof.

In some embodiments, the parenteral delivery can comprise an intravenous injection.

In some embodiments, the recombinant zika virus as described herein can be suitable for intratumoral delivery.

In some embodiments, the method of treating a cancer can comprise administering to a subject a therapeutically effective amount of the recombinant zika virus as disclosed herein, or a pharmaceutical composition as disclosed herein, wherein the method further can comprise administration of a further therapy.

In some embodiments, the further therapy can comprise chemotherapy, radiation, oncolytic viral therapy with an additional virus, treatment with immunomodulatory proteins, a CAR T cellular therapy (chimeric antigen receptor T cell therapy), an anti-cancer agent, or any combinations thereof.

Another aspect of the present disclosure provides a method of producing a toxic effect in a cancer cell, comprising: administering to a cancer cell a therapeutically effective amount of the recombinant zika virus as disclosed herein. In some embodiments, the cancer cell can be present in a subject. In some embodiments, the subject can be in need of the method that producing the toxic effect in the cancer cell.

Another aspect of the present disclosure provides a method of treating a cancer in a subject, comprising: administering the recombinant zika virus as disclosed herein or pharmaceutical composition comprising the same in combination with a chemotherapeutic prodrug.

Another aspect of the present disclosure provides a method of treating a cancer in a subject, comprising: (i) administering the recombinant zika virus as disclosed herein or a pharmaceutical compositions comprising the same; (ii) assaying a viral titer in a first and a second biological sample isolated from the subject, wherein the first biological sample can comprise a cancer cell and the second biological sample can comprise a non-cancer cell; and (iii) administering a chemotherapeutic prodrug if the viral titer can be equal to or higher in the second sample than the first sample, wherein administration of the chemotherapeutic prodrug results in inhibition of replication of the recombinant zika virus in the subject.

Another aspect of the present disclosure provides a method of treating a cancer in a subject, comprising: (i) administering the recombinant zika virus as described herein or a pharmaceutical compositions comprising the same; (ii) assaying a viral titer in a first and a second biological sample isolated from the subject, wherein the first biological sample can comprise a cancer cell and the second biological sample can comprise a non-cancer cell; and (iii) administering a chemotherapeutic prodrug if the viral titer can be equal to or higher in the second sample than the first sample, wherein administration of the chemotherapeutic prodrug results in inhibition of replication of the recombinant zika virus as described herein in the subject.

In some embodiments, the method of treating a cancer in a subject can comprise administering the recombinant zika virus as disclosed herein, or a pharmaceutical composition as disclosed herein, wherein the recombinant zika virus, or a pharmaceutical composition can be administered as a bolus injection or a slow infusion.

In some embodiments, the method of treating a cancer in a subject can comprise the administration of the recombinant zika virus as disclosed herein, or the pharmaceutical composition as disclosed herein to a subject in need thereof, wherein the subject can be human. In some embodiments, the method of treating a cancer in a subject can comprise the administration of the recombinant zika virus as disclosed herein, or the pharmaceutical composition as disclosed herein to the subject in need thereof, wherein prior to administration of the recombinant zika virus, or the pharmaceutical composition the subject has been diagnosed with a cancer. In some embodiments, the method of treating a subject can comprise the administration of the recombinant zika virus as disclosed herein, or the pharmaceutical composition as disclosed herein to the subject in need thereof, wherein prior to administration of the recombinant zika virus, or the pharmaceutical composition the subject has been diagnosed with a tumor. In some embodiments, the method of treating a cancer in a subject can comprise the administration of the recombinant zika virus as disclosed herein, or a pharmaceutical composition as disclosed herein to the subject in need thereof in combination with the further therapy, wherein prior to administration of the recombinant zika virus, or the pharmaceutical composition or the further therapy the subject has been diagnosed with a cancer or a tumor. In some embodiments, the method of treating a subject can comprise the administration of the recombinant zika virus as disclosed herein, or a pharmaceutical composition as disclosed herein to the subject in need thereof in combination with the further therapy, wherein prior to administration of the recombinant zika virus, or the pharmaceutical composition or the further therapy the subject has been diagnosed with a tumor.

This disclosure provides methods for treating a subject by administration of one or more recombinant zika viruses, as disclosed herein. An “individual” or “subject,” as used interchangeably herein, refers to a human or a non-human subject. Non-limiting examples of non-human subjects include non-human primates, dogs, cats, mice, rats, guinea pigs, rabbits, pigs, fowl, horses, cows, goats, sheep, cetaceans, etc. In some embodiments, the subject is human.

Provided is a method of producing a toxic effect in a cancer cell comprising administering, to the cancer cell, a therapeutically effective amount of the recombinant zika virus as described herein or a pharmaceutical composition containing the same. This disclosure further provides a method of inhibiting at least one of growth and proliferation of a second cancer cell comprising administering, to a first cancer cell, the recombinant zika virus as described above such that the first cancer cell is infected with said virus. Thus, in some embodiments of the methods disclosed here, it is contemplated that not every cancer or tumor cell is infected upon administering a therapeutically effective amount of the recombinant zika virus as described herein, or a pharmaceutical composition containing the same, and growth of non-infected cells can be inhibited without direct infection.

In some examples, to induce oncolysis, kill cells, inhibit growth, inhibit metastases, decrease tumor size and otherwise reverse or reduce the malignant phenotype of tumor cells, using the methods and compositions of the present disclosure, a cancer cell or a tumor can be contacted with a therapeutically effective dose of the recombinant zika virus as described herein or a pharmaceutical composition containing the same. In certain embodiments, an effective amount of the recombinant zika virus of the present disclosure, such as the recombinant zika virus as described herein or a pharmaceutical composition thereof, can include an amount sufficient to induce oncolysis, the disruption or lysis of a cancer cell or the inhibition or reduction in the growth or size of a cancer cell. Reducing the growth of a cancer cell may be manifested, for example, by cell death or a slower replication rate or reduced growth rate of a tumor comprising the cell or a prolonged survival of a subject containing the cancer cell.

Provided, in some embodiments, is a method of treating a subject having a cancer or a tumor comprising administering, to the subject, an effective amount of a modified virus, as described above. An effective amount in such method can include an amount that reduces growth rate or spread of the cancer or that prolongs survival in the subject. This disclosure provides a method of reducing the growth of a tumor, which method can comprise administering, to the tumor, an effective amount of the recombinant zika virus as described above. In certain embodiments, an effective amount of a modified virus, or a pharmaceutical composition thereof, can include an amount sufficient to induce the slowing, inhibition or reduction in the growth or size of a tumor and can include the eradication of the tumor. Reducing the growth of a tumor may be manifested, for example, by reduced growth rate or a prolonged survival of a subject containing the tumor.

This disclosure also provides a method of determining the infectivity or anti-tumor activity, or amount of tumor specific viral replication of the recombinant zika virus as described herein, which method can comprise; (i) administering to a subject a therapeutically effective amount of the recombinant zika virus as described herein or a pharmaceutical composition according to the present disclosure, which further expresses a luciferase reporter gene, alone or in combination with a further therapy; (ii) collecting a first biological sample from the subject immediately after administering the virus and determining the level of the luciferase reporter in the first biological sample (iii) collecting a second biological sample from the subject following the administration in step (ii) and (iii) detecting the level of the luciferase reporter in the second biological sample, wherein the recombinant zika virus as described herein is determined to be infective, demonstrate anti-tumor activity, exhibit tumor specific viral replication if the level of luciferase is higher in step (iii) than in step (ii). The second biological sample is collected about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i). In some embodiments, the method of mentioned above can further comprise, detecting in steps (i) and (iii), the level of one or more assaying cytokine levels, e.g., IL-2, IL-7, IL-8, IL-10, IFN-γ, GM-CSF, TNF-α, IL-6, IL-4, IL-5, and IL-13, in plasma samples collected from a subject after administering to said subject a therapeutically effective amount of the recombinant zika virus of the present disclosure, such as the recombinant zika virus as described herein or a pharmaceutical composition comprising the same. In some embodiments of this disclosure, the increase in luciferase bioluminescence between steps (ii) and (iv) mentioned above is higher for the recombinant zika virus as described herein, compared to that in an otherwise identical virus that does not comprise the modifications in the recombinant zika virus. Other exemplary techniques for detecting and monitoring viral load after administration of the recombinant zika viruses include real-time quantitative PCR.

Further provided is a method of monitoring the pharmacokinetics following administration of a therapeutically effective amount of the recombinant zika virus described herein according to the present disclosure, such as the recombinant zika virus as described herein or a pharmaceutical composition containing the zika virus, as described herein. An exemplary method for monitoring the pharmacokinetics can comprise the following steps: (i) administering to the subject a therapeutically effective amount of the recombinant zika virus as described herein or a pharmaceutical composition comprising the same, alone or in combination with a further therapy; (ii) collecting biological samples from the subject at one or more time points selected from about 15 minutes, about 30 minutes, about 45 mins, about 60 mins, about 75 mins, about 90 mins, about 120 mins, about 180 mins, and about 240 mins, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i) and (iii) detecting the quantity of the viral genome (or a reporter gene inserted within the viral genome, such as luciferase) in the biological samples collected at the above mentioned time points. In some instances, viral genome copies/mL can be highest in the sample collected at the 15 mins time point and further the sample collected at the 240 mins time point may not contain a detectable quantity of the viral genome. Therefore, in some instances, a viral peak can be observed at about 15 mins following administration and majority of the viruses can be cleared from the subject's system after about 240 mins (or 4 hours). In some instances, a first viral peak can be observed after about 15 mins following administration and a second viral peak can be observed in the biological samples collected in the subsequent time points, e.g., at about 30 mins, about 45 mins, about 60 mins, or about 90 mins. The biological sample can be, in exemplar embodiments, blood, and the quantity of viral genome/mL can be determined by quantitative PCR or other appropriate techniques. In some examples, a first viral peak can be observed after about 15 mins following administration and a second viral peak can be observed after about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months following administration of the recombinant zika virus of the present disclosure.

In some instances, tumor-selective replication of the recombinant zika virus as described herein can be measured through use of a reporter gene, such as a luciferase gene. In some embodiments, the luciferase gene can be inserted into the genome of a virus, and a tumor cell can be infected with the virus. Bioluminescence in infected tumor cells can be measured to monitor tumor-selective replication. Some examples show an increase in luciferase reporter bioluminescence in the recombinant zika virus of this disclosure, compared to that in an otherwise identical zika virus that does not contain the modifications in the recombinant zika virus.

### Methods of Production

The recombinant zika viruses of this disclosure can be produced by methods known to one of skill in the art. In certain embodiments, the recombinant zika virus can be propagated in suitable host cells, e.g., HeLa cells, HEK 293 cells, Aedes albopictus clone C6/36 cells, CHO cells or Vero cells, isolated from host cells and stored in conditions that promote stability and integrity of the virus, such that loss of infectivity over time is minimized. In certain exemplary methods, the recombinant zika viruses are propagated in host cells using cell stacks, roller bottles, or perfusion bioreactors. In some examples, downstream methods for purification of the recombinant zika viruses can comprise filtration (e.g., depth filtration, tangential flow filtration, or a combination thereof), ultracentrifugation, or chromatographic capture. The recombinant zika virus can be stored, e.g., by freezing or drying, such as by lyophilization. In certain embodiments, prior to administration, the stored recombinant zika virus described herein can be reconstituted (if dried for storage) and diluted in a pharmaceutically acceptable carrier for administration.

Some embodiments provide that the recombinant zika virus as described herein, exhibit a higher titer in Hela cells, HEK 293 cells, Aedes albopictus clone C6/36 cells, CHO cells and Vero cells compared to an otherwise identical virus that does not comprise the modifications in the recombinant zika virus. In certain instances, a higher titer in Hela cells, HEK 293 cells, Aedes albopictus clone C6/36 cells, CHO cells and Vero cells is seen in the recombinant zika virus described herein.

### Cancer

As used herein, cancer is a class of diseases characterized by uncontrolled cellular growth. Cancer includes all types of hyperproliferative growth, hyperplastic growth, neoplastic growth, cancerous growths or oncogenic processes, metastatic tissues or malignantly transformed cells, tissues, or organs, irrespective of histopathologic type or stage of invasiveness.

In some embodiments, a composition provided herein prevents and/or attenuates tumor cell growth when administered to a patient having cancer. In some embodiments, a composition herein prevents and/or attenuates tumor cell invasion when administered to a patient having cancer. In some embodiments, a composition herein prevents and/or attenuates tumor cell metastasis when administered to a patient having cancer. In some embodiments, a composition herein an oncolytic virus comprising or derived from a zika virus. In some embodiments, a composition herein an oncolytic virus comprising a recombinant zika virus.

Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, retinal, testicle, skin (melanoma or basal cell cancer)) and hematological tumors (such as the leukemias and lymphomas) at any stage of the disease, with or without metastases. In some embodiments, the cancer comprises a brain tumor. Non-limiting examples of tumors treatable with a composition provided herein include adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma, and teratoma. Glioma refers to a tumor originating in the neuroglia of the brain or spinal cord. Gliomas are derived from the glial cell types such as astrocytes and oligodendrocytes, thus gliomas include astrocytomas and oligodendrogliomas, as well as anaplastic gliomas, glioblastomas, and ependymomas. Additional brain tumors include meningiomas, ependymomas, pineal region tumors, choroid plexus tumors, neuroepithelial tumors, embryonal tumors, peripheral neuroblastic tumors, tumors of cranial nerves, tumors of the hemopoietic system, germ cell tumors, and tumors of the sellar region.

In an embodiment of this disclosure, a method of treatment for a hyperproliferative disease, such as a cancer or a tumor, by the delivery of the recombinant zika virus, is contemplated. Cancers that can be treated by the recombinant zika virus, as described herein, can include, but are not limited to, melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, and sarcoma.

Cancer cells that can be treated by the methods of this disclosure include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma;
mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; the coma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some cases, solid cancers that are metastatic can be treated using the recombinant zika viruses of this disclosure, that is advantageous for systemic delivery. In some cases, solid cancers that are inaccessible or difficult to access, such as for purpose of intratumoral delivery of therapeutic agents, can be treated using the recombinant zika viruses of this disclosure, that is advantageous for systemic delivery. Cancers that are associated with increased expression of free fatty acids can, in some examples, be treated using the recombinant zika viruses of this disclosure, that is advantageous for systemic delivery and forms increased amounts of EEV.

This disclosure also contemplates methods for inhibiting or preventing local invasiveness or metastasis, or both, of any type of primary cancer. For example, the primary cancer can be melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the primary cancer can be lung cancer. For example, the lung cancer can be non-small cell lung carcinoma. Moreover, this disclosure can be used to prevent cancer or to treat pre-cancers or premalignant cells, including metaplasias, dysplasias, and hyperplasias. It can also be used to inhibit undesirable but benign cells, such as squamous metaplasia, dysplasia, benign prostate hyperplasia cells, hyperplastic lesions, and the like. In some embodiments, the progression to cancer or to a more severe form of cancer can be halted, disrupted, or delayed by methods of this disclosure involving the recombinant zika virus as discussed herein.

In some embodiments, the cancer is a brain cancer, a retinal cancer, a testicle cancer, or a prostate cancer. In some specific embodiments, the brain cancer is an astrocytoma, oligodendroglioma, ependymoma, meningioma, schwannoma, craniopharyngioma, germinoma, pineocytoma, or a combination thereof.

### Dosing and Treatment Regimens

Administration frequencies for a pharmaceutical composition comprising the recombinant zika virus provided herein may vary based on the method being practiced, the physical characteristics of the subject, the severity of the cancer, cancer type, and the formulation and the means used to administer the composition.

The duration of treatment will be based on the condition being treated and may be determined by the attending physician. The duration of administration, in many instances, varies depending on a number of factors. Exemplary factors include, without limitation, patient response, severity of symptoms, and cancer type. Under some conditions, treatment is continued for a number of days, weeks, or months. Under other conditions, complete treatment is achieve through administering one, two or three dose of the pharmaceutical composition over the entire course of treatment. In certain aspects, complete treatment can be achieved using a single dose of the pharmaceutical composition.

In certain embodiments wherein a patient's status does improve, the dose of the recombinant zika virus or pharmaceutical composition thereof described herein being administered may be temporarily reduced or temporarily suspended for a certain length of time (i.e. a “drug holiday”).

In certain embodiments the dose of the composition being administered is temporarily reduced or temporarily suspended for a certain length of time (i.e. a “drug diversion”).

In some embodiments, once improvement of the patient's conditions has occurred, a maintenance dose is administered if necessary. Subsequently, in certain embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of the recombinant zika virus provided herein varies depending upon factors such as the particular virus, disease condition and its severity, the identity (e.g., weight, sex) of the subject in need of treatment, but can nevertheless be determined according to the particular circumstances surrounding the case. In some embodiments, the desired dose is conveniently presented in a single dose or in divided doses administered simultaneously (or over a short period of time) or at appropriate intervals, for example as two, three, four or more sub-doses per day.

In some embodiments, administration of the recombinant zika virus provided herein depending on the titer, and the titer of the recombinant zika virus is about 10⁵ PFU/mL to about 10¹⁰ PFU/mL.

In some embodiments, amount of the recombinant zika virus of this disclosure, administered to a subject can be between about 10³ and 10¹² infectious viral particles or plaque forming units (PFU).

In some embodiments, the recombinant zika virus of this disclosure can be administered at a dose that can comprise about 10³ viral particles/dose to about 10¹⁴ viral particles/dose.

In some embodiments, the recombinant zika virus of this disclosure can be administered at a dose that can comprise about 10³ PFU/kg to about 10¹⁴ PFU/kg.

In some embodiments, the recombinant zika virus of this disclosure can be administered at a dose that can comprise about 10³ viral particles/kg to about 10¹⁴ viral particles/kg.

### Pharmaceutical Compositions and Formulations

Provided herein are recombinant zika viruses formulated into pharmaceutical compositions. Pharmaceutical compositions are formulated in a conventional manner using one or more pharmaceutically acceptable inactive ingredients that facilitate processing of the active agent into preparations that can be used pharmaceutically. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure. For some virus delivery methods, a pharmaceutically acceptable vehicle is selected from known pharmaceutically acceptable vehicles for delivery, and should be one in which the virus is stable.

Further provided herein are pharmaceutical compositions that include a virus; a pharmaceutically acceptable inactive ingredient; other medicinal or pharmaceutical agent; carrier; adjuvant; preserving, stabilizing, wetting or emulsifying agent; solution promoter; salt; buffer; excipients; binder; filling agent; suspending agent; flavoring agent; sweetening agents; disintegrating agent; dispersing agent; surfactants; lubricant; colorant; diluent; solubilizer; moistening agent; plasticizers; penetration enhancer; anti-foam agent; antioxidant; preservative; or a combination thereof.

Another aspect of the present disclosure provides a pharmaceutical composition comprising a recombinant zika virus as described herein. In some embodiments, the pharmaceutical composition can comprise a solubilizing agent and an excipient. In some embodiments, the excipient can comprise one or more of a buffering agent, a stabilizer, an antioxidant, a binder, a diluent, a dispersing agent, a rate controlling agent, a lubricant, a glidant, a disintegrant, a plasticizer, a preservative, or any combinations thereof. In some embodiments, the excipient can comprise di-sodium hydrogen phosphate dihydrate, sodium dihydrogen phosphate dihydrate, sodium chloride, myo-inositol, sorbitol, or any combinations thereof. In some embodiments, the pharmaceutical composition does not comprise a preservative. In some embodiments, the pharmaceutical composition can comprise one or more of a preservative, a diluent, and a carrier. In some embodiments, the pharmaceutical composition can comprise an additional active ingredient or a salt thereof. In some embodiments, the solubilizing agent can be sterile water. In some embodiments, the pharmaceutical composition can comprise an additional active ingredient, wherein the additional active ingredient can be a further oncolytic virus.

Another aspect of the present disclosure provides a method of enhancing therapeutic effect of an oncolytic virus upon systemic delivery of the virus to a subject, comprising a systemic administration of the recombinant zika virus as disclosed herein, the recombinant zika virus as described herein, or a pharmaceutical composition as disclosed herein.

Pharmaceutical compositions containing a modified virus, the recombinant zika virus as described herein, can be prepared as solutions, dispersions in glycerol, liquid polyethylene glycols, in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, a pharmaceutical composition as described herein can comprise a stabilizer and a buffer. In some embodiments, a pharmaceutical composition as described herein can comprise a solubilizer, such as sterile water, Tris-buffer. In some embodiments, a pharmaceutical composition as described herein can comprise an excipient. An excipient can be an excipient described in the Handbook of Pharmaceutical Excipients, American Pharmaceutical Association (1986). Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient can be a buffering agent. In some embodiments an excipient can comprise a preservative. Non-limiting examples of suitable preservatives can include antioxidants and antimicrobials. In some embodiments a pharmaceutical composition as described herein can comprise a binder as an excipient. In some embodiments a pharmaceutical composition as described herein can comprise a lubricant as an excipient. In some embodiments a pharmaceutical formulation can comprise a dispersion enhancer as an excipient. In some embodiments a pharmaceutical composition as described herein can comprise a disintegrant as an excipient. In some instances, a pharmaceutical composition as described herein can comprise a chelator.

Also contemplated are combination products that include one or more recombinant zika virus described herein.

Under ordinary conditions of storage and use, the pharmaceutical compositions as described herein can comprise a preservative to prevent the growth of microorganisms. In certain examples, the pharmaceutical compositions as described herein may not comprise a preservative. The pharmaceutical forms suitable for injectable use can include sterile aqueous solutions or dispersions and sterile powders for the extemporaneous preparation of sterile injectable solutions or dispersions. The prevention of the action of microorganisms can be brought about by various antibacterial and antifungal agents.

For parenteral administration in an aqueous solution, for example, the liquid dosage form can be suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed will be known to those of skill in the art in light of the present disclosure. For example, one dosage may be dissolved in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions can be prepared by incorporating the recombinant zika virus according to the present disclosure, the recombinant zika virus as described herein or a pharmaceutical composition containing the same, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein may be formulated in a neutral or salt form. Upon formulation, the pharmaceutical compositions can be administered in a manner compatible with the dosage formulation and in such amount as is therapeutically effective.

In certain embodiments, a pharmaceutical composition of this disclosure can comprise an effective amount of a recombinant virus, disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers can include gels, bioabsorbable matrix materials, implantation elements containing the recombinant zika virus or any other suitable vehicle, delivery or dispensing means or material. Such carriers can be formulated by conventional methods and can be administered to the subject at an effective amount.

### Kits

In one aspect of the disclosure, provided herein are kits which include one or more reagents or devices for the performance of the methods disclosed herein. In some embodiments, the kit comprises the recombinant zika virus provided herein. In some embodiments, the kit comprises a means to administrate the recombinant zika virus provided herein.

In some embodiments, the kit comprises suitable instructions in order to perform the methods of the kit. The instructions may provide information of performing any of the methods disclosed herein, whether or not the methods may be performed using only the reagents provided in the kit. The kit and instructions may require additional reagents or systems.

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of the recombinant zika virus described herein. Non-limiting examples of such materials include, but not limited to, buffers, diluents, filters, needles, syringes, carrier, package, container, vial and/or tube labels listing contents and/or instructions for use, and package inserts with instructions for use. A set of instructions will also typically be included.

In some embodiments, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a pharmaceutical composition comprising the recombinant zika virus provided herein and optional additional active agent is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing the recombinant zika virus described herein formulated in a compatible pharmaceutical carrier can also be prepared, placed in an appropriate container, and labeled for treatment of an indicated condition.

### Non-Limiting Numbered Embodiments

(1) A method of treating cancer, comprising administering to a subject in need thereof a recombinant zika virus. (2) The method of embodiment 1, wherein the recombinant zika virus produces higher level of subgenomic flaviviral RNA (sfRNA) than a wild-type zika virus in a cancer cell of the subject. (3) The method of embodiment 1, wherein the higher level of sfRNA is by at least 10%, at least 20%, at least 30%, at least 40% at least 50%, at least 60%, at least 70%, at least 80%, at least 90%, at least 100% higher. (4) The method of the preceding embodiments, wherein the cancer is a brain cancer, a retinal cancer, a testicle cancer, breast cancer, or a prostate cancer. (5) The method of embodiment 4, wherein the brain cancer is an astrocytoma, an oligodendroglioma, an ependymoma, a meningioma, a schwannoma, a craniopharyngioma, a germinoma, a pineocytoma, or a combination thereof. (6) The method of the preceding embodiments, wherein the recombinant zika virus is produced from a nucleic acid composition comprising a polynucleotide encoding a zika virus capsid protein ancC/C or the derivative of the zika virus ancC/C, a polynucleotide encoding a zika virus viral membrane protein (prM/M) or the derivative of the zika virus prM/M, a polynucleotide encoding a zika virus viral envelope protein E or the derivative of the zika virus E, or any combination thereof. (7) The method of embodiment 6, wherein the nucleic acid composition comprises the polynucleotide encoding the zika virus ancC/C or the derivative of the zika virus ancC/C, the polynucleotide encoding the zika virus prM/M or the derivative of the zika virus prM/M, and the polynucleotide encoding the zika virus E or the derivative of the zika virus E. (8) The method of embodiment 7, wherein the polynucleotide encoding the zika virus ancC/C or the derivative of the zika virus ancC/C, the polynucleotide encoding the zika virus prM/M or the derivative of the zika virus prM/M, and the polynucleotide encoding the zika virus E or the derivative of the zika virus E are expressed on one or more separate nucleic acids. (9) The method of one of embodiments 6-8, wherein the polynucleotide encoding the derivative of the zika virus ancC/C comprises at least one substitution, at least one deletion, and/or at least one insertion as compared to a wild-type zika virus ancC/C. (10) The method of embodiment 9, wherein the zika virus ancC/C or the derivative of the zika virus ancC/C comprises an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 1. (11) The method of one of embodiments 6-8, wherein the polynucleotide encoding the derivative of the zika virus prM/M comprises at least one substitution, at least one deletion, and/or at least one insertion as compared to a wild-type zika virus prM/M. (12) The method of embodiment 11, wherein the zika virus prM/M or the derivative of the zika virus prM/M comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 2. (13) The method of one of embodiments 6-8, wherein the polynucleotide encoding the derivative of the zika virus E comprises at least one substitution, at least one deletion, and/or at least one insertion as compared to a wild-type zika virus E. (14) The method of embodiment 13, wherein the polynucleotide encoding E is translated into a wild zika virus type E. (15) The method of embodiment 13 or 14, wherein the zika virus E or the derivative of the zika virus E comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to sequence of Table 3. (16) The method of one of embodiments 6-15, the nucleic acid composition further comprising a 5′ untranslated region (5′ UTR) of the zika virus. (17) The method of one of embodiments 6-16, the nucleic acid composition further comprising a 3′ untranslated region (3′ UTR) of the zika virus. (18) The method of one of embodiments 6-17, wherein the nucleic acid composition does not comprise a polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). (19) The method of one of embodiments 6-17, wherein the nucleic acid composition comprises polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). (20) The method of one of embodiments 18-19, wherein the NS1 is a zika virus NS1, the NS2A is a zika virus NS2A, the NS2B is a zika virus NS2B, the NS3 is a zika virus NS3, the NS4A is a zika virus NS4A, the NS4B is a zika virus NS4B, or the NS5 is a zika virus NS5, or any combination of two or more thereof. (21) The method of one of embodiments 6-20, wherein the components of the nucleic acid composition comprise African zika virus components, Asian zika virus components, or Brazilian zika virus components, or a combination thereof. (22) The method of embodiment 21, wherein the zika virus is African MR766 strain. (23) The method of one of embodiments 6-22, wherein the components of nucleic acid composition are expressed on one or more separate nucleic acids. (24) The method of one of embodiments 6-23, wherein the nucleic acid further comprises one or more expression control elements in operable linkage that confers expression of the nucleic acid composition in vitro or in vivo. (25) The method of embodiment 24, wherein the expression control element is a promoter that drives expression of the nucleic acid composition in vitro. (26) The method of embodiment 25, wherein the promoter is T7, T3, SP6 or any phage promoter. (27) The method of embodiment 24, wherein the expression control element is a promoter that drives expression of the nucleic acid composition in a target cell. (28) The method of embodiment 27, wherein the promoter is CMV, SV40 or any eukaryotic promoter. (29) The method of embodiment 27, wherein the target cell is a neuron, or a non-neuron cell, Vero, CHLA, COS, CHO, C6/36, HeLa, HEK, or HepG2. (30) The method of embodiment 27, wherein the target cell is an oligodendrocyte, microglia, or astrocyte. (31) The method of one of embodiments 6-30, wherein the recombinant zika virus is generated from expressing the nucleic acid composition in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus. (32) The method of embodiment 31, wherein the second zika virus is a wild-type zika virus. (33) The method of embodiment 32, wherein the wild-type zika virus is an African, an Asian and a Brazilian strain. (34) The method of embodiment 31, wherein the second zika virus is a modified zika virus. (35) The method of embodiment 34, wherein the modified zika virus comprises one or more microRNA-based gene-silencing machineries. (36) The method of embodiment 35, wherein the one or more microRNA-based gene-silencing machineries control viral replication. (37) The method of one of embodiments 6-30, wherein the recombinant zika virus that is generated from expressing the nucleic acid composition in a producer cell, without an infection of a second zika virus, wherein the zika virus C or the derivative of the zika virus C, the zika virus prM/M or the derivative of the zika virus prM/M, and the zika virus E or the derivative of the zika virus E are present in the recombinant zika virus or the zika-virus-like particle. (38) The method of one of embodiments 31-37, wherein the producer cell is a Vero E6 or C6/36 cell. (39) The method of one of embodiments 6-38, wherein the recombinant zika virus is replication competent. (40) The method of one of embodiments 6-38, wherein the recombinant zika virus is replication incompetent without lowering the vector titer or impairing expression of the exogeneous polynucleotide. (41) The method of one of embodiments 6-40, wherein the recombinant zika virus has decreased insertional mutagenesis. (42) The method of one of embodiments 6-41, wherein the recombinant zika virus has decreased immune response. (43) The method of embodiment 2, wherein the recombinant zika virus produces higher level of subgenomic flaviviral RNA (sfRNA) than a wild-type zika virus in a cancer cell of the subject through the presence of a microRNA (miRNA) of SEQ ID NO: 8, SEQ ID NO: 9, SEQ ID NO: 10, SEQ ID NO: 11, SEQ ID NO: 12, SEQ ID NO: 13, or a combination thereof. (44) The method of any one of embodiments 1-43, wherein the recombinant zika virus comprises an exogenous polynucleotide. (45) The method of embodiment 44, wherein the exogenous polynucleotide anneals with a miRNA present in the subject. (46) The method of embodiment 45, wherein the miRNA is present in a non-cancer cell of the subject. (47) The method of embodiment 45 or embodiment 46, wherein the miRNA is selected from Table 6B. (48) The method of any one of embodiments 45-47, wherein the exogenous polynucleotide is at least 80%, 81%, 82%, 83%, 84%, 85%, 86%, 87%, 88%, 89%, 90%, 91%, 92%, 93%, 94%, 95%, 96%, 97%, 98% or 99% identical to a sequence of Table 6A. (49) The method of any one of embodiments 45-47, wherein the exogenous polynucleotide comprises a sequence, wherein the sequence is at least 70% identical to a complementary sequence of a miRNA sequence of Table 6B. (50) The method of any one of embodiments 44-49, wherein the zika virus comprises a nucleic acid encoding for a 2k protein, and the exogenous polynucleotide is positioned within the nucleic acid encoding for the 2k protein. (51) The method of any one of embodiments 6-49, wherein the zika virus comprises a 5′ UTR and a sequence encoding for one or more structural proteins, and the exogenous polynucleotide is positioned between the 5′ UTR and the sequence encoding for the one or more structural proteins. (52) The method of any one of embodiments 44-49, wherein the zika virus comprises a 5′ UTR and the exogenous polynucleotide is positioned downstream of the 5′ UTR. (53) The method of any one of embodiments 44-49, wherein the zika virus comprises a 3′ UTR and a sequence encoding for one or more non-structural proteins, and the exogenous polynucleotide is positioned between the 3′ UTR and the sequence encoding for the one or more structural proteins. (54) The method of any one of embodiments 44-49, wherein the zika virus comprises a 3′ UTR and the exogenous polynucleotide is positioned upstream of the 5′ UTR. (55) The method of any one of embodiments 54, wherein the exogenous polynucleotide is positioned between a coding region of wild-type zika virus and the 3′ UTR. (56) The method of any one of embodiments 16-55, wherein the 5′ UTR comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 6 or SEQ ID NO: 239. (57) The method of any one of embodiments 17-55, wherein the 3′ UTR comprises a sequence at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NO: 7, SEQ ID NO: 241, or SEQ ID NO: 244. (58) The method of any one of embodiments 44-55, wherein the sequence coding for one or more non-structural proteins comprises a sequence at least 70% identical to any one of SEQ ID NO: 240, SEQ ID NO: 243, SEQ ID NO: 245, SEQ ID NO: 247, SEQ ID NO: 248, or SEQ ID NO: 249. (59) The method of any one of embodiments 44-55, wherein the zika virus further comprises a noncoding region. (60) The method of embodiment 59, wherein the noncoding region comprises a sequence at least 70% identical to any one of SEQ ID NO: 238, SEQ ID NO: 242, SEQ ID NO: 246, or ACTGAT. (61) The method of any one of embodiments 44-60, wherein the zika virus further comprises at least one ribozyme. (62) The method of embodiment 60, wherein the at least one ribozyme comprises hammerhead, HDV, or a combination thereof. (63) The method of embodiment 44, wherein the recombinant zika virus comprises at least one copy of the exogenous polynucleotide. (64) The method of any one of embodiments 1-63, wherein administering to a subject in need thereof comprises introducing the recombinant zika virus via intracerebroventricular injection. (65) The method of any one of 1-63, wherein the subject is a mammal.

### Examples

### Example 1: Design and Generation of Recombinant Zika Virus Particles

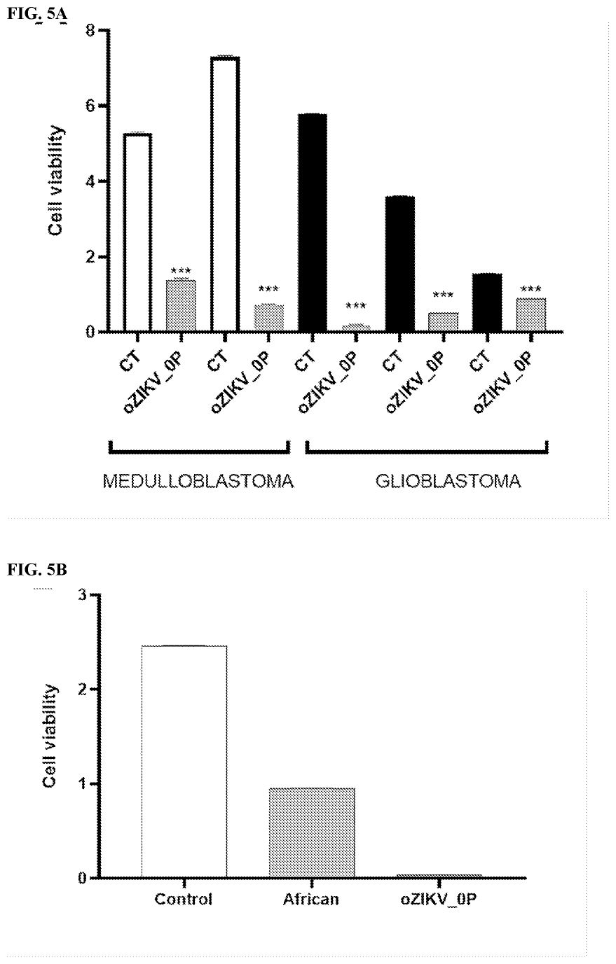

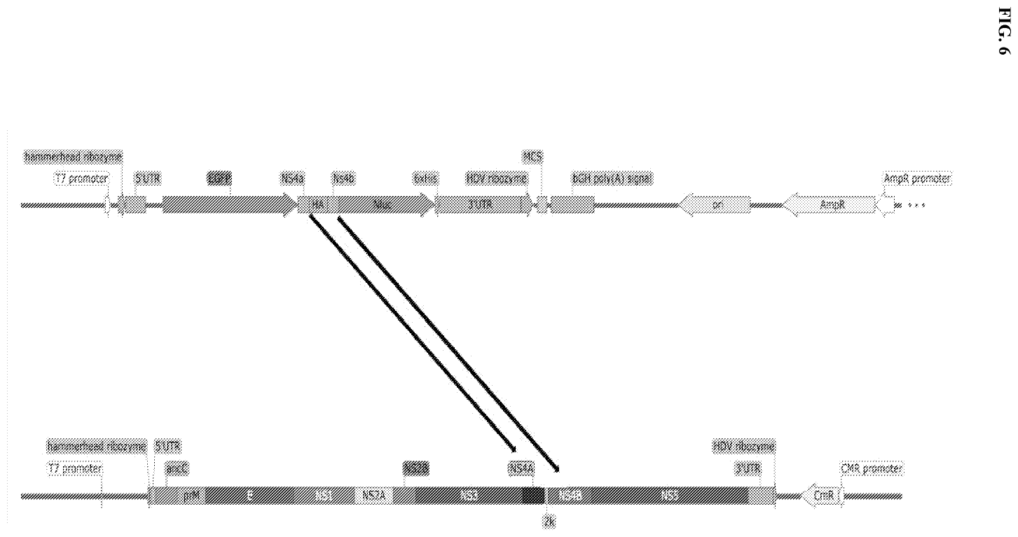

The recombinant zika virus genome was cloned into a plasmid. The viral RNA was produced by an in vitro transcription. The viral RNA was transfected into a Vero cell. The recombinant zika virus was produced and released in the culture supernatant. As shown in FIG. 4A and FIG. 4B, the recombinant zika virus derived from an in vitro transcription and Vero cell transfection kept its oncolytic effect in medulloblastoma cell lines 48 hours after infection. Example engineered zika viruses were generated from the Brazilian zika viral strain. The viral RNA was designed according to genomic sequences deposited in the MH882527.1 (ZIKV-17) and KU365780.1 (ZIKV-IEC) databases. The ZIKV viral genome, containing 10,806 base pairs, was acquired through a DNA synthesis service (GenScript). In addition to the complete ZIKV genome, the viruses were designed to contain the following additional items to increase the efficiency of the modified virus generation process: (a) HammerHead ribozyme at 5′ UTR upstream; (b) HDV ribozyme at 3′ UTR downstream; and (c) T7 promoter. FIG. 1A is an example schematic of this construct, which was cloned into the bacmid pCC1, totaling 21.8 kb.

The T7 promoter allows the in vitro transcription (RNA synthesis in a tube) derived from plasmid DNA. This method was conceived to increase the quality of the final product by avoiding mutations and attenuation of the virus, which could occur after successive infections. Another eukaryote promoter, CMV, was also considered in the genetic engineering strategy to for RNA production efficiency during in vitro transcription. In this way, the production of the active viral concentrate is carried out from the in vitro transcription of the linearized plasmid and transfection (insertion) of this RNA into Vero cells or another lineage of interest.

The ribozymes allow the automatic cleavage of viral RNA once it is transfected (inserted) into the cell, releasing the ZIKV RNA automatically. Once the viral RNA contacts the host cell's translation machinery, it produces a polyprotein, which will be cleaved, giving rise to structural proteins, which will be part of the viral particle, and non-structural proteins, responsible for genome virus replication.

After replication, encapsulation occurs, with the release of modified and active zika viruses. The efficiency of this process is initially confirmed by a clear observation of the cytopathic effect (cell destruction or damage, confirming the presence of viral activity). Analysis of zika NS1 protein presence is also performed by chromatographic test. Subsequently, other analytical tests are performed, such as titration of active viral particles (PFU) and copies of viral RNA (RT-qPCR).

Genetically engineered (or recombinant) viruses were also designed to allow for insertion of exogenous sequences into the viruses without altering the oncolytic ability of the viruses. Example viruses are shown in FIGS. 1B-1D, where the exogenous nucleotide is inserted in the coding region of the 2k protein (FIG. 1B), after the 5′ UTR but before the coding sequences (FIG. 1C), and before the 3′ UTR but after the coding sequences (FIG. 1D).

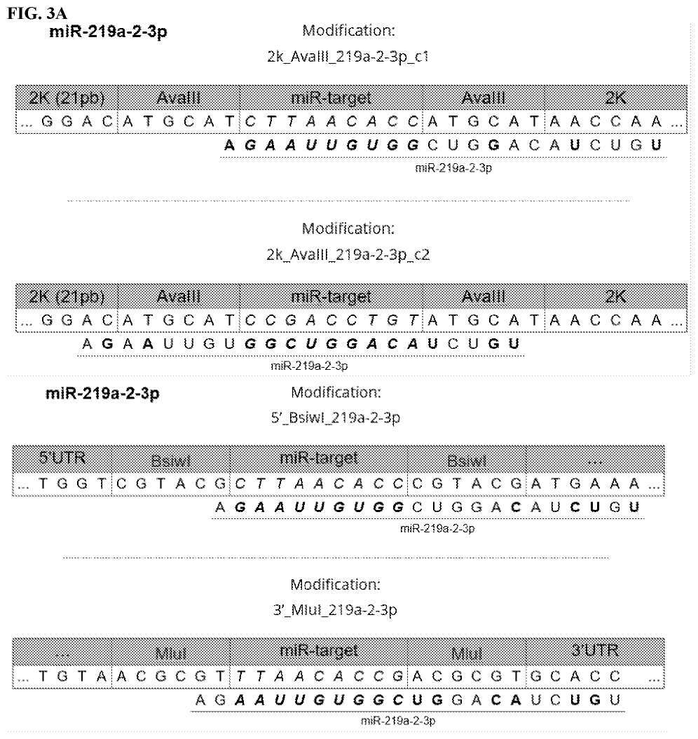

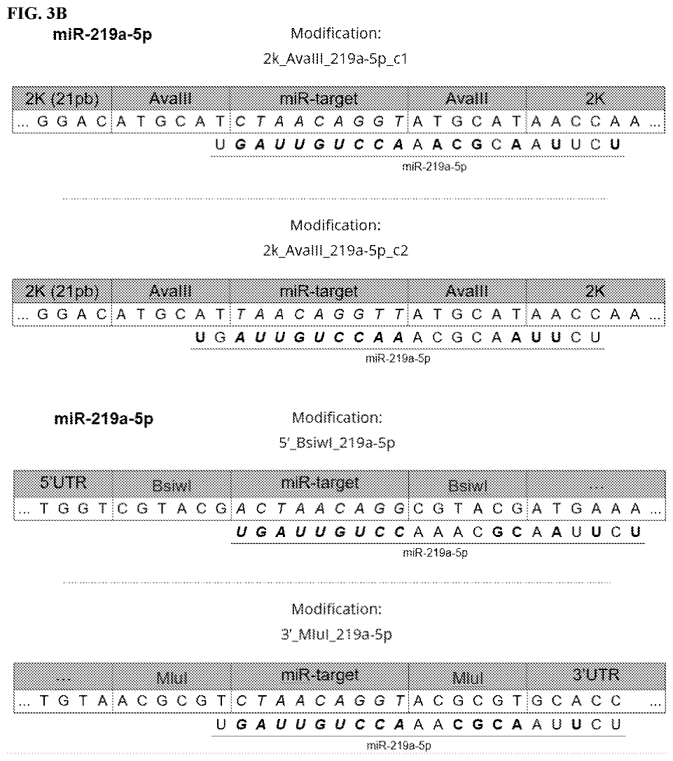

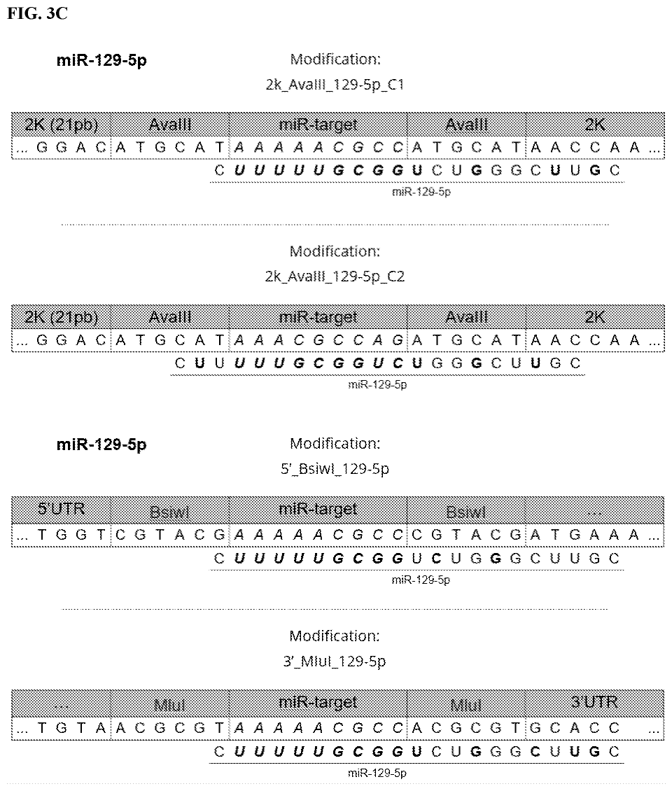

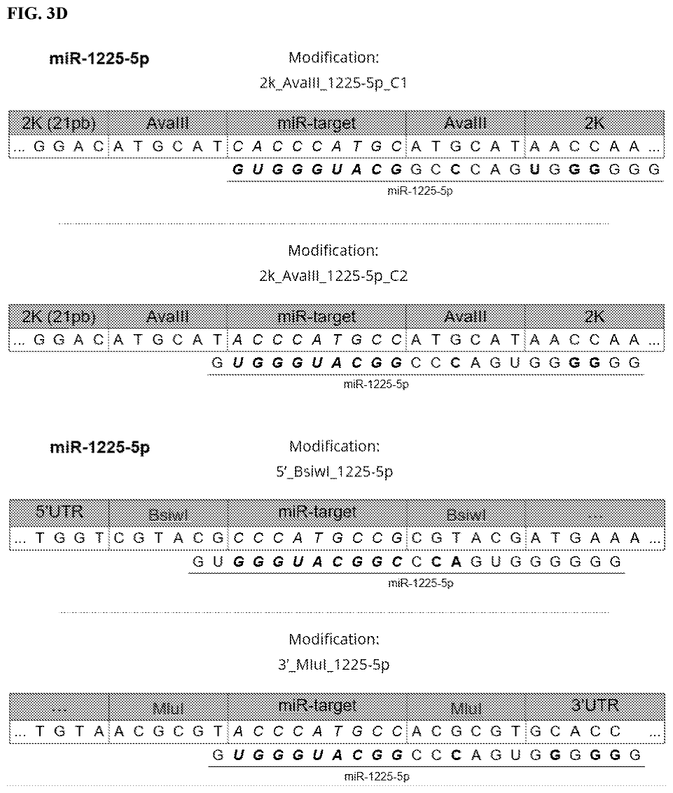

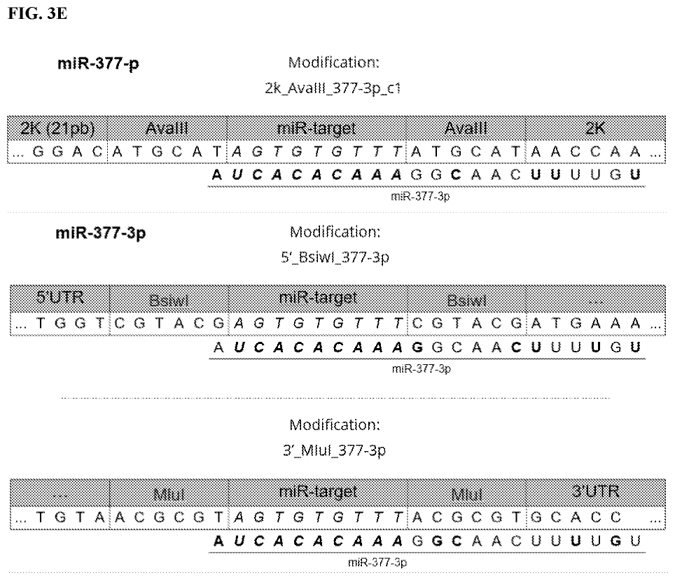

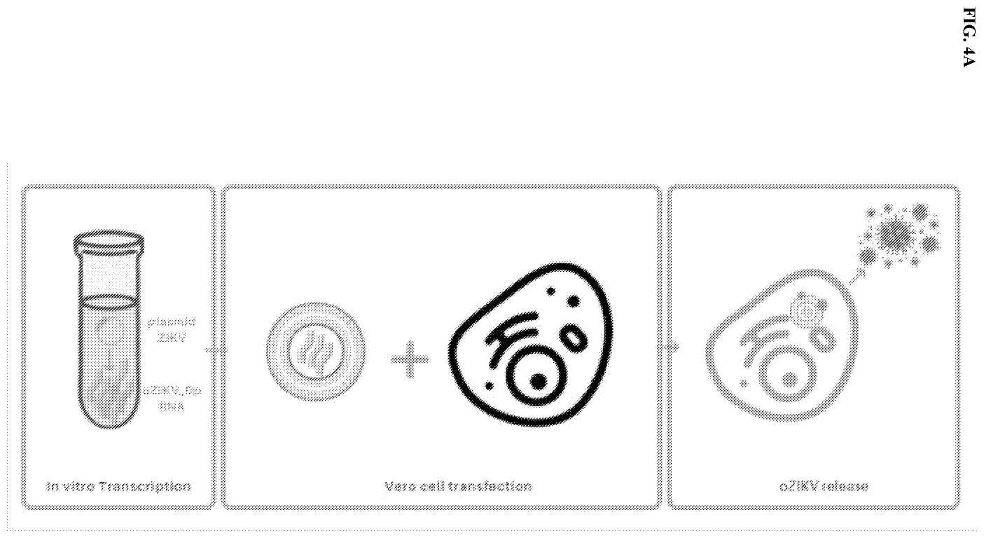

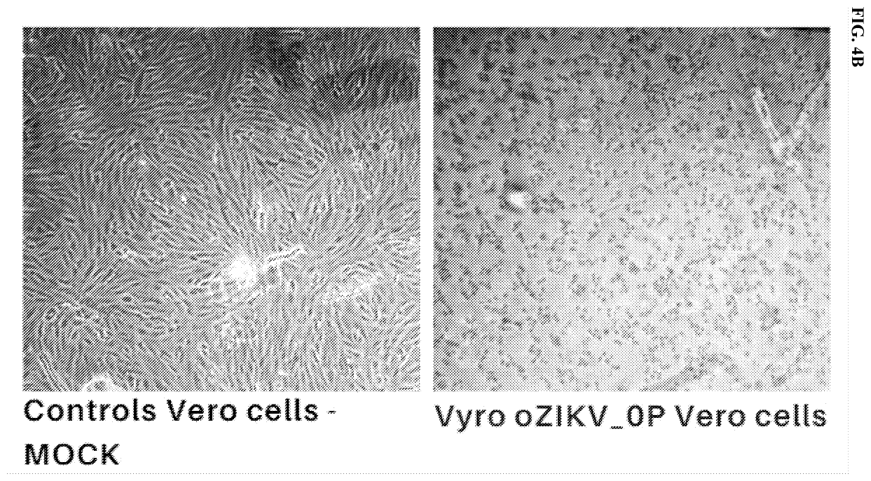

FIGS. 1E-1G provide example schematics of an engineered virus with an exogenous polynucleotide positioned within the 2k coding sequence and before the 3′ UTR (FIG. 1G). FIG. 3A provides examples for the design of miRNA target insertion sequence and restriction sites for miR-219a-2-3p at three example modification regions (2k, 5′ UTR, and 3′ UTR). Additional miRNA targets were also tested as shown in FIGS. 3B-3E for miR-219a-5p, miR-129-5p, miR-1225-5p, miR-377-p, respectively.

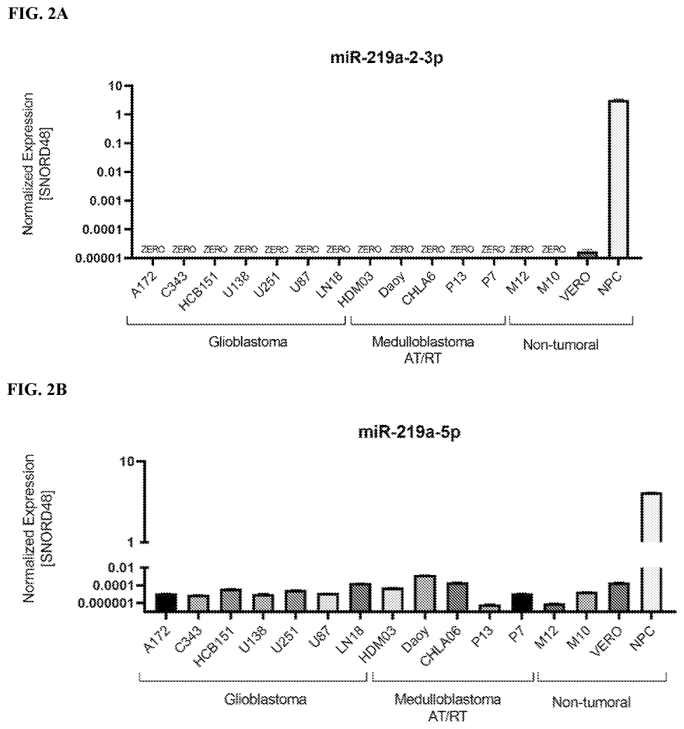

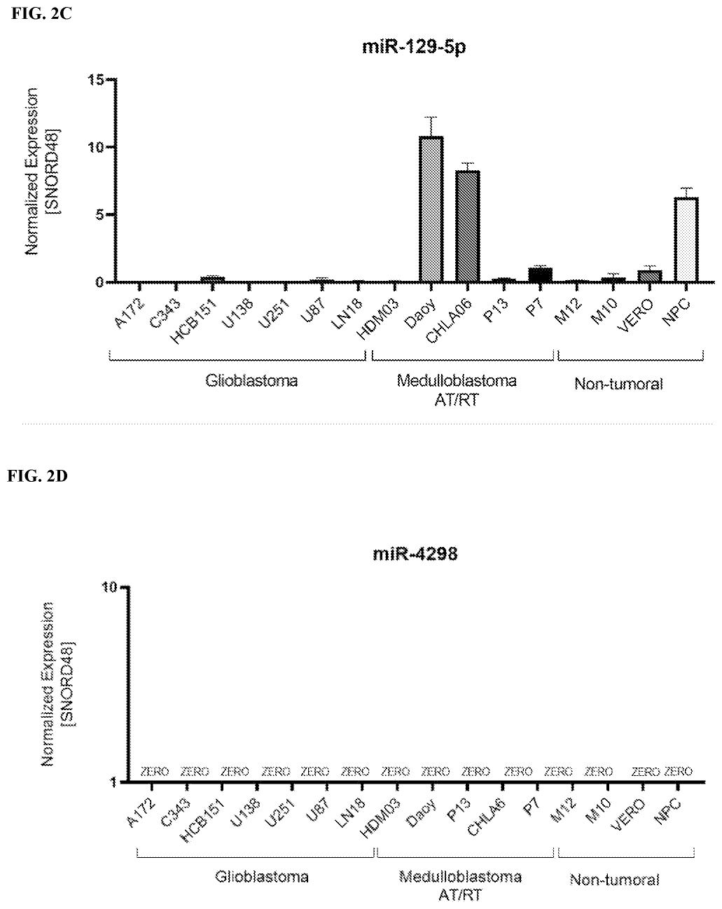

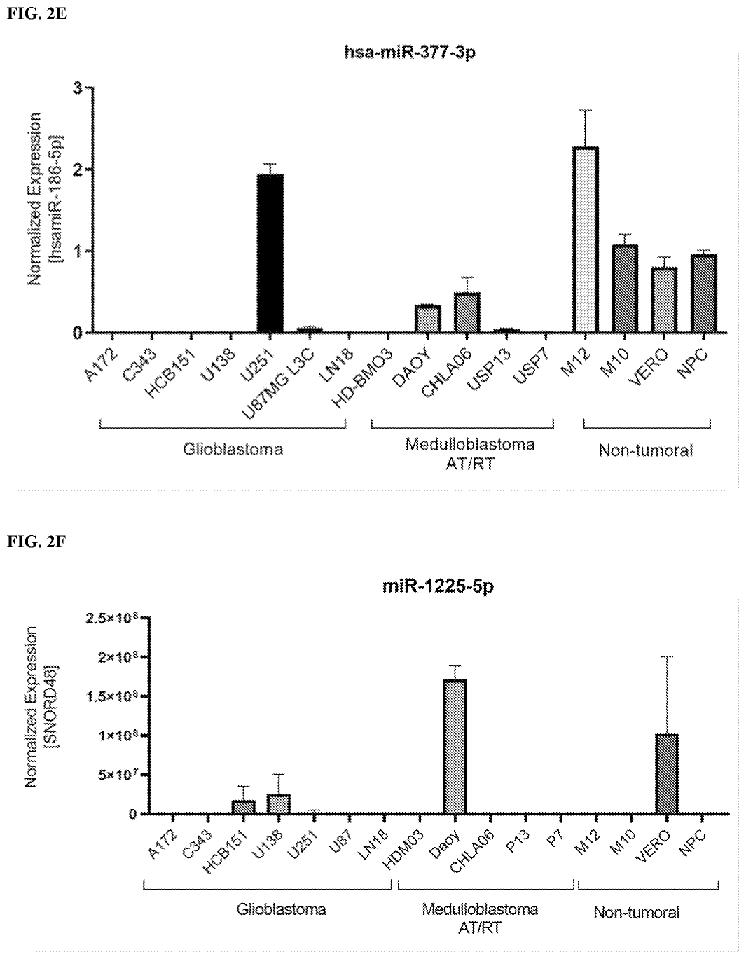

An initial set of miRNAs of interest were identified according to their expression levels in different patient tissues, and the initial miRNA sequences are presented in Tables 6A-6B. The miRNA were inserted into reporter plasmids, and expression analysis was performed on seven glioblastoma cell lines (A172, C343, HCB151, U138, U251, U87, LN18), five CNS embryonic tumor cell lines (medulloblastoma: HDM03, Daoy, P13; ATRT: CHLA06 and P7), three normal human strains (mesenchymal: M12 and M10; Neural progenitor: NPC) and the Vero cell, renal epithelial cells derived from African green monkeys, which were used to produce the modified virus. As shown in FIGS. 2A-2F, the expression levels of each miRNA using TaqMan RT-qPCR in the various tumor cell lines show the viability of targeting miR-219a-2-3p, miR-219a-5p, miR-129-5p, and miR-377-3p (Table 6A) with target sequences in a recombinant zika virus. The target sequences of these four miRNAs were subsequently inserted into a model plasmid. miR-1225-5p was included as a negative control. Further details are provided in Example 3.

TABLE 6A
miRNAs tested and the corresponding SEQ ID NO in parentheses.

| | | Example Target Sequences for |
|---|---|---|
| | | Insertion into Recombinant |
| miRNA ID | Sequence (SEQ ID NO) | Zika Virus (miRNA identifier) |
| miR-219a-2-3p | 5′UAGAAUUGUGGCUGGACAUCUGU | 5′ CCGACCTGT 3′ (m13)/ |
| | 3′ (13) | 5′ CTTAACACC 3′ (m12)/ |
| | | 5′ TTAACACCG 3′ (m14)/ |
| | | 5′ CTTAACAGC 3′ (m15) |
| | | 5′ TGCCGGTCA 3′ (m16) |
| hsa-miR-129-5p | 5′CUUUUUGCGGUCUGGGCUUGC | 5′ AAAAACGCC 3′ (m1)/ |
| | 3′(8) | 5′ AAACGCCAG 3′ (m2)/ |
| | | 5′ GGCGTTTT 3′ (m3) |
| hsa-miR-219a-5p | 5′UGAUUGUCCAAACGCAAUUCU | 5′ CTAACAGGT 3′ (m4)/ |
| | 3′(9) | 5′ TAACAGGTT 3′ (m5)/ |
| | | 5′ ACTAACAGG 3′ (m6) |
| hsa-miR-4298 | 5′CUGGGACAGGAGGAGGAGGCAG | 5′ ACCCTGTCC 3′ (m7) |
| | 3′ (10) | |
| hsa-miR-1225-5p | 5′GUGGGUACGGCCCAGUGGGGGG | 5′ CACCCATGC 3′ (m8)/ |
| | 3′ (11) | 5′ CCCATGCCG 3′ (m9)/ |
| | | 5′ ACCCATGCC 3′ (m10) |
| hsa-miR-377-3p | 5′ | 5′ AGTGTGTTT 3′ (m11) |
| | AUCACACAAAGGCAACUUUUGU | |
| | 3′ (12) | |

TABLE 6B
Example miRNA sequences for use as regulatory polynucleotides.

| Name | Sequence (5′ to 3′) | SEQ ID NO: |
|---|---|---|
| hsa-let-7c-5p | UGAGGUAGUAGGUUGUAUGGUU | 55 |
| hsa-let-7d-5p | AGAGGUAGUAGGUUGCAUAGUU | 56 |
| hsa-let-7e-5p | UGAGGUAGGAGGUUGUAUAGUU | 57 |
| hsa-let-7g-5p | UGAGGUAGUAGUUUGUACAGUU | 58 |
| hsa-miR-100-5p | AACCCGUAGAUCCGAACUUGUG | 59 |
| hsa-miR-101-3p | UACAGUACUGUGAUAACUGAA | 60 |
| hsa-miR-105-5p | UCAAAUGCUCAGACUCCUGUGGU | 61 |
| hsa-miR-107 | AGCAGCAUUGUACAGGGCUAUCA | 62 |
| hsa-miR-10b-5p | UACCCUGUAGAACCGAAUUUGUG | 63 |
| hsa-miR-1202 | GUGCCAGCUGCAGUGGGGGAG | 64 |
| hsa-miR-122-5p | UGGAGUGUGACAAUGGUGUUUG | 65 |
| hsa-miR-124-3p | UAAGGCACGCGGUGAAUGCCAA | 66 |
| hsa-miR-1250-5p | ACGGUGCUGGAUGUGGCCUUU | 67 |
| hsa-miR-125b-2-3p | UCACAAGUCAGGCUCUUGGGAC | 68 |
| hsa-miR-125b-5p | UCCCUGAGACCCUAACUUGUGA | 69 |
| hsa-miR-1265 | CAGGAUGUGGUCAAGUGUUGUU | 70 |
| hsa-miR-126-5p | CAUUAUUACUUUUGGUACGCG | 71 |
| hsa-miR-128-3p | UCACAGUGAACCGGUCUCUUU | 72 |
| hsa-miR-129-1-3p | AAGCCCUUACCCCAAAAAGUAU | 73 |
| hsa-miR-129-2-3p | AAGCCCUUACCCCAAAAAGCAU | 74 |
| hsa-miR-129-5p | CUUUUUGCGGUCUGGGCUUGC | 75 |
| hsa-miR-132-3p | UAACAGUCUACAGCCAUGGUCG | 76 |
| hsa-miR-135a-5p | UAUGGCUUUUUAUUCCUAUGUGA | 77 |
| hsa-miR-137-3p | UUAUUGCUUAAGAAUACGCGUAG | 78 |
| hsa-miR-137-5p | ACGGGUAUUCUUGGGUGGAUAAU | 79 |
| hsa-miR-138-5p | AGCUGGUGUUGUGAAUCAGGCCG | 80 |
| hsa-miR-139-3p | UGGAGACGCGGCCCUGUUGGAGU | 81 |
| hsa-miR-139-5p | UCUACAGUGCACGUGUCUCCAGU | 82 |
| hsa-miR-1-3p | UGGAAUGUAAAGAAGUAUGUAU | 83 |
| hsa-miR-140-5p | CAGUGGUUUUACCCUAUGGUAG | 84 |
| hsa-miR-143-3p | UGAGAUGAAGCACUGUAGCUC | 85 |
| hsa-miR-145-5p | GUCCAGUUUUCCCAGGAAUCCCU | 86 |
| hsa-miR-148a-3p | UCAGUGCACUACAGAACUUUGU | 87 |
| hsa-miR-148b-3p | UCAGUGCAUCACAGAACUUUGU | 88 |
| hsa-miR-149-5p | UCUGGCUCCGUGUCUUCACUCCC | 89 |
| hsa-miR-150-3p | CUGGUACAGGCCUGGGGGACAG | 90 |
| hsa-miR-150-5p | UCUCCCAACCCUUGUACCAGUG | 91 |
| hsa-miR-151a-5p | UCGAGGAGCUCACAGUCUAGU | 92 |
| hsa-miR-153-3p | UUGCAUAGUCACAAAAGUGAUC | 93 |
| hsa-miR-154-5p | UAGGUUAUCCGUGUUGCCUUCG | 94 |
| hsa-miR-181a-5p | AACAUUCAACGCUGUCGGUGAGU | 95 |
| hsa-miR-181b-5p | AACAUUCAUUGCUGUCGGUGGGU | 96 |
| hsa-miR-181c-5p | AACAUUCAACCUGUCGGUGAGU | 97 |
| hsa-miR-184 | UGGACGGAGAACUGAUAAGGGU | 98 |
| hsa-miR-185-5p | UGGAGAGAAAGGCAGUUCCUGA | 99 |
| hsa-miR-186-5p | CAAAGAAUUCUCCUUUUGGGCU | 100 |
| hsa-miR-187-3p | UCGUGUCUUGUGUUGCAGCCGG | 101 |
| hsa-miR-188-5p | CAUCCCUUGCAUGGUGGAGGG | 102 |
| hsa-miR-18a-5p | UAAGGUGCAUCUAGUGCAGAUAG | 103 |
| hsa-miR-18b-5p | UAAGGUGCAUCUAGUGCAGUUAG | 104 |
| hsa-miR-190a-5p | UGAUAUGUUUGAUAUAUUAGGU | 105 |
| hsa-miR-1914-3p | GGAGGGGUCCCGCACUGGGAGG | 106 |
| hsa-miR-191-5p | CAACGGAAUCCCAAAAGCAGCUG | 107 |
| hsa-miR-194-5p | UGUAACAGCAACUCCAUGUGGA | 108 |
| hsa-miR-197-3p | UUCACCACCUUCUCCACCCAGC | 109 |
| hsa-miR-197-5p | CGGGUAGAGAGGGCAGUGGGAGG | 110 |
| hsa-miR-200a-3p | UAACACUGUCUGGUAACGAUGU | 111 |
| hsa-miR-200b-3p | UAAUACUGCCUGGUAAUGAUGA | 112 |
| hsa-miR-202-3p | AGAGGUAUAGGGCAUGGGAA | 113 |
| hsa-miR-203a-3p | GUGAAAUGUUUAGGACCACUAG | 114 |
| hsa-miR-205-5p | UCCUUCAUUCCACCGGAGUCUG | 115 |
| hsa-miR-206 | UGGAAUGUAAGGAAGUGUGUGG | 116 |
| hsa-miR-20a-5p | UAAAGUGCUUAUAGUGCAGGUAG | 117 |
| hsa-miR-211-5p | UUCCCUUUGUCAUCCUUCGCCU | 118 |
| hsa-miR-212-3p | UAACAGUCUCCAGUCACGGCC | 119 |
| hsa-miR-215-5p | AUGACCUAUGAAUUGACAGAC | 120 |
| hsa-miR-218-5p | UUGUGCUUGAUCUAACCAUGU | 121 |
| hsa-miR-219a-5p | UGAUUGUCCAAACGCAAUUCU | 122 |
| hsa-miR-224-5p | UCAAGUCACUAGUGGUUCCGUUUAG | 123 |
| hsa-miR-26b-5p | UUCAAGUAAUUCAGGAUAGGU | 124 |
| hsa-miR-28-5p | AAGGAGCUCACAGUCUAUUGAG | 125 |
| hsa-miR-297 | AUGUAUGUGUGCAUGUGCAUG | 126 |
| hsa-miR-298 | AGCAGAAGCAGGGAGGUUCUCCCA | 127 |
| hsa-miR-299-5p | UGGUUUACCGUCCCACAUACAU | 128 |
| hsa-miR-29b-3p | UAGCACCAUUUGAAAUCAGUGUU | 129 |
| hsa-miR-29c-3p | UAGCACCAUUUGAAAUCGGUUA | 130 |
| hsa-miR-300 | UAUACAAGGGCAGACUCUCUCU | 131 |
| hsa-miR-302b-3p | UAAGUGCUUCCAUGUUUUAGUAG | 132 |
| hsa-miR-30a-5p | UGUAAACAUCCUCGACUGGAAG | 133 |
| hsa-miR-30c-5p | UGUAAACAUCCUACACUCUCAGC | 134 |
| hsa-miR-30e-3p | UGUAAACAUCCUUGACUGGAAG | 135 |
| hsa-miR-30e-5p | CUUUCAGUCGGAUGUUUACAGC | 136 |
| hsa-miR-31-5p | AGGCAAGAUGCUGGCAUAGCU | 137 |
| hsa-miR-320a-5p | GCCUUCUCUUCCCGGUUCUUCC | 138 |
| hsa-miR-320a-3p | AAAAGCUGGGUUGAGAGGGCGA | 139 |
| hsa-miR-320b | AAAAGCUGGGUUGAGAGGGCAA | 140 |
| hsa-miR-323a-3p | AGGUGGUCCGUGGCGCGUUCGC | 141 |
| hsa-miR-323a-5p | AGGUGGUCCGUGGCGCGUUCGC | 142 |
| hsa-miR-324-5p | CGCAUCCCCUAGGGCAUUGGUG | 143 |
| hsa-miR-328-3p | CUGGCCCUCUCUGCCCUUCCGU | 144 |
| hsa-miR-329-3p | GAGGUUUUCUGGGUUUCUGUUUC | 145 |
| hsa-miR-330-3p | GCAAAGCACACGGCCUGCAGAGA | 146 |
| hsa-miR-331-3p | GCCCCUGGGCCUAUCCUAGAA | 147 |
| hsa-miR-338-3p | UCCAGCAUCAGUGAUUUUGUUG | 148 |
| hsa-miR-338-5p | AACAAUAUCCUGGUGCUGAGUG | 149 |
| hsa-miR-339-5p | UCCCUGUCCUCCAGGAGCUCACG | 150 |
| hsa-miR-33a-5p | GUGCAUUGUAGUUGCAUUGCA | 151 |
| hsa-miR-340-5p | UUAUAAAGCAAUGAGACUGAUU | 152 |
| hsa-miR-346 | UGUCUGCCCGCAUGCCUGCCUCU | 153 |
| hsa-miR-34c-5p | AGGCAGUGUAGUUAGCUGAUUGC | 154 |
| hsa-miR-369-5p | AGAUCGACCGUGUUAUAUUCGC | 155 |
| hsa-miR-370-3p | GCCUGCUGGGGUGGAACCUGGU | 156 |
| hsa-miR-372-3p | AAAGUGCUGCGACAUUUGAGCGU | 157 |
| hsa-miR-373-3p | GAAGUGCUUCGAUUUUGGGGUGU | 158 |
| hsa-miR-374a-5p | UUAUAAUACAACCUGAUAAGUG | 159 |
| hsa-miR-375-3p | UUUGUUCGUUCGGCUCGCGUGA | 160 |
| hsa-miR-375-5p | GCGACGAGCCCCUCGCACAAACC | 161 |
| hsa-miR-376b-3p | AUCAUAGAGGAAAAUCCAUGUU | 162 |
| hsa-miR-376c-3p | CGUGGAUAUUCCUUCUAUGUUU | 163 |
| hsa-miR-377-3p | AUCACACAAAGGCAACUUUUGU | 164 |
| hsa-miR-378a-3p | ACUGGACUUGGAGUCAGAAGGC | 165 |
| hsa-miR-379-5p | UGGUAGACUAUGGAACGUAGG | 166 |
| hsa-miR-380-3p | UAUGUAAUAUGGUCCACAUCUU | 167 |
| hsa-miR-381-3p | UAUACAAGGGCAAGCUCUCUGU | 168 |
| hsa-miR-382-5p | GAAGUUGUUCGUGGUGGAUUCG | 169 |
| hsa-miR-383-5p | AGAUCAGAAGGUGAUUGUGGCU | 170 |
| hsa-miR-409-3p | GAAUGUUGCUCGGUGAACCCCU | 171 |
| hsa-miR-410-3p | AAUAUAACACAGAUGGCCUGU | 172 |
| hsa-miR-411-5p | UAGUAGACCGUAUAGCGUACG | 173 |
| hsa-miR-421-3p | UAUGUAACACGGUCCACUAACC | 174 |
| hsa-miR-421-5p | UAGUAGACCGUAUAGCGUACG | 175 |
| hsa-miR-431-5p | UGUCUUGCAGGCCGUCAUGCA | 176 |
| hsa-miR-432-5p | UCUUGGAGUAGGUCAUUGGGUGG | 177 |
| hsa-miR-433-3p | AUCAUGAUGGGCUCCUCGGUGU | 178 |
| hsa-miR-448 | UUGCAUAUGUAGGAUGUCCCAU | 179 |
| hsa-miR-452-5p | AACUGUUUGCAGAGGAAACUGA | 180 |
| hsa-miR-485-3p | AGAGGCUGGCCGUGAUGAAUUC | 181 |
| hsa-miR-486-5p | UCCUGUACUGAGCUGCCCCGAG | 182 |
| hsa-miR-487b-3p | AAUCGUACAGGGUCAUCCACUU | 183 |
| hsa-miR-495-3p | AAACAAACAUGGUGCACUUCUU | 184 |
| hsa-miR-497-5p | CAGCAGCACACUGUGGUUUGU | 185 |
| hsa-miR-505-3p | CGUCAACACUUGCUGGUUUCCU | 186 |
| hsa-miR-513a-5p | UUCACAGGGAGGUGUCAU | 187 |
| hsa-miR-515-3p | GAGUGCCUUCUUUUGGAGCGUU | 188 |
| hsa-miR-515-5p | UUCUCCAAAAGAAAGCACUUUCUG | 189 |
| hsa-miR-517a-3p | AUCGUGCAUCCCUUUAGAGUGU | 190 |
| hsa-miR-518b | CAAAGCGCUCCCCUUUAGAGGU | 191 |
| hsa-miR-518c-5p | UCUCUGGAGGGAAGCACUUUCUG | 192 |
| hsa-miR-519d-3p | CAAAGUGCCUCCCUUUAGAGUG | 193 |
| hsa-miR-523-3p | GAACGCGCUUCCCUAUAGAGGGU | 194 |
| hsa-miR-525-3p | GAAGGCGCUUCCCUUUAGAGCG | 195 |
| hsa-miR-539-5p | GGAGAAAUUAUCCUUGGUGUGU | 196 |
| hsa-miR-557 | GUUUGCACGGGUGGGCCUUGUCU | 197 |
| hsa-miR-564 | AGGCACGGUGUCAGCAGGC | 198 |
| hsa-miR-572 | GUCCGCUCGGCGGUGGCCCA | 199 |
| hsa-miR-575 | GAGCCAGUUGGACAGGAGC | 200 |
| hsa-miR-584-5p | UUAUGGUUUGCCUGGGACUGAG | 201 |
| hsa-miR-585-3p | UGGGCGUAUCUGUAUGCUA | 202 |
| hsa-miR-623 | AUCCCUUGCAGGGGCUGUUGGGU | 203 |
| hsa-miR-628-3p | UCUAGUAAGAGUGGCAGUCGA | 204 |
| hsa-miR-638 | AGGGAUCGCGGGCGGGUGGCGGCCU | 205 |
| hsa-miR-665 | ACCAGGAGGCUGAGGCCCCU | 206 |
| hsa-miR-668-3p | UGUCACUCGGCUCGGCCCACUAC | 207 |
| hsa-miR-744-5p | UGCGGGGCUAGGGCUAACAGCA | 208 |
| hsa-miR-7-5p | UGGAAGACUAGUGAUUUUGUUGUU | 209 |
| hsa-miR-760 | CGGCUCUGGGUCUGUGGGGA | 210 |
| hsa-miR-765 | UGGAGGAGAAGGAAGGUGAUG | 211 |
| hsa-miR-766-3p | ACUCCAGCCCCACAGCCUCAGC | 212 |
| hsa-miR-769-5p | UGAGACCUCUGGGUUCUGAGCU | 213 |
| hsa-miR-873-5p | GCAGGAACUUGUGAGUCUCCU | 214 |
| hsa-miR-874-3p | CUGCCCUGGCCCGAGGGACCGA | 215 |
| hsa-miR-93-5p | CAAAGUGCUGUUCGUGCAGGUAG | 216 |
| hsa-miR-939-5p | UGGGGAGCUGAGGCUCUGGGGGUG | 217 |
| hsa-miR-940 | AAGGCAGGGCCCCCGCUCCCC | 218 |
| hsa-miR-95-3p | UUCAACGGGUAUUUAUUGAGCA | 219 |
| hsa-miR-98-5p | UGAGGUAGUAAGUUGUAUUGUU | 220 |
| hsa-miR-99a-5p | AACCCGUAGAUCCGAUCUUGUG | 221 |

### Example 2: Oncolytic Effect in Medulloblastoma and Glioblastoma Cells after Infection with Recombinant Zika Virus Particles

Oncolytic effect was evaluated in both glioblastoma and medulloblastoma cell lines in vitro. Briefly, cell viability of medulloblastoma and glioblastoma cell lines, U138MG, U251MG, U343MG, ONS-76, Daoy, with or without the recombinant zika virus particles was measured with cell growth kinetics assay using the xCELLigence Real-Time Cell System (Agilent). As shown in FIG. 5A, viability of glioblastoma cell (U138MG, U251MG and U343MG cell lines) and medulloblastoma cells (ONS-76 and Daoy cell lines) was inhibited after recombinant zika virus infection in MOI 2 when compared with control (CT). As shown in FIG. 5B, the infection of recombinant zika virus (oZIKV-0p) induced a higher oncolytic effect in Daoy medulloblastoma cell line when compared with the African wild-type zika virus infection.

### Example 3. Oncolytic Effect of Recombinant Zika Particles

Confirmation of Inhibition of RNA Replication and Translation after Insertion of miRNA Sequences

Model assays were performed to confirm the inhibition of RNA replication and translation after insertion of the miRNA target sequences. These assays used a small reporter plasmid in place of the entire ZIKV genome, which is a model system for the ZIKV genome for studies prior to modifying the virus containing the entire genome of ZIKV. The modification region comprising a miRNA target sequence was inserted using a reporter plasmid containing some structures of the ZIKV (5′ UTR, Capsid, 3′ UTR) (FIG. 6).

After selecting the desired miRNA sequences for the assay, plasmid constructs having the recombinant zika viral proteins were synthesized. FIG. 6 shows a representative construct of the Replicon plasmid (pRep) having part of the ZIKV genome (NS4B, 2k, NS4A) and reporter sequence (NanoLuciferase, or “NanoLuc”). The BswiI site was introduced into pRep to replace HA (hemagglutinin), which was performed by fusion PR (overlap extension PCR). A region between the T7 promoter and HA (in the middle of the 2k region) and the 3′ UTR was amplified. The amplicon (2.2 kb) was necessary as there were no unique restriction sites within this region. To minimize mutations, a high-fidelity Taq polymerase was used. In this vector, the targets were directly cloned. For each target sequence, a pair of oligos were designed that partially complement each other to recompose the target sequence (miRNA seed) and the cleaved restriction sites. The resulting constructs are presented in Table 7. Integration of the miRNA target (or seed) inserts were confirmed via sequencing. Some plasmids showed two or more copies of the insert of interest. All variations were tested in cells to assess the relationship between the copy number present in the plasmid and the regulatory effect of miRNA.

TABLE 7
The different plasmids having the selected miRNA and corresponding
sequencing details (e.g., number of target (or seed) copies)

| pREP ID | miRNA | Sequencing Details |
|---|---|---|
| 1c | 129-5p | 1 target copy |
| 1e | 129-5p | 2 target copies |
| 2b | 219a-2-3p | 1 target copy |
| 3a | 219a-5p | 1 target copy |
| 3d | 219a-5p | 5 target copies |
| 4c | 377-3p | 1 target copy |
| 4e | 377-3p | 2 targets copies/region with low specificity |
| pRep | 0 | Empty plasmid used as control |

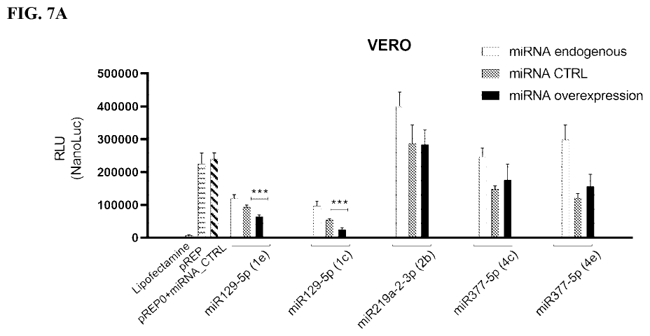

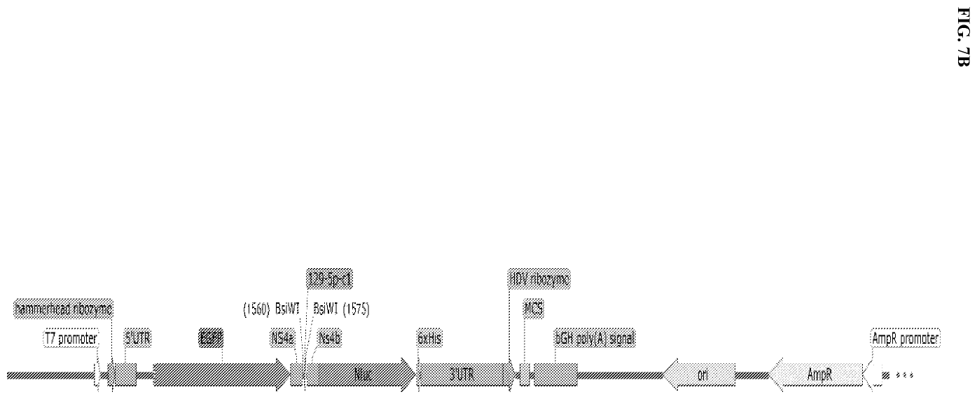

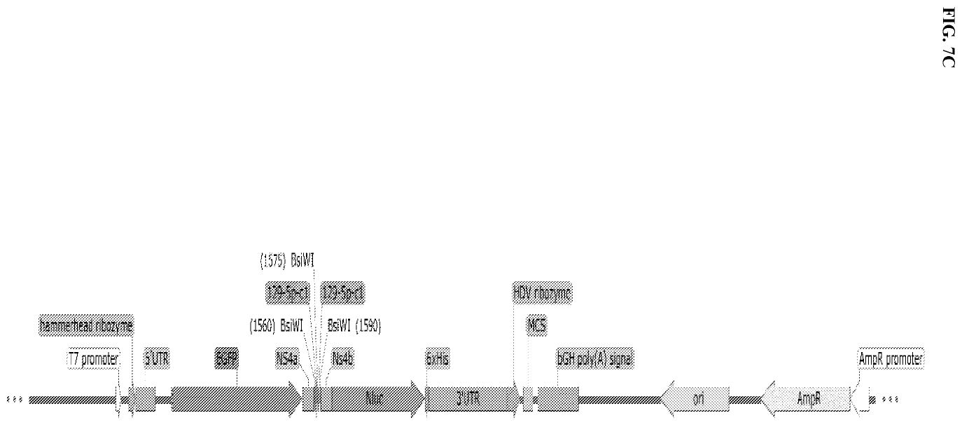

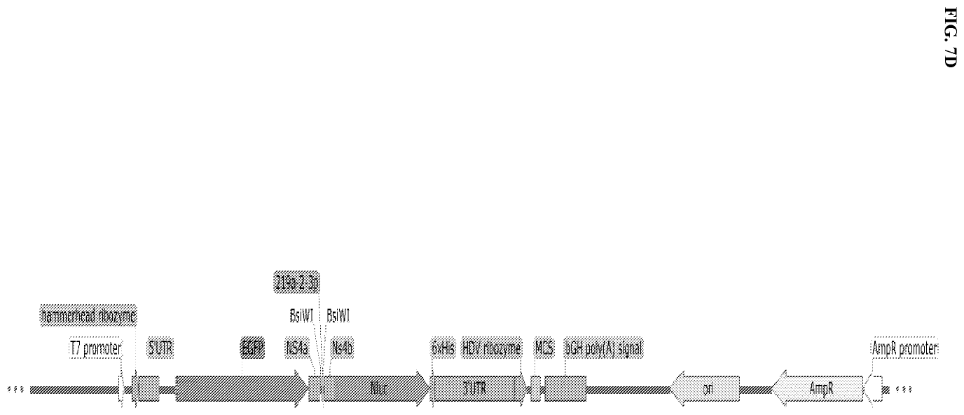

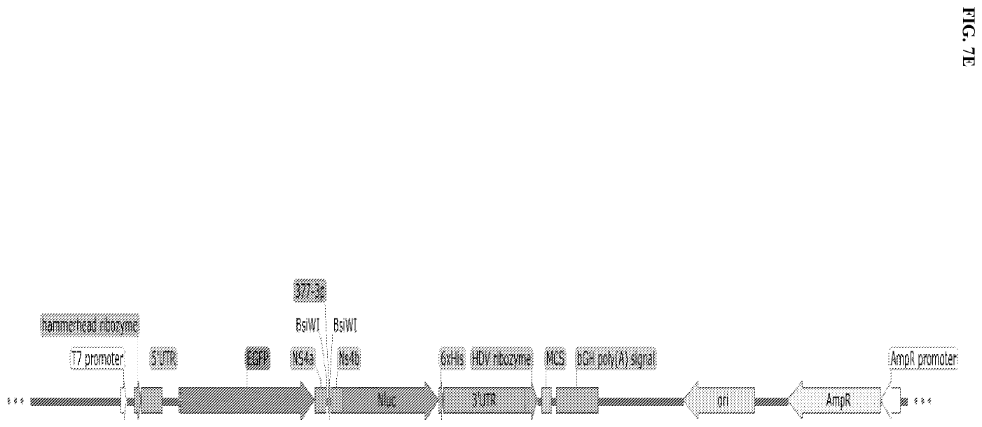

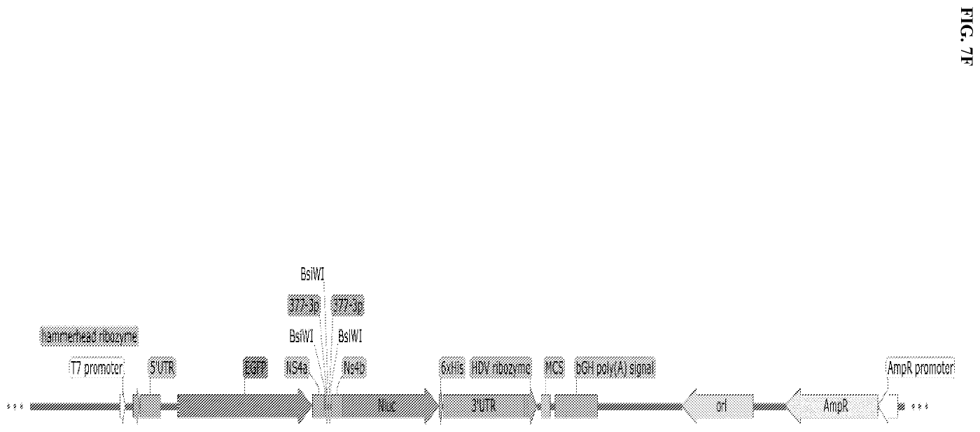

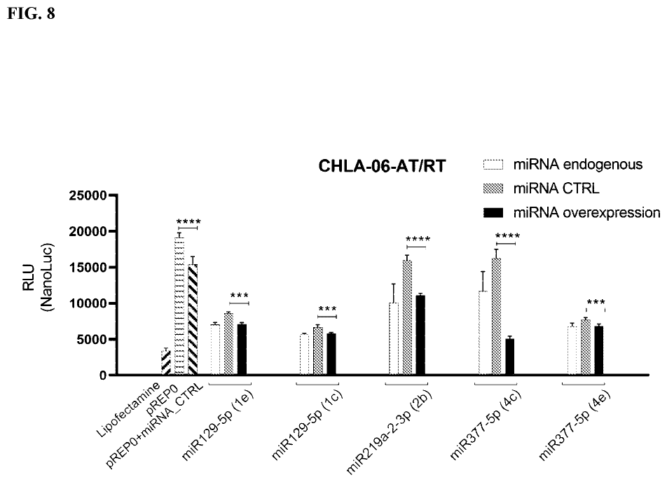

After plasmid generation containing the target sequences, transfection was performed in Vero and CHLA cells in order to observe the inhibition of plasmid expression through the NanoLuc reporter gene. To confirm the inhibitory effect of the inserted sequence, the miRNA expression modulation (overexpression and inhibition of the endogenous miRNAs) was performed in order to observe the reversal of the effect, as shown in FIGS. 7 and 8. Clones whose translation was silenced in Vero (1d/1c) and CHLA06 (4c) were detected, indicating the potential for several sequences to silence the entire ZIKV genome (pCC1_clone) because the mimentic miRNAs were, in fact, capable of inhibiting NanoLuc translation by pairing with the inserted target sequence (FIGS. 7 and 8). Furthermore, absolute silencing in clone 1c was observed because the luminescence difference was not significant as compared to the baseline of a non-transfected cell (FIG. 7). Thus, the 1c clone exhibited about 100% efficiency.

In the CHLA-06-ATRT tumor cell line, the transfection of the control miRNA altered the translation of NanoLuc even in empty pRep, which interfered with the analysis of the results. However, a comparison of the NanoLuc translation of miRNA controls after miRNA overexpression shows upregulation in all clones (FIG. 8).

To confirm its efficiency, a second assay was performed on the CHLA-06-ATRT with miRNA inhibitor transfection. The aim was to inhibit the cell's endogenous miRNA and thus observe a NanoLuc translation increase. As shown in FIG. 8, the inhibition of endogenous miRNA had the effect of increasing NanoLuc translation when compared to the control. Thus, the miR-129-5p target sequence inhibitory effect was confirmed. Among the miR-129-5p clones (1c and 1e), the one with the most potent regulatory effect was 1c, which had only one copy of the target sequence.

Generation of Several oZIKV Candidates

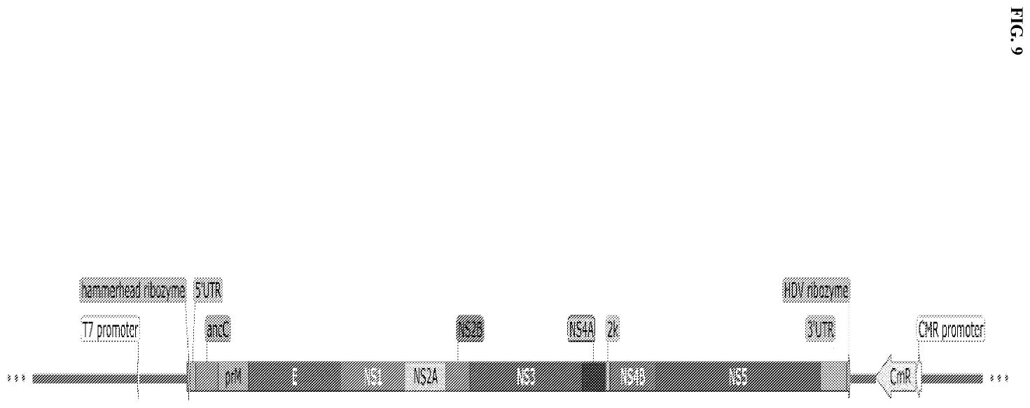

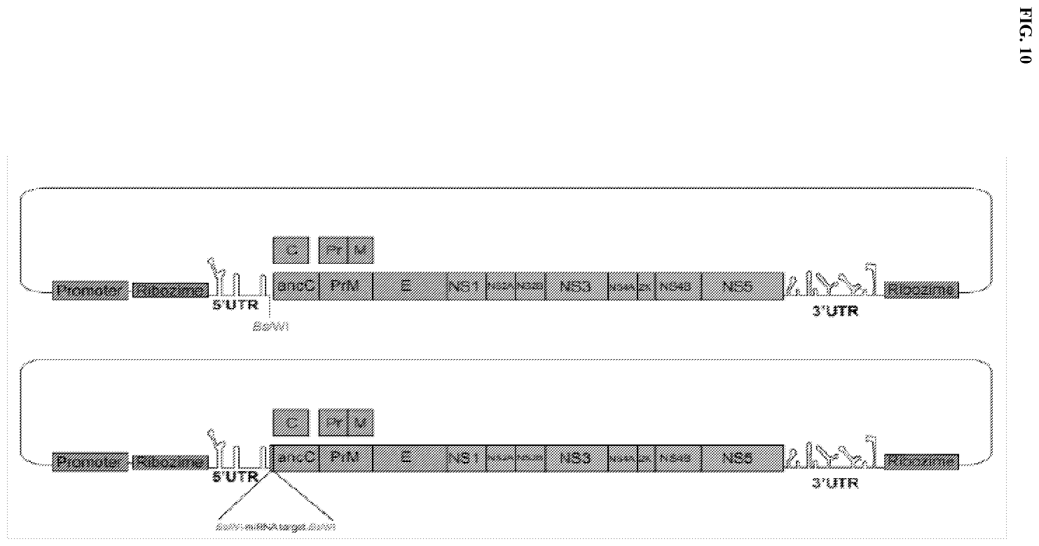

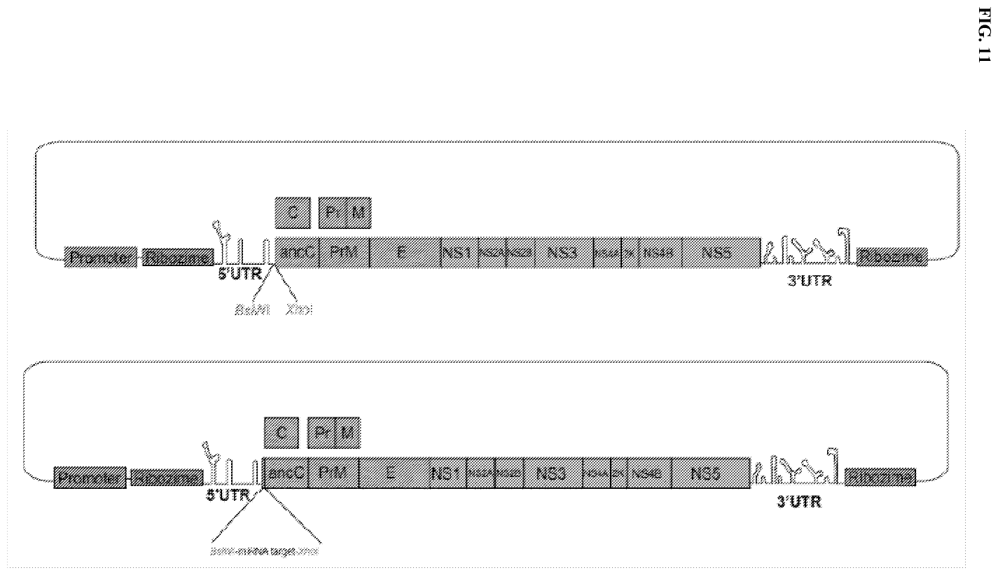

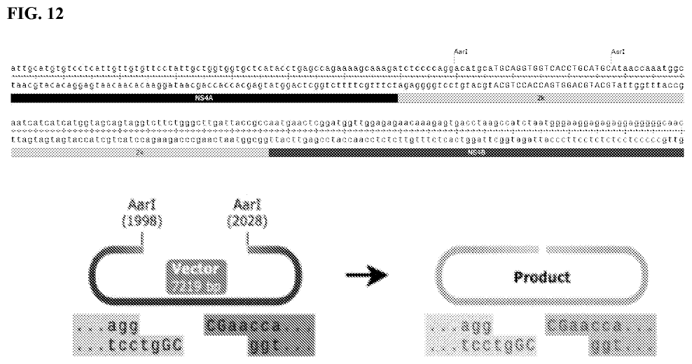

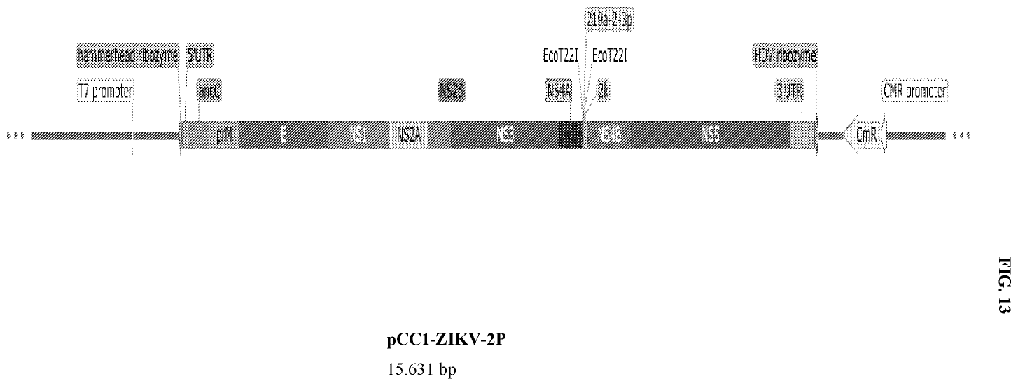

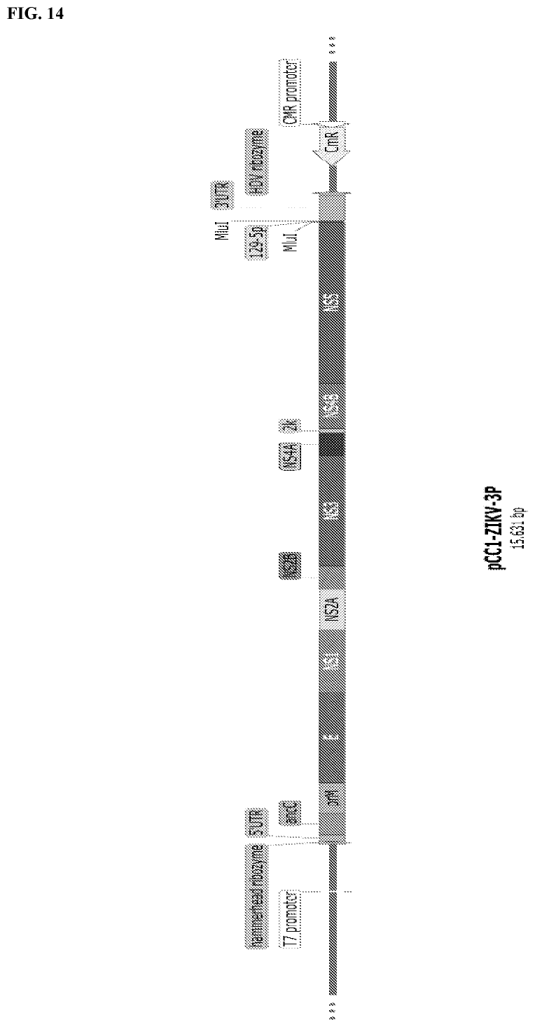

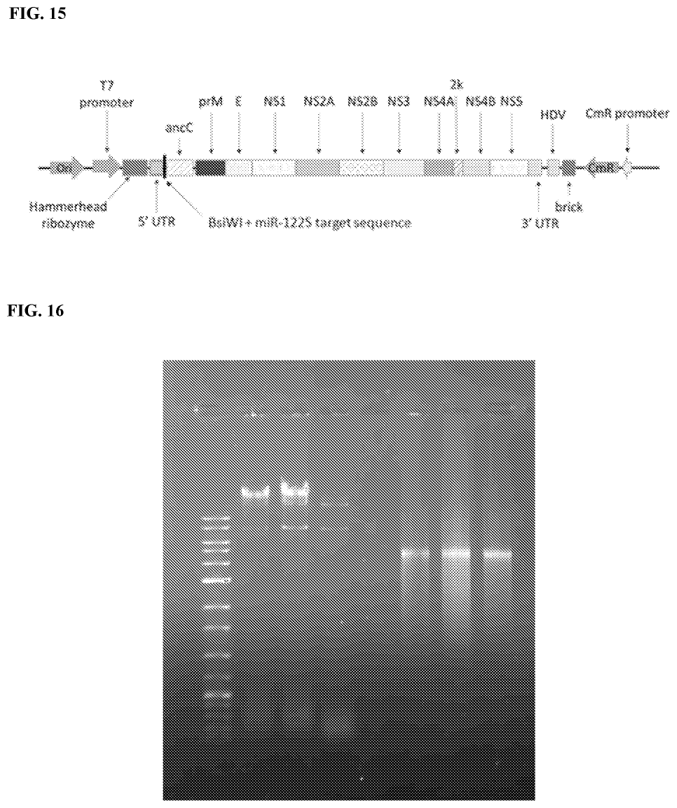

oZIKV (oncolytic ZIKV) candidates having the entire ZIKV genome in the low-copy plasmid (PCC1-ZIKVBR) were created. A representative plasmid containing the ZIKV genome, ribozymes, and the T7 promoter at 2000 base pairs of the 5′ UTR is shown in FIG. 9. The miRNA target was inserted into the 5′ UTR region of different plasmids using BsiWI sites (FIG. 10), a combination of BsiWI and XhoI (FIG. 11) sites, and AarI sites (FIG. 12). Different pCC1-ZIKV constructs were synthesized with at least the T7 promoter, Hammerhead ribozyme, miRNA target insert, regions of the ZIKV genome (in whole or in part), and 3′ UTR. The constructs are generally presented in Table 8. FIG. 13 is a representative image of plasmid 013 (Table 8), which has the entire ZIKV genome, ribozymes at its ends, the T7 promoter at 800 base pairs from the 5′ UTR, and modification inserted into the 2k region. FIG. 14 is a representative image of plasmid 014, which has the entire ZIKV genome, ribozymes at its ends, the T7 promoter at 800 base pairs from the 5′ UTR, and modification inserted into the 3′ UTR. FIG. 15 is a representative image of plasmid 017, which has the entire ZIKV genome, ribozymes at its ends, the T7 promoter next to the 5′ UTR, and modification inserted into the 5′ UTR.

TABLE 8
Recombinant zika virus constructs in the pCC1-ZIKV vector.

| Plasmid | | |
|---|---|---|
| ID | Details | Modifications |
| 001 | pCC1-ZIKV-0P (FIG. 9) | T7 (2000pb) - HammerHead - ZIKBR - HDV |
| 013 | pCC1-ZIKV-2P-T7 (FIG. 13) | T7 (800pb)-HammerHead-ZIKBR-mod2k (EcoT22I + |
| | | miR219a-2-3p)-HDV |
| 014 | pCC1-ZIKV-3P-T7 (FIG. 14) | T7 (800pb)-HammerHead-ZIKBR-mod3′ UTR |
| | | (MluI + miR129-5P)-HDV |
| 001_T7 | pCC1-ZIKV-0P-T7 | T7 (aproximado)-HammerHead-ZIKBR-HDV |
| 013_T7 | pCC1-ZIKV-2P- | T7 (aproximado)-HammerHead-ZIKBR-mod2k |
| | T7aproximado | (EcoT22I + miR219a-2-3p)-HDV |
| 014_T7 | pCC1-ZIKV-3P- | T7 (aproximado)-HammerHead- |
| | T7aproximado | ZIKBR- mod3′ UTR (MluI + miR129-5P)-HDV |
| 015 | pCC1-ZIKV-2P-T7-AarI | T7 (800pb)-HammerHead-ZIKBR-mod2k (EcoT22I + |
| | | miR219a-2-3p)-mut AarI-HDV |
| 016 | pCC1-ZIKV-3P-T7-AarI | T7 (800pb)-HammerHead-ZIKBR-mod3′ UTR |
| | | (MluI + miR1225)-mut-AarI-HDV |
| 017 | pCC1-ZIKV-5P-T7 | T7-HammerHead-ZIKBR- |
| | (FIG. 15) | mod5′ UTR-(BswI + miR1225)-HDV |
| 019 | pCC1-ZIKV-3P-129-5p-1g | T7 (800pb)-HammerHead-ZIKBR-mod3′ UTR |
| | | (MluI + miR129-5p)-mut AarI-HDV |
| 020 | pCC1-ZIKV-3P-129-5p-1I | T7 (800pb)-HammerHead-ZIKBR-mod3′ UTR |
| | | (MluI + miR129-5p)-mut AarI-HDV |
| 021 | pCC1-ZIKV-3P-129-5p-1j | T7 (800pb)-HammerHead-ZIKBR-mod3′ UTR |
| | | (MluI + miR129-5p)-mut AarI-HDV |
| 022 | pCC1-ZIKV-3P-219a-2-3p-Pac | T7 (800pb)-HammerHead-ZIKBR-mod3′ UTR |
| | | (MluI + miR129-5p + Pac)- mut AarI-HDV |

TABLE 9A
Compositions of the recombinant zika virus constructs in the pCC1-ZIKV
vector of Table 8, wherein each construct comprises a promoter,
noncoding region, 5′ UTR, coding region, 3′ UTR, miRNA target flanked by
two restriction sites. SEQ ID NO's are presented in parentheses.

| | | | Non- | | | | | | Restric- | | Restric- |
|---|---|---|---|---|---|---|---|---|---|---|---|
| | Pro- | Ribo- | coding | 5′ | Coding | 3′ | Ribo- | Insertion | tion | miRNA | tion |
| Name | moter | zyme 1 | region | UTR | region | UTR | zyme 2 | location | site 1 | target | site 2 |
| pCC1- | (16) | (14) | (238) | (239) | (240) | (241) | (15) | N/A | N/A | N/A | N/A |
| ZIKV-0P- | | | | | | | | | | | |
| T7 (17) | | | | | | | | | | | |
| pCC1- | (16) | (14) | (242) | (239) | (243) | (244) | (15) | 2 k | EcoT | miR- | EcoT |
| ZIKV-2P- | | | | | | | | | 22I | 219a-2- | 22I |
| T7 (18) | | | | | | | | | | 3p target | |
| | | | | | | | | | | (miRNA | |
| | | | | | | | | | | target 13) | |
| pCC1- | (16) | (14) | (242) | (239) | (245) | (244) | (15) | Between | MluI | miR-129- | MluI |
| ZIKV-3P- | | | | | | | | coding | | 5p target | |
| T7 (19) | | | | | | | | and 3′ | | (miRNA | |
| | | | | | | | | UTR | | target 1) | |
| pCC1- | (16) | (14) | Actgat | (239) | (243) | (244) | (15) | N/A | N/A | N/A | N/A |
| ZIKV-0P- | | | | | | | | | | | |
| T7aprox- | | | | | | | | | | | |
| imado | | | | | | | | | | | |
| (20) | | | | | | | | | | | |
| pCC1- | (16) | (14) | Actgat | (239) | (243) | (244) | (15) | 2 k | EcoT | miR- | EcoT |
| ZIKV-2P- | | | | | | | | | 22I | 219a-2- | 22I |
| T7 aprox- | | | | | | | | | | 3p target | |
| imado | | | | | | | | | | (miRNA | |
| (21) | | | | | | | | | | target 13) | |
| pCC1- | (16) | (14) | Actgat | (239) | (245) | (244) | (15) | between | MluI | miR-129- | MluI |
| ZIKV-3P- | | | | | | | | coding | | 5p target | |
| T7aprox- | | | | | | | | and 3′ | | (miRNA | |
| imado | | | | | | | | UTR | | target 1) | |
| (22) | | | | | | | | | | | |
| pCC1- | (16) | (14) | (242) | (239) | (243) | (244) | (15) | 2 k | EcoT | miR- | EcoT |
| ZIKV-2P- | | | | | | | | | 22I | 219a-2- | 22I |
| T7-AarI | | | | | | | | | | 3p target | |
| (23) | | | | | | | | | | (miRNA | |
| | | | | | | | | | | target 13) | |
| pCC1- | (16) | (14) | (242) | (239) | (247) | (244) | (15) | between | MluI | miR- | MluI |
| ZIKV-3P- | | | | | | | | coding | | 1225-5p | |
| T7-AarI | | | | | | | | and 3′ | | target | |
| (24) | | | | | | | | UTR | | (miRNA | |
| | | | | | | | | | | target 16) | |
| pCC1- | (16) | (14) | (242) | (239) | (248) | (244) | (15) | between | BsiWI | miR- | BsiWI |
| ZIKV-5P- | | | | | | | | 5′ | | 219a-2- | |
| T7 (25) | | | | | | | | UTR and | | 3p target | |
| | | | | | | | | ZIKV | | (miRNA | |
| | | | | | | | | Capsid | | target 16) | |
| pCC1- | (16) | (14) | (242) | (239) | (245) | (244) | (15) | between | MluI | miR-129- | MluI |
| ZIKV-3P- | | | | | | | | coding | | 5p target | |
| 129-5p-1g | | | | | | | | and 3′ | | (miRNA | |
| (30) | | | | | | | | UTR | | target 1) | |
| pCC1- | (16) | (14) | (242) | (239) | (245) | (244) | (15) | between | MluI | miR-129- | MluI |
| ZIKV-3P- | | | | | | | | coding | | 5p target | |
| 129-5p-1i | | | | | | | | and 3′ | | (miRNA | |
| (35) | | | | | | | | UTR | | target 3) | |
| pCC1- | (16) | (14) | (242) | (239) | (245) | (244) | (15) | between | MluI | miR-129- | MluI |
| ZIKV-3P- | | | | | | | | coding | | 5p target | |
| 129-5p-1j | | | | | | | | and 3′ | | (miRNA | |
| (44) | | | | | | | | UTR | | target 1) | |
| pCC1- | (16) | (14) | (242) | (239) | (249) | (244) | (15) | between | PacI | miR- | MluI |
| ZIKV-3P- | | | | | | | | coding | | 219a-3p | |
| 219a-2- | | | | | | | | and 3′ | | (miRNA | |
| 3p-Pac | | | | | | | | UTR | | target 15) | |
| (225) | | | | | | | | | | | |
| pRep-129- | (16) | (14) | (246) | (239) | (247) | (244) | (15) | 2 k | BsiWI | miR-129- | BsiWI |
| 5p-BsiWI- | | | | | | | | | | 5p target | |
| 1c | | | | | | | | | | (miRNA | |
| | | | | | | | | | | target 1) | |
| pRep-129- | (16) | (14) | (246) | (239) | (247) | (244) | (15) | 2 k | BsiWI | miR-129- | BsiWI |
| 5p-BsiWI- | | | | | | | | | | 5p target | |
| 1e | | | | | | | | | | (miRNA | |
| | | | | | | | | | | target 1) | |
| pRep219a- | (16) | (14) | (246) | (239) | (247) | (244) | (15) | 2 k | BsiWI | 219a-2- | BsiWI |
| 2-3p- | | | | | | | | | | 3p | |
| BsiWI-2b | | | | | | | | | | miRNA | |
| | | | | | | | | | | (miRNA | |
| | | | | | | | | | | target 15) | |
| pRep377- | (16) | (14) | (246) | (239) | (247) | (244) | (15) | 2 k | BsiWI | 377-3p | BsiWI |
| 3p-BsiWI- | | | | | | | | | | (miRNA | |
| 4e | | | | | | | | | | target 11) | |
| pRep-377- | (16) | (14) | (246) | (239) | (247) | (244) | (15) | 2 k | BsiWI | 377-3p | BsiWI |
| 3p-BsiWI- | | | | | | | | | | (miRNA | |
| 4c | | | | | | | | | | target 11) | |

TABLE 9B
Sequences of the vectors in Table 9A.

| | | SEQ |
|---|---|---|
| | | ID |
| Name | Sequence (5′ to 3′) | NO: |
| pCC1-ZIKV-0P | | 17 |
| promoter | taatacgactcactatagg | 16 |
| noncoding | gcgcatgctatttttttcctcaacataacgagaacacacaggggcgctatcgcacagaatcaaattcgatgactggaaattttttgtt | 238 |
| | aatttcagaggtcgcctgacgcatatacctttttcaactgaaaaattgggagaaaaaggaaaggtgagaggccggaaccggctt | |
| | ttcatatagaatagagaagcgttcatgactaaatgcttgcatcacaatacttgaagttgacaatattatttaaggacctattgttttttc | |
| | caataggtggttagcaatcgtcttactttctaacttttcttaccttttacatttcagcaatatatatatatatttcaaggatataccattcta | |
| | atgtctgcccctatgtctgcccctaagaagatcgtcgttttgccaggagaccacgttggtcaagaaatcacagccgaagccatta | |
| | aggttcttaaagctatttctgatgttcgttccaatgtcaagttcgatttcgaaaatcatttaattggtggtgctgctatcgatgctacagg | |
| | tgtcccacttccagatgaggcgctggaagcctccaagaaggttgatgccgttttgttaggtgctgtgggtggtcctaaatggggtac | |
| | cggtagtgttagacctgaacaaggtttactaaaaatccgtaaagaacttcaattgtacgccaacttaagaccatgtaactttgcatc | |
| | cgactctcttttagacttatctccaatcaagccacaatttgctaaaggtactgacttcgttgttgtcagagaattagtgggaggtattta | |
| | ctttggtaagagaaaggaagacgatggtgatggtgtcgcttgggatagtgaacaatacaccgttccagaagtgcaaagaatca | |
| | caagaatggccgctttcatggccctacaacatgagccaccattgcctatttggtccttggataaagctaatgttttggcctcttcaag | |
| | attatggagaaaaactgtggaggaaaccatcaagaacgaattccctacattgaaggttcaacatcaattgattgattctgccgcc | |
| | atgatcctagttaagaacccaacccacctaaatggtattataatcaccagcaacatgtttggtgatattatctccgatgaagcctcc | |
| | gttatcccaggttccttgggtttgttgccatctgcgtccttggcctctttgccagacaagaacaccgcatttggtttgtacgaaccatgt | |
| | cacggttctgctccagatttgccaaagaataaggttgaccctatcgccactatcttgtctgctgcaatgatgttgaaattgtcattgaa | |
| | cttgcctgaagaaggtaaggccattgaagatgcagttaaaaaggttttggatgcaggtatcagaactggtgatttaggtggttcca | |
| | acagtaccaccgaagtcggtgatgctgtcgccgaagaagttaagaaaatccttgcttaaaaagattctctttttttatgatatttgtac | |
| | ataaactttataaatgaaattcataatagaaacgacacgaaattacaaaatggaatatgttcatagggtagacgaaactatatac | |
| | gcaatctacatacatttatcaagaaggagaaaaaggaggatagtaaaggaatacaggtaagcaaattgatactaatggctcaa | |
| | cgtgataaggaaaaagaattgcactttaacattaatattgacaaggaggagggcaccacacaaaaagttaggtgtaacagaa | |
| | aatcatgaaactacgattcctaatttgatattggaggattttctctaaaaaaaaaaaaatacgacgtctaagaaaccattattatca | |
| | tgacattaacctataaaaataggcgtatcacgaggccctttcgtctcgcgcgtttcggtgatgacggtgaaaacctctgacacatg | |
| | cagctcccggagacggtcacagcttgtctgtaagcggatgccgggagcagacaagcccgtcagggcgcgtcagcgggtgttg | |
| | gcgggtgtcggggctggcttaaagctgtttcctgtgtgaaattgttatccgctcacaattccacacaacatacgagccggaagcat | |
| | aaagtgtaaagcctggggtgcctaatgagtgagctaactcacattaattgcgttgcgctcactgcccgctttccagtcgggaaacc | |
| | tgtcgtgccagctgcattaatgaatcggccaacgcgcggggagagggtccttttcatcacgtgctataaaaataattataatttaa | |
| | attttttaatataaatatataaattaaaaatagaaagtaaaaaaagaaattaaagaaaaaatagtttttgttttccgaagatgtaaa | |
| | agactctagggggatcgccaacaaatactaccttttatcttgctcttcctgctctcaggtattaatgccgaattgtttcatcttgtctgtg | |
| | tagaagaccacacacgaaaatcctgtgattttacattttacttatcgttaatcgaatgtatatctatttaatctgcttttcttgtctaataa | |
| | atatatatgtaaagtacgctttttgttgaaattttttaaacctttgtttatttttttttcttcattccgtaactcttctaccttctttatttactttcta | |
| | aaatccaaatacaaaacataaaaataaataaacacagagtaaattcccaaattattccatcattaaaagatacgaggcgcgtgt | |
| | aagttacaggcaagcgatccgtcccctcagcctatgcggatccgatcggaccgctcgccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| Coding | ATGAAAAACCCAAAAAAGAAATCCGGAGGATTCCGGATTGTCAATATGCTAAA | 240 |
| region | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTG | |
| 3′ UTR | TAAGCACCAATCTTAATGTTGTCAGGCCTGCTAGTCAGCCACAGCTTGGGGAAAG | 241 |
| | CTGTGCAGCCTGTGACCCCCCCAGGAGAAGCTGGGAAACCAAGCCTATAGTCAGG | |
| | CCGAGAACGCCATGGCACGGAAGAAGCCATGCTGCCTGTGAGCCCCTCAGAGGA | |
| | CACTGAGTCAAAAAACCCCACGCGCTTGGAGGCGCAGGATGGGAAAAGAAGGTG | |
| | GCGACCTTCCCCACCCTTCAATCTGGGGCCTGAACTGGAGATCAGCTGTGGATCT | |
| | CCAGAAGAGGGACTAGTGGTTAGAGGAGACCCCCCGGAAAACGCAAAACAGCAT | |
| | ATTGACGCTGGGAAAGACCAGAGACTCCATGAGTTTCCACCACGCTGGCCGCCAG | |
| | GCACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| ribozyme | | |
| pCC1-ZIKV-2P-T7 | | 18 |
| T7 | taatacgactcactatagg | 16 |
| promoter | | |
| Noncoding | gtgaaattgttatccgctcacaattccacacaacatacgagccggaagcataaagtgtaaagcctggggtgcctaatgagtgag | 242 |
| | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacaaatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 243 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCCAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTIGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTAA | |
| EcoT22I | ATGCAT | |
| miR- | CCGACCTGT | miR |
| 219a-2- | | NA |
| 3p target | | target 13 |

| EcoT22I | ATGCAT | |
|---|---|---|
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| ribozyme | | |
| pCC1-ZIKV-3P-T7 | | 19 |
| T7 | Taatacgactcactatagg | 16 |
| promoter | | |
| Noncoding | gtgaaattgttatccgctcacaattccacacaacatacgagccggaagcataaagtgtaaagcctggggtgcctaatgagtgag | 242 |
| | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacaaatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| Coding | ATGAAAAACCCAAAAAAGAAATCCGGAGGATTCCGGATTGTCAATATGCTAAA | 245 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTA | |
| MluI | ACGCGT | |
| miR- | AAAAACGCC | miR |
| 129-5p | | NA |
| target | | taret 1 |
| MluI | ACGCGT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| ribozyme | | |
| pCC1-ZIKV-0P-T7aproximado | | 20 |
| Noncoding | Actgat | |
| T7 | Taatacgactcactatagg | 16 |
| promoter | | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| Coding | ATGAAAAACCCAAAAAAGAAATCCGGAGGATTCCGGATTGTCAATATGCTAAA | 243 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTIGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTAA | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pCC1-ZIKV-2P-T7aproximado | | 21 |
| Noncoding | actgat | |
| T7 | taatacgactcactatagg | 16 |
| promoter | | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| Coding | ATGAAAAACCCAAAAAAGAAATCCGGAGGATTCCGGATTGTCAATATGCTAAA | 243 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCCAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTAA | |
| EcoT22I | ATGCAT | |
| miR- | CCGACCTGT | miR |
| 219a-2- | | NA |
| 3p target | | target 13 |
| EcoT22I | ATGCAT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | GGCCGGCATGGTCCCAGCCTCCTCGCTGGCGCCGGCTGGGCAACATGCTTCGGC | 15 |
| Ribozyne | ATGGCGAATGGGAC | |
| pCC1-ZIKV-3P-T7aproximado | | 22 |
| noncoding | Actgat | |
| T7 | taatacgactcactatagg | 16 |
| promoter | | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 245 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTA | |
| MluI | ACGCGT | |
| miRNA | AAAAACGCC | miR |
| | | NA |
| | | target 1 |
| MluI | ACGCGT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctgctggtcatggcgaatgggac | 15 |
| Ribozyme | | |
| pCC1-ZIKV-2P-T7-AarI | | 23 |
| T7 | Taatacgactcactatagg | 16 |
| promoter | | |
| Noncoding | gtgaaattgttatccgctcacaattccacacaacatacgagccggaagcataaagtgtaaagcctggggtgcctaatgagtgag | 242 |
| | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacadatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 243 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCCAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGCGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTAA | |
| EcoT22I | ATGCAT | |
| miR- | CCGACCTGT | miR |
| 219a-2- | | NA |
| 3p target | | target 13 |
| EcoT22I | ATGCAT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pCC1-ZIKV-3P-T7-AarI | | 24 |
| T7 | Taatacgactcactatagg | 16 |
| promoter | | |
| Noncoding | gtgaaattgttatccgctcacaattccacacaacatacgagccggaagcataaagtgtaaagcctggggtgcctaatgagtgag | 242 |
| | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacaaatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 247 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCTGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTA | |
| MluI | ACGCGT | |
| miR- | TGCCGGTCA | miR |
| 1225-5p | | NA |
| | | target 16 |
| MluI | ACGCGT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pCC1-ZIKV-5P-T7 | | 25 |
| T7 | Taatacgactcactatagg | 16 |
| Noncoding | gtgaaattgttatccgctcacaattccacacaacatacgagccggaagcataaagtgtaaagcctggggtgcctaatgagtgag | 242 |
| | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacaaatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| Coding | ATGAAAAACCCAAAAAAGAAATCCGGAGGATTCCGGATTGTCAATATGCTAAA | 248 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCTGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCCAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTAA | |
| BsiWI | ATGCAT | |
| miR- | CCGACCTGT | miR |
| 219a-2- | | NA |
| 3p target | | target 13 |
| BsiWI | ATGCAT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pCC1-ZIKV-3P-129-5p-1g | | 30 |
| T7 | taatacgactcactatagg | 16 |
| promoter | | |
| noncoding | gtgaaattgttatccgctcacaattccacacaacatacgagccggaagcataaagtgtaaagcctggggtgcctaatgagtgag | 242 |
| | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacaaatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| Coding | ATGAAAAACCCAAAAAAGAAATCCGGAGGATTCCGGATTGTCAATATGCTAAA | 245 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTA | |
| MluI | ACGCGT | |
| miR- | AAAAACGCC | miR |
| 129-5p | | NA |
| target | | target 1 |
| MluI | ACGCGT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pCC1-ZIKV-3P-129-5p-1I | | 35 |
| T7 | Taatacgactcactatagg | 16 |
| promoter | | |
| Noncoding | gtgaaattgttatccgctcacaattccacacaacatacgagccggaagcataaagtgtaaagcctggggtgcctaatgagtgag | 242 |
| | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacaaatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| Coding | ATGAAAAACCCAAAAAAGAAATCCGGAGGATTCCGGATTGTCAATATGCTAAA | 245 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTA | |
| MluI | ACGCGT | |
| 129-5p | GGCGTTTT | miR |
| target | | NA |
| | | target 3 |
| MluI | ACGCGT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pCC1-ZIKV-3P-129-5p-1j | | 44 |
| T7 | taatacgactcactatagg | 16 |
| promoter | | |
| Noncoding | gtgaaattgttatccgctcacaattccacacaacatacgagccggaagcataaagtgtaaagcctggggtgcctaatgagtgag | 242 |
| region | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacaaatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| Coding | ATGAAAAACCCAAAAAAGAAATCCGGAGGATTCCGGATTGTCAATATGCTAAA | 245 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTA | |
| MluI | ACGCGT | |
| miR- | AAAAACGCC | miR |
| 129-5p | | NA |
| target | | target 1 |
| MluI | ACGCGT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pCC1-ZIKV-3P-219a-2-3p-Pac | | 225 |
| T7 | taatacgactcactatagg | 16 |
| promoter | | |
| Noncoding | gtgaaattgttatccgctcacaattccacacaacatacgagccggaagcataaagtgtaaagcctggggtgcctaatgagtgag | 242 |
| | ctaactcacattaattgcgttgcgctcactgcccgctttccagtcgggaaacctgtcgtgccagctgcattaatgaatcggccaacg | |
| | cgcggggagagggtccttttcatcacgtgctataaaaataattataatttaaattttttaatataaatatataaattaaaaatagaaa | |
| | gtaaaaaaagaaattaaagaaaaaatagtttttgttttccgaagatgtaaaagactctagggggatcgccaacaaatactacctt | |
| | ttatcttgctcttcctgctctcaggtattaatgccgaattgtttcatcttgtctgtgtagaagaccacacacgaaaatcctgtgattttac | |
| | attttacttatcgttaatcgaatgtatatctatttaatctgcttttcttgtctaataaatatatatgtaaagtacgctttttgttgaaatttttta | |
| | aacctttgtttatttttttttcttcattccgtaactcttctaccttctttatttactttctaaaatccaaatacaaaacataaaaataaataaa | |
| | cacagagtaaattcccaaattattccatcattaaaagatacgaggcgcgtgtaagttacaggcaagcgatccgtcccctcagcct | |
| | atgcggatccgatcggaccgctcgagaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 249 |
| | ACGCGGAGTAGCCCGTGTGAGCCCCTTTGGGGGCTTGAAGAGGCTGCCAGCCGG | |
| | ACTTCTGCTGGGTCATGGGCCCATCAGGATGGTCTTGGCAATTCTAGCCTTTTTG | |
| | AGATTCACGGCAATCAAGCCATCACTGGGTCTCATCAATAGATGGGGTTCAGTG | |
| | GGGAAAAAAGAGGCTATGGAAATAATAAAGAAGTTCAAGAAAGATCTGGCTGC | |
| | CATGCTGAGAATAATCAATGCTAGGAAGGAGAAGAAGAGACGGGGCGCAGATA | |
| | CTAGTGTCGGAATTGTTGGCCTCCTGCTGACCACAGCTATGGCAGCGGAGGTCA | |
| | CTAGACGTGGGAGTGCATACTATATGTACTTGGACAGAAACGATGCTGGGGAG | |
| | GCCATATCTTTTCCAACCACATTGGGGATGAATAAGTGTTATATACAGATCATG | |
| | GATCTTGGACACATGTGTGATGCCACCATGAGCTATGAATGCCCTATGCTGGAT | |
| | GAGGGGGTGGAACCAGATGACGTCGATTGTTGGTGCAACACGACGTCAACTTG | |
| | GGTTGTGTACGGAACCTGCCATCACAAAAAAGGTGAAGCACGGAGATCTAGAA | |
| | GAGCTGTGACGCTCCCCTCCCATTCCACTAGGAAGCTGCAAACGCGGTCGCAAA | |
| | CCTGGTTGGAATCAAGAGAATACACAAAGCACTTGATTAGAGTCGAAAATTGG | |
| | ATATTCAGGAACCCTGGCTTCGCGTTAGCAGCAGCTGCCATCGCTTGGCTTTTGG | |
| | GAAGCTCAACGAGCCAAAAAGTCATATACTTGGTCATGATACTGCTGATTGCCC | |
| | CGGCATACAGCATCAGGTGCATAGGAGTCAGCAATAGGGACTTTGTGGAAGGT | |
| | ATGTCAGGTGGGACTTGGGTTGATATTGTCTTGGAACATGGAGGTTGTGTCACC | |
| | GTAATGGCACAGGACAAACCGACTGTCGACATAGAGCTGGTTACAACAACAGT | |
| | CAGCAACATGGCGGAGGTAAGATCCTACTGCTATGAGGCATCAATATCAGACAT | |
| | GGCTTCGGACAGCCGCTGCCCAACACAAGGTGAAGCCTACCTTGACAAGCAATC | |
| | AGACACTCAATATGTCTGCAAAAGAACGTTAGTGGACAGAGGCTGGGGAAATG | |
| | GATGTGGACTTTTTGGCAAAGGGAGTCTGGTGACATGCGCTAAGTTTGCATGCT | |
| | CCAAGAAAATGACCGGGAAGAGCATCCAGCCAGAGAATCTGGAGTACCGGATA | |
| | ATGCTGTCAGTTCATGGCTCCCAGCACAGTGGGATGATCGTTAATGACACAGGA | |
| | CATGAAACTGATGAGAATAGAGCGAAGGTTGAGATAACGCCCAATTCACCAAG | |
| | AGCCGAAGCCACCCTGGGGGGTTTTGGAAGCCTAGGACTTGATTGTGAACCGAG | |
| | GACAGGCCTTGACTTTTCAGATTTGTATTACTTGACTATGAATAACAAGCACTGG | |
| | TTGGTTCACAAGGAGTGGTTCCACGACATTCCATTACCTTGGCACGCTGGGGCA | |
| | GACACCGGAACTCCACACTGGAACAACAAAGAAGCACTGGTAGAGTTCAAGGA | |
| | CGCACATGCCAAAAGGCAAACTGTCGTGGTTCTAGGGAGTCAAGAAGGAGCAG | |
| | TTCACACGGCCCTTGCTGGAGCTCTGGAGGCTGAGATGGATGGTGCAAAGGGAA | |
| | GGCTGTCCTCTGGCCACTTGAAATGTCGCCTGAAAATGGATAAACTTAGATTGA | |
| | AGGGCGTGTCATACTCCTTGTGTACCGCAGCGTTCACATTCACCAAGATCCCGG | |
| | CTGAAACACTGCACGGGACAGTCACAGTGGAGGTACAGTACGCAGGGACAGAT | |
| | GGACCTTGCAAGGTTCCAGCTCAGATGGCGGTGGACATGCAAACTCTGACCCCA | |
| | GTTGGGAGGTTGATAACCGCTAACCCCGTAATCACTGAAAGCACTGAGAACTCT | |
| | AAGATGATGCTGGAACTTGATCCACCATTTGGGGACTCTTACATTGTCATAGGA | |
| | GTCGGGGAGAAGAAGATCACCCACCACTGGCACAGGAGTGGCAGCACCATTGG | |
| | AAAAGCATTTGAAGCCACTGTGAGAGGTGCCAAGAGAATGGCAGTCTTGGGAG | |
| | ACACAGCCTGGGACTTTGGATCAGTTGGAGGCGCTCTCAACTCATTGGGCAAGG | |
| | GCATCCATCAAATTTTTGGAGCAGCTTTCAAATCATTGTTTGGAGGAATGTCCTG | |
| | GTTCTCACAAATTCTCATTGGAACGTTGCTGATGTGGTTGGGTCTGAACACAAA | |
| | GAATGGATCTATTTCCCTTATGTGCTTGGCCTTAGGGGGAGTGTTGATCTTCTTA | |
| | TCCACAGCCGTCTCTGCTGATGTGGGGTGCTCGGTGGACTTCTCAAAGAAGGAG | |
| | ACGAGATGCGGTACAGGGGTGTTCGTCTATAACGACGTTGAAGCCTGGAGGGA | |
| | CAGGTACAAGTACCATCCTGACTCCCCCCGTAGATTGGCAGCAGCAGTCAAGCA | |
| | AGCCTGGGAAGATGGTATCTGCGGGATCTCCTCTGTTTCAAGAATGGAAAACAT | |
| | CATGTGGAGATCAGTAGAAGGGGAGCTCAACGCAATCCTGGAAGAGAATGGAG | |
| | TTCAACTGACGGTCGTTGTGGGATCTGTAAAAAACCCCATGTGGAGAGGTCCAC | |
| | AGAGATTGCCCGTGCCTGTGAACGAGCTGCCCCACGGCTGGAAGGCTTGGGGG | |
| | AAATCGCACTTCGTCAGAGCAGCAAAGACAAATAACAGCTTTGTCGTGGATGGT | |
| | GACACACTGAAGGAATGCCCACTCAAACATAGAGCATGGAACAGCTTTCTTGTG | |
| | GAGGATCATGGGTTCGGGGTATTTCACACTAGTGTCTGGCTCAAGGTTAGAGAA | |
| | GATTATTCATTAGAGTGTGATCCAGCCGTTATTGGAACAGCTGTTAAGGGAAAG | |
| | GAGGCTGTACACAGTGATCTAGGCTACTGGATTGAGAGTGAGAAGAATGACAC | |
| | ATGGAGGCTGAAGAGGGCCCATCTGATCGAGATGAAAACATGTGAATGGCCAA | |
| | AGTCCCACACATTGTGGACAGATGGAATAGAAGAGAGTGATCTGATCATACCCA | |
| | AGTCTTTAGCTGGGCCACTCAGCCATCACAATACCAGAGAGGGCTACAGGACCC | |
| | AAATGAAAGGGCCATGGCACAGTGAAGAGCTTGAAATTCGGTTTGAGGAATGC | |
| | CCAGGCACTAAGGTCCACGTGGAGGAAACATGTGGAACAAGAGGACCATCTCT | |
| | GAGATCAACCACTGCAAGCGGAAGGGTGATCGAGGAATGGTGCTGCAGGGAGT | |
| | GCACAATGCCCCCACTGTCGTTCCGGGCTAAAGATGGCTGTTGGTATGGAATGG | |
| | AGATAAGGCCCAGGAAAGAACCAGAAAGCAACTTAGTAAGGTCAATGGTGACT | |
| | GCAGGATCAACTGATCACATGGATCACTTCTCCCTTGGAGTGCTTGTGATTCTGC | |
| | TCATGGTGCAGGAAGGGCTGAAGAAGAGAATGACCACAAAGATCATCATAAGC | |
| | ACATCAATGGCAGTGCTGGTAGCTATGATCCTGGGAGGATTTTCAATGAGTGAC | |
| | CTGGCTAAGCTTGCAATTTTGATGGGTGCCACCTTCGCGGAAATGAACACTGGA | |
| | GGAGATGTAGCTCATCTGGCGCTGATAGCGGCATTCAAAGTCAGACCAGCGTTG | |
| | CTGGTATCTTTCATCTTCAGAGCTAATTGGACACCCCGTGAAAGCATGCTGCTGG | |
| | CCTTGGCCTCGTGTCTTTTGCAAACTGCGATCTCCGCCTTGGAAGGCGACCTGAT | |
| | GGTTCTCATCAATGGTTTTGCTTTGGCCTGGTTGGCAATACGAGCGATGGTTGTT | |
| | CCACGCACTGATAACATCACCTTGGCAATCCTGGCTGCTCTGACACCACTGGCC | |
| | CGGGGCACACTGCTTGTGGCGTGGAGAGCAGGCCTTGCTACTTGCGGGGGGTTT | |
| | ATGCTCCTCTCTCTGAAGGGAAAAGGCAGTGTGAAGAAGAACTTACCATTTGTC | |
| | ATGGCCCTGGGACTAACCGCTGTGAGGCTGGTCGACCCCATCAACGTGGTGGGG | |
| | CTGCTGTTGCTCACAAGGAGTGGGAAGCGGAGCTGGCCCCCTAGCGAAGTACTC | |
| | ACAGCTGTTGGCCTGATATGCGCATTGGCTGGAGGGTTCGCCAAGGCAGATATA | |
| | GAGATGGCTGGGCCCATGGCCGCGGTCGGTCTGCTAATTGTCAGTTACGTGGTC | |
| | TCAGGAAAGAGTGTGGACATGTACATTGAAAGAGCAGGTGACATCACATGGGA | |
| | AAAAGATGCGGAAGTCACTGGAAACAGTCCCCGGCTCGATGTGGCGCTAGATG | |
| | AGAGTGGTGATTTCTCCCTGGTGGAGGATGACGGTCCCCCCATGAGAGAGATCA | |
| | TACTCAAGGTGGTCCTGATGACCATCTGTGGCATGAACCCAATAGCCATACCCT | |
| | TTGCAGCTGGAGCGTGGTACGTATACGTGAAGACTGGAAAAAGGAGTGGTGCT | |
| | CTATGGGATGTGCCTGCTCCCAAGGAAGTAAAAAAGGGGGAGACCACAGATGG | |
| | AGTGTACAGAGTAATGACTCGTAGACTGCTAGGTTCAACACAAGTTGGAGTGGG | |
| | AGTTATGCAAGAGGGGGTCTTTCACACTATGTGGCACGTCACAAAAGGATCCGC | |
| | GCTGAGAAGCGGTGAAGGGAGACTTGATCCATACTGGGGAGATGTCAAGCAGG | |
| | ATCTGGTGTCATACTGTGGTCCATGGAAGCTAGATGCCGCCTGGGACGGGCACA | |
| | GCGAGGTGCAGCTCTTGGCCGTGCCCCCCGGAGAGAGAGCGAGGAACATCCAG | |
| | ACTCTGCCCGGAATATTTAAGACAAAGGATGGGGACATTGGAGCGGTTGCGCTG | |
| | GATTACCCAGCAGGAACTTCAGGATCTCCAATCCTAGACAAGTGTGGGAGAGTG | |
| | ATAGGACTTTATGGCAATGGGGTCGTGATCAAAAATGGGAGTTATGTTAGTGCC | |
| | ATCACCCAAGGGAGGAGGGAGGAAGAGACTCCTGTTGAGTGCTTCGAGCCTTC | |
| | GATGCTGAAGAAGAAGCAGCTAACTGTCTTAGACTTGCATCCTGGAGCTGGGAA | |
| | AACCAGGAGAGTTCTTCCTGAAATAGTCCGTGAAGCCATAAAAACAAGACTCCG | |
| | TACTGTGATCTTAGCTCCAACCAGGGTTGTCGCTGCTGAAATGGAGGAAGCCCT | |
| | TAGAGGGCTTCCAGTGCGTTATATGACAACAGCAGTCAATGTCACCCACTCTGG | |
| | AACAGAAATCGTCGACTTAATGTGCCATGCCACCTTCACTTCACGTCTACTACA | |
| | GCCAATCAGAGTCCCCAACTATAATCTGTATATTATGGATGAGGCCCACTTCAC | |
| | AGATCCCTCAAGTATAGCAGCAAGAGGATACATTTCAACAAGGGTTGAGATGG | |
| | GCGAGGCGGCTGCCATCTTCATGACCGCCACGCCACCAGGAACCCGTGACGCAT | |
| | TTCCGGACTCCAACTCACCAATTATGGACACCGAAGTGGAAGTCCCAGAGAGAG | |
| | CCTGGAGCTCAGGCTTTGATTGGGTGACGGATTATTCTGGAAAAACAGTTTGGT | |
| | TTGTTCCAAGCGTGAGGAACGGCAATGAGATCGCAGCTTGTCTGACAAAGGCTG | |
| | GAAAACGGGTCATACAGCTCAGCAGAAAGACTTTTGAGACAGAGTTCCAGAAA | |
| | ACAAAACATCAAGAGTGGGACTTTGTCGTGACAACTGACATTTCAGAGATGGGC | |
| | GCCAACTTTAAAGCTGACCGTGTCATAGATTCCAGGAGATGCCTAAAGCCGGTC | |
| | ATACTTGATGGCGAGAGAGTCATTCTGGCTGGACCCATGCCTGTCACACATGCC | |
| | AGCGCTGCCCAGAGGAGGGGGCGCATAGGCAGGAATCCCAACAAACCTGGAGA | |
| | TGAGTATCTGTATGGAGGTGGGTGCGCAGAGACTGACGAAGACCATGCACACT | |
| | GGCTTGAAGCAAGAATGCTCCTTGACAATATTTACCTCCAAGATGGCCTCATAG | |
| | CCTCGCTCTATCGACCTGAGGCCGACAAAGTAGCAGCCATTGAGGGAGAGTTCA | |
| | AGCTTAGGACGGAGCAAAGGAAGACCTTTGTGGAACTCATGAAAAGAGGAGAT | |
| | CTTCCTGTTTGGCTGGCCTATCAGGTTGCATCTGCCGGAATAACCTACACAGATA | |
| | GAAGATGGTGCTTTGATGGCACGACCAACAACACCATAATGGAAGACAGTGTG | |
| | CCGGCAGAGGTGTGGACCAGACACGGAGAGAAAAGAGTGCTCAAACCGAGGTG | |
| | GATGGACGCCAGAGTTTGTTCAGATCATGCGGCCCTGAAGTCATTCAAGGAGTT | |
| | TGCCGCTGGGAAAAGAGGAGCGGCTTTTGGAGTGATGGAAGCCCTGGGAACAC | |
| | TGCCAGGACACATGACAGAGAGATTCCAGGAAGCCATTGACAACCTCGCTGTGC | |
| | TCATGCGGGCAGAGACTGGAAGCAGGCCTTACAAAGCCGCGGCGGCCCAATTG | |
| | CCGGAGACCCTAGAGACCATTATGCTTTTGGGGTTGCTGGGAACAGTCTCGCTG | |
| | GGAATCTTTTTCGTCTTGATGAGGAACAAGGGCATAGGGAAGATGGGCTTTGGA | |
| | ATGGTGACTCTTGGGGCCAGCGCATGGCTCATGTGGCTCTCGGAAATTGAGCCA | |
| | GCCAGAATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGGAAGGAGAGAGGAGGGGG | |
| | CAACCATGGGATTCTCAATGGACATTGACCTGCGGCCAGCCTCAGCTTGGGCCA | |
| | TCTATGCTGCCTTGACAACTTTCATTACCCCAGCCGTCCAACATGCAGTGACCAC | |
| | TTCATACAACAACTACTCCTTAATGGCGATGGCCACGCAAGCTGGAGTGTTGTT | |
| | TGGTATGGGCAAAGGGATGCCATTCTACGCATGGGACTTTGGAGTCCCGCTGCT | |
| | AATGATAGGTTGCTACTCACAATTAACGCCCCTGACCCTAATAGTGGCCATCAT | |
| | TTTGCTCGTGGCGCACTACATGTACTTGATCCCAGGGCTGCAGGCAGCAGCTGC | |
| | GCGTGCTGCCCAGAAGAGAACGGCAGCTGGCATCATGAAGAACCCTGTTGTGG | |
| | ATGGAATAGTGGTGACTGACATTGACACAATGACAATTGACCCCCAAGTGGAG | |
| | AAAAAGATGGGACAGGTGCTACTCATGGCAGTAGCCGTCTCCAGCGCCATACTG | |
| | TCGCGGACCGCCTGGGGGTGGGGGGAGGCTGGGGCCCTGATCACAGCCGCAAC | |
| | TTCCACTTTGTGGGAAGGCTCTCCGAACAAGTACTGGAACTCCTCTACAGCCAC | |
| | TTCACTGTGTAACATTTTTAGGGGAAGTTACTTGGCTGGAGCTTCTCTAATCTAC | |
| | ACAGTAACAAGAAACGCTGGCTTGGTCAAGAGACGTGGGGGTGGAACAGGAGA | |
| | GACCCTGGGAGAGAAATGGAAGGCCCGCTTGAACCAGATGTCGGCCCTGGAGT | |
| | TCTACTCCTACAAAAAGTCAGGCATCACCGAGGTGTGCAGAGAAGAGGCCCGC | |
| | CGCGCCCTCAAGGACGGTGTGGCAACGGGAGGCCATGCTGTGTCCCGAGGAAG | |
| | TGCAAAGCTGAGATGGTTGGTGGAGCGGGGATACCTGCAGCCCTATGGAAAGG | |
| | TCATTGATCTTGGATGTGGCAGAGGGGGCTGGAGTTACTACGCCGCCACCATCC | |
| | GCAAAGTTCAAGAAGTGAAAGGATACACAAAAGGAGGCCCTGGTCATGAAGAA | |
| | CCCGTGTTGGTGCAAAGCTATGGGTGGAACATAGTCCGTCTTAAGAGTGGGGTG | |
| | GACGTCTTTCATATGGCGGCTGAGCCGTGTGACACGTTGCTGTGTGACATAGGT | |
| | GAGTCATCATCTAGTCCTGAAGTGGAAGAAGCACGGACGCTCAGAGTCCTCTCC | |
| | ATGGTGGGGGATTGGCTTGAAAAAAGACCAGGAGCCTTTTGTATAAAAGTGTTG | |
| | TGCCCATACACCAGCACTATGATGGAAACCCTGGAGCGACTGCAGCGTAGGTAT | |
| | GGGGGAGGACTGGTCAGAGTGCCACTCTCCCGCAACTCTACACATGAGATGTAC | |
| | TGGGTCTCTGGAGCGAAAAGCAACACCATAAAAAGTGTGTCCACCACGAGCCA | |
| | GCTCCTCTTGGGGCGCATGGACGGGCCTAGGAGGCCAGTGAAATATGAGGAGG | |
| | ATGTGAATCTCGGCTCTGGCACGCGGGCTGTGGTAAGCTGCGCTGAAGCTCCCA | |
| | ACATGAAGATCATTGGTAACCGCATTGAAAGGATCCGCAGTGAGCACGCGGAA | |
| | ACGTGGTTCTTTGACGAGAACCACCCATATAGGACATGGGCTTACCATGGAAGC | |
| | TATGAGGCCCCCACACAAGGGTCAGCGTCCTCTCTAATAAACGGGGTTGTCAGG | |
| | CTCCTGTCAAAACCCTGGGATGTGGTGACTGGAGTCACAGGAATAGCCATGACC | |
| | GACACCACACCGTATGGTCAGCAAAGAGTTTTCAAGGAAAAAGTGGACACTAG | |
| | GGTGCCAGACCCCCAAGAAGGTACTCGTCAGGTTATGAGCATGGTCTCTTCCTG | |
| | GTTGTGGAAAGAGCTAGGCAAACACAAACGGCCACGAGTCTGTACCAAAGAAG | |
| | AGTTCATCAACAAGGTTCGTAGCAATGCAGCATTAGGGGCAATATTTGAAGAGG | |
| | AAAAAGAGTGGAAGACTGCAGTGGAAGCTGTGAACGATCCAAGGTTCTGGGCT | |
| | CTAGTGGACAAGGAAAGAGAGCACCACCTGAGAGGAGAGTGCCAGAGTTGTGT | |
| | GTACAACATGATGGGAAAAAGAGAAAAGAAACAAGGGGAATTTGGAAAGGCC | |
| | AAGGGCAGCCGCGCCATCTGGTATATGTGGCTAGGGGCTAGATTTCTAGAGTTC | |
| | GAAGCCCTTGGATTCTTGAACGAGGATCACTGGATGGGGAGAGAGAACTCAGG | |
| | AGGTGGTGTTGAAGGGCTGGGATTACAAAGACTCGGATATGTCCTAGAAGAGA | |
| | TGAGTCGCATACCAGGAGGAAGGATGTATGCAGATGACACTGCTGGCTGGGAC | |
| | ACCCGCATCAGCAGGTTTGATCTGGAGAATGAAGCTCTAATCACCAACCAAATG | |
| | GAGAAAGGGCACAGGGCCTTGGCATTGGCCATAATCAAGTACACATACCAAAA | |
| | CAAAGTGGTAAAGGTCCTTAGACCAGCTGAAAAAGGGAAAACAGTTATGGACA | |
| | TTATTTCGAGACAAGACCAAAGGGGGAGCGGACAAGTTGTCACTTACGCTCTTA | |
| | ACACATTTACCAACCTAGTGGTGCAACTCATTCGGAATATGGAGGCTGAGGAAG | |
| | TCCTAGAGATGCAAGACTTGTGGCTGCTGCGGAGGTCAGAGAAAGTGACCAACT | |
| | GGTTGCAGAGCAACGGATGGGATAGGCTCAAACGAATGGCAGTCAGTGGAGAT | |
| | GATTGCGTTGTGAAGCCAATTGATGATAGGTTTGCACATGCCCTCAGGTTCTTGA | |
| | ATGATATGGGAAAAGTTAGGAAGGACACACAAGAGTGGAAACCCTCAACTGGA | |
| | TGGGACAACTGGGAAGAAGTTCCGTTTTGCTCCCACCACTTCAACAAGCTCCAT | |
| | CTCAAGGACGGGAGGTCCATTGTGGTTCCCTGCCGCCACCAAGATGAACTGATT | |
| | GGCCGGGCCCGCGTCTCTCCAGGGGGGGGATGGAGCATCCGGGAGACTGCTTGC | |
| | CTAGCAAAATCATATGCGCAAATGTGGCAGCTCCTTTATTTCCACAGAAGGGAC | |
| | CTCCGACTGATGGCCAATGCCATTTGTTCATCTGTGCCAGTTGACTGGGTTCCAA | |
| | CTGGGAGAACTACCTGGTCAATCCATGGAAAGGGAGAATGGATGACCACTGAA | |
| | GACATGCTTGTGGTGTGGAACAGAGTGTGGATTGAGGAGAACGACCACATGGA | |
| | AGACAAGACCCCAGTTACGAAATGGACAGACATTCCCTATTTGGGAAAAAGGG | |
| | AAGACTTGTGGTGTGGATCTCTCATAGGGCACAGACCGCGCACCACCTGGGCTG | |
| | AGAACATTAAAAACACAGTCAACATGGTGCGCAGGATCATAGGTGATGAAGAA | |
| | AAGTACATGGACTACCTATCCACCCAAGTTCGCTACTTGGGTGAAGAAGGGTCT | |
| | ACACCTGGAGTGCTGTAACGCGG | |
| PacI | TTAATTAA | |
| 219a-2-3p | TCTTAACACC | miR |
| miRNA | | NA |
| target | | target 15 |
| MluI | ACGCGT | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pReplicon-129-5p-BsiWI-1c | | |
| T7 | taatacgactcactatagg | 16 |
| promoter | | |
| Noncoding | GAGACCCAAGCTGGCTAGCGTTTAAACTTAAGCTTGGTACCGAGCTCGgatccctcgagctc | 246 |
| | gaaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 247 |
| | ACGCGGAGTAGCCCGTGTGAGCCCCTTTGGGGGCTTGATGGTGAGCAAGGGCG | |
| | AGGAGCTGTTCACCGGGGTGGTGCCCATCCTGGTCGAGCTGGACGGCGACGTAA | |
| | ACGGCCACAAGTTCAGCGTGTCCGGCGAGGGCGAGGGCGATGCCACCTACGGC | |
| | AAGCTGACCCTGAAGTTCATCTGCACCACCGGCAAGCTGCCCGTGCCCTGGCCC | |
| | ACCCTCGTGACCACCCTGACCTACGGCGTGCAGTGCTTCAGCCGCTACCCCGAC | |
| | CACATGAAGCAGCACGACTTCTTCAAGTCCGCCATGCCCGAAGGCTACGTCCAG | |
| | GAGCGCACCATCTTCTTCAAGGACGACGGCAACTACAAGACCCGCGCCGAGGT | |
| | GAAGTTCGAGGGCGACACCCTGGTGAACCGCATCGAGCTGAAGGGCATCGACT | |
| | TCAAGGAGGACGGCAACATCCTGGGGCACAAGCTGGAGTACAACTACAACAGC | |
| | CACAACGTCTATATCATGGCCGACAAGCAGAAGAACGGCATCAAGGTGAACTT | |
| | CAAGATCCGCCACAACATCGAGGACGGCAGCGTGCAGCTCGCCGACCACTACC | |
| | AGCAGAACACCCCCATCGGCGACGGCCCCGTGCTGCTGCCCGACAACCACTACC | |
| | TGAGCACCCAGTCCGCCCTGAGCAAAGACCCCAACGAGAAGCGCGATCACATG | |
| | GTCCTGCTGGAGTTCGTGACCGCCGCCGGGATCACTCTCGGCATGGACGAGCTG | |
| | TACAAGATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGTCTTCACACTCGAAGATTTC | |
| | GTTGGGGACTGGCGACAGACAGCCGGCTACAACCTGGACCAAGTCCTTGAACA | |
| | GGGAGGTGTGTCCAGTTTGTTTCAGAATCTCGGGGTGTCCGTAACTCCGATCCA | |
| | AAGGATTGTCCTGAGCGGTGAAAATGGGCTGAAGATCGACATCCATGTCATCAT | |
| | CCCGTATGAAGGTCTGAGCGGCGACCAAATGGGCCAGATCGAAAAAATTTTTAA | |
| | GGTGGTGTACCCTGTGGATGATCATCACTTTAAGGTGATCCTGCACTATGGCAC | |
| | ACTGGTAATCGACGGGGTTACGCCGAACATGATCGACTATTTCGGACGGCCGTA | |
| | TGAAGGCATCGCCGTGTTCGACGGCAAAAAGATCACTGTAACAGGGACCCTGTG | |
| | GAACGGCAACAAAATTATCGACGAGCGCCTGATCAACCCCGACGGCTCCCTGCT | |
| | GTTCCGAGTAACCATCAACGGAGTGACCGGCTGGCGGCTGTGCGAACGCATTCT | |
| | GGCGCATCATCACCATCACCACTAA | |
| BsiWI | CTGACG | |
| miRNA | AAAAACGCC | miR |
| | | NA |
| | | target 1 |
| BsiWI | CTGACG | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggca acatgcttcggcatggcgaatgggac | 15 |
| Ribozyme | | |
| pReplicon-129-5p-BsiWI-le | | |
| T7 | taatacgactcactatagg | 16 |
| promoter | | |
| Noncoding | GAGACCCAAGCTGGCTAGCGTTTAAACTTAAGCTTGGTACCGAGCTCGgatccctcgagctc | 246 |
| | gaaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 247 |
| | ACGCGGAGTAGCCCGTGTGAGCCCCTTTGGGGGCTTGATGGTGAGCAAGGGCG | |
| | AGGAGCTGTTCACCGGGGTGGTGCCCATCCTGGTCGAGCTGGACGGCGACGTAA | |
| | ACGGCCACAAGTTCAGCGTGTCCGGCGAGGGCGAGGGCGATGCCACCTACGGC | |
| | AAGCTGACCCTGAAGTTCATCTGCACCACCGGCAAGCTGCCCGTGCCCTGGCCC | |
| | ACCCTCGTGACCACCCTGACCTACGGCGTGCAGTGCTTCAGCCGCTACCCCGAC | |
| | CACATGAAGCAGCACGACTTCTTCAAGTCCGCCATGCCCGAAGGCTACGTCCAG | |
| | GAGCGCACCATCTTCTTCAAGGACGACGGCAACTACAAGACCCGCGCCGAGGT | |
| | GAAGTTCGAGGGCGACACCCTGGTGAACCGCATCGAGCTGAAGGGCATCGACT | |
| | TCAAGGAGGACGGCAACATCCTGGGGCACAAGCTGGAGTACAACTACAACAGC | |
| | CACAACGTCTATATCATGGCCGACAAGCAGAAGAACGGCATCAAGGTGAACTT | |
| | CAAGATCCGCCACAACATCGAGGACGGCAGCGTGCAGCTCGCCGACCACTACC | |
| | AGCAGAACACCCCCATCGGCGACGGCCCCGTGCTGCTGCCCGACAACCACTACC | |
| | TGAGCACCCAGTCCGCCCTGAGCAAAGACCCCAACGAGAAGCGCGATCACATG | |
| | GTCCTGCTGGAGTTCGTGACCGCCGCCGGGATCACTCTCGGCATGGACGAGCTG | |
| | TACAAGATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGTCTTCACACTCGAAGATTTC | |
| | GTTGGGGACTGGCGACAGACAGCCGGCTACAACCTGGACCAAGTCCTTGAACA | |
| | GGGAGGTGTGTCCAGTTTGTTTCAGAATCTCGGGGTGTCCGTAACTCCGATCCA | |
| | AAGGATTGTCCTGAGCGGTGAAAATGGGCTGAAGATCGACATCCATGTCATCAT | |
| | CCCGTATGAAGGTCTGAGCGGCGACCAAATGGGCCAGATCGAAAAAATTTTTAA | |
| | GGTGGTGTACCCTGTGGATGATCATCACTTTAAGGTGATCCTGCACTATGGCAC | |
| | ACTGGTAATCGACGGGGTTACGCCGAACATGATCGACTATTTCGGACGGCCGTA | |
| | TGAAGGCATCGCCGTGTTCGACGGCAAAAAGATCACTGTAACAGGGACCCTGTG | |
| | GAACGGCAACAAAATTATCGACGAGCGCCTGATCAACCCCGACGGCTCCCTGCT | |
| | GTTCCGAGTAACCATCAACGGAGTGACCGGCTGGCGGCTGTGCGAACGCATTCT | |
| | GGCGCATCATCACCATCACCACTAA | |
| BsiWI | CTGACG | |
| miRNA | AAAAACGCC | miR |
| | | NA |
| | | target 1 |
| BsiWI | CTGACG | |
| miRNA | AAAAACGCC | miR |
| | | NA |
| | | target 1 |
| BsiWI | CTGACG | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | ggccggcatggtcccagcctcctcgctggcgccggctgggcaacatgcttcggcatggcgaatgggac | 15 |
| Ribozyme | | |
| pReplicon-219a-2-3p-BsiWI-2b | | |
| T7 | taatacgactcactatagg | 16 |
| promoter | | |
| Noncoding | GAGACCCAAGCTGGCTAGCGTTTAAACTTAAGCTTGGTACCGAGCTCGgatccctcgagctc | 246 |
| | gaaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 247 |
| | ACGCGGAGTAGCCCGTGTGAGCCCCTTTGGGGGCTTGATGGTGAGCAAGGGCG | |
| | AGGAGCTGTTCACCGGGGTGGTGCCCATCCTGGTCGAGCTGGACGGCGACGTAA | |
| | ACGGCCACAAGTTCAGCGTGTCCGGCGAGGGCGAGGGCGATGCCACCTACGGC | |
| | AAGCTGACCCTGAAGTTCATCTGCACCACCGGCAAGCTGCCCGTGCCCTGGCCC | |
| | ACCCTCGTGACCACCCTGACCTACGGCGTGCAGTGCTTCAGCCGCTACCCCGAC | |
| | CACATGAAGCAGCACGACTTCTTCAAGTCCGCCATGCCCGAAGGCTACGTCCAG | |
| | GAGCGCACCATCTTCTTCAAGGACGACGGCAACTACAAGACCCGCGCCGAGGT | |
| | GAAGTTCGAGGGCGACACCCTGGTGAACCGCATCGAGCTGAAGGGCATCGACT | |
| | TCAAGGAGGACGGCAACATCCTGGGGCACAAGCTGGAGTACAACTACAACAGC | |
| | CACAACGTCTATATCATGGCCGACAAGCAGAAGAACGGCATCAAGGTGAACTT | |
| | CAAGATCCGCCACAACATCGAGGACGGCAGCGTGCAGCTCGCCGACCACTACC | |
| | AGCAGAACACCCCCATCGGCGACGGCCCCGTGCTGCTGCCCGACAACCACTACC | |
| | TGAGCACCCAGTCCGCCCTGAGCAAAGACCCCAACGAGAAGCGCGATCACATG | |
| | GTCCTGCTGGAGTTCGTGACCGCCGCCGGGATCACTCTCGGCATGGACGAGCTG | |
| | TACAAGATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGTCTTCACACTCGAAGATTTC | |
| | GTTGGGGACTGGCGACAGACAGCCGGCTACAACCTGGACCAAGTCCTTGAACA | |
| | GGGAGGTGTGTCCAGTTTGTTTCAGAATCTCGGGGTGTCCGTAACTCCGATCCA | |
| | AAGGATTGTCCTGAGCGGTGAAAATGGGCTGAAGATCGACATCCATGTCATCAT | |
| | CCCGTATGAAGGTCTGAGCGGCGACCAAATGGGCCAGATCGAAAAAATTTTTAA | |
| | GGTGGTGTACCCTGTGGATGATCATCACTTTAAGGTGATCCTGCACTATGGCAC | |
| | ACTGGTAATCGACGGGGTTACGCCGAACATGATCGACTATTTCGGACGGCCGTA | |
| | TGAAGGCATCGCCGTGTTCGACGGCAAAAAGATCACTGTAACAGGGACCCTGTG | |
| | GAACGGCAACAAAATTATCGACGAGCGCCTGATCAACCCCGACGGCTCCCTGCT | |
| | GTTCCGAGTAACCATCAACGGAGTGACCGGCTGGCGGCTGTGCGAACGCATTCT | |
| | GGCGCATCATCACCATCACCACTAA | |
| BsiWI | CTGACG | |
| 219a-2- | CTTAACAGC | miR |
| 3p | | NA |
| miRNA | | target 15 |
| BsiWI | CTGACG | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | Ggccggcatggtcccagcctcctcgctggcgccggctgggcaacat cttcggcatggcgaatgggac | 15 |
| Ribozyme | | |
| pReplicon-377-3p-BsiWI-4e | | |
| T7 | taatacgactcactatagg | 16 |
| promoter | | |
| Noncoding | GAGACCCAAGCTGGCTAGCGTTTAAACTTAAGCTTGGTACCGAGCTCGgatccctcgagctc | 246 |
| | gaaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 247 |
| | ACGCGGAGTAGCCCGTGTGAGCCCCTTTGGGGGCTTGATGGTGAGCAAGGGCG | |
| | AGGAGCTGTTCACCGGGGTGGTGCCCATCCTGGTCGAGCTGGACGGCGACGTAA | |
| | ACGGCCACAAGTTCAGCGTGTCCGGCGAGGGCGAGGGCGATGCCACCTACGGC | |
| | AAGCTGACCCTGAAGTTCATCTGCACCACCGGCAAGCTGCCCGTGCCCTGGCCC | |
| | ACCCTCGTGACCACCCTGACCTACGGCGTGCAGTGCTTCAGCCGCTACCCCGAC | |
| | CACATGAAGCAGCACGACTTCTTCAAGTCCGCCATGCCCGAAGGCTACGTCCAG | |
| | GAGCGCACCATCTTCTTCAAGGACGACGGCAACTACAAGACCCGCGCCGAGGT | |
| | GAAGTTCGAGGGCGACACCCTGGTGAACCGCATCGAGCTGAAGGGCATCGACT | |
| | TCAAGGAGGACGGCAACATCCTGGGGCACAAGCTGGAGTACAACTACAACAGC | |
| | CACAACGTCTATATCATGGCCGACAAGCAGAAGAACGGCATCAAGGTGAACTT | |
| | CAAGATCCGCCACAACATCGAGGACGGCAGCGTGCAGCTCGCCGACCACTACC | |
| | AGCAGAACACCCCCATCGGCGACGGCCCCGTGCTGCTGCCCGACAACCACTACC | |
| | TGAGCACCCAGTCCGCCCTGAGCAAAGACCCCAACGAGAAGCGCGATCACATG | |
| | GTCCTGCTGGAGTTCGTGACCGCCGCCGGGATCACTCTCGGCATGGACGAGCTG | |
| | TACAAGATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGTCTTCACACTCGAAGATTTC | |
| | GTTGGGGACTGGCGACAGACAGCCGGCTACAACCTGGACCAAGTCCTTGAACA | |
| | GGGAGGTGTGTCCAGTTTGTTTCAGAATCTCGGGGTGTCCGTAACTCCGATCCA | |
| | AAGGATTGTCCTGAGCGGTGAAAATGGGCTGAAGATCGACATCCATGTCATCAT | |
| | CCCGTATGAAGGTCTGAGCGGCGACCAAATGGGCCAGATCGAAAAAATTTTTAA | |
| | GGTGGTGTACCCTGTGGATGATCATCACTTTAAGGTGATCCTGCACTATGGCAC | |
| | ACTGGTAATCGACGGGGTTACGCCGAACATGATCGACTATTTCGGACGGCCGTA | |
| | TGAAGGCATCGCCGTGTTCGACGGCAAAAAGATCACTGTAACAGGGACCCTGTG | |
| | GAACGGCAACAAAATTATCGACGAGCGCCTGATCAACCCCGACGGCTCCCTGCT | |
| | GTTCCGAGTAACCATCAACGGAGTGACCGGCTGGCGGCTGTGCGAACGCATTCT | |
| | GGCGCATCATCACCATCACCACTAA | |
| BsiWI | CGTACG | |
| 377-3p | AGTGTGTTT | miR |
| | | NA |
| | | target 11 |
| BsiWI | CGTACG | |
| 377-3p | AGTGTGTTT | miR |
| | | NA |
| | | target 11 |
| BsiWI | CGTACG | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | Ggccggcatggtcccagcctcctcgctggcgccggctgggcaacatgcttcggcatggcgaatgggac | 15 |
| Ribozyme | | |
| pReplicon-377-3p-BsiWI-4c | | |
| T7 | taatacgactcactatagg | 16 |
| promoter | | |
| Noncoding | GAGACCCAAGCTGGCTAGCGTTTAAACTTAAGCTTGGTACCGAGCTCGgatccctcgagctc | 246 |
| | gaaccggtatcgatgttaacacaact | |
| Hammer | ctgatgagtccgtgaggacgaaacgagtaagctcgtc | 14 |
| head | | |
| Ribozyme | | |
| 5′ UTR | AGTTGTTACTGTTGCTGACTCAGACTGCGACAGTTCGAGTTTGAAGCGAAA | 239 |
| | GCTAGCAACAGTATCAACAGGTTTTATTTGGATTTGGAAACGAGAGTTTCT | |
| | GGTC | |
| coding | ATGAAAAACCCAAAAAAGAAATCCGGAGGATTCCGGATTGTCAATATGCTAAA | 247 |
| | ACGCGGAGTAGCCCGTGTGAGCCCCTTTGGGGGCTTGATGGTGAGCAAGGGCG | |
| | AGGAGCTGTTCACCGGGGTGGTGCCCATCCTGGTCGAGCTGGACGGCGACGTAA | |
| | ACGGCCACAAGTTCAGCGTGTCCGGCGAGGGCGAGGGCGATGCCACCTACGGC | |
| | AAGCTGACCCTGAAGTTCATCTGCACCACCGGCAAGCTGCCCGTGCCCTGGCCC | |
| | ACCCTCGTGACCACCCTGACCTACGGCGTGCAGTGCTTCAGCCGCTACCCCGAC | |
| | CACATGAAGCAGCACGACTTCTTCAAGTCCGCCATGCCCGAAGGCTACGTCCAG | |
| | GAGCGCACCATCTTCTTCAAGGACGACGGCAACTACAAGACCCGCGCCGAGGT | |
| | GAAGTTCGAGGGCGACACCCTGGTGAACCGCATCGAGCTGAAGGGCATCGACT | |
| | TCAAGGAGGACGGCAACATCCTGGGGCACAAGCTGGAGTACAACTACAACAGC | |
| | CACAACGTCTATATCATGGCCGACAAGCAGAAGAACGGCATCAAGGTGAACTT | |
| | CAAGATCCGCCACAACATCGAGGACGGCAGCGTGCAGCTCGCCGACCACTACC | |
| | AGCAGAACACCCCCATCGGCGACGGCCCCGTGCTGCTGCCCGACAACCACTACC | |
| | TGAGCACCCAGTCCGCCCTGAGCAAAGACCCCAACGAGAAGCGCGATCACATG | |
| | GTCCTGCTGGAGTTCGTGACCGCCGCCGGGATCACTCTCGGCATGGACGAGCTG | |
| | TACAAGATTGCATGTGTCCTCATTGTTGTGTTCCTATTGCTGGTGGTGCTCATAC | |
| | CTGAGCCAGAAAAGCAAAGATCTCCCCAGGACAACCAAATGGCAATCATCATC | |
| | ATGGTAGCAGTAGGTCTTCTGGGCTTGATTACCGCCAATGAACTCGGATGGTTG | |
| | GAGAGAACAAAGAGTGACCTAAGCCATCTAATGGTCTTCACACTCGAAGATTTC | |
| | GTTGGGGACTGGCGACAGACAGCCGGCTACAACCTGGACCAAGTCCTTGAACA | |
| | GGGAGGTGTGTCCAGTTTGTTTCAGAATCTCGGGGTGTCCGTAACTCCGATCCA | |
| | AAGGATTGTCCTGAGCGGTGAAAATGGGCTGAAGATCGACATCCATGTCATCAT | |
| | CCCGTATGAAGGTCTGAGCGGCGACCAAATGGGCCAGATCGAAAAAATTTTTAA | |
| | GGTGGTGTACCCTGTGGATGATCATCACTTTAAGGTGATCCTGCACTATGGCAC | |
| | ACTGGTAATCGACGGGGTTACGCCGAACATGATCGACTATTTCGGACGGCCGTA | |
| | TGAAGGCATCGCCGTGTTCGACGGCAAAAAGATCACTGTAACAGGGACCCTGTG | |
| | GAACGGCAACAAAATTATCGACGAGCGCCTGATCAACCCCGACGGCTCCCTGCT | |
| | GTTCCGAGTAACCATCAACGGAGTGACCGGCTGGCGGCTGTGCGAACGCATTCT | |
| | GGCGCATCATCACCATCACCACTAA | |
| BsiWI | CTGACG | |
| 377-3p | AGTGTGTTT | miR |
| | | NA |
| | | target 11 |
| BsiWI | CTGACG | |
| 3′ UTR | GCACCAATCTTAATGTTGTCAGGCCTGCTAGTCAGCCACAGCTTGGGGAAAGCTG | 244 |
| | TGCAGCCTGTGACCCCCCCAGGAGAAGCTGGGAAACCAAGCCTATAGTCAGGCC | |
| | GAGAACGCCATGGCACGGAAGAAGCCATGCTGCCTGTGAGCCCCTCAGAGGACA | |
| | CTGAGTCAAAAAACCCCACGCGCTTGGAGGCGCAGGATGGGAAAAGAAGGTGGC | |
| | GACCTTCCCCACCCTTCAATCTGGGGCCTGAACTGGAGATCAGCTGTGGATCTCC | |
| | AGAAGAGGGACTAGTGGTTAGAGGAGACCCCCCGGAAAACGCAAAACAGCATAT | |
| | TGACGCTGGGAAAGACCAGAGACTCCATGAGTTTCCACCACGCTGGCCGCCAGGC | |
| | ACAGATCGCCGAATAGCGGCGGCCGGTGTGGGGAAATCCATGGGTCT | |
| HDV | Ggccggcatggtcccagcctcctcgctggcgccggctgggcaacatgcttcggcatggcgaatgggac | 15 |
| Ribozyme | | |

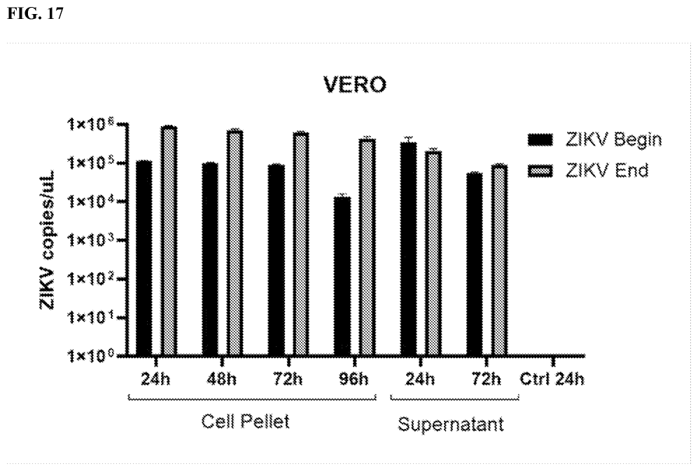

The viral RNA was produced by an in vitro transcription. The viral RNA was transfected into a Vero cell via electroporation, according to the scheme in FIG. 4A. The recombinant zika virus was produced and released in the culture supernatant. The RNA transcription products produced and released into the supernatant were analyzed and visualized using RNA electrophoresis. Analyses were performed using the T7-HIGH kit (NewEngland), where samples were pretreated with DNase, and HiScribe™ T7 ARCA mRNA Kit (with tailing; ThermoFisher), as shown in FIG. 16. RT-qPCR was performed to compare the amount of ZIKV genome viral copies produced at various time intervals, as shown in FIG. 17. The amount of ZIKV copies/μL in the cell pellet and supernatant were compared. The amount of ZIKV copies/μL in the Vero cell pellet was quantified at 24 h, 48h, 72h, and 96h after transfection. The amount of ZIKV copies in the supernatant was quantified at 24 h and 72h after transfection. Whole RNA (beginning (black) and end (gray)) was observed in the culture supernatant.

Transfection of the viral RNA was accomplished by co-transfecting with lipofectamine (a CMV promoter) and electroporation with an initial amount of RNA of about 10 μg to about 35 μg RNA. The culture was subsequently reinfected in another T25 after concentrating the virus, enabling observation of viral production (FIG. 18).

Vero cells transfected with the modified ZIKV RNA were observed after 1-, 4-, 5-, and 6-days post infection (DPI), as shown in FIG. 18. Images marked with (*) indicate an evident cytopathic effect of ZIKV RNA on the cell population. Cells transfected with samples TI_15 (pCC1-ZIKV-3P-T7), TI_16 (pCC1-ZIKV-0P-T7), TI_17 (pCC1-ZIKV-2P-T7), and TI_18 (pCC1-ZIKV-3P-T7) exhibited cell death after 5 DPI, whereas cells transfected with TI_14 exhibited cell death after 6 DPI. During the process of in vitro transcription protocol establishment, 24 RNA products were produced. All RNAs produced were transfected, resulting in the production of eight modified viruses so far. FIG. 18 shows the virus generation process compared to the negative control. It was possible to observe the cytopathic effect (generation of infective virus) from the fourth day post-reinfection in all RNAs tested (TI_14, TI_15, TI_16, TI_17, TI_18).

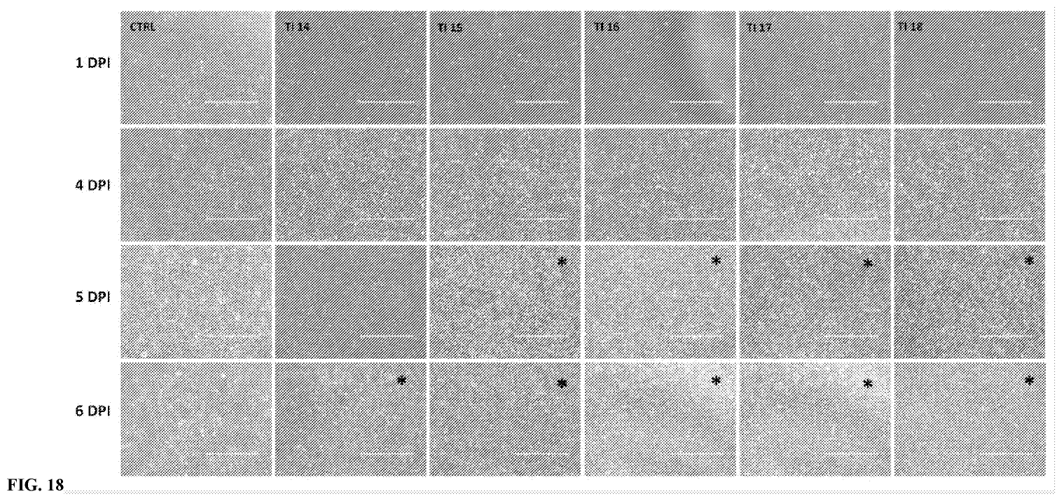

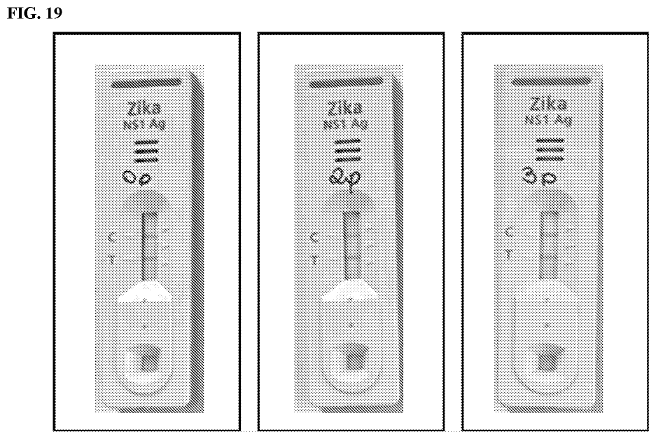

To confirm the presence of the virus, a chromatographic test was used to identify the ZIKV NS1 protein in the culture supernatant. As shown in FIG. 19, the two positive bars (one control, “C”, and one test, “T”) indicated the presence of ZIKV in the supernatant.

### Oncolytic Effect and Safety in Normal Cells

Virus safety was tested in neural progenitor cells (NPCs) derived from iPS human cells. A Go/No-go phenotype test using tumoral cell lines to examine the oncolytic effect was used to determine virus safety. After tumoral and non-tumoral virus infection, supernatant samples were collected to evaluate virus replication by quantifying the amount of RNA copies using RT-qPCR and Virus Spread by Plaque Assay (PFU) to quantify the amount of infective virus. The results are shown in Tables 10 and 11.

TABLE 10
Different recombinant zika virus constructs and effect on cell titer.

| | | | Titer | | |
|---|---|---|---|---|---|
| | | Modification | PFU | RT-PCR | |
| ID | miRNA | location | (ip) | (vp) | vp/ip |
| oZIKV_4 (similar | — | — | 4.12e5 | 6.1e6 | 14.81* |
| to FIG. 9) | | | | | |
| oZIKV 5 (similar | miR-219a-2-3p_c2 | 2k | 8.75e4 | 4.03e6 | 46.05** |
| to FIG. 13) | | | | | |
| oZIKV_6 (similar | miR-129-5p | 3′ UTR | 1.12e5 | 9.54e6 | 85.14** |
| to FIG. 14) | | | | | |
| oZIKV_14 (similar | miR-219a-2-3p | 2k | 5.25e5 | 1.39e7 | 26.47* |
| to FIG. 13) see | | | | | |
| Table 9A and 9B | | | | | |
| pCC1-ZIKV-2P-T7 | | | | | |
| oZIKV_15 (similar | miR-129-5p | 3′ UTR | 1.30e6 | 1.83e7 | 14.06* |
| to FIG. 14) | | | | | |
| oZIKV_16 (similar | — | — | 1.70e6 | 2.45e7 | 14.42* |
| to FIG. 9) | | | | | |
| oZIKV_17 (similar | miR-219a-2-3p | 2k | 5.13e6 | 1.44e7 | 28.03* |
| to FIG. 13) | | | | | |
| oZIKV_18 (similar | miR-129-5p | 3′ UTR | 3.38e5 | 1.24e7 | 36.70* |
| to FIG. 14) | | | | | |
| ZIKV_17_wildtype | | | | 1.5e5 | |

*0-30;
**30-50;
***>50

TABLE 11
Effect of different recombinant zika virus particles on tumor cells (CHLA, DAOY)
and normal cells (neural progenitor cells, “NPC”) as quantified by RT-PCR and PFU.

| | Effectiveness (Score) | | | | | | Replication | | | Virus Spread | | |
|---|---|---|---|---|---|---|---|---|---|---|---|---|
| | Viability CHLA | | Viability DAOY | | Safety | | after infection | | | after infection | | |
| | MOI 1 (relative- | | MOI 1 (relative- | | Viability NPC | | MOI 1 | | | MOI 1 | | |
| | MOCK) | | MOCK) | | (Score) | | RT-PCR | | | PFU | | |
| ID | Number | Score | Number | Score | Number | Score | CHLA | DAOY | NPC | CHLA | DAOY | NPC |
| oZIKV_4 | 6.90% | xxxx | 0.79% | xxx | 104% | 0 | 2.09e6 | 7.65e4 | 2.68e5 | 2.06e5 | 1.28e4 | 1.25e3 |
| oZIKV_5 | 95% | — | 1.40% | xxx | — | — | 5.06e5 | 2.66e5 | — | 8.61e5 | 0 | — |
| oZIKV_6 | 72% | — | 1.20% | xxx | — | — | 7.74e5 | 3.10e5 | — | 2.26e6 | 0 | — |
| oZIKV_14 | 26% | xx | 1.90% | xxx | 91% | 0 | 4.15e6 | 4.70e5 | 3.17e5 | 5.21e6 | 0 | 5.25e3 |
| oZIKV_15 | 68% | — | 3.70% | xx | — | — | 3.69e6 | 6.49e5 | — | 2.09e6 | 0 | — |
| oZIKV_16 | 56% | x | 14.70% | x | — | — | 4.15e6 | 4.90e5 | — | 5.73e6 | 8.25e4 | — |
| oZIKV_17 | 23.70% | xx | 3.20% | xx | 30% | xx | 3.44e6 | 2.60e5 | 2.11e5 | 6.17e5 | 9.75e4 | 1.28e3 |
| oZIKV_18 | 14% | xxx | 2.40% | xxx | 134% | 0 | 2.74e6 | 2.88e5 | 3.11e5 | 1.51e6 | 0 | 1.99e4 |
| ZIKV_17_ | 19% | xx | 4.20% | xx | 76% | x | | 1.33e6 | 3.07e6 | | | 1.50e2 |
| wildtype | | | | | | | | | | | | |

### Oncolytic Effect-Tumoral Cell Lines

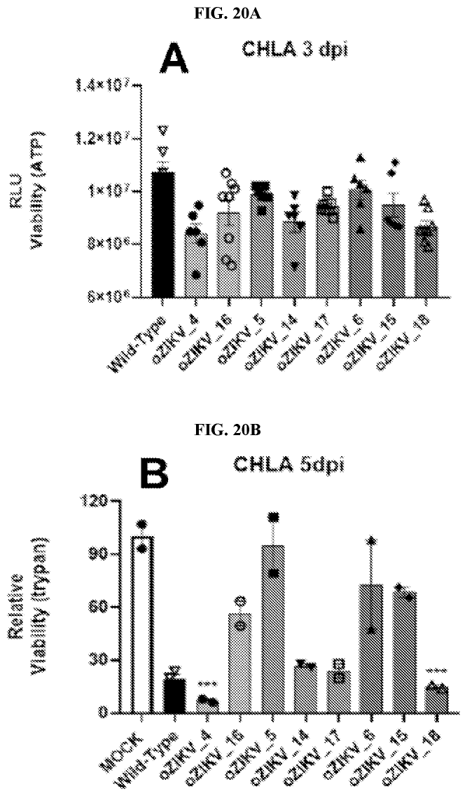

Cell viability tests were performed to confirm the oncolytic effect of the 8 modified viruses. The cells were tested after viral infection in the tumor cell line CHLA-06-ATRT after 3 DPI (FIG. 20A) and 5 DPI (FIG. 20B). FIG. 20A shows the results of a luciferase-based, ATP-dependent assay (CELLTITER GLO® 2.0 Cell Viability Assay, PROMEGA). As shown in FIG. 20B, the different recombinant zika virus particles had varying effects on cell viability as evaluated using a trypan blue exclusion test. The results confirm the oncolytic effect of several samples, including ZIKV_4, ZIKV_14, ZIKV_17, and ZIKV_18. The recombinant zika viruses ZIKV 4 and ZIKV_18 showed a higher oncolytic effect when compared against the wild-type virus.

### Safety

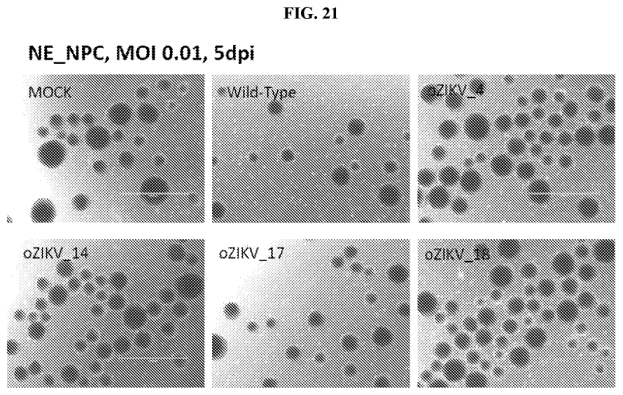

After determining the oncolytic effect of several recombinant zika viruses, the safety of using these recombinant zika viruses was tested in NPC cells, as shown in FIG. 21. NPCs were imaged 5 DPI. When compared against the control (MOCK), the ZIKV_4 and ZIKV_18 strains did not demonstrate an apparent cytotoxic effect on NPCs. As a second control, the wild-type zika virus exhibited a cytotoxic effect on NPCs. The recombinant zika virus ZIKV_17 had an apparent cytotoxic effect on NPCs but to a lesser degree than wild-type zika virus. Therefore, the ZIKV 4 and ZIKV_18 strains demonstrated an oncolytic effect on CHLA tumor cells while exhibiting little to no cytotoxic effect on normal NPCs.

Neurosphere analysis of the NPCs transfected with wildtype and recombinant zika virus was performed, as shown in FIG. 22. FIG. 22A shows the measured neurosphere area of the different NPCs, and FIG. 22B shows the perimeter of the different NPCs.

miRNA Modulation: Replication Inhibition System

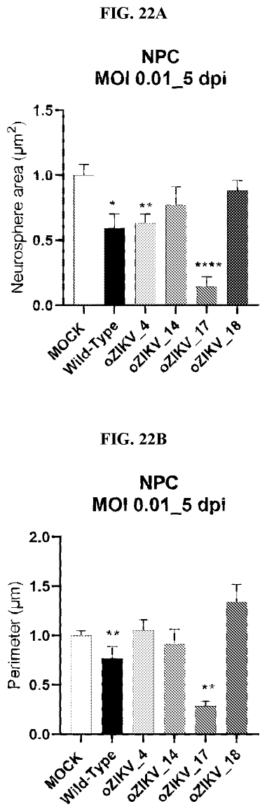

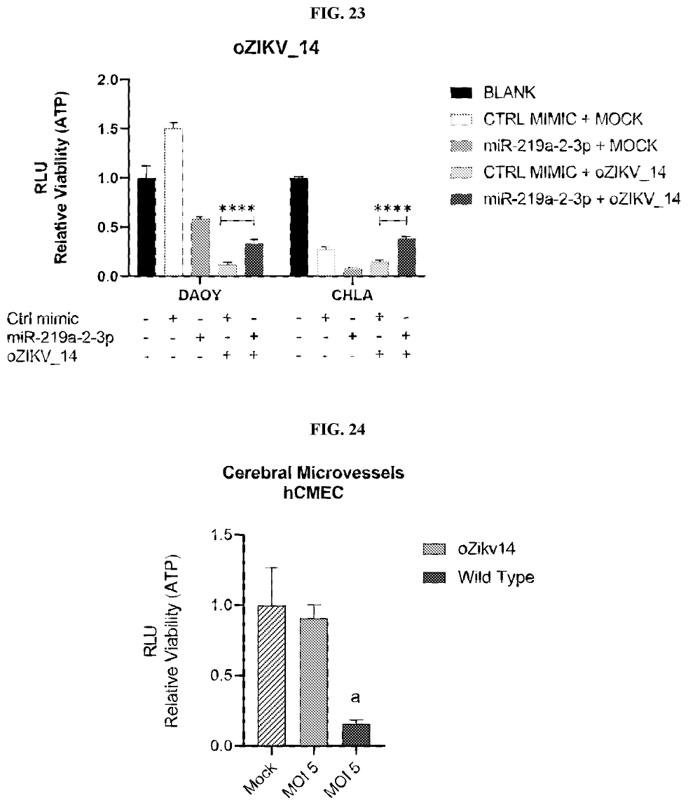

Based on the cell lines' miRNA expression profile and zika candidates' infection/safety, the effectiveness of the selective inhibition mechanism based on microRNA regulation to control viral replication was tested. For that, we overexpressed the correspondent miRNA to observe virus inhibition and viability tumor cell increase. FIG. 23 shows that ZIKV_14 was oncolytic in tumoral cells and safe in NPC (equal to MOCK) and can be modulated by miR-219a-2-3p overexpression. FIG. 23 shows a cell viability analysis with a relative comparison against the negative control after viral infection in Daoy cells and CHLA cells with the recombinant ZIKV14 virus miRNA overexpression. These results illustrate that ZIKV_14 is oncolytic, and safe in NPC.

### Example 4: Recombinant ZIKA Virus Spectrum of Action

Using the same assays as described above, the viability of tumoral and non-tumoral cells were tested after ZIKV 14 infection in a curve of virus concentration of MOI 5, 1.667, 0.556, 0.185, 0.062, 0.021, 0.007, 0.002. Tumoral cell lines tested included hCMEC, Hs1.Tes, Daoy, ONS, ATRT, glioblastoma, prostate, triple-negative breast tumor cells, colon tumor cells, and luminal breast tumor cells. Cells were plated in a 96-well plate with an initial seeding density of 2×10³ cells/well. After 24h, wild-type ZIKA virus, recombinant ZIKA virus, or MOCK were added to the cells for a final volume of 20 μL. After incubating the cells for 30 minutes at room temperature, wild-type ZIKA virus, recombinant ZIKA virus, or MOCK were removed and replaced with 100 μL fresh media. Viable cells were assayed 3 DPI using CELLTITER GLO® 2.0 Cell Viability Assay (PROMEGA). 8 replicates of each cell line were run.

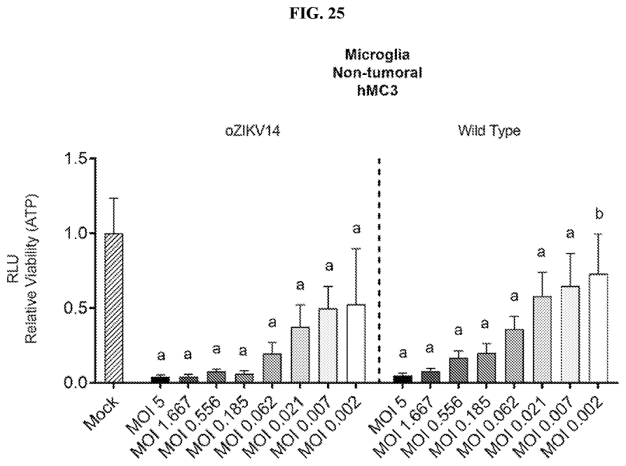

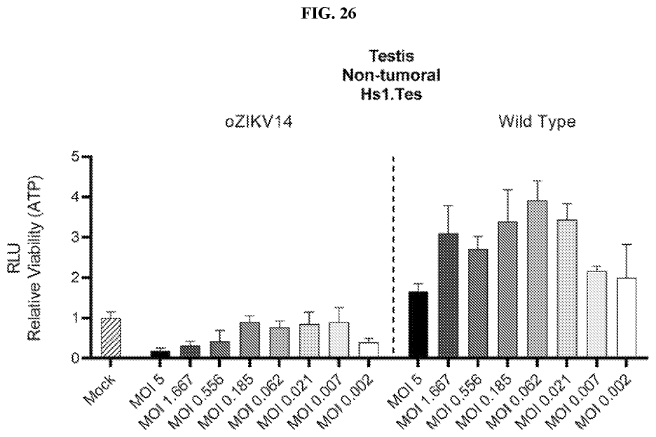

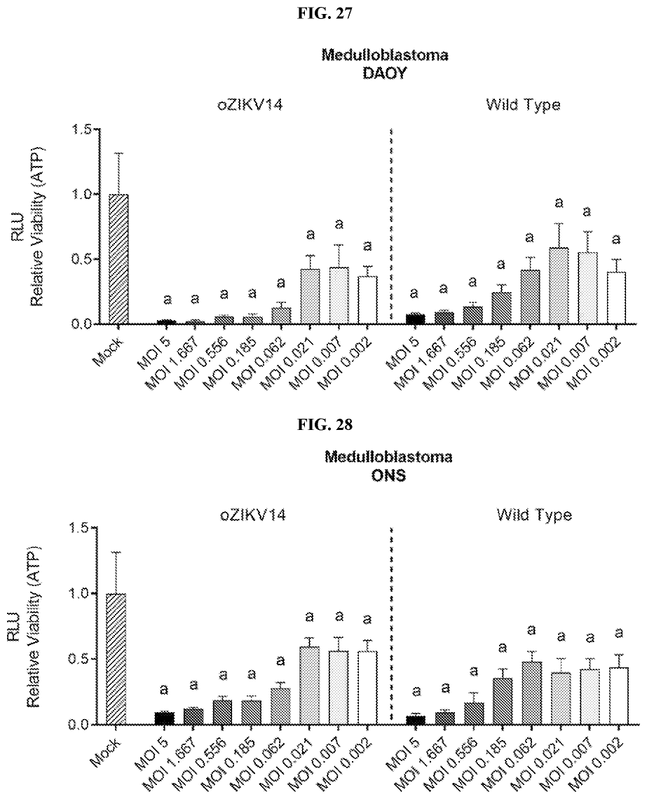

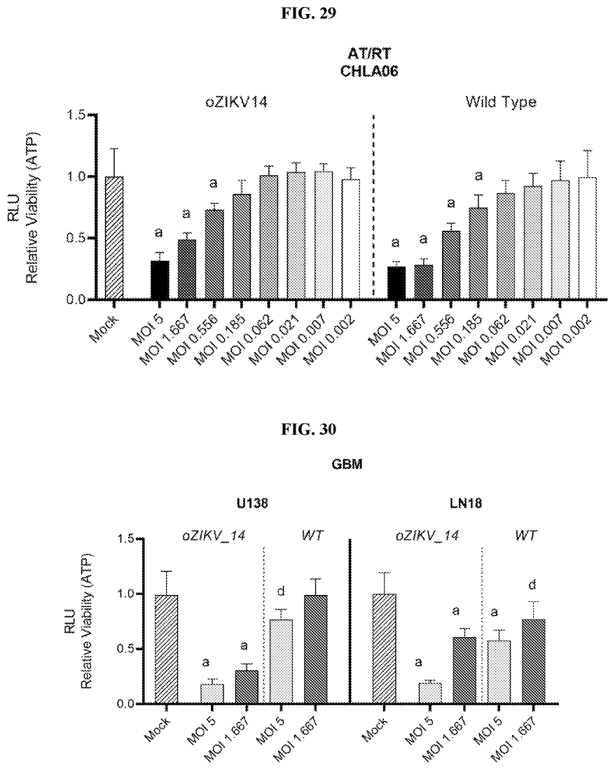

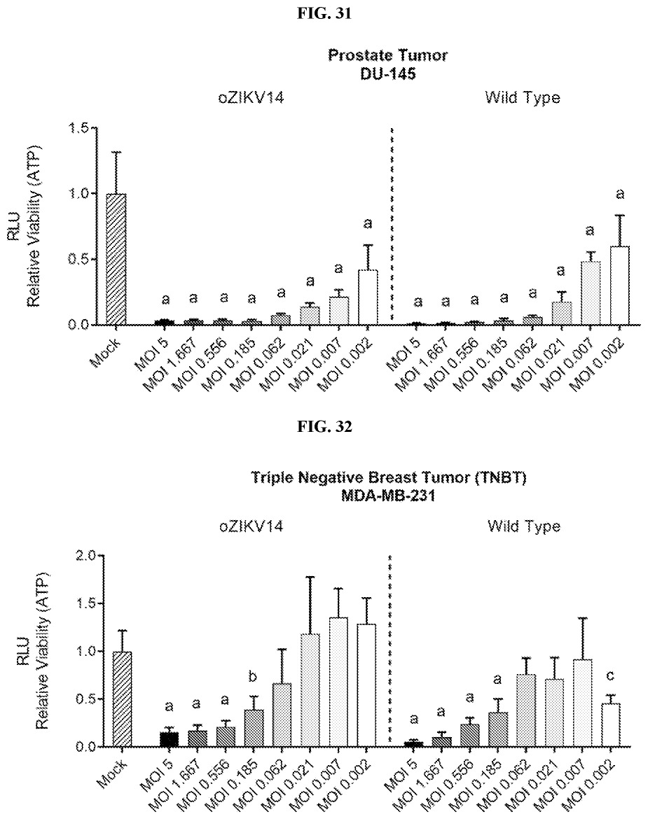

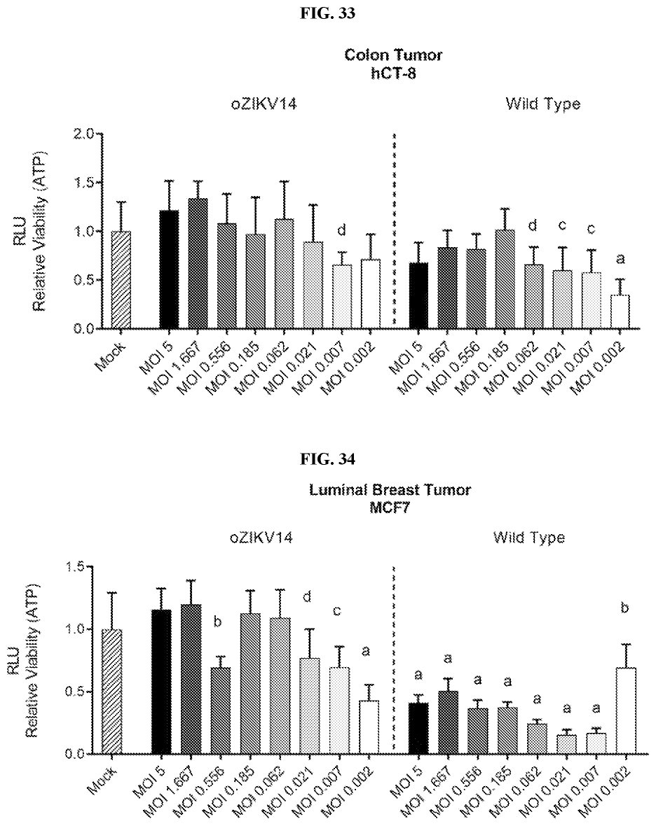

The results of the assays are presented in FIGS. 24-34. FIG. 24 shows the recombinant ZIKA virus (ZIKV14) did not decrease cell viability in cerebral microvessel (hCMEC cell line) showing safety of ZIKV14 when compared with wild-type ZIKV. FIG. 25 shows the ability of recombinant ZIKA virus (ZIKV14) and wild-type ZIKV to decrease cell viability in microglia (hMC3 cell line). FIG. 26 shows that recombinant ZIKA virus (ZIKV14) and wild-type ZIKV did not decrease cell viability in testis cancer (Hs1.Tes cell line). FIG. 30 shows the oncolytic effect of ZIKV14 was more efficient in glioblastoma cell lines (U138 and LN18) when compared with ZIKV wild-type. FIGS. 27-29 show that recombinant zika virus (ZIKV14) infection decreased cell viability when compared with wild-type ZIKV in the embryonal CNS tumors, medulloblastoma (DAOY and ONS) and ATRT (CHLA-06-ATRT). FIGS. 31-34 show that infection with ZIKV14 and wild-type ZIKV decreased the viability in prostate cancer (DU-145 cell lines) (FIG. 31) and triple negative breast tumor (MDA-MB-231 cell line) (FIG. 32) but were not oncolytic in MCF7 cell line (luminal breast cancer) (FIG. 34) and hCT-8 cell lines (colon tumor) (FIG. 33).

### Example 5: Mouse Model

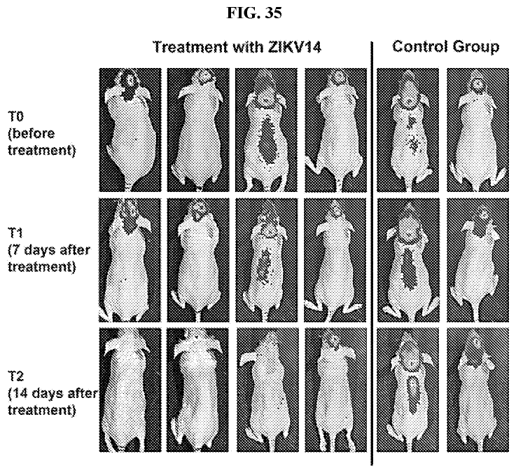

The ZIKV_14 was validated in an in vivo model (orthotopic and metastatic) of nervous system tumor. FIG. 35 shows that the modified zika virus (ZIKV14) was able to stabilize tumor growth, decrease tumor development, and even eradicate the tumor sites. Additionally, the modified zika viruses were capable of inhibiting the development of neuraxial metastases, as well as eliminating pre-existing tumor metastases (FIG. 35). Briefly, Balb/c Nude mice were administered tumor cells and that had or had not metastasized in the neuraxis. In the control group, mice received intracerebroventricular (i.c.v.) vehicle of MOCK or wild-type zika virus. Other mice were treated with ZIKV14 via i.c.v. injections (6×10³ PFU) and monitored. Images were collected before treatment (TO), 7 days post treatment (T1), and 14 days post treatment (T2) with recombinant ZIKV14.

TABLE 5
Additional example sequences

| SEQ ID NO | |
|---|---|
| Name | Sequence |
| SEQ ID NO: 26 | tctccccaggacatgcatccgacctgtatgcataaccaaatggcaatcatc |
| Modified 2k | |
| forward region | |
| with EcoT22I- | |
| 1225-5p-EcoT22I | |
| (FIG. 1F) | |
| SEQ ID NO: 27 | gatgattgccatttggttatgcatacaggtcggatgcatgtcctggggaga |
| Modified 2k | |
| reverse region with | |
| EcoT22I-1225-5p- | |
| EcoT22I (FIG. 1F) | |
| SEQ ID NO: 28 | acacctggagtgctg |
| NS5 sense | |
| (forward) (FIG. | |
| 1G) | |
| SEQ ID NO: 29 | cagcactccaggtgt |
| NS5 antisense | |
| (complementary) | |
| (FIG. 1G) | |
| SEQ ID NO: 31 | ggacatgcatcttaacaccatgcataaccaa |
| 2k modification | |
| (FIG. 3A) | |
| SEQ ID NO: 32 | ggacatgcatccgacctgtatgcataaccaa |
| 2k modification | |
| (FIG. 3A) | |
| SEQ ID NO: 33 | tggtcgtacgcttaacacccgtacgatgaaa |
| 5′ UTR-miRNA | |
| target (FIG. 3A) | |
| SEQ ID NO: 34 | tgtaacgcgtttaacaccgacgcgtgcacc |
| 3′ UTR-miRNA | |
| target (FIG. 3A) | |
| SEQ ID NO: 36 | ggacatgcatctaacaggtatgcataaccaa |
| 2k modification | |
| (FIG. 3B) | |
| SEQ ID NO: 37 | ggacatgcattaacaggttatgcataaccaa |
| 2k modification | |
| (FIG. 3B) | |
| SEQ ID NO: 38 | Tggtcgtacgactaacaggcgtacgatgaaa |
| 5′ UTR-miRNA | |
| target (FIG. 3B) | |
| SEQ ID NO: 39 | Tgtaacgcgtctaacaggtacgcgtgcacc |
| 3′ UTR-miRNA | |
| target (FIG. 3B) | |
| SEQ ID NO: 40 | ggacatgcataaaaacgccatgcataaccaa |
| 2k modification | |
| (FIG. 3C) | |
| SEQ ID NO: 41 | ggacatgcataaacgccagatgcataaccaa |
| 2k modification | |
| (FIG. 3C) | |
| SEQ ID NO: 42 | tggtcgtacgaaaaacgcccgtacgatgaaa |
| 5′ UTR-miRNA | |
| target (FIG. 3C) | |
| SEQ ID NO: 43 | tgtaacgcgtaaaaacgccacgcgtgcacc |
| 3′ UTR-miRNA | |
| target (FIG. 3C) | |
| SEQ ID NO: 45 | ggacatgcatcacccatgcatgcataaccaa |
| 2k modification | |
| (FIG. 3D) | |
| SEQ ID NO: 46 | ggacatgcatacccatgccatgcataaccaa |
| 2k modification | |
| (FIG. 3D) | |
| SEQ ID NO: 47 | tggtcgtacgcccatgccgcgtacgatgaaa |
| 5′ UTR-miRNA | |
| target (FIG. 3D) | |
| SEQ ID NO: 48 | tgtaacgcgtacccatgccacgcgtgcacc |
| 3′ UTR-miRNA | |
| target (FIG. 3D) | |
| SEQ ID NO: 49 | ggacatgcatagtgtgtttatgcataaccaa |
| 2k modification | |
| (FIG. 3E) | |
| SEQ ID NO: 50 | tggtcgtacgagtgtgtttcgtacgatgaaa |
| 5′ UTR-miRNA | |
| target (FIG. 3E) | |
| SEQ ID NO: 51 | tgtaacgcgtagtgtgtttacgcgtgcacc |
| 3′ UTR-miRNA | |
| target (FIG. 3E) | |
| SEQ ID NO: 52 | attgcatgtgtcctcattgttgtgttcctattgctggtggtgctcatacctgagccagaaaagcaaagat |
| Modified construct | ctccccaggacatgcaTGCAGGTGGTCACCTGCATGCAtaaccaaatggcaatcatcatcat |
| (FIG. 12) | ggtagcagtaggtcttctgggcttgattaccgccaatgaactcggatggttggagagaacaaagagtgac |
| | ctaagccatctaatgggaaggagagaggagggggcaac |
| SEQ ID NO: 53 | aggcgaacca |
| AarI motif (FIG. | |
| 12) | |
| SEQ ID NO: 54 | tggcggtcct |
| miRNA target | |
| (FIG. 12) | |
| SEQ ID NO: 223 | tctccccaggacaaccaaatggcaatcatc |
| 2k forward | |
| (FIG. 1E) | |
| SEQ ID NO: 224 | gatgattgccatttggttgtcctggggaga |
| 2k reverse (FIG. | |
| 1E) | |
| SEQ ID NO: 228 | tctccccaggac |
| first region of 2k | |
| (FIG. 1F) | |
| SEQ ID NO: 229 | gtcctggggaga |
| first region of 2k | |
| (complementary) | |
| (FIG. 1F) | |
| SEQ ID NO: 230 | aaccaaatggcaatcatc |
| second region of 2k | |
| (FIG. 1F) | |
| SEQ ID NO: 231 | gatgattgccatttggt |
| second region of 2k | |
| (complementary) | |
| (FIG. 1F) | |
| SEQ ID NO: 232 | acacctggagtgctg |
| NS5 (FIG. 1G) | |
| SEQ ID NO: 233 | cagcactccaggtgt |
| NS5 reverse | |
| complement (RC) | |
| (FIG. 1G) | |
| SEQ ID NO: 234 | gcaccaatcttaatgttgtcaggcctgctagtc |
| 3′ UTR (FIG. 1G) | |
| SEQ ID NO: 235 | gactagcaggcctgacaacattaagattggtgc |
| 3′ UTR RC (FIG. | |
| 1G) | |
| SEQ ID NO: 236 | taacgcgttgccggtcaacgcgtgcaccaatcttaatgttgtcaggcctgctagtc |
| modified 3′UTR | |
| forward with MluI- | |
| miR-1225-5p-MluI | |
| (FIG. 1G) | |
| SEQ ID NO: 237 | gactagcaggcctgacaacattaagattggtgcacgcgttgaccggcaacgcgtta |
| modified 3′UTR | ATGAAAAACCCAAAAAAGAAATCCGGAGGATTCCGGATTGTCA |
| RC with MluI- | ATATGCTAAAACGCGGAGTAGCCCGTGTGAGCCCCTTTGGGGG |
| miR-1225-5p-MluI | CTTGAAGAGGCTGCCAGCCGGACTTCTGCTGGGTCATGGGCCCA |
| (FIG. 1G) | TCAGGATGGTCTTGGCAATTCTAGCCTTTTTGAGATTCACGGCA |
| SEQ ID NO: 250 | ATCAAGCCATCACTGGGTCTCATCAATAGATGGGGTTCAGTGGG |
| coding region | GAAAAAAGAGGCTATGGAAATAATAAAGAAGTTCAAGAAAGA |
| | TCTGGCTGCCATGCTGAGAATAATCAATGCTAGGAAGGAGAAG |
| | AAGAGACGGGGCGCAGATACTAGTGTCGGAATTGTTGGCCTCC |
| | TGCTGACCACAGCTATGGCAGCGGAGGTCACTAGACGTGGGAG |
| | TGCATACTATATGTACTTGGACAGAAACGATGCTGGGGAGGCC |
| | ATATCTTTTCCAACCACATTGGGGATGAATAAGTGTTATATACA |
| | GATCATGGATCTTGGACACATGTGTGATGCCACCATGAGCTATG |
| | AATGCCCTATGCTGGATGAGGGGGTGGAACCAGATGACGTCGA |
| | TTGTTGGTGCAACACGACGTCAACTTGGGTTGTGTACGGAACCT |
| | GCCATCACAAAAAAGGTGAAGCACGGAGATCTAGAAGAGCTGT |
| | GACGCTCCCCTCCCATTCCACTAGGAAGCTGCAAACGCGGTCGC |
| | AAACCTGGTTGGAATCAAGAGAATACACAAAGCACTTGATTAG |
| | AGTCGAAAATTGGATATTCAGGAACCCTGGCTTCGCGTTAGCAG |
| | CAGCTGCCATCGCTTGGCTTTTGGGAAGCTCAACGAGCCAAAA |
| | AGTCATATACTTGGTCATGATACTGCTGATTGCCCCGGCATACA |
| | GCATCAGGTGCATAGGAGTCAGCAATAGGGACTTTGTGGAAGG |
| | TATGTCAGGTGGGACTTGGGTTGATATTGTCTTGGAACATGGAG |
| | GTTGTGTCACCGTAATGGCACAGGACAAACCGACTGTCGACAT |
| | AGAGCTGGTTACAACAACAGTCAGCAACATGGCGGAGGTAAGA |
| | TCCTACTGCTATGAGGCATCAATATCAGACATGGCTTCGGACAG |
| | CCGCTGCCCAACACAAGGTGAAGCCTACCTTGACAAGCAATCA |
| | GACACTCAATATGTCTGCAAAAGAACGTTAGTGGACAGAGGCT |
| | GGGGAAATGGATGTGGACTTTTTGGCAAAGGGAGTCTGGTGAC |
| | ATGCGCTAAGTTTGCATGCTCCAAGAAAATGACCGGGAAGAGC |
| | ATCCAGCCAGAGAATCTGGAGTACCGGATAATGCTGTCAGTTC |
| | ATGGCTCCCAGCACAGTGGGATGATCGTTAATGACACAGGACA |
| | TGAAACTGATGAGAATAGAGCGAAGGTTGAGATAACGCCCAAT |
| | TCACCAAGAGCCGAAGCCACCCTGGGGGGTTTTGGAAGCCTAG |
| | GACTTGATTGTGAACCGAGGACAGGCCTTGACTTTTCAGATTTG |
| | TATTACTTGACTATGAATAACAAGCACTGGTTGGTTCACAAGGA |
| | GTGGTTCCACGACATTCCATTACCTTGGCACGCTGGGGCAGACA |
| | CCGGAACTCCACACTGGAACAACAAAGAAGCACTGGTAGAGTT |
| | CAAGGACGCACATGCCAAAAGGCAAACTGTCGTGGTTCTAGGG |
| | AGTCAAGAAGGAGCAGTTCACACGGCCCTTGCTGGAGCTCTGG |
| | AGGCTGAGATGGATGGTGCAAAGGGAAGGCTGTCCTCTGGCCA |
| | CTTGAAATGTCGCCTGAAAATGGATAAACTTAGATTGAAGGGC |
| | GTGTCATACTCCTTGTGTACCGCAGCGTTCACATTCACCAAGAT |
| | CCCGGCTGAAACACTGCACGGGACAGTCACAGTGGAGGTACAG |
| | TACGCAGGGACAGATGGACCTTGCAAGGTTCCAGCTCAGATGG |
| | CGGTGGACATGCAAACTCTGACCCCAGTTGGGAGGTTGATAAC |
| | CGCTAACCCCGTAATCACTGAAAGCACTGAGAACTCTAAGATG |
| | ATGCTGGAACTTGATCCACCATTTGGGGACTCTTACATTGTCAT |
| | AGGAGTCGGGGAGAAGAAGATCACCCACCACTGGCACAGGAGT |
| | GGCAGCACCATTGGAAAAGCATTTGAAGCCACTGTGAGAGGTG |
| | CCAAGAGAATGGCAGTCTTGGGAGACACAGCCTGGGACTTTGG |
| | ATCAGTTGGAGGCGCTCTCAACTCATTGGGCAAGGGCATCCATC |
| | AAATTTTTGGAGCAGCTTTCAAATCATTGTTTGGAGGAATGTCC |
| | TGGTTCTCACAAATTCTCATTGGAACGTTGCTGATGTGGTTGGG |
| | TCTGAACACAAAGAATGGATCTATTTCCCTTATGTGCTTGGCCT |
| | TAGGGGGAGTGTTGATCTTCTTATCCACAGCCGTCTCTGCTGAT |
| | GTGGGGTGCTCGGTGGACTTCTCAAAGAAGGAGACGAGATGCG |
| | GTACAGGGGTGTTCGTCTATAACGACGTTGAAGCCTGGAGGGA |
| | CAGGTACAAGTACCATCCTGACTCCCCCCGTAGATTGGCAGCAG |
| | CAGTCAAGCAAGCCTGGGAAGATGGTATCTGCGGGATCTCCTCT |
| | GTTTCAAGAATGGAAAACATCATGTGGAGATCAGTAGAAGGGG |
| | AGCTCAACGCAATCCTGGAAGAGAATGGAGTTCAACTGACGGT |
| | CGTTGTGGGATCTGTAAAAAACCCCATGTGGAGAGGTCCACAG |
| | AGATTGCCCGTGCCTGTGAACGAGCTGCCCCACGGCTGGAAGG |
| | CTTGGGGGAAATCGCACTTCGTCAGAGCAGCAAAGACAAATAA |
| | CAGCTTTGTCGTGGATGGTGACACACTGAAGGAATGCCCACTCA |
| | AACATAGAGCATGGAACAGCTTTCTTGTGGAGGATCATGGGTTC |
| | GGGGTATTTCACACTAGTGTCTGGCTCAAGGTTAGAGAAGATTA |
| | TTCATTAGAGTGTGATCCAGCCGTTATTGGAACAGCTGTTAAGG |
| | GAAAGGAGGCTGTACACAGTGATCTAGGCTACTGGATTGAGAG |
| | TGAGAAGAATGACACATGGAGGCTGAAGAGGGCCCATCTGATC |
| | GAGATGAAAACATGTGAATGGCCAAAGTCCCACACATTGTGGA |
| | CAGATGGAATAGAAGAGAGTGATCTGATCATACCCAAGTCTTT |
| | AGCTGGGCCACTCAGCCATCACAATACCAGAGAGGGCTACAGG |
| | ACCCAAATGAAAGGGCCATGGCACAGTGAAGAGCTTGAAATTC |
| | GGTTTGAGGAATGCCCAGGCACTAAGGTCCACGTGGAGGAAAC |
| | ATGTGGAACAAGAGGACCATCTCTGAGATCAACCACTGCAAGC |
| | GGAAGGGTGATCGAGGAATGGTGCTGCAGGGAGTGCACAATGC |
| | CCCCACTGTCGTTCCGGGCTAAAGATGGCTGTTGGTATGGAATG |
| | GAGATAAGGCCCAGGAAAGAACCAGAAAGCAACTTAGTAAGG |
| | TCAATGGTGACTGCAGGATCAACTGATCACATGGATCACTTCTC |
| | CCTTGGAGTGCTTGTGATTCTGCTCATGGTGCAGGAAGGGCTGA |
| | AGAAGAGAATGACCACAAAGATCATCATAAGCACATCAATGGC |
| | AGTGCTGGTAGCTATGATCCTGGGAGGATTTTCAATGAGTGACC |
| | TGGCTAAGCTTGCAATTTTGATGGGTGCCACCTTCGCGGAAATG |
| | AACACTGGAGGAGATGTAGCTCATCTGGCGCTGATAGCGGCAT |
| | TCAAAGTCAGACCAGCGTTGCTGGTATCTTTCATCTTCAGAGCT |
| | AATTGGACACCCCGTGAAAGCATGCTGCTGGCCTTGGCCTCGTG |
| | TCTTTTGCAAACTGCGATCTCCGCCTTGGAAGGCGACCTGATGG |
| | TTCTCATCAATGGTTTTGCTTTGGCCTGGTTGGCAATACGAGCG |
| | ATGGTTGTTCCACGCACTGATAACATCACCTTGGCAATCCTGGC |
| | TGCTCTGACACCACTGGCCCGGGGCACACTGCTTGTGGCGTGGA |
| | GAGCAGGCCTTGCTACTTGCGGGGGGTTTATGCTCCTCTCTCTG |
| | AAGGGAAAAGGCAGTGTGAAGAAGAACTTACCATTTGTCATGG |
| | CCCTGGGACTAACCGCTGTGAGGCTGGTCGACCCCATCAACGTG |
| | GTGGGGCTGCTGTTGCTCACAAGGAGTGGGAAGCGGAGCTGGC |
| | CCCCTAGCGAAGTACTCACAGCTGTTGGCCTGATATGCGCATTG |
| | GCTGGAGGGTTCGCCAAGGCAGATATAGAGATGGCTGGGCCCA |
| | TGGCCGCGGTCGGTCTGCTAATTGTCAGTTACGTGGTCTCAGGA |
| | AAGAGTGTGGACATGTACATTGAAAGAGCTGGTGACATCACAT |
| | GGGAAAAAGATGCGGAAGTCACTGGAAACAGTCCCCGGCTCGA |
| | TGTGGCGCTAGATGAGAGTGGTGATTTCTCCCTGGTGGAGGATG |
| | ACGGTCCCCCCATGAGAGAGATCATACTCAAGGTGGTCCTGAT |
| | GACCATCTGTGGCATGAACCCAATAGCCATACCCTTTGCAGCTG |
| | GAGCGTGGTACGTATACGTGAAGACTGGAAAAAGGAGTGGTGC |
| | TCTATGGGATGTGCCTGCTCCCAAGGAAGTAAAAAAGGGGGAG |
| | ACCACAGATGGAGTGTACAGAGTAATGACTCGTAGACTGCTAG |
| | GTTCAACACAAGTTGGAGTGGGAGTTATGCAAGAGGGGGTCTT |
| | TCACACTATGTGGCACGTCACAAAAGGATCCGCGCTGAGAAGC |
| | GGTGAAGGGAGACTTGATCCATACTGGGGAGATGTCAAGCAGG |
| | ATCTGGTGTCATACTGTGGTCCATGGAAGCTAGATGCCGCCTGG |
| | GACGGGCACAGCGAGGTGCAGCTCTTGGCCGTGCCCCCCGGAG |
| | AGAGAGCGAGGAACATCCAGACTCTGCCCGGAATATTTAAGAC |
| | AAAGGATGGGGACATTGGAGCGGTTGCGCTGGATTACCCAGCA |
| | GGAACTTCAGGATCTCCAATCCTAGACAAGTGTGGGAGAGTGA |
| | TAGGACTTTATGGCAATGGGGTCGTGATCAAAAATGGGAGTTAT |
| | GTTAGTGCCATCACCCAAGGGAGGAGGGAGGAAGAGACTCCTG |
| | TTGAGTGCTTCGAGCCTTCGATGCTGAAGAAGAAGCAGCTAACT |
| | GTCTTAGACTTGCATCCTGGAGCTGGGAAAACCAGGAGAGTTCT |
| | TCCTGAAATAGTCCGTGAAGCCATAAAAACAAGACTCCGTACT |
| | GTGATCTTAGCTCCAACCAGGGTTGTCGCTGCTGAAATGGAGGA |
| | AGCCCTTAGAGGGCTTCCAGTGCGTTATATGACAACAGCAGTCA |
| | ATGTCACCCACTCTGGAACAGAAATCGTCGACTTAATGTGCCAT |
| | GCCACCTTCACTTCACGTCTACTACAGCCAATCAGAGTCCCCAA |
| | CTATAATCTGTATATTATGGATGAGGCCCACTTCACAGATCCCT |
| | CAAGTATAGCAGCAAGAGGATACATTTCAACAAGGGTTGAGAT |
| | GGGCGAGGCGGCTGCCATCTTCATGACCGCCACGCCACCAGGA |
| | ACCCGTGACGCATTTCCGGACTCCAACTCACCAATTATGGACAC |
| | CGAAGTGGAAGTCCCAGAGAGAGCCTGGAGCTCAGGCTTTGAT |
| | TGGGTGACGGATTATTCTGGAAAAACAGTTTGGTTTGTTCCAAG |
| | CGTGAGGAACGGCAATGAGATCGCAGCTTGTCTGACAAAGGCT |
| | GGAAAACGGGTCATACAGCTCAGCAGAAAGACTTTTGAGACAG |
| | AGTTCCAGAAAACAAAACATCAAGAGTGGGACTTTGTCGTGAC |
| | AACTGACATTTCAGAGATGGGCGCCAACTTTAAAGCTGACCGT |
| | GTCATAGATTCCAGGAGATGCCTAAAGCCGGTCATACTTGATGG |
| | CGAGAGAGTCATTCTGGCTGGACCCATGCCTGTCACACATGCCA |
| | GCGCTGCCCAGAGGAGGGGGCGCATAGGCAGGAATCCCAACAA |
| | ACCTGGAGATGAGTATCTGTATGGAGGTGGGTGCGCAGAGACT |
| | GACGAAGACCATGCACACTGGCTTGAAGCAAGAATGCTCCTTG |
| | ACAATATTTACCTCCAAGATGGCCTCATAGCCTCGCTCTATCGA |
| | CCTGAGGCCGACAAAGTAGCAGCCATTGAGGGAGAGTTCAAGC |
| | TTAGGACGGAGCAAAGGAAGACCTTTGTGGAACTCATGAAAAG |
| | AGGAGATCTTCCTGTTTGGCTGGCCTATCAGGTTGCATCTGCCG |
| | GAATAACCTACACAGATAGAAGATGGTGCTTTGATGGCACGAC |
| | CAACAACACCATAATGGAAGACAGTGTGCCGGCAGAGGTGTGG |
| | ACCAGACACGGAGAGAAAAGAGTGCTCAAACCGAGGTGGATG |
| | GACGCCAGAGTTTGTTCAGATCATGCGGCCCTGAAGTCATTCAA |
| | GGAGTTTGCCGCTGGGAAAAGAGGAGCGGCTTTTGGAGTGATG |
| | GAAGCCCTGGGAACACTGCCAGGACACATGACAGAGAGATTCC |
| | AGGAAGCCATTGACAACCTCGCTGTGCTCATGCGGGCAGAGAC |
| | TGGAAGCAGGCCTTACAAAGCCGCGGCGGCCCAATTGCCGGAG |
| | ACCCTAGAGACCATTATGCTTTTGGGGTTGCTGGGAACAGTCTC |
| | GCTGGGAATCTTTTTCGTCTTGATGAGGAACAAGGGCATAGGGA |
| | AGATGGGCTTTGGAATGGTGACTCTTGGGGCCAGCGCATGGCTC |
| | ATGTGGCTCTCGGAAATTGAGCCAGCCAGAATTGCATGTGTCCT |
| | CATTGTTGTGTTCCTATTGCTGGTGGTGCTCATACCTGAGCCAG |
| | AAAAGCAAAGATCTCCCCAGGACAACCAAATGGCAATCATCAT |
| | CATGGTAGCAGTAGGTCTTCTGGGCTTGATTACCGCCAATGAAC |
| | TCGGATGGTTGGAGAGAACAAAGAGTGACCTAAGCCATCTAAT |
| | GGGAAGGAGAGAGGAGGGGGCAACCATGGGATTCTCAATGGA |
| | CATTGACCTGCGGCCAGCCTCAGCTTGGGCCATCTATGCTGCCT |
| | TGACAACTTTCATTACCCCAGCCGTCCAACATGCAGTGACCACT |
| | TCATACAACAACTACTCCTTAATGGCGATGGCCACGCAAGCTGG |
| | AGTGTTGTTTGGTATGGGCAAAGGGATGCCATTCTACGCATGGG |
| | ACTTTGGAGTCCCGCTGCTAATGATAGGTTGCTACTCACAATTA |
| | ACGCCCCTGACCCTAATAGTGGCCATCATTTTGCTCGTGGCGCA |
| | CTACATGTACTTGATCCCAGGGCTGCAGGCAGCAGCTGCGCGTG |
| | CTGCCCAGAAGAGAACGGCAGCTGGCATCATGAAGAACCCTGT |
| | TGTGGATGGAATAGTGGTGACTGACATTGACACAATGACAATT |
| | GACCCCCAAGTGGAGAAAAAGATGGGACAGGTGCTACTCATGG |
| | CAGTAGCCGTCTCCAGCGCCATACTGTCGCGGACCGCCTGGGG |
| | GTGGGGGGAGGCTGGGGCCCTGATCACAGCCGCAACTTCCACT |
| | TTGTGGGAAGGCTCTCCGAACAAGTACTGGAACTCCTCTACAGC |
| | CACTTCACTGTGTAACATTTTTAGGGGAAGTTACTTGGCTGGAG |
| | CTTCTCTAATCTACACAGTAACAAGAAACGCTGGCTTGGTCAAG |
| | AGACGTGGGGGTGGAACAGGAGAGACCCTGGGAGAGAAATGG |
| | AAGGCCCGCTTGAACCAGATGTCGGCCCTGGAGTTCTACTCCTA |
| | CAAAAAGTCAGGCATCACCGAGGTGTGCAGAGAAGAGGCCCGC |
| | CGCGCCCTCAAGGACGGTGTGGCAACGGGAGGCCATGCTGTGT |
| | CCCGAGGAAGTGCAAAGCTGAGATGGTTGGTGGAGCGGGGATA |
| | CCTGCAGCCCTATGGAAAGGTCATTGATCTTGGATGTGGCAGAG |
| | GGGGCTGGAGTTACTACGCCGCCACCATCCGCAAAGTTCAAGA |
| | AGTGAAAGGATACACAAAAGGAGGCCCTGGTCATGAAGAACCC |
| | GTGTTGGTGCAAAGCTATGGGTGGAACATAGTCCGTCTTAAGAG |
| | TGGGGTGGACGTCTTTCATATGGCGGCTGAGCCGTGTGACACGT |
| | TGCTGTGTGACATAGGTGAGTCATCATCTAGTCCTGAAGTGGAA |
| | GAAGCACGGACGCTCAGAGTCCTCTCCATGGTGGGGGATTGGC |
| | TTGAAAAAAGACCAGGAGCCTTTTGTATAAAAGTGTTGTGCCCA |
| | TACACCAGCACTATGATGGAAACCCTGGAGCGACTGCAGCGTA |
| | GGTATGGGGGAGGACTGGTCAGAGTGCCACTCTCCCGCAACTC |
| | TACACATGAGATGTACTGGGTCTCTGGAGCGAAAAGCAACACC |
| | ATAAAAAGTGTGTCCACCACGAGCCAGCTCCTCTTGGGGCGCAT |
| | GGACGGGCCCAGGAGGCCAGTGAAATATGAGGAGGATGTGAAT |
| | CTCGGCTCTGGCACGCGGGCTGTGGTAAGCTGCGCTGAAGCTCC |
| | CAACATGAAGATCATTGGTAACCGCATTGAAAGGATCCGCAGT |
| | GAGCACGCGGAAACGTGGTTCTTTGACGAGAACCACCCATATA |
| | GGACATGGGCTTACCATGGAAGCTATGAGGCCCCCACACAAGG |
| | GTCAGCGTCCTCTCTAATAAACGGGGTTGTCAGGCTCCTGTCAA |
| | AACCCTGGGATGTGGTGACTGGAGTCACAGGAATAGCCATGAC |
| | CGACACCACACCGTATGGTCAGCAAAGAGTTTTCAAGGAAAAA |
| | GTGGACACTAGGGTGCCAGACCCCCAAGAAGGTACTCGTCAGG |
| | TTATGAGCATGGTCTCTTCCTGGTTGTGGAAAGAGCTAGGCAAA |
| | CACAAACGGCCACGAGTCTGTACCAAAGAAGAGTTCATCAACA |
| | AGGTTCGTAGCAATGCAGCATTAGGGGCAATATTTGAAGAGGA |
| | AAAAGAGTGGAAGACTGCAGTGGAAGCTGTGAACGATCCAAGG |
| | TTCTGGGCTCTAGTGGACAAGGAAAGAGAGCACCACCTGAGAG |
| | GAGAGTGCCAGAGTTGTGTGTACAACATGATGGGAAAAAGAGA |
| | AAAGAAACAAGGGGAATTTGGAAAGGCCAAGGGCAGCCGCGC |
| | CATCTGGTATATGTGGCTAGGGGCTAGATTTCTAGAGTTCGAAG |
| | CCCTTGGATTCTTGAACGAGGATCACTGGATGGGGAGAGAGAA |
| | CTCAGGAGGTGGTGTTGAAGGGCTGGGATTACAAAGACTCGGA |
| | TATGTCCTAGAAGAGATGAGTCGCATACCAGGAGGAAGGATGT |
| | ATGCAGATGACACTGCTGGCTGGGACACCCGCATCAGCAGGTT |
| | TGATCTGGAGAATGAAGCTCTAATCACCAACCAAATGGAGAAA |
| | GGGCACAGGGCCTTGGCATTGGCCATAATCAAGTACACATACC |
| | AAAACAAAGTGGTAAAGGTCCTTAGACCAGCTGAAAAAGGGAA |
| | AACAGTTATGGACATTATTTCGAGACAAGACCAAAGGGGGAGC |
| | GGACAAGTTGTCACTTACGCTCTTAACACATTTACCAACCTAGT |
| | GGTGCAACTCATTCGGAATATGGAGGCTGAGGAAGTCCTAGAG |
| | ATGCAAGACTTGTGGCTGCTGCGGAGGTCAGAGAAAGTGACCA |
| | ACTGGTTGCAGAGCAACGGATGGGATAGGCTCAAACGAATGGC |
| | AGTCAGTGGAGATGATTGCGTTGTGAAGCCAATTGATGATAGGT |
| | TTGCACATGCCCTCAGGTTCTTGAATGATATGGGAAAAGTTAGG |
| | AAGGACACACAAGAGTGGAAACCCTCAACTGGATGGGACAACT |
| | GGGAAGAAGTTCCGTTTTGCTCCCACCACTTCAACAAGCTCCAT |
| | CTCAAGGACGGGAGGTCCATTGTGGTTCCCTGCCGCCACCAAG |
| | ATGAACTGATTGGCCGGGCCCGCGTCTCTCCAGGGGGGGGATG |
| | GAGCATCCGGGAGACTGCTTGCCTAGCAAAATCATATGCGCAA |
| | ATGTGGCAGCTCCTTTATTTCCACAGAAGGGACCTCCGACTGAT |
| | GGCCAATGCCATTTGTTCATCTGTGCCAGTTGACTGGGTTCCAA |
| | CTGGGAGAACTACCTGGTCAATCCATGGAAAGGGAGAATGGAT |
| | GACCACTGAAGACATGCTTGTGGTGTGGAACAGAGTGTGGATT |
| | GAGGAGAACGACCACATGGAAGACAAGACCCCAGTTACGAAAT |
| | GGACAGACATTCCCTATTTGGGAAAAAGGGAAGACTTGTGGTG |
| | TGGATCTCTCATAGGGCACAGACCGCGCACCACCTGGGCTGAG |
| | AACATTAAAAACACAGTCAACATGGTGCGCAGGATCATAGGTG |
| | ATGAAGAAAAGTACATGGACTACCTATCCACCCAAGTTCGCTA |
| | CTTGGGTGAAGAAGGGTCTACACCTGGAGTGCTGTAA |

The preceding merely illustrates the principles of this disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of this disclosure and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof.

Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of the present disclosure is embodied by the appended claims.

What is claimed is:

1. A nucleic acid comprising a zika virus 5′ untranslated region (5′ UTR), a sequence complementary to a regulatory polynucleotide, and a zika virus 3′ untranslated region (3′ UTR), wherein the regulatory polynucleotide comprises (i) miR-219a-2-3p, hsa-miR-377-3p, hsa-miR-1225-5p, hsa-miR-4298, hsa-miR-219a-5p, or hsa-miR-129-5p, (ii) a sequence at least 80% identical to a sequence of Table 6A or Table 6B, or (iii) (i) and (ii).

2. The nucleic acid of claim 1, wherein the sequence complementary to the regulatory polynucleotide comprises CCGACCTGT, AAAAACGCC, AAACGCCAG, GGCGTTTT, CTAACAGGT, TAACAGGTT, ACTAACAGG, ACCCTGTCC, CACCCATGC, CCCATGCCG, ACCCATGCC, AGTGTGTTT, CTTAACACC, TTAACACCG, CTTAACAGC, or TGCCGGTCA, or a combination of two or more thereof.

3. The nucleic acid of claim 1 or claim 2, wherein the 5′ UTR is at least 80% homologous or identical to SEQ ID NO: 239
(AGTTGTTACTGTTGCTGACTCAGACTGCGACAGTTCGAGTTTGAAGCG
AAAGCTAGCAACAGTATCAACAGGTTTTATTTGGATTTGGAAACGAGAG
TTTCTGGTC).

4. The nucleic acid of any one of claims 1-3, wherein the 3′ UTR is at least 80% homologous or identical to SEQ ID NO: 241
(TAAGCACCAATCTTAATGTTGTCAGGCCTGCTAGTCAGCCACAGCTTG
GGGAAAGCTGTGCAGCCTGTGACCCCCCCAGGAGAAGCTGGGAAACCAA
GCCTATAGTCAGGCCGAGAACGCCATGGCACGGAAGAAGCCATGCTGCC
TGTGAGCCCCTCAGAGGACACTGAGTCAAAAAACCCCACGCGCTTGGAG
GCGCAGGATGGGAAAAGAAGGTGGCGACCTTCCCCACCCTTCAATCTGG
GGCCTGAACTGGAGATCAGCTGTGGATCTCCAGAAGAGGGACTAGTGGT
TAGAGGAGACCCCCCGGAAAACGCAAAACAGCATATTGACGCTGGGAAA
GACCAGAGACTCCATGAGTTTCCACCACGCTGGCCGCCAGGCACAGATC
GCCGAATAGCGGCGGCCGGTGTGGGGAAATCCATGGGTCT).

5. The nucleic acid of claim 1 or claim 2, wherein the 5′ UTR is selected from a zika virus of Table 12, and the 3′ UTR is selected from a zika virus of Table 13.

6. The nucleic acid of any one of claims 1-5, wherein the regulatory polynucleotide is expressed in non-cancer cells, optionally wherein the regulatory polynucleotide has higher expression in non-cancer cells as compared to cancer cells of a subject.

7. The nucleic acid of any one of claims 1-6, wherein the regulatory polynucleotide is a microRNA.

8. The nucleic acid of any one of claims 1-7 wherein the sequence complementary to the regulatory polynucleotide is between 9 nucleotides and 22 nucleotides in length, optionally about 9, 10, 11, 12, 13, 14, 15, 16, 17, 18, 19, 20, 21, or 22 nucleotides in length.

9. A nucleic acid comprising:
(a) a zika virus 5′ untranslated region (5′ UTR);
(b) a polynucleotide encoding at least one zika virus structural protein selected from capsid protein (ancC/C), membrane protein (prM/M), and envelope protein (E), and/or a polynucleotide encoding at least one zika virus non-structural protein selected from NS1, NS2A, NS2B, NS3, NS4A, 2k peptide, NS4B, and NS5;
(c) a zika virus 3′ untranslated region (3′UTR); and
(d) a sequence complementary to a regulatory polynucleotide;
wherein:
(i) the nucleic acid comprises the polynucleotide encoding at least one zika virus non-structural protein, and the polynucleotide encoding the at least one zika virus non-structural protein comprises a polynucleotide encoding the 2k peptide, wherein the sequence complementary to the regulatory polynucleotide is positioned within the polynucleotide encoding the 2k peptide;
(ii) the nucleic acid comprises the polynucleotide encoding at least one zika virus non-structural protein, and the polynucleotide encoding the at least one zika virus non-structural protein comprises a polynucleotide encoding the NS4A and a polynucleotide encoding the NS4B, and the sequence complementary to the regulatory polynucleotide is positioned between the polynucleotide encoding the NS4A and the polynucleotide encoding the NS4B;
(iii) the sequence complementary to the regulatory polynucleotide is positioned between the 5′ UTR and the polynucleotide encoding at least one zika virus structural protein and/or the polynucleotide encoding at least one zika virus non-structural protein; and/or
(iv) the sequence complementary to the regulatory polynucleotide is positioned between the 3′ UTR and the polynucleotide encoding at least one zika virus structural protein and/or the polynucleotide encoding at least one zika virus non-structural protein.

10. The nucleic acid of claim 9, wherein the nucleic acid comprises the polynucleotide encoding at least one zika virus non-structural protein, and the polynucleotide encoding the at least one zika virus non-structural protein comprises a polynucleotide encoding the 2k peptide, wherein the sequence complementary to the regulatory polynucleotide is positioned within the polynucleotide encoding the 2k peptide.

11. The nucleic acid of claim 9, wherein the nucleic acid comprises the polynucleotide encoding at least one zika virus non-structural protein, and the polynucleotide encoding the at least one zika virus non-structural protein comprises a polynucleotide encoding the NS4A and a polynucleotide encoding the NS4B, and the sequence complementary to the regulatory polynucleotide is positioned between the polynucleotide encoding the NS4A and the polynucleotide encoding the NS4B.

12. The nucleic acid of claim 9, wherein the sequence complementary to the regulatory polynucleotide is positioned between the 5′ UTR and the polynucleotide encoding at least one zika virus structural protein and/or the polynucleotide encoding at least one zika virus non-structural protein.

13. The nucleic acid of claim 9, wherein the sequence complementary to the regulatory polynucleotide is positioned between the 3′ UTR and the polynucleotide encoding at least one zika virus structural protein and/or the polynucleotide encoding at least one zika virus non-structural protein.

14. The nucleic acid of any one of claims 9-13, wherein the 5′ UTR is at least 80% homologous or identical to SEQ ID NO: 239.

15. The nucleic acid of any one of claims 9-14, wherein the 3′ UTR is at least 80% homologous or identical to SEQ ID NO: 241.

16. The nucleic acid of any one of claims 9-15, wherein the 5′ UTR is selected from a zika virus of Table 12, and the 3′ UTR is selected from a zika virus of Table 13.

17. The nucleic acid of any one of claims 9-16, wherein the nucleic acid comprises the polynucleotide encoding at least one zika virus structural protein, and the nucleic acid comprising the polynucleotide encoding at least one zika virus structural protein comprises: a polynucleotide encoding the capsid protein (ancC/C), a polynucleotide encoding the membrane protein (prM/M), and a polynucleotide encoding the envelope protein (E).

18. The nucleic acid of any one of claims 9-17, wherein the nucleic acid comprises the polynucleotide encoding at least one zika virus non-structural protein, and the polynucleotide encoding at least one zika virus non-structural protein comprises: a polynucleotide encoding the NS1, a polynucleotide encoding the NS2A, a polynucleotide encoding the NS2B, a polynucleotide encoding the NS3, a polynucleotide encoding the NS4A, a polynucleotide encoding the 2k peptide, a polynucleotide encoding the NS4B, and a polynucleotide encoding the NS5.

19. The nucleic acid of any one of claims 9-18, wherein the regulatory polynucleotide is expressed in non-cancer cells, optionally wherein the regulatory polynucleotide has higher expression in non-cancer cells as compared to cancer cells of a subject.

20. The nucleic acid of any one of claims 9-19, wherein the regulatory polynucleotide is a microRNA.

21. The nucleic acid of any one of claims 9-20, wherein the sequence complementary to the regulatory polynucleotide is between 9 nucleotides and 22 nucleotides in length, optionally about 9, 10, 11, 12, 13, 14, 15, 16, 17, 18, 19, 20, 21, or 22 nucleotides in length.

22. A recombinant zika virus produced from the nucleic acid of any one of claims 1-21.

23. The recombinant zika virus of claim 22, wherein the recombinant zika virus is an oncolytic zika virus having oncolytic activity.

24. A recombinant zika virus comprising a polynucleotide complementary to a regulatory polynucleotide, the regulatory polynucleotide comprising (i) miR-219a-2-3p, hsa-miR-377-3p, hsa-miR-1225-5p, hsa-miR-4298, hsa-miR-219a-5p, or hsa-miR-129-5p, (ii) a sequence at least 80% identical to a sequence of Table 6A or Table 6B, or (iii) (i) and (ii).

25. The recombinant zika virus of claim 24, wherein the sequence complementary to the regulatory polynucleotide comprises CCGACCTGT, AAAAACGCC, AAACGCCAG, GGCGTTTT, CTAACAGGT, TAACAGGTT, ACTAACAGG, ACCCTGTCC, CACCCATGC, CCCATGCCG, ACCCATGCC, AGTGTGTTT, CTTAACACC, TTAACACCG, CTTAACAGC, or TGCCGGTCA, or a combination of two or more thereof.

26. The recombinant zika virus of claim 24 or claim 25, wherein the recombinant zika virus is an oncolytic zika virus having oncolytic activity.

27. A method of treating cancer in a subject in need thereof, the method comprising administering to the subject the recombinant zika virus of any one of claims 22-26.

28. The method of claim 27, wherein the cancer is a central nervous system (CNS) cancer.

29. The method of claim 28, wherein the CNS cancer is a brain cancer.

30. The method of claim 29, wherein the brain cancer is an astrocytoma, an oligodendroglioma, an ependymoma, a meningioma, a schwannoma, a craniopharyngioma, a germinoma, or a pineocytoma.

31. The method of claim 27, wherein the cancer is breast cancer, prostate cancer, colon cancer, retinal cancer, or testicular cancer.

32. The method of any one of claims 27-31, wherein the recombinant zika virus does not infect and/or does not replicate in a non-cancer cell of the subject; or wherein the recombinant zika virus infects and/or replicates less in a non-cancer cell of the subject as compared to a cancer cell of the subject.
